(19) United States
(12) Patent Application Publication
(10) Pub. No.: US 2024/0279201 A1
(43) Pub. Date: Aug. 22, 2024

(54) DUAL WNT SIGNALING PATHWAY INHIBITORS AND AMPK ACTIVATORS FOR TREATMENTS OF DISEASE

(71) Applicants: Fengtian Xue, Potomac, MD (US); Yan Shu, Clarksville, MD (US); Yong Ai, Halethorpe, MD (US)

(72) Inventors: Fengtian Xue, Potomac, MD (US); Yan Shu, Clarksville, MD (US); Yong Ai, Halethorpe, MD (US)

(21) Appl. No.: 18/565,426

(22) PCT Filed: Jun. 1, 2022
(86) PCT No.: PCT/US2022/031798

§ 371 Date: Nov. 29, 2023

Related U.S. Application Data
(60) Provisional application No. 63/195,668, filed on Jun. 1, 2021.

(51) Int. Cl. C07D 401/12; A61K 31/423; A61K 31/427; A61K 31/428; A61K 31/4375; A61K 31/4439; A61K 31/4709; A61K 31/496; A61K 31/498; A61K 31/517; A61K 31/69; A61K 45/06; A61K 47/55; A61K 47/60; A61P 1/16; A61P 35/00; C07D 401/14; C07D 403/12; C07D 413/12; C07D 417/12; C07D 417/14; C07D 471/04; C07F 5/02

(52) U.S. Cl. CPC C07D 401/12; A61K 31/423; A61K 31/427; A61K 31/428; A61K 31/4375; A61K 31/4439; A61K 31/4709; A61K 31/496; A61K 31/498; A61K 31/517; A61K 31/69; A61K 45/06; A61K 47/551; A61K 47/60; A61P 1/16; A61P 35/00; C07D 401/14; C07D 403/12; C07D 413/12; C07D 417/12; C07D 417/14; C07D 471/04; C07F 5/025

(57) ABSTRACT

Compounds and compositions are provided as inhibitors of the Wnt/beta-catenin pathway and/or activators of the adenosine monophosphate-activated kinase (AMPK) pathway for the treatment of diseases that implicate the same. Such diseases include cancer or a metabolic disease. Cancers that may be treated by these compounds and compositions include adrenocortical cancer, hepatocellular cancer, hepatoblastoma, malignant melanoma, ovarian cancer, Wilm's tumor, Barrett's esophageal cancer, prostate cancer, colon cancer, colorectal cancer, rectal cancer, pancreatic cancer, bladder cancer, breast cancer (e.g. triple negative breast cancer), gastric cancer, head & neck cancer, lung cancer, mesothelioma, cervical cancer, uterine cancer, myeloid leukemia cancer, lymphoid leukemia cancer, pilometricoma cancer, medulloblastoma cancer, glioblastoma, and familial adenomatous polyposis. Metabolic diseases include type 2 diabetes, obesity, hyperlipidemia, alcoholic or non-alcoholic fatty liver disease, and liver fibrosis.

### CROSS-REFERENCE TO RELATED APPLICATIONS

This Application claims the benefit of priority to U.S. Provisional Application No. 63/195,668, filed Jun. 1, 2021, the entirety of which is incorporated herein by reference.

### FIELD

The present disclosure relates generally to compounds that dually inhibit the Wnt signaling pathway and activate the AMPK pathway and more particularly, but not exclusively, to compounds that inhibit the Wnt/β-catenin pathway and activate the AMPK pathway for the treatment of diseases that implicate the Wnt/β-catenin pathway and/or the AMPK pathway.

### BACKGROUND

A number of individuals are affected each year by diseases that implicate aberrant activity in Wnt signalling, which may result in abnormal levels of β-catenin. These diseases include metabolic diseases and cancer, for example. Among the known crossing pathways, the adenosine monophosphate-activated kinase (AMPK) pathway plays a role in maintaining energy homeostasis at the cellular and whole-body levels.

There is a need in the field for new and potent therapeutics that inhibit the Wnt/β-catenin pathway and/or activate the AMPK pathway as treatments for disease. The present disclosure meets those needs.

### SUMMARY

The present disclosure meets the needs in the field by providing compounds and methods for the treatment of diseases that implicate the Wnt/β-catenin signaling pathway and/or the AMPK pathway.

In one aspect the compounds of the disclosure may include a compound of formula (I):

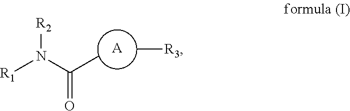

wherein ring A is a 5-membered substituted or unsubstituted heterocycle or heteroaryl;
wherein R₁ is selected from substituted or unsubstituted cycloalkyl, substituted or unsubstituted aryl, substituted or unsubstituted heterocycle, and substituted or unsubstituted heteroaryl;
R₂ is selected from H and substituted or unsubstituted alkyl; and
R₃ is selected from substituted or unsubstituted aryl and substituted or unsubstituted heteroaryl; or a pharmaceutically acceptable salt thereof.

In some embodiments, ring A is selected from substituted or unsubstituted triazole, substituted or unsubstituted tetrazole, substituted or unsubstituted pyrazole, substituted or unsubstituted pyrrole, and substituted or unsubstituted thiazole.

In some embodiments, the compound of formula (I) is a compound of any one of formula (10)-(15), or a pharmaceutically acceptable salt thereof.

In some embodiments, R₁ is selected from substituted or unsubstituted quinoline, substituted or unsubstituted quinoxaline, substituted or unsubstituted benzothiazole, substituted or unsubstituted isoquinoline, substituted or unsubstituted pyridine, substituted or unsubstituted quinazoline, substituted or unsubstituted 1,5-naphthyridine, substituted or unsubstituted 1,8-naphthyridine, substituted or unsubstituted thiazole, and substituted or unsubstituted benzoxazole.

In some embodiments, R₁ is selected from

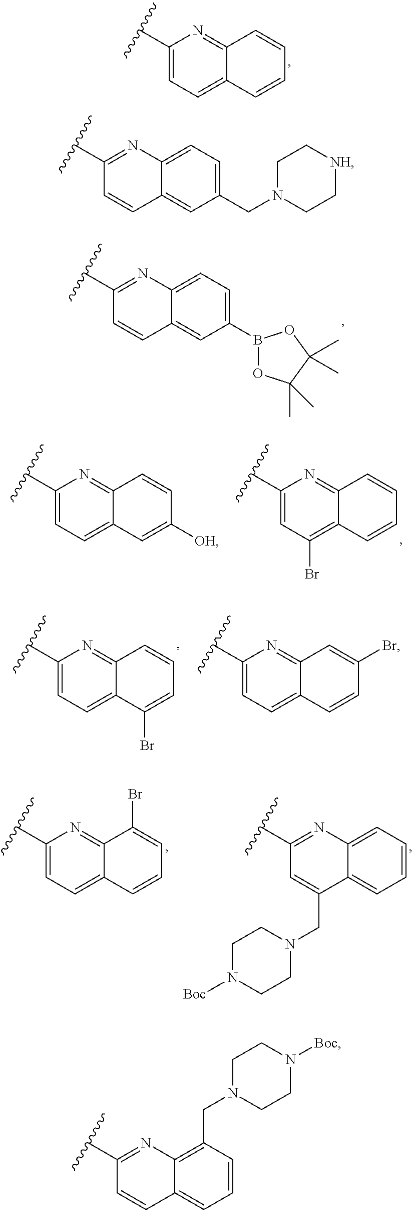

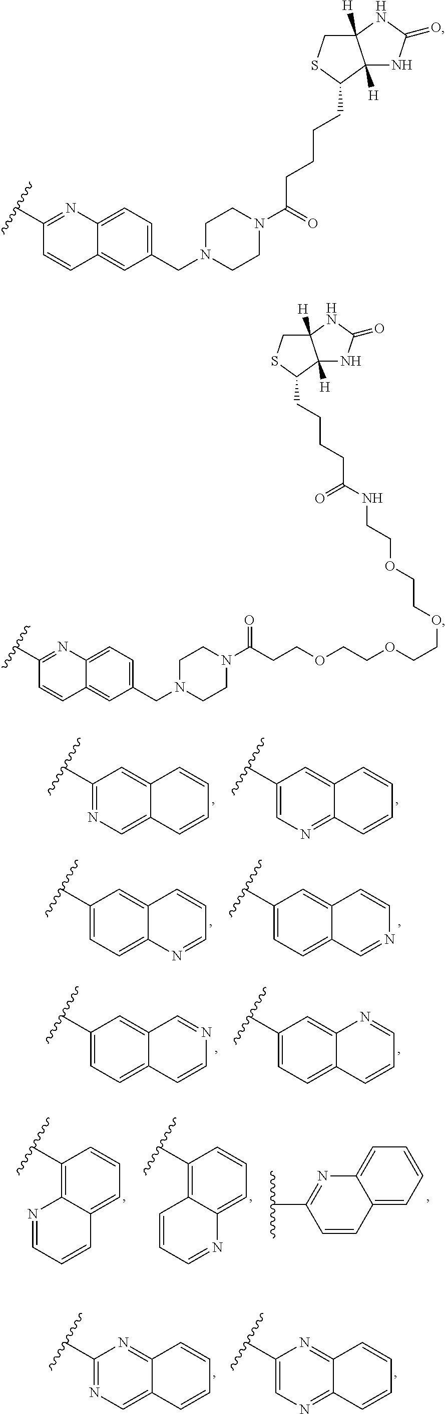

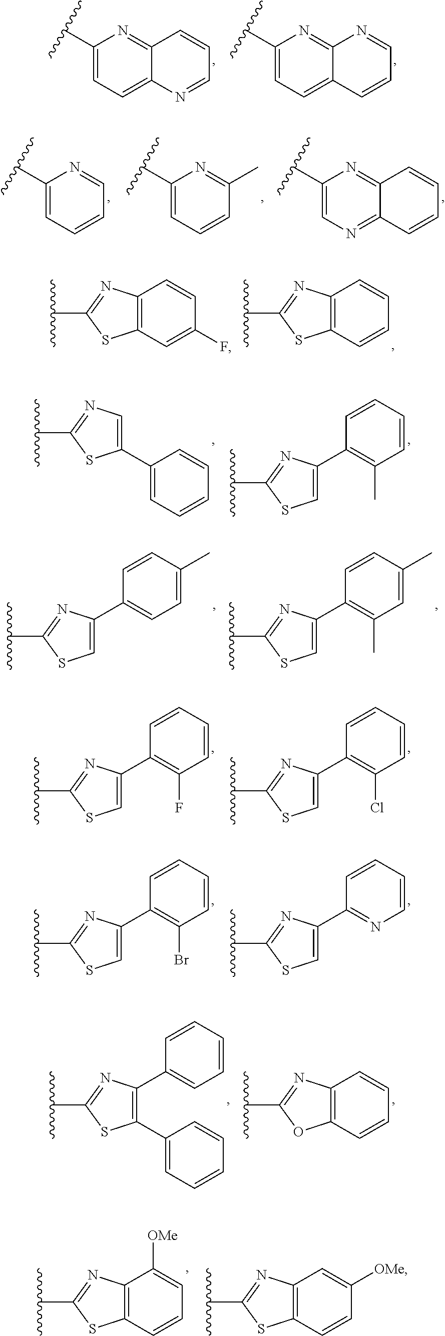

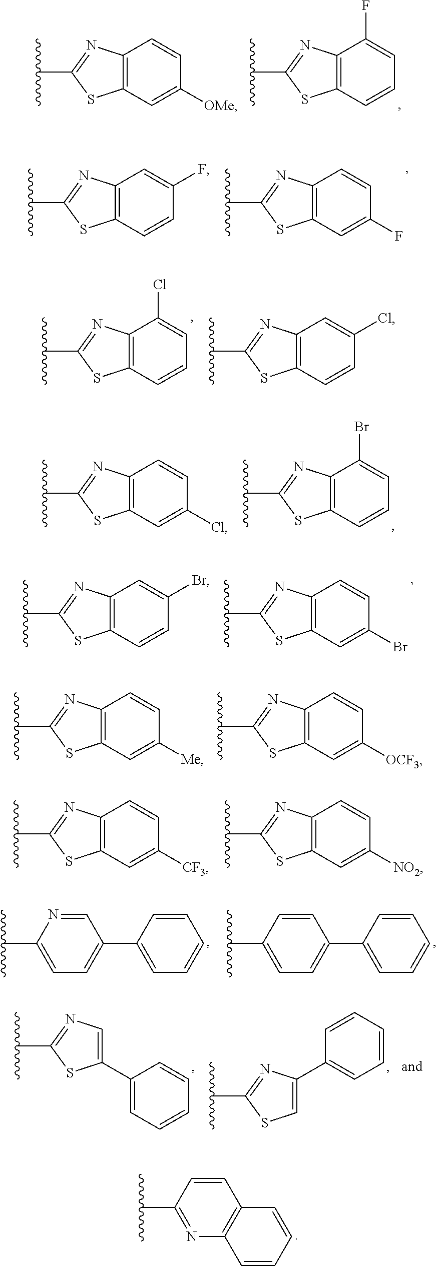

In some embodiments, R₃ is selected from substituted or unsubstituted pyridyl, unsubstituted aryl, and aryl substituted with one or more groups selected from halo, —CN, alkyl, fluroroalkyl, alkoxy, and fluoroalkoxy.

In some embodiments, R₃ is selected from

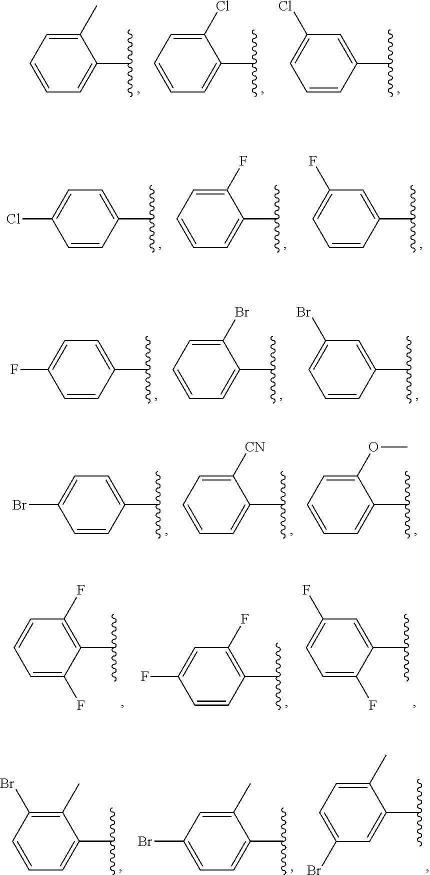

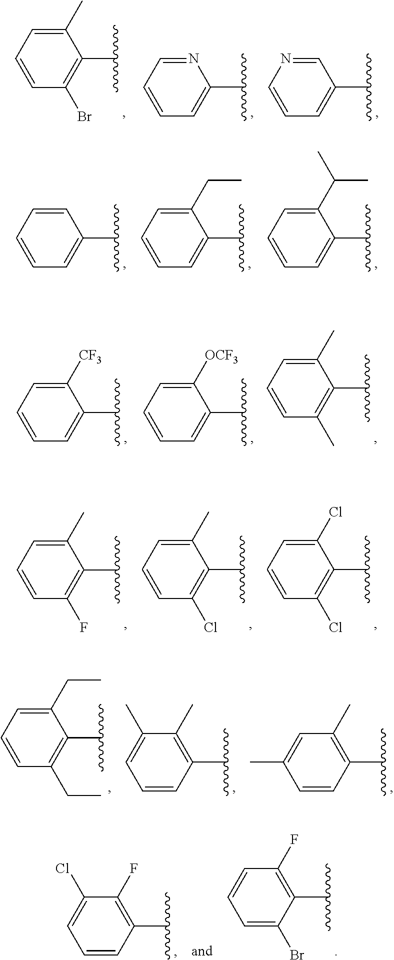

In some embodiments, each R₄, R₅, and R₆ is independently selected from H, methyl, ethyl, i-propyl, t-butyl, and —CF₃.

In some embodiments, the compound of formula (I) is a compound of any one of formula (20)-(29), or a pharmaceutically acceptable salt thereof.

In some embodiments, R₁ is selected from

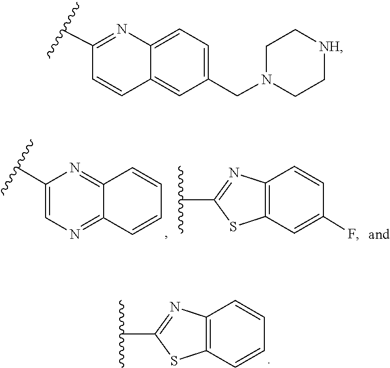

In some embodiments, R₂ is H.

In some embodiments, R₃ is selected from

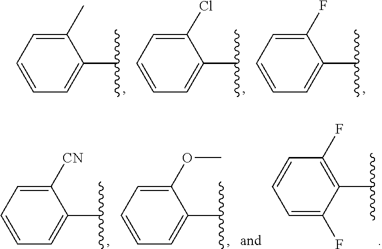

In some embodiments, R₄ is selected from methyl and ethyl.

In some embodiments, R₅ is

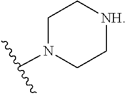 [SMILES: *N1CCNCC1]

In some embodiments, the compound is a compound of any one of formula 1001-1126, or a pharmaceutically acceptable salt thereof. In some embodiments, the compound is a compound of any one of formula 1001, 1002, 1024, 1032-1036, 1078, 1096, 1100, 1101, 1114, 1121, 1122, or 1124, or a pharmaceutically acceptable salt thereof. In some embodiments, the compound is a compound of formula 1001.

In some embodiments, the pharmaceutically acceptable salt is selected from valproic acid, maleic acid, tartaric acid, oxalic acid, pamoic acid, phosphonic acid, benzoic acid, citric acid, salicylic acid, succinic acid, methanesulfonic acid, malic acid, and p-toluenesulfonic acid.

In one aspect, the disclosure provides a compound of formula (II):

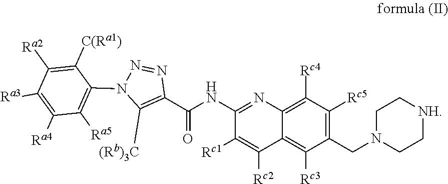

wherein in formula (II):
R^{a1}, R^{a2}, R^{a3}, R^{a4}, R^{a5}, R^{b}, R^{c1}, R^{c2}, R^{c3}, R^{c4}, and R^{c5} at each occurrence is independently selected from H, halo, alkyl, alkoxy, and aryl,
with the proviso that one or more R^{a1}, R^{a2}, R^{a3}, R^{a4}, R^{a5}, R^{b}, R^{c1}, R^{c2}, R^{c3}, R^{c4}, and R^{c5} is fluoro;
or a pharmaceutically acceptable salt thereof.

In some embodiments, one or more R^{a1} is fluoro. In some embodiments, each occurrence of R^{a1} is fluoro. In some embodiments, R^{a3} is fluoro. In some embodiments, one or more R^{b} is fluoro. In some embodiments, each occurrence of R^{b} is fluoro. In some embodiments, R^{c2} is fluoro. In some embodiments, R^{c5} is fluoro. In some embodiments, the compound comprises only one fluoro group. In some embodiments, the only one fluoro group is at R^{a2}, R^{a3}, R^{a4}, R^{a5}, R^{c1}, R^{c2}, R^{c3}, R^{c4}, or R^{5c}. In some embodiments, the remaining R^{a2}, R^{a3}, R^{a4}, R^{a5}, R^{c1}, R^{c2}, R^{c3}, R^{c4}, and R^{c5} are hydrogen, and each occurrence of Rai is hydrogen, and each occurrence of R^{b} is hydrogen. In some embodiments, the compound comprises exactly three fluoro groups, wherein the three fluoro groups are at each occurrence of Rai or at each occurrence of R^{b}. In some embodiments, the remaining R^{a1} or R^{b} are at each occurrence hydrogen, and R^{a2}, R^{a3}, R^{a4}, R^{a5}, R^{c1}, R^{c2}, R^{c3}, R^{c4}, or R^{c5} are each hydrogen.

In some embodiments, the compound of formula (II) is a compound of any one of formula 2001-2031, or a pharmaceutically acceptable salt thereof.

In one aspect, the disclosure includes a pharmaceutical composition comprising one or more of compounds of a compound of formula (I), formula (10)-(15), formula (20)-(29), formula 1001-1126, formula (II), formula (2001-2031), formula (III), formula 3001-3018, or a pharmaceutically acceptable salt thereof, and a physiologically compatible carrier medium.

In one aspect, the disclosure provides a compound of formula (III):

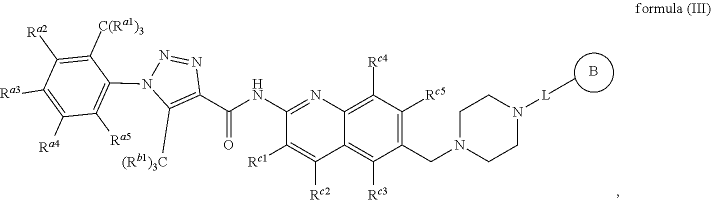

wherein in formula (III):
R^{a1}, R^{a2}, R^{a3}, R^{a4}, R^{a5}, R^{b1}, R^{c1}, R^{c2}, R^{c3}, R^{c4}, and R^{c5} at each occurrence is independently selected from hydrogen, halo, alkyl, alkoxy, and aryl;
L is a linking group; and
B is a targeting moiety,
or a pharmaceutically acceptable salt thereof.

In some embodiments, R^{a1}, R^{a2}, R^{a3}, R^{a4}, R^{a5}, R^{b}, R^{c1}, R^{c2}, R^{c3}, R^{c4}, and R^{c5} are at each occurrence hydrogen. In some embodiments, L comprises one or more linking groups selected from optionally substituted —C₁₋₁₀ alkyl-, —O—C₁₋₁₀ alkyl-, —C₁₋₁₀ alkenyl-, —O—C₁₋₁₀ alkenyl-, —C₁₋₁₀ cycloalkenyl-, —O—C₁₋₁₀ cycloalkenyl-, —C₁₋₁₀ alkynyl-, —O—C₁₋₁₀ alkynyl-, —C₁₋₁₀ aryl-, —O—C₁₋₁₀—, -aryl-, —O—, —S—, —S—S—, —S(O)_{w}—, —C(O)—, —C(O)O—, —OC(O)—, —C(O)S—, —SC(O)—, —OC(O)O—, —N(R^{b})—, —C(O)N(R^{b})—, —N(R^{b})C(O)—, —OC(O)N(R^{b})—, —N(R^{b})C(O)O—, —SC(O)N(R^{b})—, —N(R^{b})C(O)S—, —N(R^{b})C(O)N(R^{b})—, —N(R^{b})C(NR^{b})N(R^{b})—, —N(R^{b})S(O)_{w}—, —S(O)_{w}N(R^{b})—, —S(O)_{w}O—, —OS(O)_{w}—, —OS(O)_{w}O—, —O(O)P(OR^{b})O—, (O)P(O—)₃, —O(S)P(OR^{b})O—, and (S)P(O—)₃, wherein w is 1 or 2, and R^{b} is independently hydrogen, optionally substituted alkyl, or optionally substituted aryl. In some embodiments, L comprises one or more linking groups selected from —C₁₋₁₀ alkyl-N(R^{b})—, —O—C₁₋₁₀ alkyl-, —O—, —N(R^{b})—, and —S—S—. In some embodiments, L comprises one or more linking group selected from and

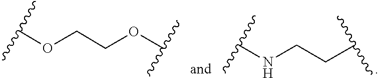 [SMILES: *OCCO* ; *CCN*]

In some embodiments, L comprises

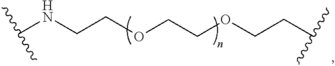

wherein n is an integer selected from 0-5. In some embodiments, L comprises

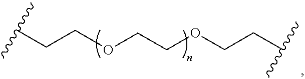

wherein n is an integer selected from 0-5. In some embodiments, the linker is a cleavable linker. In some embodiments, the linker is a non-cleavable linker. In some embodiments, the targeting moiety comprises one or more moieties selected from biotin, folic acid, and biguanide.

In some embodiments, the targeting moiety is selected from:

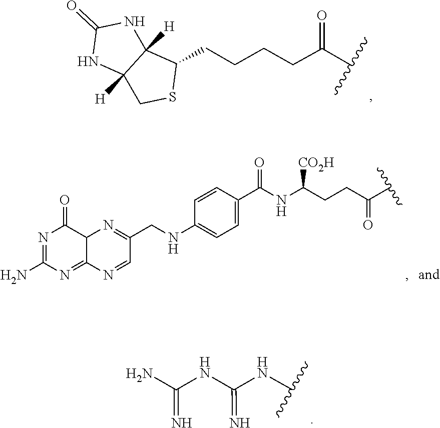 [SMILES: *C(=O)CCCC[C@@H]1SC[C@@H]2NC(=O)N[C@@H]21 ; *C(=O)CC[C@@H](NC(=O)c1ccc(NCC2=NC3C(=O)N=C(N)N=C3N=C2)cc1)C(=O)O ; *NC(=N)NC(=N)N]

In some embodiments, the compound of formula (III) is a compound of any one of formula 3001-3018, or a pharmaceutically acceptable salt thereof.

In one aspect, the disclosure includes a pharmaceutical composition for treating or preventing a disease alleviated by inhibiting Wnt/β-catenin signaling, the pharmaceutical composition comprising one or more compounds of formula (I), formula (10)-(15), formula (20)-(29), formula 1001-1126, formula (II), formula 2001-2031, formula (III), formula 3001-3018, or a pharmaceutically acceptable salt thereof, and a physiologically compatible carrier medium.

In one aspect, the disclosure includes a pharmaceutical composition for treating or preventing a disease alleviated by activating adenosine monophosphate-activated kinase (AMPK) signaling, the pharmaceutical composition comprising one or more compounds of formula (I), formula (10)-(15), formula (20)-(29), formula 1001-1126, formula (II), formula 2001-2031, formula (III), formula 3001-3018, or a pharmaceutically acceptable salt thereof, and a physiologically compatible carrier medium.

In one aspect, the disclosure includes a pharmaceutical composition for treating or preventing a disease alleviated by both inhibiting Wnt/β-catenin signaling and activating adenosine monophosphate-activated kinase (AMPK) signaling, the pharmaceutical composition comprising one or more compounds of formula (I), formula (10)-(15), formula (20)-(29), formula 1001-1126, formula (II), formula 2001-2031, formula (III), formula 3001-3018, or a pharmaceutically acceptable salt thereof, and a physiologically compatible carrier medium. In some embodiments, the disease or disorder is cancer or a metabolic disease. In some embodiments, the disease or disorder is selected from the group consisting of type 2 diabetes, obesity, hyperlipidemia, fatty liver disease, adrenocortical cancer, hepatocellular cancer, hepatoblastoma, malignant melanoma, ovarian cancer, Wilm's tumor, Barrett's esophageal cancer, prostate cancer, colon cancer, colorectal cancer, rectal cancer, pancreatic cancer, bladder cancer, breast cancer, gastric cancer, head & neck cancer, lung cancer, mesothelioma, cervical cancer, uterine cancer, myeloid leukemia cancer, lymphoid leukemia cancer, pilometricoma cancer, medulloblastoma cancer, glioblastoma, and familial adenomatous polyposis. In some embodiments, the breast cancer is triple negative breast cancer.

In one aspect, the disclosure includes a pharmaceutical composition for treating liver fibrosis, the pharmaceutical composition comprising one or more compounds of formula (I), formula (10)-(15), formula (20)-(29), formula 1001-1126, formula (II), formula 2001-2031, formula (III), formula 3001-3018, or a pharmaceutically acceptable salt thereof, and a physiologically compatible carrier medium.

In one aspect, the disclosure includes a pharmaceutical composition for treating colorectal cancer (CRC), the pharmaceutical composition comprising one or more compounds of formula (I), formula (10)-(15), formula (20)-(29), formula 1001-1126, formula (II), formula 2001-2031, formula (III), formula 3001-3018, or a pharmaceutically acceptable salt thereof, and a physiologically compatible carrier medium.

In one aspect, the disclosure includes a pharmaceutical composition for treating alcoholic fatty liver disease (ALD) or non-alcoholic fatty liver disease (NAFLD), the pharmaceutical composition comprising one or more compounds of formula (I), formula (10)-(15), formula (20)-(29), formula 1001-1126, formula (II), formula 2001-2031, formula (III), formula 3001-3018, or a pharmaceutically acceptable salt thereof, and a physiologically compatible carrier medium. In some embodiments, the non-alcoholic fatty liver disease is selected from the group consisting of simple fatty liver (steatosis), non-alcoholic steatohepatitis (NASH), and liver cirrhosis.

In some embodiments, the pharmaceutical composition includes one or more additional therapeutic agents. In some embodiments, the additional therapeutic agent is selected from the group consisting of a RAF inhibitor, an MEK inhibitor, an ERK inhibitor, a VEGFR inhibitor, EGFR inhibitor, and a combination thereof. In some embodiments, the VEGFR inhibitor is selected from the group consisting of Bevacizumab (AVASTIN), Aflibercept (ZALTRAP), Regorafenib (STIVARGA), and a combination thereof. In some embodiments, the EGFR inhibitor is selected from the group consisting of Cetuximab (ERBITUX), Panitumumab (VECTIBIX), Gefitinib, and a combination thereof. In some embodiments, the additional therapeutic agent is selected from angiotensin II receptor antagonists, angiotensin converting enzyme (ACE) inhibitors, caspase inhibitors, cathepsin B inhibitors, CCR2 chemokine antagonists, CCR5 chemokine antagonists, chloride channel stimulators, cholesterol solubilizers, diacylglycerol O-acyltransferase 1 (DGAT1) inhibitors, dipeptidyl peptidase IV (DPPIV) inhibitors, farnesoid X receptor (FXR) agonists, FXR/TGR5 dual agonists, galectin-3 inhibitors, glucagon-like peptide 1 (GLPl) agonists, glutathione precursors, hepatitis C virus NS3 protease inhibitors, HMG CoA reductase inhibitors, 1 Iβ-hydroxy steroid dehydrogenase (I Iβ-HSDl) inhibitors, IL-Iβ antagonists, IL-6 antagonists, IL-10 agonists, IL-17 antagonists, ileal sodium bile acid cotransporter inhibitors, leptin analogs, 5-lipoxygenase inhibitors, LPL gene stimulators, lysyl oxidase homolog 2 (LOXL2) inhibitors, PDE3 inhibitors, PDE4 inhibitors, phospholipase C (PLC) inhibitors, PPARa agonists, PPARy agonists, PPAR5 agonists, Rho associated protein kinase 2 (ROCK2) inhibitors, sodium glucose transporter-2 (SGLT2) inhibitors, stearoyl CoA desaturase-1 inhibitors, thyroid hormone receptor β agonists, tumor necrosis factor a (TNFa) ligand inhibitors, transglutaminase inhibitors, transglutaminase inhibitor precursors, PTPlb inhibitors, and ASK1 inhibitors. In some embodiments, the additional therapeutic agent is selected from capecitabine; cetuximab; bevacizumab; a MEK inhibitor such as N-[(R)-2,3-dihydroxy-propoxy]-3,4-difluoro-2-(2-fluoro-4-iodo-phenylamino)-benzamide, or a pharmaceutically acceptable salt thereof; a FOLFOX4 combination including oxaliplatin, 5-fluorouracil and leucovorin; and a FOLFIRI combination include irinotecan, 5-fluorouracil and leucovorin and the like.

In one aspect, the disclosure includes a method of treating or preventing a disease or disorder alleviated by inhibiting Wnt/β-catenin signaling in a patient in need of said treatment or prevention, the method comprising administering a therapeutically effective amount of one or more compounds of formula (I), formula (10)-(15), formula (20)-(29), formula 1001-1126, formula (II), formula 2001-2031, formula (III), formula 3001-3018, or a pharmaceutically acceptable salt thereof.

In one aspect, the disclosure provides a method of treating or preventing a disease or disorder alleviated by activating adenosine monophosphate-activated kinase (AMPK) signaling in a patient in need of said treatment or prevention, the method comprising administering a therapeutically effective amount of one or more compounds of formula (I), formula (10)-(15), formula (20)-(29), formula 1001-1126, formula (II), formula 2001-2031, formula (III), formula 3001-3018, or a pharmaceutically acceptable salt thereof.

In one aspect, the disclosure includes method of treating or preventing a disease or disorder alleviated by both inhibiting Wnt/β-catenin signaling and activating adenosine monophosphate-activated kinase (AMPK) signaling in a patient in need of said treatment or prevention, the method comprising administering a therapeutically effective amount of one or more compounds of formula (I), formula (10)-(15), formula (20)-(29), formula 1001-1126, formula (II), formula 2001-2031, formula (III), formula 3001-3018, or a pharmaceutically acceptable salt thereof.

In some embodiments, the disease is cancer or a metabolic disease. In some embodiments, the disease is selected from the group consisting of type 2 diabetes, obesity, hyperlipidemia, fatty liver disease, adrenocortical cancer, hepatocellular cancer, hepatoblastoma, malignant melanoma, ovarian cancer, Wilm's tumor, Barrett's esophageal cancer, prostate cancer, colon cancer, colorectal cancer, rectal cancer, pancreatic cancer, bladder cancer, breast cancer, gastric cancer, head & neck cancer, lung cancer, mesothelioma, cervical cancer, uterine cancer, myeloid leukemia cancer, lymphoid leukemia cancer, pilometricoma cancer, medulloblastoma cancer, glioblastoma, and familial adenomatous polyposis. In some embodiments, the disease or disorder is type 2 diabetes. In some embodiments, the disease or disorder is colon cancer and/or colorectal cancer. In some embodiments, the disease or disorder is fatty liver disease. In some embodiments, the fatty liver disease comprises a nonalcoholic fatty liver disease (NAFLD) or nonalcoholic steatohepatitis (NASH). In some embodiments, the breast cancer is triple negative breast cancer.

In one aspect, the disclosure includes a method of treating or preventing liver fibrosis in a patient in need of said treatment or prevention, the method comprising administering a therapeutically effective amount of one or more compounds of formula (I), formula (10)-(15), formula (20)-(29), formula 1001-1126, formula (II), formula 2001-2031, formula (III), formula 3001-3018, or a pharmaceutically acceptable salt thereof.

In one aspect, the disclosure includes a method of treating or preventing fatty liver disease in a patient in need of said treatment or prevention, the method comprising administering a therapeutically effective amount of one or more compounds of formula (I), formula (10)-(15), formula (20)-(29), formula 1001-1126, formula (II), formula 2001-2031, formula (III), formula 3001-3018, or a pharmaceutically acceptable salt thereof.

In some embodiments, the disease comprises alcoholic fatty liver disease (ALD) or a non-alcoholic fatty liver disease (NAFLD). In some embodiments, the disease is selected from the group consisting of simple fatty liver (steatosis), non-alcoholic steatohepatitis (NASH), and cirrhosis. In some embodiments, the disease is non-alcoholic steatohepatitis (NASH). In some embodiments, the one or more compounds are administered orally. In some embodiments, the one or more compounds are administered in combination with one or more additional therapeutic agents. In some embodiments, the additional therapeutic agent is selected from the group consisting of a RAF inhibitor, an MEK inhibitor, an ERK inhibitor, a VEGFR inhibitor, EGFR inhibitor, and a combination thereof. In some embodiments, the VEGFR inhibitor is selected from the group consisting of Bevacizumab (AVASTIN), Aflibercept (ZALTRAP), Regorafenib (STIVARGA), and a combination thereof. In some embodiments, the EGFR inhibitor is selected from the group consisting of Cetuximab (ERBITUX), Panitumumab (VECTIBIX), Gefitinib, and a combination thereof. In some embodiments, the additional therapeutic agent is selected from angiotensin II receptor antagonists, angiotensin converting enzyme (ACE) inhibitors, caspase inhibitors, cathepsin B inhibitors, CCR2 chemokine antagonists, CCR5 chemokine antagonists, chloride channel stimulators, cholesterol solubilizers, diacylglycerol O-acyltransferase 1 (DGAT1) inhibitors, dipeptidyl peptidase IV (DPPIV) inhibitors, farnesoid X receptor (FXR) agonists, FXR/TGR5 dual agonists, galectin-3 inhibitors, glucagon-like peptide 1 (GLPl) agonists, glutathione precursors, hepatitis C virus NS3 protease inhibitors, HMG CoA reductase inhibitors, 1 Iβ-hydroxy steroid dehydrogenase (I Iβ-HSDl) inhibitors, IL-Iβ antagonists, IL-6 antagonists, IL-10 agonists, IL-17 antagonists, ileal sodium bile acid cotransporter inhibitors, leptin analogs, 5-lipoxygenase inhibitors, LPL gene stimulators, lysyl oxidase homolog 2 (LOXL2) inhibitors, PDE3 inhibitors, PDE4 inhibitors, phospholipase C (PLC) inhibitors, PPARa agonists, PPARy agonists, PPAR5 agonists, Rho associated protein kinase 2 (ROCK2) inhibitors, sodium glucose transporter-2 (SGLT2) inhibitors, stearoyl CoA desaturase-1 inhibitors, thyroid hormone receptor β agonists, tumor necrosis factor a (TNFa) ligand inhibitors, transglutaminase inhibitors, transglutaminase inhibitor precursors, PTPlb inhibitors, and ASK1 inhibitors. In some embodiments, the additional therapeutic agent is selected from capecitabine; cetuximab; bevacizumab; a MEK inhibitor such as N-[(R)-2,3-dihydroxy-propoxy]-3,4-difluoro-2-(2-fluoro-4-iodo-phenylamino)-benzamide, or a pharmaceutically acceptable salt thereof; a FOLFOX4 combination including oxaliplatin, 5-fluorouracil and leucovorin; and a FOLFIRI combination include irinotecan, 5-fluorouracil and leucovorin and the like.

### BRIEF DESCRIPTION OF THE DRAWINGS

The foregoing summary and the following detailed description of the exemplary embodiments of the present disclosure may be further understood when read in conjunction with the appended drawings, in which:

FIG. 1 illustrates a scheme of a design strategy of YA6060 from FX1128 and YW2065.

FIG. 2 illustrates the structure of YA6060.

FIG. 3A-FIG. 3B illustrate the pharmacokinetic and toxicity evaluation for YA6060. FIG. 3A is a graph of experimental data illustrating that YA6060 was well tolerated without any notable toxicity. FIG. 3B is a graph of experimental data illustrating that YA6060 exhibited desirable pharmacokinetics (PK) and is bioavailable.

FIG. 4 illustrates that YA6060 protects liver from damage caused by dimethylnitrosamine (DMNA). Vehicle (top panels) or 10 mg/kg DMNA (bottom panels) was given on a Monday-Tuesday-Wednesday schedule each week; YA6060 or vehicle was given daily. After sacrifice of mice (6/group) at 4 weeks, paraffin sections were stained with Sirius Red to visualize collagen. The top-left panel displays the histology of healthy liver. Intense staining in the bottom-left panel shows fibrosis induced by DMNA. YA6060 at 0.5 mg/kg (bottom-center) or 5 mg/kg (bottom-right) exhibited strong protective effects. By itself, YA6060 did not disrupt liver histology (top-center, top-right). Bars, 100 μm.

FIG. 5 is a schematic illustrating the role of Axin stabilization in hepatic metabolism and the mechanism underlying YA6060 pharmacological action.

FIG. 6A-FIG. 6E illustrate the development of Axin stabilizers with dual activities of Wnt inhibition and AMPK activation. FIG. 6A is a scheme illustrating the discovery of YA6060 from previous compound YW2065. FIG. 6B is an image of a gel illustrating the effects of YW2065 on Axin stabilization and subsequent β-catenin degradation. FIG. 6B is an image of a gel illustrating the effects of YW2065 and YA6060 on Axin stabilization and phosphorylation of AMPK (p-AMPK) FIG. 6A and FIG. 6B were validated by Western blotting. HEK293 cells were used. B&C: 100 nM compounds, 24 h. FIG. 6D is a table showing the activity profile of YA6060. F=bioavailability; soubility is aqueous solubility. FIG. 6E is a graph of experimental data demonstrating the dose response of YA6060 in the luciferase gene reporter assay.

FIG. 7A-FIG. 7H illustrate the identification of TAB182 as a protein target. FIG. 7A illustrates the structures of biotinylated YA6060 analogs YA2103 and YA6023 and their IC₅₀. FIG. 7B is a graph of experimental data illustrating protein hits pulled down by biotinylated affinity chromatography with YA2103 (100 μM) as analyzed by mass spectrometry for HEK293 cell lysate. Arrow: TAB182. FIG. 7C is an image illustrating immunoblotting validation of TAB182 as a hit from the pulldown with YA2103 and YA6023. Lysates of HEK293 cells overexpressing c-myc tagged TAB182 were incubated with the biotinylated compounds or vehicle and streptavidin beads. Pulldown was detected by c-myc antibody. *250 kda protein marker. Red arrow, TAB182. FIG. 7D is a graph of experimental data illustrating that protein TAB182 mRNA level was reduced by a siRNA. FIG. 7E is a graph of experimental data illustrating that protein TAB182 knockdown significantly reduced both the base and Li-stimulated Wnt signaling activities in HEK293 cells as determined by TOPflash reporter activity. FIG. 7F is a graph of experimental data illustrating that in the presence of TAB182 knockdown, no significant inhibition by YW2065 (100 nM), an active analog of YA6060, on Wnt signaling activity in the reporter assay in HEK293 cells. Unlike in FIG. 7D with absolute values of RLU, the percentage relative to vehicle treatment is plotted in panel FIG. 7E for a clear comparison between scramble and siTAB182. FIG. 7G is an image illustrating that protein TAB182 knockdown enhanced the base level of Axin proteins while abolishing the effect of YW2065 on Axin increase. V, vehicle; F, YW2065 (100 nM). FIG. 7H is an image illustrating that TAB182 knockdown leads to AMPK activation in HEK293 cells.

FIG. 8 illustrates the preliminary toxicity evaluated for YA6060 in major mouse organs. YA6060 was well tolerated by oral gavage with YA6060 (500 mg/kg once) and vehicle (saline), respectively, and observed without any notable toxicity in major tissues. 11 weeks C57BL/6 mice were treated for 21 consecutive. The isolated tissues were stained by Haematoxylin and Eosin (H&E). The images were taken under an amplification of 20×. No histological changes are noted in major tissues from the mice received YA6060 treatment.

FIG. 9A-FIG. 9C illustrate the anti-fibrotic efficacy of YA6060 in LX-2 cells. LX-2 cells were exposure to TGF-beta with and without different concentrations of YA6060 for 24 hours. FIG. 9A is a gel illustrating Western blot results. FIG. 9B and FIG. 9C are graphs of experimental data illustrating RT-PCR results.

FIG. 10 is a graph of experimental data illustrating anti-fibrotic efficacy of YA6060 in mice. C57BL/6 mice received dimethylnitrosamine (DMNA, 10 mg/kg/d i.p. for 3d and then rest for 4d) or saline, with or without YA6060 (0.5 mg/kg/d) for 4 weeks. The fibrosis and inflammation biomarkers were examined by RT-PCR with the liver tissues.

FIG. 11A-FIG. 11C illustrate the effect of YA6060 on liver function in mice. C57BL/6 mice received dimethylnitrosamine (DMNA, 10 mg/kg/d i.p. for 3d and then rest for 4d) or saline, with or without YA6060 (0.5 mg/kg/d) for 4 weeks. The serum biochemistry was determined for alanine aminotransferase (ALT) and aspartate aminotransferase (AST) (FIG. 11A), albumin (FIG. 11B), and total bilirubin (FIG. 11C).

FIG. 12A-FIG. 12B illustrate a schematic of the role of axin proteins in cellular signaling. FIG. 12A illustrates the Wnt signaling pathway and known inhibitors. FIG. 12B illustrates AMPK activation and activators.

FIG. 13 is a graph of experimental data demonstrating YA6060 acute toxicity test in mice. Body weight change was measured over time in mice received YA6060. 11 weeks C57BL/6 mice were treated for 14 consecutive days by oral garage with YA6060 at the indicated doses once daily and vehicle (saline), respectively.

FIG. 14A-FIG. 14C illustrate the anti-fibrotic efficacy of YA6060 in LX-2 cells. The effect of YA6060 treatment on the expression of fibrotic markers in LX-2 cells was examined. LX-2 cells were treated with TGF-beta to model fibrosis. FIG. 14A is an image of a Western blot illustrating protein expression of collagen 1, vimentin, and alpha-SMA. FIG. 14B is a graph of experimental data illustrating mRNA expression of collagen 1, vimentin, and alpha-SMA by RT-PCR. FIG. 14C is a graph of experimental data illustrating mRNA expression of genes associated with inflammation by RT-PCR. The cells were treated with the indicated concentrations of YA6060 for 24 hours. *P<0.05, **P<0.01.

FIG. 15 is am image illustrating the effect of YA6060 treatment on liver fibrosis in mice. C57BL mice were treated with 4 weeks of dimethylnitrosamine (DMNA) (for each week: 10 ug/g/day for 3 days, then no treatment for 4 days) to establish the animal model of liver fibrosis. The mice received either saline control or YA6060 with or without DMNA. The liver tissues were stained by Sirius red (SR) to show fibrosis.

FIG. 16 is a graph of experimental data illustrating the anti-fibrotic efficacy of YA606 in mice. Fibrosis in the liver tissues of the mice that received YA6060 treatment was quantified. C57BL mice were treated with 4 weeks of dimethylnitrosamine (DMNA) (for each week: 10 ug/g/day for 3 days, then no treatment for 4 days) to establish the animal model of liver fibrosis. The mice received either saline control or YA6060 with or without DMNA. The collagen of liver tissues was stained by Sirius red (SR) and quantitated by Image J software. ***P<0.001.

FIG. 17 is an image illustrating the effect of DMNA and YA6060 treatment on hepatic histology in mice. C57BL mice were treated with 4 weeks of dimethylnitrosamine (DMNA) (for each week: 10 ug/g/day for 3 days, then no treatment for 4 days) to establish the animal model of liver fibrosis. The mice received either saline control or YA6060 with or without DMNA. The liver tissues were stained by H&E. DMNA treatment led to inflammation infiltration which could be ameliorated by YA6060 treatment.

FIG. 18 is a table illustrating the number of mice in Ishak fibrosis stage. C57BL mice were treated with 4 weeks of dimethylnitrosamine (DMNA) (for each week: 10 ug/g/day for 3 days, then no treatment for 4 days) to establish the animal model of liver fibrosis. The mice received either saline control or YA6060 with or without DMNA. The histology of liver tissues was scored according to Ishak Fibrosis Stage for each mouse.

FIG. 19 is a graph illustrating the effect of YA6060 treatment on the hepatic expression of fibrotic and inflammatory markers in mice. C57BL mice were treated with 4 weeks of dimethylnitrosamine (DMNA) (for each week: 10 ug/g/day for 3 days, then no treatment for 4 days) to establish the animal model of liver fibrosis. The mice received either saline control or YA6060 with or without DMNA. The mRNA expression of fibrotic and inflammatory marker genes was determined by RT-PCR. *P<0.05, **P<0.01, ***P<0.001.

FIG. 20A-FIG. 20B illustrate the effect of DMNA and YA6060 treatment on body weight change (FIG. 20A) and liver/body weight (FIG. 20B) in mice. C57BL mice were treated with 4 weeks of dimethylnitrosamine (DMNA) (for each week: 10 ug/g/day for 3 days, then no treatment for 4 days) to establish the animal model of liver fibrosis. The mice received either saline control or YA6060 with or without DMNA. *P<0.05, **P<0.01, ***P<0.001.

FIG. 21A-FIG. 21C illustrate the effect of DMNA and YA6060 treatment on liver function in mice. C57BL mice were treated with 4 weeks of dimethylnitrosamine (DMNA) (for each week: 10 ug/g/day for 3 days, then no treatment for 4 days) to establish the animal model of liver fibrosis. The mice received either saline control or YA6060 with or without DMNA. The liver function was determined by analyzing the serum biochemistry of albumin (FIG. 21A), total bilirubin (FIG. 21B), and ALT and AST levels (FIG. 21C). *P<0.05, **P<0.01 as compared to the control.

FIG. 22A-FIG. 22E illustrate the inhibition of Wnt/β-catenin pathway by compounds of the disclosure in CRC cells. FIG. 22A is a graph of experimental data illustrating TOPflash assay results in SW480 and SW620 cells. FIG. 22B is an image of a gel illustrating that YW2065 altered the protein levels of β-catenin and Axin. FIG. 22C is a graph of experimental data illustrating decreased the mRNA levels of Wnt target genes in SW620 cells. Cells were treated for 24 h. FIG. 22D is a table of experimental data illustrating relative viability (IC50) by YW2065. Cells were treated for 72 h. FIG. 22E is a graph of experimental data illustrating the effect of YW2065 and YA6060 treatments on the viability of HT29 cells.

FIG. 23A-FIG. 23C illustrates that YW2065 inhibited CRC cell proliferation and growth. FIG. 23A is an image illustrating a colony formation assay. The cells (500 cells/well) were treated with vehicle or YW2065 until visible colonies formed. FIG. 23B is an image of experimental data illustrating SW620 cells were treated with YW2065 for 36 h, stained with AAD-7 and annexin V, and analyzed by flow cytometry. The orange circle indicates cells in early apoptosis. FIG. 23C is a graph of experimental data illustrating that SW620 cells became resistant to YW2065 (72 h) cytotoxicity when Axin was knocked down by lentivirus encoding shRNA against Axin-1 or a constitutively active β-catenin mutant S33Y overexpressed.

FIG. 24A-FIG. 24D illustrate that YW2065 suppressed SW620 xenograft growth in mice. 6-week old nude mice were injected with 2×106 SW620 cells S.C. 10 days later, mice received YW2065 (i.p.) or vehicle each day (n=6-8). FIG. 24A is a graph of experimental data illustrating tumor growth, which was measured every 2 days. The mice were euthanized at day 32 and tumors were weighed (FIG. 24B). FIG. 24C is a graph of experimental data illustrating the expression of Wnt target genes (c-Myc & Cyclin D1) in tumors detected by RT-PCR. FIG. 24D is an image of a gel illustrating the phosphorylation of AMPK (p-AMPK) and its downstream target ACC (p-ACC) as determined by immunoblotting.

FIG. 25A-FIG. 25D illustrate a murine model of metastatic colon cancer. FIG. 25A is an image illustrating the HT-29 xenograft fragment sutured into cecal wall. FIG. 25B is an image illustrating the resected locally-invasive cecal tumor (left) and hematogenous liver metastases (arrows; right) from the same mouse. FIG. 25C is an image illustrating the cecal MC38luc1 tumor bioluminescent signal 2 weeks after implantation and confirmed after necropsy (FIG. 25D, circled).

FIG. 26A-FIG. 26D illustrate that axin is key to AMPK activation by YW2065. FIG. 26A is an image demonstrating enhanced Axin expression by YW2065 (1 μM). HEK293 cells were stained with anti-axin-1 or DAPI and studied under fluorescence microscopy. FIG. 26B is an image illustrating enhanced AMPK phosphorylation by Axin overexpression (empty pcDNA3 vs. pcDNA3-Axin-1) in HEK293 cells. FIG. 26C is an image illustrating Axin knockdown abolished AMPK activation by YW2065 (10 μM) in HEK293 cells. See FIG. 23A-FIG. 23C for Axin knockdown. FIG. 26D is an image illustrating that p-AMPK and cytoplasmic AMPK were increased by YW2065 (10 μM) and YW1128 (10 μM) in SW620 cells. Cells were treated for 24 h.

FIG. 27 is an image of illustrating the activation of AMPK pathway by the potent YW2065 analog YW2049 in CRC cells. YW2049 treatment (three hours) increased p-AMPK and its downstream p-ACC levels, dose-dependently. Treatment of compound YW2049 indicated no effects on total protein levels of AMPK and ACC in SW620 cells.

FIG. 28A-FIG. 28B illustrate the effects of YW1128 and XAV939 on proteomic changes in HEK293 cells. The cells were treated with compound (10 μM) or vehicle for 6 h to catch early protein changes by mass spectrometry. FIG. 28A is a graph illustrating significantly downregulated pathways by each compound. Wnt pathway is indicated. FIG. 28B is a table illustrating the top ten pathways affected by compound. Mitochondrial signaling is the most affected by both (red box).

FIG. 29A-FIG. 29D illustrate the effects of YW2065 treatment on mitochondrial. FIG. 29A is a graph of experimental data illustrating that YW2065 reduced oxygen consumption rate (OCR) in 4 h. FIG. 29B is a graph of experimental data illustrating ATP production estimated from OCR measurement. FIG. 29C is an image illustrating that YW2065 (10 μM) caused short and punctuated mitochondria in SW480 cells. FIG. 29D illustrates that YW2065 increased ROS generation in SW480 cells as evaluated by staining the cells with fluorescent DHE and quantified by ImageJ (˜300 cells counted each).

FIG. 30A-FIG. 30B illustrate a comparison of YW2065 and XAV939 for inhibitory effects on Wnt signaling and TNKS1. FIG. 30A is a graph of experimental data illustrating TOPflash assay for Wnt signaling activity in SW480 cells. FIG. 30B is a graph of experimental data illustrating TNKS1 activity was determined by TNKS1 Histone Ribosylation Colorimetric Assay.

FIG. 31 is a table of experimental data illustrating inhibitory activity (% Inh) of YW2065 for a panel of 273 potential off-target kinases. Only the ones that indicated >10% inhibition are shown.

FIG. 32A-FIG. 32H illustrate the identification of TAB182 as a protein target. FIG. 32A is illustrates the structures of biotinylated YW2065 analogs YA2103 and YA6023 and their IC50. FIG. 32B is a graph of experimental data of protein hits pulled down by biotinylated affinity chromatography with YA2103 (100 μM) as analyzed by mass spectrometry for HEK293 cell lysate. Arrow: TAB182. FIG. 32C is an image of immunoblotting validation of TAB182 as a hit from the pulldown with YA2103 and YA6023. Lysates of HEK293 cells overexpressing c-myc tagged TAB182 were incubated with the biotinylated compounds or vehicle and streptavidin beads. Pulldown was detected by c-myc antibody. *250 kda protein marker. Red arrow, TAB182. FIG. 32D is a graph of experimental data illustrating that TAB182 mRNA level was reduced by a siRNA. FIG. 32E is a graph of experimental data illustrating that TAB182 knockdown significantly reduced both the base and Li-stimulated Wnt signaling activities in HEK293 cells as determined by TOPflash reporter activity. FIG. 32F is a graph of experimental data illustrating that in the presence of TAB182 knockdown, no significant inhibition by YW2065 (100 nM) on Wnt signaling activity in the reporter assay in HEK293 cells. Note: unlike in FIG. 32D with absolute values of RLU, the percentage relative to vehicle treatment is plotted in FIG. 32E for a clear comparison between scramble and siTAB182. FIG. 32G is an image illustrating that TAB182 knockdown enhanced the base level of Axin proteins while abolishing the effect of YW2065 on Axin increase. V, vehicle; F, YW2065 (100 nM). FIG. 32H is an image illustrating that TAB182 knockdown leads to AMPK activation in HEK293 cells.

FIG. 33 is a schematic of a non-limiting example of an experimental design strategy for studying anti-CRC effects of YA6060 in vitro and in vivo.

FIG. 34 is a schematic of a non-limiting example of an experimental design and mechanism to manipulate the identified pathways at the indicated points.

FIG. 35 is a schematic of a strategic outline for the synthesis, evaluation, and characterization of compounds of the disclosure.

FIG. 36 is an image illustrating the structure of F-containing YA6060 analogues.

FIG. 37 is a scheme illustrating a non-limiting synthesis of folate receptor (FR)-targeting folic acid-YA6060 hybrids.

FIG. 38 is an image illustrating a non-limiting example of biguanide-YA6060 hybrids.

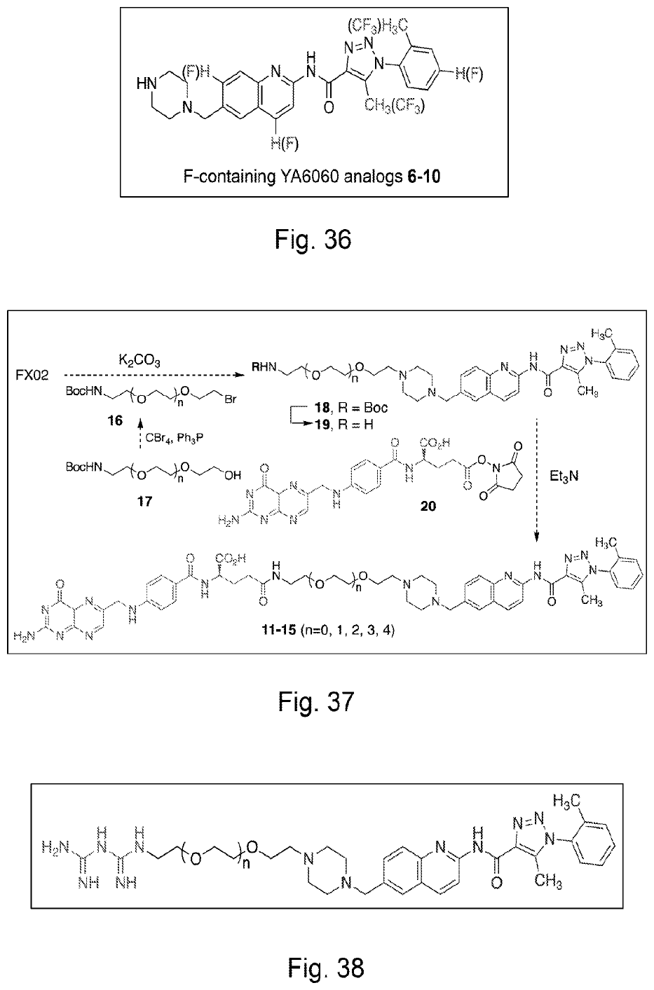

### DETAILED DESCRIPTION

The present disclosure relates generally to compounds, and methods of using such compounds, that may inhibit the Wnt/β-catenin signaling pathway and/or activate the AMPK pathway. More specifically, the compounds of the disclosure may be used in treating diseases that implicate the Wnt/β-catenin signaling pathway and/or activate the AMPK pathway.

The Wnt/β-catenin signaling pathway plays a pivotal role in cell proliferation, differentiation, and organ development. Moreover, it regulates the transcription of its target genes through the transcriptional factor β-catenin. In the “off state” without Wnt ligands, β-catenin forms a cytoplasmic “destruction complex” with adenomatous polyposis coli (APC) and Axin, which facilitates the phosphorylation of β-catenin by glycogen synthase kinase 3 (GSK3) and casein kinase 1 (CK1) at the N-terminal residues Ser45, Thr41, Ser37, and Ser33. Phosphorylated β-catenin is recognized and ubiquitinated by the F-box β-transducin repeat-containing protein β-TrCP), and then degraded by the proteasome. On the other hand, the “on-state” of the pathway involves increased post-translational stability and thus accumulation of β-catenin, through Wnt-dependent degradation of Axin and inhibition of GSK3 by various mechanisms. As the β-catenin level increases, it translocates to the nucleus where it binds to LEF/TCF such as TCF7L2 and activates expression of downstream genes. Increased expression of β-catenin and malfunction of the Wnt-signaling pathway are implicated in a variety of diseases.

As an emerging research area, however, the link between Wnt/β-catenin pathway and metabolic diseases has only been appreciated recently. For example, a strong association exists between type 2 diabetes risk and single nucleotide polymorphisms (SNPs) in TCF7L2, a classic effector of Wnt/β-catenin pathway. Similar genetic evidence exists for additional modulators of Wnt signaling pathway such as WNT5B, WNT10B, and LRP6. Follow-up genetic studies have indicated that global downregulation of Wnt/β-catenin signaling activity leads to overall improved metabolic homeostasis in diabetic animal models

The metabolic disease NAFLD is the most common form of chronic liver disease and ranges in severity from relatively simple benign steatosis to NASH, which is highly prevalent in type 2 diabetes or obese patients, and is a burgeoning public health problem due to the global diabetes and obesity epidemic. Dietary control and exercise are currently the recommendation to reverse NAFLD/NASH; however, their long-term effectiveness is uncertain because many patients are unable to comply. Thus, an effective pharmacological therapeutic is highly in demand.

Reducing β-catenin expression by antisense oligonucleotides decreases expression of enzymes involved in hepatic fatty acid esterification and ameliorates diet-induced hepatic steatosis and insulin resistance. Indeed, an antisense oligonucleotide against β-catenin could totally reverse diet-induced fatty liver and obesity back to normal, improve glucose tolerance, and reduce fasting glucose levels in the blood of mice.

Nonalcoholic fatty liver disease (NAFLD) affects more than 30% of Americans. At present, there are no pharmacological options approved for NAFLD and its clinical sequelae including nonalcoholic steatohepatitis (NASH), fibrosis, and cirrhosis. Steatosis is the most outstanding feature of NAFLD/NASH, while progressive accumulation of fibrosis is the hallmark of the disease progression. Beta-catenin and adenosine monophosphate-activated kinase (AMPK) are crucial proteins in regulation of hepatic metabolism that interact with the scaffold protein Axin. Genetic evidence supports a metabolic benefit by β-catenin downregulation on hepatic metabolism; meanwhile, AMPK has emerged as a target against metabolic disorders.

Nonalcoholic fatty liver disease (NAFLD) is defined as fat accumulation in the liver exceeding 5-10% by weight in individuals that do not drink alcohol (<2 drinks/day). NAFLD affects 34-46% of Americans. While NAFLD is a generally benign condition, NASH, the most severe form, causes liver swelling, fibrosis and scarring of the liver. In 25% NASH patients, fibrosis leads to cirrhosis. NASH affects approximately 12% of Americans. Control of lipids by diet and exercise has shown some benefits for NASH; but adherence to long-term lifestyle changes remains a large challenge for patients. There are currently no approved pharmacological therapies for the treatment of NAFLD/NASH.

An investigation into drugs with metabolic side effects indicated that many of these drugs were an activator of Wnt/β-catenin pathway. In fact, increasing evidence indicates that dysregulation of Wnt/β-catenin pathway is a causative pathological mechanism for metabolic diseases (ref). Inhibition of this pathway may hold therapeutic benefits for the treatment of metabolic diseases including NASH. In one aspect, the present disclosure provides a class of compounds that can stabilize cellular Axin levels. Axin is an important scaffold protein that interacts with β-catenin and other proteins to form a destruction complex. It facilitates degradation of/i-catenin, leading to inhibition of Wnt/β-catenin pathway. Axin has been recently found to also interact with adenosine monophosphate-activated kinase (AMPK), which is a crucial cellular energy sensor.

Globally, the prevalence of NAFLD is estimated at ˜25%. NAFLD/NASH causes a growing economic challenge, with an estimated direct annual cost of $103 billion in the US. In the US, NAFLD cases are projected to expand from 83.1 million in 2015 (˜25% of the population) to 100.9 million in 2030. An increased proportion of these cases will be NASH, rising from 20% to 27% of adults with NAFLD during this interval. NASH is the second indication for liver transplantation. To date, no evidence-based drug therapy has been approved for NASH management and NASH is classified as a medical condition with high-unmet therapeutic need.

It is estimated that the drug market for NAFLD/NASH will be worth $20 billion to $35 billion per year by 2025. According to a very recent report, the global NASH drug market will reach $84.34 billion in 2029 with a CAGR of 39% (2018-2029). The current NASH drug market is wholly occupied by off-label drugs as there are no clinically approved medicines for NASH. Non-pharmacological treatments remain the first line of management for NASH patients. However, there are over 300 on-going clinical trials investigating approximate 200 candidate drugs for NASH. Because there has been a high failure rate for late-stage drug candidates that provides a significant opportunity for new approaches, there remains a significant need for new pathways to treat NASH.

There is thus a need to provide NASH patients with a novel drug therapy. In one aspect, the compounds of the instant disclosure, such as YA6060, are useful as a novel drug therapy for NASH, and for other diseases as well.

Five candidates have entered into phase III trials. While data for the other four have not been released, the likelihood of approval for Allergan's cenicriviroc is low due to recent Part I results showing lacking efficacy. The mechanisms for these phase III candidate drugs, and others in earlier phase development include FXR agonization, PPAR-α agonization, ASK1 inhibition, CCR2/5 antagonization, and THRβ agonization.

In contrast, the compounds of the instant disclosure have a unique mechanism. In a non-limiting example, YA6060 is competitive as it has dual activities of β-catenin degradation and AMPK activation via Axin stabilization that is critical in liver metabolism but not claimed by any of on-going candidate drugs. NCE's with multiple modes of action and/or targets, such as YA6060 could prove most promising, as NASH pathogenesis involves many disease drivers. Metformin is currently the only AMPK activator (e.g., metformin). However, the efficacy of metformin in NASH patients is disputed because of widely varying efficacy outcomes in various clinical trials, suggesting AMPK activation alone may not deliver a consistent efficacy in NASH patients. Known Axin stabilizers, such as XAV939 and IWR1, are tankyrase inhibitors, to which the efficacy against NASH is unknown. Concerns have arisen for GI toxicity for these class of compounds. Tankyrase inhibitors may have limited efficacy due to their undesirable ability to stabilize the protein level of tankyrase. Instead YA6060 stabilizes Axin by binding to TAB182, a protein that binds and activates tankyrase, which provides a strategy to bypass the side effects associated with tankyrase inhibitors.

One in every 23 people is expected to be diagnosed with colorectal cancer (CRC) during her/his lifetime. As the third most common cancer, CRC is the second leading cause of cancer-related death in the United States. The 5-year survival rate for patients with metastatic CRC is less than 12%. Chemotherapies 5-FU, oxaliplatin, and regimens based on them continue to be a first-line treatment despite their significant limitations and high toxicities. Emerging biologicals targeting VEGF and EGFR do not significantly improve survival rate. These underline the urgent need to discover novel targeted therapeutics against CRC.

More than 92% of CRC are characterized by aberrant activation of Wnt/β-catenin signaling due to genetic mutations or epigenetic changes. Activated Wnt signaling represents a preferred target for the treatment of CRC. However, the genetic/epigenetic changes associated with Wnt signaling upregulation are also a major reason for high heterogeneity of CRC and pose challenges to target this pathway. Meanwhile, metabolic reprogramming has been well demonstrated as a hallmark of cancer progress and adenosine monophosphate-activated kinase (AMPK), a crucial energy sensor, has emerged as a target against cancer metabolism. Thus, simultaneously targeting the Wnt signaling and AMPK pathways represents a novel therapeutic strategy for the treatment of CRC.

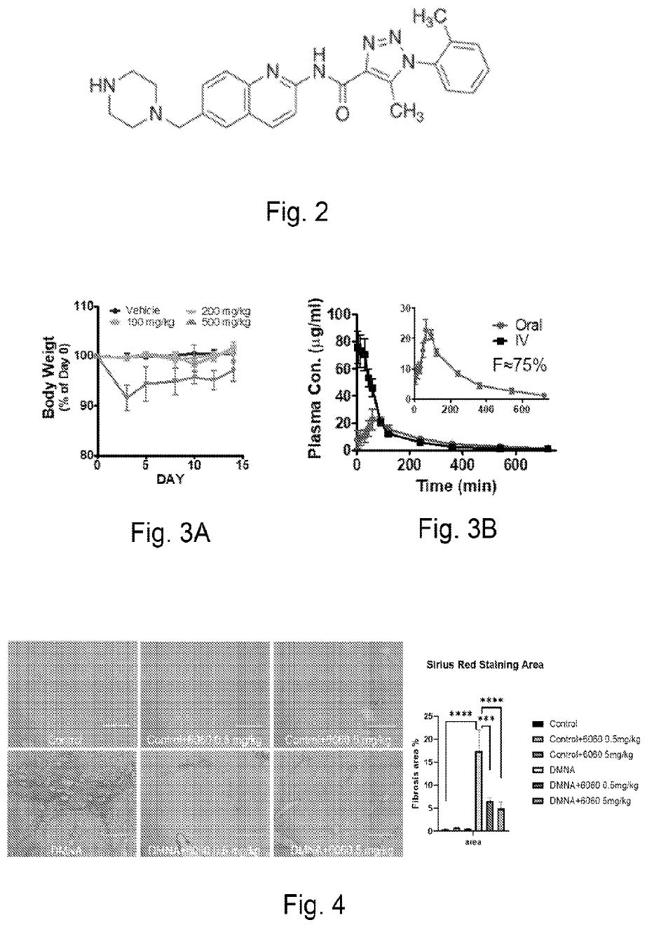

The Wnt/β-catenin pathway plays a key role in cell proliferation, differentiation and growth. It regulates the expression of target genes via the transcriptional factor β-catenin that forms a cytoplasmic destruction complex with the scaffolding protein Axin and adenomatous polyposis coli (APC). This destruction complex facilitates the phosphorylation of β-catenin by casein kinase 1α (CK1α) and glycogen synthase kinase β(GSK3β), causing β-catenin degradation and therefore suppressed Wnt signaling (FIG. 2A). The aberrant Wnt signaling is associated with many diseases including various cancers, developmental disorders, fibrosis, neurological diseases, and metabolic diseases. The Wnt signaling pathway also talks to others via intermediate effector proteins Axin, β-catenin, and GSK3β. Among the known crossing pathways, the adenosine monophosphate-activated kinase (AMPK) pathway plays a key role in maintaining energy homeostasis at the cellular and whole-body levels (FIG. 2B). Activators of the AMPK pathway has emerged as a forefront of therapeutic agents for cancer and metabolic disorders. In one aspect, the present disclosure provides compounds as a novel targeted therapy for colorectal cancer (CRC) by simultaneously inhibiting Wnt signaling and activating AMPK pathways.

CRC is characterized by aberrant Wnt signaling and metabolic dysregulation. CRC causes over 50,000 deaths yearly and may soon surpass lung cancer to become the leading cause of cancer-related death in the U.S. Metastatic CRC has a 5-year survival rate of ˜12%. First-line treatments for metastatic CRC include the thymidylate synthase inhibitor 5-FU, DNA-alkylating agent oxaliplatin, and their combinations (e.g., FOLFOX, FOLFIRI, and FOLFOXIRI) despite the limited therapeutic index. New biologicals targeting vascular endothelial growth factor (VEGF) and epidermal growth factor receptor (EGFR) (e.g., bevacizumab, cetuximab and panitumumab) do not significantly improve survival rate. Therefore, novel CRC therapeutics are urgently needed. Over 92% of CRC are characterized by aberrant activation of Wnt signaling due to genetic mutations or epigenetic silencing of Wnt antagonist genes. On the other hand, metabolic reprogramming has been well demonstrated as a hallmark of cancer progress. Cancer cells including CRC are under selection pressure to downregulate AMPK, the crucial cellular energy sensor associated with cell viability. AMPK activation can reduce cancer cell proliferation and growth, mainly via the activation of the p53 and mTOR pathways.

Therapeutic targeting of Wnt signaling and AMPK pathways is a viable strategy. While safety concern is historically present due to the severe phenotypes in genetic knockout animal models, it has not been borne out either preclinically or clinically from approximate a dozen of pharmacological inhibitors, and Wnt signaling-targeting agents have entered clinical trials. Of note, a few FDA-approved drugs, such as glucocorticoids, retinoids, and celecoxib, are also found to have strong inhibitory activities. However, oncogenic mutations or epigenetic changes associated with Wnt signaling activation can lead to high heterogeneity of CRC, which may result in limited efficacy by targeting Wnt signaling alone. On the other hand, as a crucial energy sensor, AMPK is a preferred therapeutic target against cancer metabolism. In recent years, many AMPK activators have been identified and tested in preclinical models, and a small number have entered clinical trials. Notably, metformin, a widely prescribed anti-diabetic, exhibits anti-CRC effects by acting on tumor suppressor pathways via AMPK activation. Compared to single pathway modulation, simultaneously targeting both Wnt/β-catenin and AMPK pathways may offer anti-CRC agents that have not only higher efficacy but also broader application.

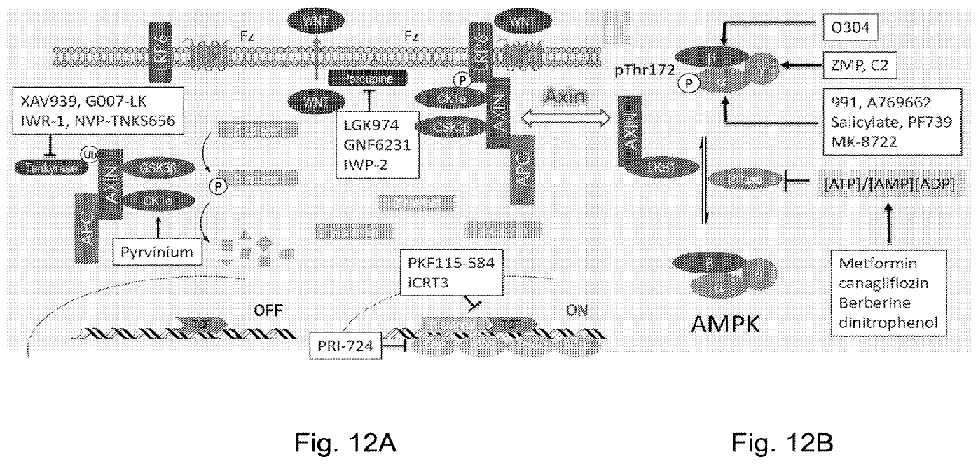

Wnt inhibitors have been developed by targeting different components of the pathway. Novartis clinical candidate LGK974 and its analog GNF-6231, as well as IWP-2 are porcupine inhibitors, which functions by decreasing the secretion of Wnt ligands (FIG. 12A). Considering the upstream location of the target, these porcupine inhibitors would not provide sufficient specificity over CRC genetic heterogeneity. PKF115-584 and iCRT3 analogs block the protein-protein interaction (PPI) of β-catenin with LEF/TCF. Prism's clinical candidate PRI-724 (Phase I) inhibits the PPI of β-catenin with coactivator CBP. However, although these downstream PPI inhibitors have improved specificity on Wnt signaling inhibition, the aberrant crosstalk with other signaling pathways via major intermediate effectors may exist, leading to unexpected toxicity. Moreover, XAV939, IWR-1, G007-LK, and NVP-TNKS656 are inhibitors of tankyrase (TNKS), a member of poly(ADP-ribose) polymerases (PARPs), which positively regulate the degradation of Axin through the mechanism of poly(ADP-ribose)ation (PARylation). Although TNKS inhibitors were proposed as Wnt signaling inhibitors, catalytic inhibition of TNKS turned out to be insufficient to effectively block Wnt signaling activity because it results in TNKS polymerization, via TNKS SAM domains, which instead promote Wnt signaling independently of the catalytic activity. Moreover, TNKS recruits and PARylates numerous protein partners via its ankyrin (ANK)-repeat domain for various biological purposes, therefore, targeting TNKS active site may also lead to other potential toxicities, In addition, the FDA approved anthelmintic drug pyrvinium (Pyr) was reported as a Wnt inhibitor by activating CK1α and has showed anti-CRC efficacy in cells and in Apc^{min/+} mice. However, application of Pyr as an anti-CRC agent is prohibited by its poor physiochemical characters such as aqueous solubility leading to extremely low oral bioavailability and high toxicity.

AMPK includes three subunits: the protein kinase catalytic a subunit, and non-catalytic β and γ subunits (FIG. 12B). Two general mechanisms have been proposed for AMPK activators. One is through inhibition of mitochondrial respiration, for example, complex I inhibition by metformin, R419, canagliflozin and berberine, which results in ATP depletion and consequent increase of AMPK phosphorylation at Thr172. The other is via directly binding to subunits of AMPK (for example, O304, ZMP, C2, 991, A769662, salicylate, PF739, and MK-8722), leading to allosteric activation, protection against dephosphorylation, and/or endogenous ligand affinity increase. While the development of AMPK activators is an emerging field, cancer including CRC is among promising indications for therapeutic AMPK activation. To date, no small molecules with dual activities of Wnt signaling inhibition and AMPK activation have been reported.

In one aspect, the present disclosure provides knowledge on Wnt signaling and AMPK pathways to provide novel Axin stabilizers with previously unrecognized mechanism of action, e.g. first-in-class small molecules that interfere with the binding of TNKS-binding protein1 (TAB182) to TNKS by using a combined approach of biochemistry, biology and state-of-art proteomics. In some embodiments, the compounds of the disclosure are useful as anti-CRC agents.

In one aspect, the disclosure provides the development of an improved generation of Wnt inhibitors with significantly enhanced aqueous solubility (>10,000 fold) and bioavailability (>3 fold) that maintain the inhibitory potency against the Wnt signaling pathway via Axin stabilization.

In one aspect, the compounds and methods of the disclosure support targeting the Wnt/β-catenin pathway and/or the AMPK pathway as a strategy to treat NAFLD.

In one aspect, the compounds and methods of the disclosure support targeting the Wnt/β-catenin pathway and/or the AMPK pathway as a strategy to treat colorectal cancer (CRC).

In one embodiment, the compounds of the disclosure may be used as treatments for metabolic disease in a patient in need thereof. As used herein, the term “metabolic disease” may refer to diseases that involve a disruption to a patient's metabolic homeostasis, including, but not limited to, type 2 diabetes, obesity, hyperlipidemia, and fatty liver disease. In some embodiments, the metabolic disease described in the disclosure may be type 2 diabetes. In some embodiments, fatty liver disease may include alcoholic fatty liver disease (ALD) or non-alcoholic fatty liver disease (NAFLD). In some embodiments, NAFLD may include one or more of simple fatty liver disease (steatosis), non-alcoholic steatohepatitis (NASH), and liver cirrhosis. In certain embodiments, the metabolic disease may be NASH.

Research in different groups has identified an association between type 2 diabetes risk and single nucleotide polymorphisms (SNPs) in TCF7L2, an effector of the Wnt/β-catenin pathway. Similar genetic evidence has been obtained for additional modulators of the Wnt signaling pathway, including WNT5B, WNT10B, and LRP6. Therefore, the Wnt/β-catenin pathway has emerged as a novel therapeutic target for treating disease, including metabolic disorders.

With respect to cancer, the Wnt pathway may be activated in a variety of cancers including, for example, colon cancer, hepatocellular carcinoma, lung cancer, ovarian cancer, prostate cancer, pancreatic cancer, and leukemias such as CML, CLL and T-ALL. The activation is due to constitutively active β-catenin, perhaps, without being limited to any one theory, due to its stabilization by interacting factors or inhibition of the degradation pathway. Accordingly, the compounds and compositions described herein may be used to treat these cancers in which the Wnt pathway is constitutively activated.

Folate receptor (FR)-targeted therapy. The FR family is a group of three folic acid (FA)-binding receptors including FRα, FRβ, and FRγ. Different to FRγ that is a soluble protein constitutively secreted by lymphoid, FRα and FRβ are glycosyl phosphatidylinositol (GPI)-anchored receptors that share ˜70% homology and a common mechanism of endocytosis-mediated folate uptake. FRs are the only known folate-specific transporters with a very high affinity (K_{D}<1 nM) for FA. Both FRα and FRβ are normally expressed low in healthy tissues, while selectively overexpressed in cancers including ovarian, kidney, lung, breast, and colon cancers, to meet the high folate demand of rapid dividing cells under low folate conditions. FRα and FRβ are able to transport a variety and broad size range of chemical conjugates of FA, antifolate drugs and immunological agents, as a result, FRs has been widely used as a target for tumor-selective drug delivery employing FA as a targeting ligand.

In some embodiments, the disclosure provides methods of treating a disease or disorder implicating the Wnt/β-catenin pathway and/or the AMPK pathway. In some embodiments, the disease or disorder is cancer or a hyperoliferative disease. In some embodiments, the cancer or hyperproliferative disease may be one or more of adrenocortical cancer, hepatocellular cancer, hepatoblastoma, malignant melanoma, ovarian cancer, Wilm's tumor, Barrett's esophageal cancer, prostate cancer, colon cancer, colorectal cancer, rectal cancer, pancreatic cancer, bladder cancer, breast cancer, gastric cancer, head & neck cancer, lung cancer, mesothelioma, cervical cancer, uterine cancer, myeloid leukemia cancer, lymphoid leukemia cancer, pilometricoma cancer, medulloblastoma cancer, glioblastoma, and familial adenomatous polyposis. In some embodiments, the cancer or hyperproliferative disease may include colon cancer. In some embodiments, the cancer or hyperproliferative disease may include colorectal cancer. In some embodiments, the cancer or hyperproliferative disease may include triple negative breast cancer.

In one aspect, the disclosure provides the development of a first in class small molecule with the dual activities of Wnt signaling inhibition and AMPK activation, via stabilization of the scaffold protein Axin. Although Axin stabilization by TNKS inhibitors are known, they have limited efficacy and gastrointestinal toxicity.

FRs are selectively overexpressed in cancers over healthy tissues. Therefore, FR-targeted FA-based hybrids provide a novel strategy to selectively deliver compounds with dual activity of Wnt inhibition and AMPK activation.

In one aspect, the disclosure provides an advanced HT-29-luc-D6 tumor Cecal implantation mouse model of CRC invasion and metastasis used to test selected compounds, which is used in addition to a commonly used animal model of intestinal neoplasia. In an embodiment, the model is highly clinically relevant to advanced human CRC for which highly effective targeted therapies are missing.

In one aspect, the disclosure provides novel Axin stabilizers with previously unrecognized mechanism of action useful as anti-CRC agents. By using a combined approach of biochemistry, biology and state-of-art proteomics, new knowledge on first-in-class small molecules that interfere with the binding of TNKS-binding protein1 (TAB182) to TNKS Wnt signaling and AMPK pathways are identified.

In some embodiments, cancers that may be treated by the compounds, compositions and methods described herein include, but are not limited to, the following:
1) Breast cancers, including, for example ER+ breast cancer, ER− breast cancer, her2− breast cancer, her2+ breast cancer, stromal tumors such as fibroadenomas, phyllodes tumors, and sarcomas, and epithelial tumors such as large duct papillomas; carcinomas of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, invasive lobular carcinoma, medullary carcinoma, colloid (mucinous) carcinoma, tubular carcinoma, and invasive papillary carcinoma; and miscellaneous malignant neoplasms. Further examples of breast cancers can include luminal A, lumina B, basal A, basal B, and triple negative breast cancer, which is estrogen receptor negative (ER−), progesterone receptor negative, and her2 negative (her2−). In some embodiments, the breast cancer may have a high risk Oncotype score;
2) Cardiac cancers, including, for example sarcoma, e.g., angiosarcoma, fibrosarcoma, rhabdomyosarcoma, and liposarcoma; myxoma; rhabdomyoma; fibroma; lipoma and teratoma;
3) Lung cancers, including, for example, bronchogenic carcinoma, e.g., squamous cell, undifferentiated small cell, undifferentiated large cell, and adenocarcinoma; alveolar and bronchiolar carcinoma; bronchial adenoma; sarcoma; lymphoma; chondromatous hamartoma; and mesothelioma;
4) Gastrointestinal cancer, including, for example, colon cancer, colorectal cancer (CRC), cancers of the esophagus, e.g., squamous cell carcinoma, adenocarcinoma, leiomyosarcoma, and lymphoma; cancers of the stomach, e.g., carcinoma, lymphoma, and leiomyosarcoma; cancers of the pancreas, e.g., ductal adenocarcinoma, insulinoma, glucagonoma, gastrinoma, carcinoid tumors, and vipoma; cancers of the small bowel, e.g., adenocarcinoma, lymphoma, carcinoid tumors, Kaposi's sarcoma, leiomyoma, hemangioma, lipoma, neurofibroma, and fibroma; cancers of the large bowel, e.g., adenocarcinoma, tubular adenoma, villous adenoma, hamartoma, and leiomyoma;
5) Genitourinary tract cancers, including, for example, cancers of the kidney, e.g., adenocarcinoma, Wilm's tumor (nephroblastoma), lymphoma, and leukemia; cancers of the bladder and urethra, e.g., squamous cell carcinoma, transitional cell carcinoma, and adenocarcinoma; cancers of the prostate, e.g., adenocarcinoma, and sarcoma; cancer of the testis, e.g., seminoma, teratoma, embryonal carcinoma, teratocarcinoma, choriocarcinoma, sarcoma, interstitial cell carcinoma, fibroma, fibroadenoma, adenomatoid tumors, and lipoma;
6) Liver cancers, including, for example, hepatoma, e.g., hepatocellular carcinoma; cholangiocarcinoma; hepatoblastoma; angiosarcoma; hepatocellular adenoma; and hemangioma;
7) Bone cancers, including, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors;
8) Nervous system cancers, including, for example, cancers of the skull, e.g., osteoma, hemangioma, granuloma, xanthoma, and osteitis deformans; cancers of the meninges, e.g., meningioma, meningiosarcoma, and gliomatosis; cancers of the brain, e.g., astrocytoma, medulloblastoma, glioma, ependymoma, germinoma (pinealoma), glioblastoma multiform, oligodendroglioma, schwannoma, retinoblastoma, and congenital tumors; and cancers of the spinal cord, e.g., neurofibroma, meningioma, glioma, and sarcoma;
9) Gynecological cancers, including, for example, cancers of the uterus, e.g., endometrial carcinoma; cancers of the cervix, e.g., cervical carcinoma, and pre tumor cervical dysplasia; cancers of the ovaries, e.g., ovarian carcinoma, including serous cystadenocarcinoma, mucinous cystadenocarcinoma, unclassified carcinoma, granulosa theca cell tumors, Sertoli Leydig cell tumors, dysgerminoma, and malignant teratoma; cancers of the vulva, e.g., squamous cell carcinoma, intraepithelial carcinoma, adenocarcinoma, fibrosarcoma, and melanoma; cancers of the vagina, e.g., clear cell carcinoma, squamous cell carcinoma, botryoid sarcoma, and embryonal rhabdomyosarcoma; and cancers of the fallopian tubes, e.g., carcinoma;
10) Hematologic cancers, including, for example, cancers of the blood, e.g., acute myeloid leukemia, chronic myeloid leukemia, acute lymphoblastic leukemia, chronic lymphocytic leukemia, myeloproliferative diseases, multiple myeloma, and myelodysplastic syndrome, Hodgkin's lymphoma, non Hodgkin's lymphoma (malignant lymphoma) and Waldenström's macroglobulinemia;
11) Skin cancers and skin disorders, including, for example, malignant melanoma, basal cell carcinoma, squamous cell carcinoma, Kaposi's sarcoma, moles dysplastic nevi, lipoma, angioma, dermatofibroma, keloids, and psoriasis;
12) Adrenal gland cancers, including, for example, neuroblastoma; and
13) Pancreatic cancer.

Cancers may be solid tumors that may or may not be metastatic. Cancers may also occur, as in leukemia, as a diffuse tissue. Thus, the term “tumor cell,” as provided herein, includes a cell afflicted by any one of the above identified disorders.

A method of treating cancer using a compound or composition as described herein may be combined with existing methods of treating cancers, for example by chemotherapy, irradiation, or surgery (e.g., oophorectomy). In some embodiments, a compound or composition can be administered before, during, or after another anticancer agent, additional therapeutic agent, or treatment.

### Compounds

In one aspect, the disclosure provides compounds of formula (I):

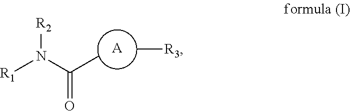

wherein ring A is a 5-membered substituted or unsubstituted heterocycle or heteroaryl;
wherein R₁ is selected from substituted or unsubstituted cycloalkyl, substituted or unsubstituted aryl, substituted or unsubstituted heterocycle, and substituted or unsubstituted heteroaryl;
R₂ is selected from H and substituted or unsubstituted alkyl; and
R₃ is selected from substituted or unsubstituted aryl and substituted or unsubstituted heteroaryl;
   or a pharmaceutically acceptable salt thereof.

In some embodiments, the compound of formula (I) comprises one or more fluoro groups. In some embodiments, the compound of formula (I) is covalently bonded to a targeting moiety, or is conjugated to a targeting moiety by way of a linking group.

In some embodiments, ring A is selected from substituted or unsubstituted triazole, substituted or unsubstituted tetrazole, substituted or unsubstituted pyrazole, substituted or unsubstituted pyrrole, and substituted or unsubstituted thiazole.

In some embodiments, the compound of formula (I) is a compound of any one of formula (10)-(15):

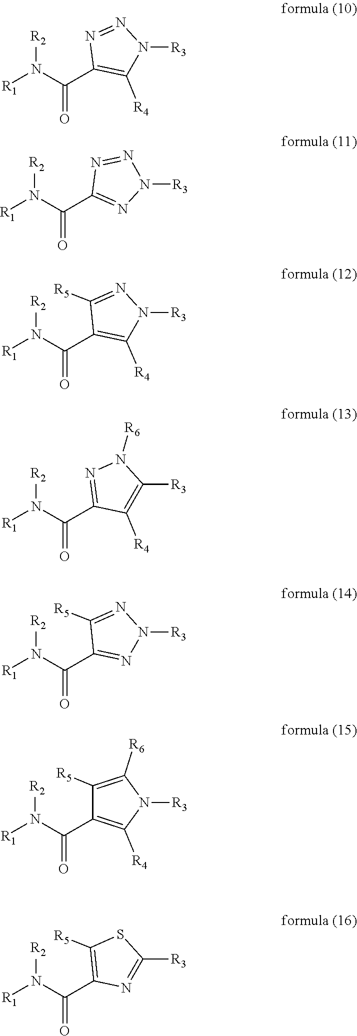

wherein each R₄, R₅, and R₆ is independently selected from H and substituted or unsubstituted alkyl; or a pharmaceutically acceptable salt thereof.

In some embodiments, R₁ is selected from substituted or unsubstituted quinoline, substituted or unsubstituted quinoxaline, substituted or unsubstituted benzothiazole, substituted or unsubstituted isoquinoline, substituted or unsubstituted pyridine, substituted or unsubstituted quinazoline, substituted or unsubstituted 1,5-naphthyridine, substituted or unsubstituted 1,8-naphthyridine, substituted or unsubstituted thiazole, and substituted or unsubstituted benzoxazole. In some embodiments, R₁ is substituted with one or more fluoro groups. In some embodiments, R₁ is covalently bonded to a targeting moiety, or is conjugated to a targeting moiety by way of a linking group.

In some embodiments, R₁ is selected from

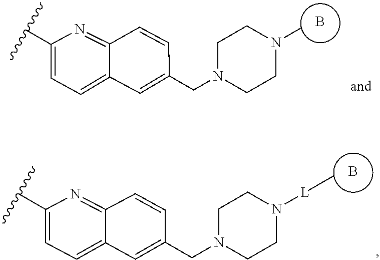

wherein L is a linking group and B is a targeting moiety. In some embodiments, the targeting moiety is selected from biotin, folic acid, and biguanide.

In some embodiments, L comprises one or more linking groups selected from optionally substituted —C₁₋₁₀ alkyl-, —O—C₁₋₁₀ alkyl-, —C₁₋₁₀ alkenyl-, —O—C₁₋₁₀ alkenyl-, —C₁₋₁₀ cycloalkenyl-, —O—C₁₋₁₀ cycloalkenyl-, —C₁₋₁₀ alkynyl-, —O—C₁₋₁₀ alkynyl-, —C₁₋₁₀ aryl-, —O—C₁₋₁₀—, -aryl-, —O—, —S—, —S—S—, —S(O)_{w}—, —C(O)—, —C(O)O—, —OC(O)—, —C(O)S—, —SC(O)—, —OC(O)O—, —N(R^{b})—, —C(O)N(R^{b})—, —N(R^{b})C(O)—, —OC(O)N(R^{b})—, —N(R)C(O)O—, —SC(O)N(R^{b})—, —N(R^{b})C(O)S—, —N(R^{b})C(O)N(R^{b})—, —N(R^{b})C(NR^{b})N(R^{b})—, —N(R^{b})S(O)_{w}—, —S(O)_{w}N(R^{b})—, —S(O)_{w}O—, —OS(O)_{w}—, —OS(O)_{w}O—, —O(O)P(OR)O—, (O)P(O—)₃, —O(S)P(OR^{b})O—, and (S)P(O—)₃, wherein w is 1 or 2, and R^{b} is independently hydrogen, optionally substituted alkyl, or optionally substituted aryl. In some embodiments, L comprises one or more linking groups selected from —C₁₋₁₀ alkyl-N(R^{b})—, —O—C₁₋₁₀ alkyl-, —O—, —N(R^{b})—, and —S—S—. In some embodiments, L comprises one or more linking group selected from

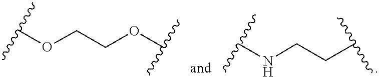 [SMILES: *OCCO* ; *CCN*]

In some embodiments, L comprises

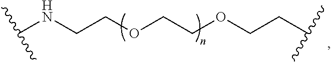

wherein n is an integer selected from 0-5. In some embodiments, L comprises

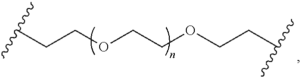

wherein n is an integer selected from 0-5.

In some embodiments, R₁ is selected from

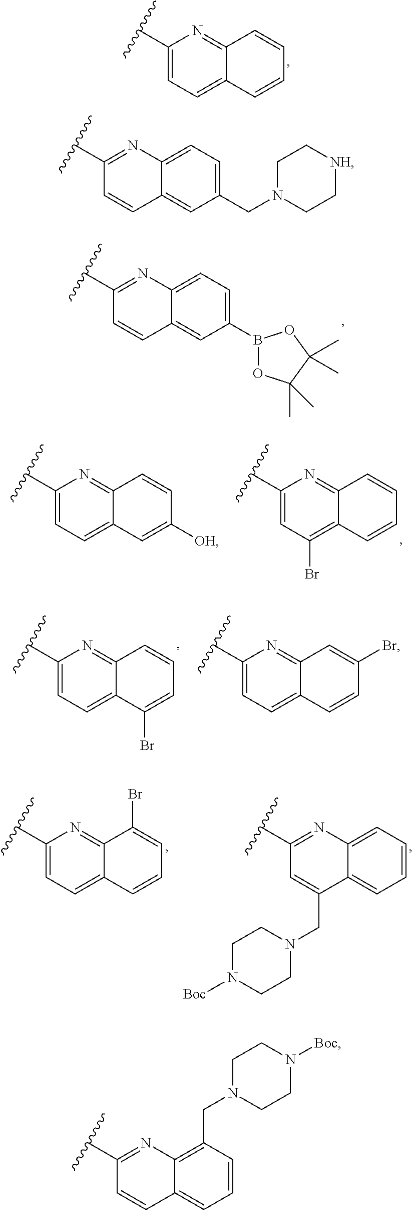

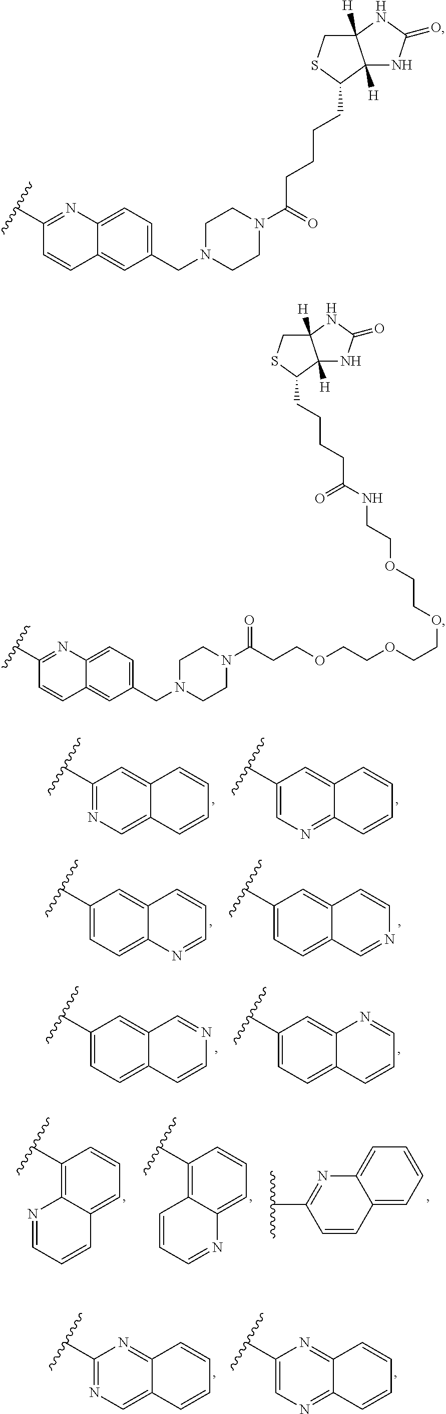

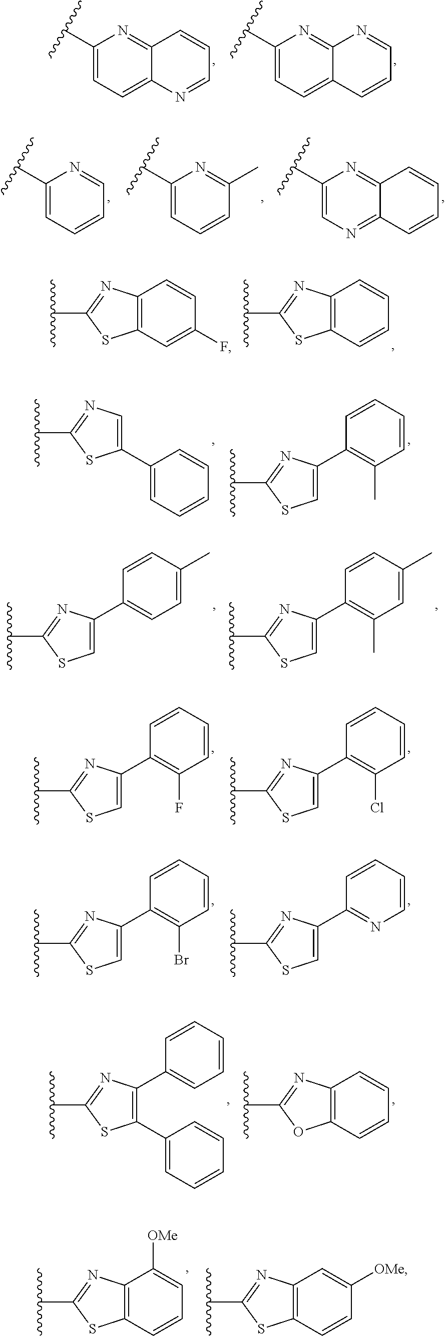

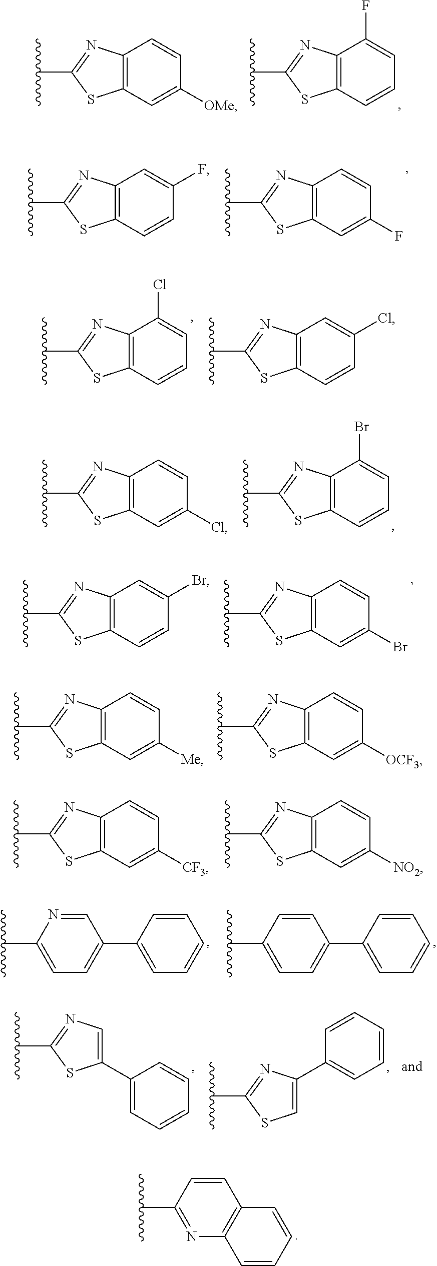

In some embodiments, R₁ is selected from

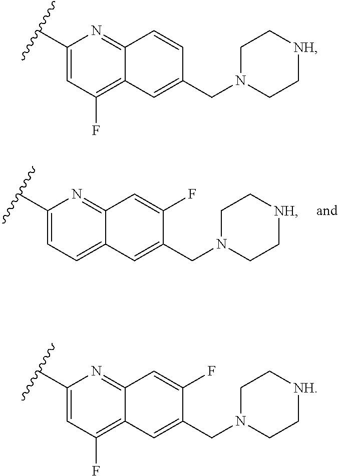 [SMILES: *c1cc(F)c2cc(CN3CCNCC3)ccc2n1 ; *c1ccc2cc(CN3CCNCC3)c(F)cc2n1 ; *c1cc(F)c2cc(CN3CCNCC3)c(F)cc2n1]

In some embodiments, R₃ is selected from substituted or unsubstituted pyridyl, unsubstituted aryl, and aryl substituted with one or more groups selected from halo, —CN, alkyl, fluroroalkyl, alkoxy, and fluoroalkoxy. In some embodiments, R₃ is substituted with one or more fluoro groups.

In some embodiments, R₃ is selected from

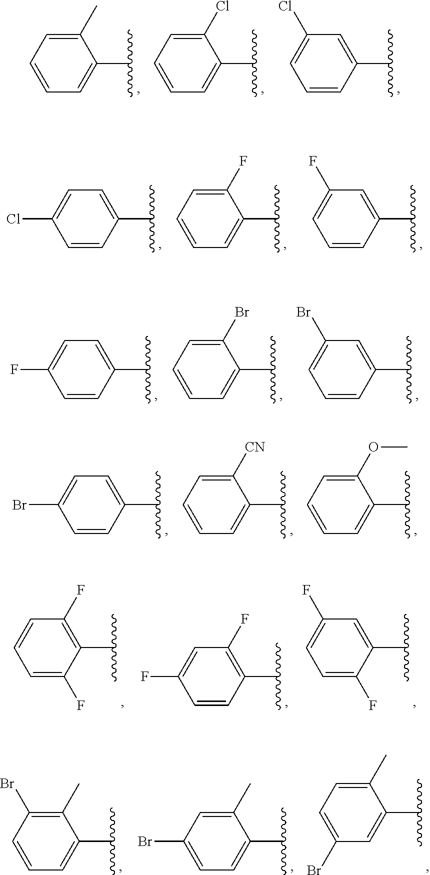

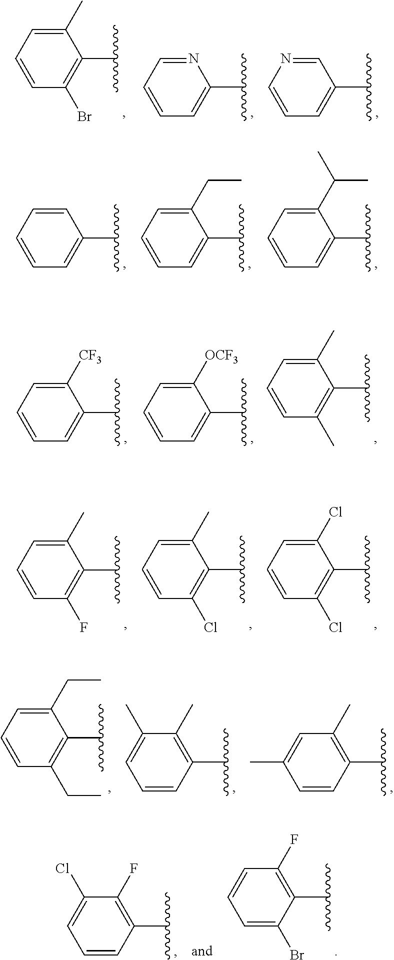

In some embodiments, R₃ is selected from

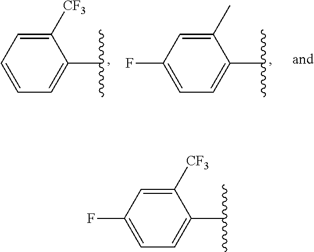

In some embodiments, each R₄, R₅, and R₆ is independently selected from H, methyl, ethyl, i-propyl, t-butyl, and —CF₃. In some embodiments, R₄ is-CF₃.

In some embodiments, the compound of formula (I) is a compound of any one of formula (20)-(29):

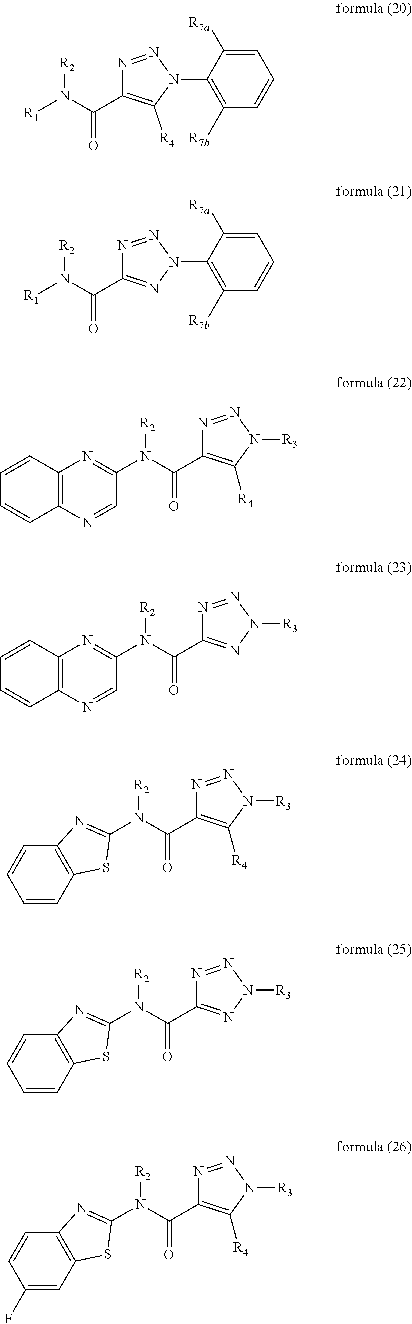

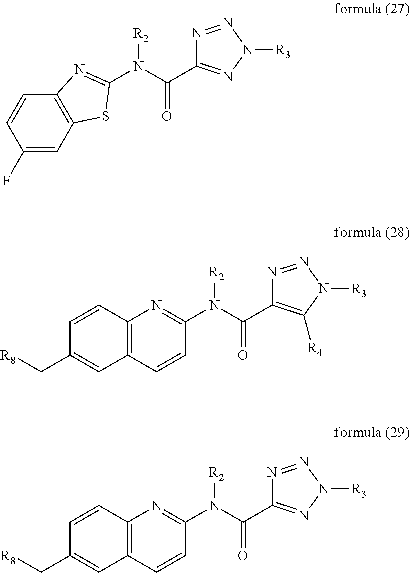

wherein R₄ is selected from H and substituted or unsubstituted alkyl;
each R₇ₐ and R_{7b} is independently selected from H, substituted or unsubstituted alkyl, halo, —CN, fluroroalkyl, alkoxy; and
R₈ is a substituted or unsubstituted heterocycle, or a pharmaceutically acceptable salt thereof.

In some embodiments, the compound of formula (I) is a compound of any one of formula (30)-(31):

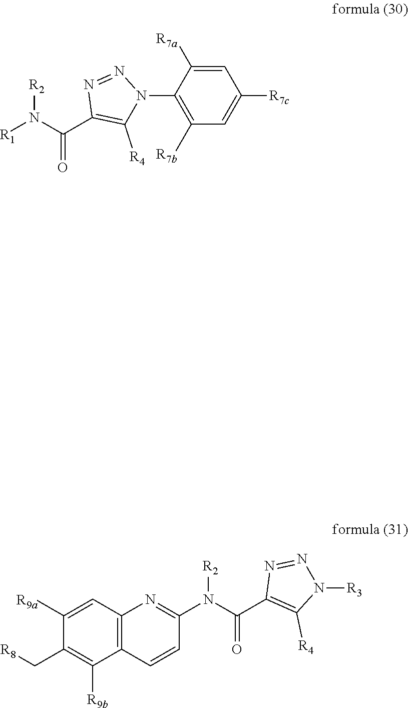

wherein R₄ is selected from H and substituted or unsubstituted alkyl; each R₇ₐ, R_{7b}, R_{7c}, R₉ₐ, and R_{9b}, is independently selected from H, substituted or unsubstituted alkyl, halo, —CN, fluroroalkyl, alkoxy; and
R₅ is a substituted or unsubstituted heterocycle, or a pharmaceutically acceptable salt thereof;
with the proviso that at least one of R₇ₐ, R_{7b}, R_{7c}, R₉ₐ, and R_{9b} is a fluoro group, and/or R₄ is —CF₃.

In some embodiments, R₅ is substituted or unsubstituted piperazine.

In some embodiments, R₁ is selected from N

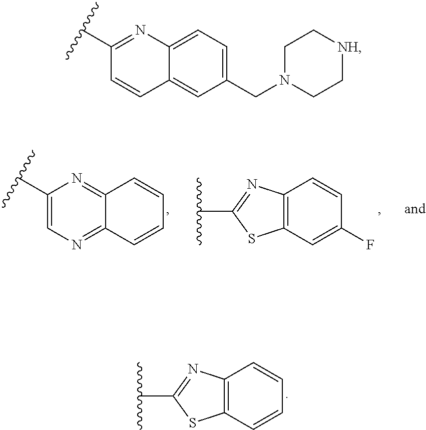

In some embodiments, R₁ is substituted with one or more fluoro groups. In some embodiments, R₁ is covalently bonded to a targeting moiety, or is conjugated to a targeting moiety by way of a linking group. In some embodiments, R₁ is selected from

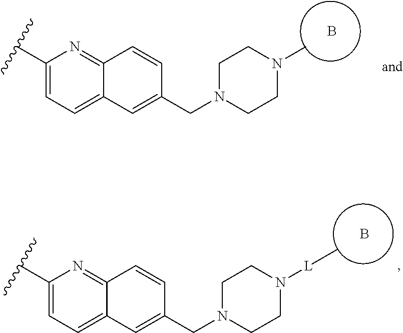

wherein L is a linking group and B is a targeting moiety. In some embodiments, the targeting moiety is selected from biotin, folic acid, and biguanide. In some embodiments, R₁ is selected from

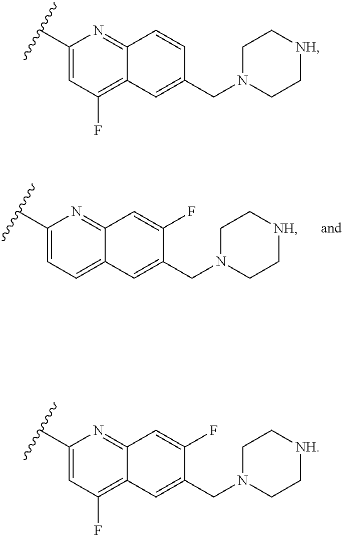 [SMILES: *c1cc(F)c2cc(CN3CCNCC3)ccc2n1 ; *c1ccc2cc(CN3CCNCC3)c(F)cc2n1 ; "and" ; *c1cc(F)c2cc(CN3CCNCC3)c(F)cc2n1]

In some embodiments, R₂ is H.

In some embodiments, R₃ is selected from

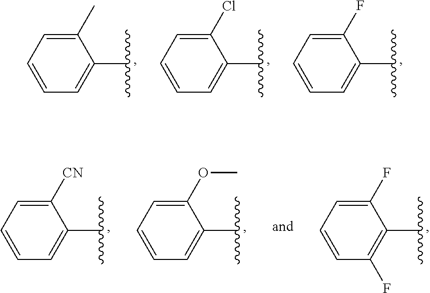

In some embodiments, R₄ is C₁-C₆ alkyl. In some embodiments, R₄ is selected from methyl and ethyl.

In some embodiments, R₈ is

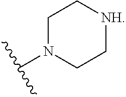 [SMILES: *N1CCNCC1]

In some embodiments, the compound is a compound of any one of formula 1001-1126, or a pharmaceutically acceptable salt thereof:

| Formula No. | Structure |
|---|---|
| 1001 (YA6060; YA6060) | 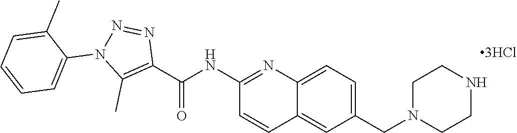 [SMILES: Cc1ccccc1-n1nnc(C(=O)Nc2ccc3cc(CN4CCNCC4)ccc3n2)c1C] |
| 1002 (YA10951) | 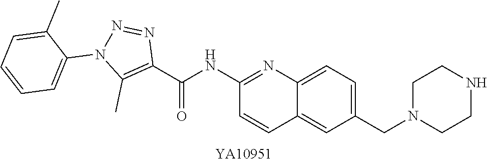 |
| 1003 (YA6150) | 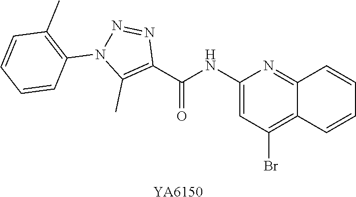 |
| 1004 (YA6167) | 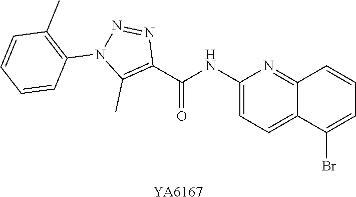 |
| 1005 (YA1126) | 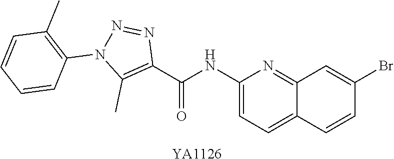 |
| 1006 (YA6146) | 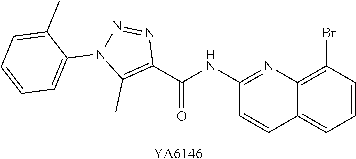 |
| 1007 (YA6159) | 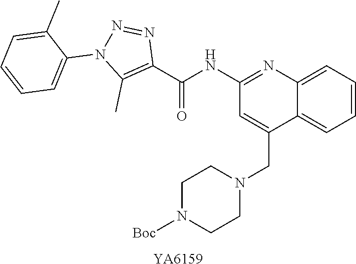 |
| 1008 (YA6160) | 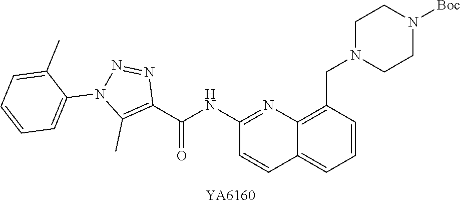 |
| 1009 (YA4182) | 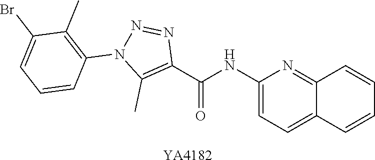 |
| 1010 (YA4179) | 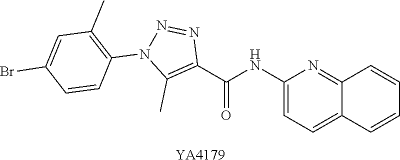 |
| 1011 (YA1130) | 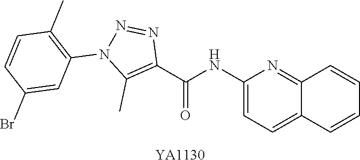 |
| 1012 (YA1128) | 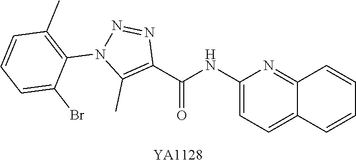 |
| 1013 (YA6023) | 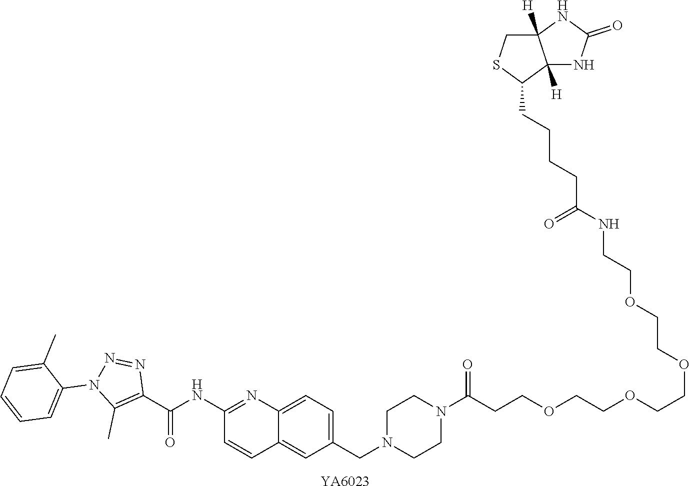 |
| 1014 (YA1103) | 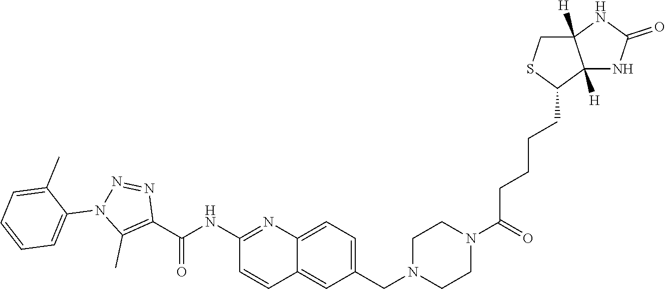 [SMILES: Cc1ccccc1-n1nnc(C(=O)Nc2ccc3cc(CN4CCN(C(=O)CCCC[C@@H]5SC[C@@H]6NC(=O)N[C@@H]65)CC4)ccc3n2)c1C] |
| 1015 (YA6164) | 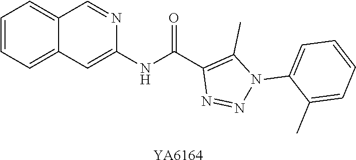 |
| 1016 (YA6161) | 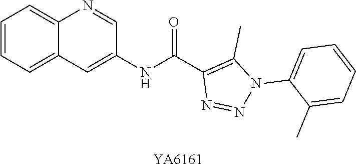 |
| 1017 (YA6056) | 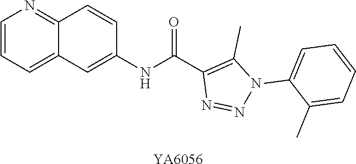 |
| 1018 (YA6169-2) | 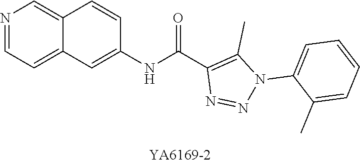 [SMILES: Cc1ccccc1-n1nnc(C(=O)Nc2ccc3cnccc3c2)c1C ; "YA6169-2"] |
| 1019 (YA6169-1) | 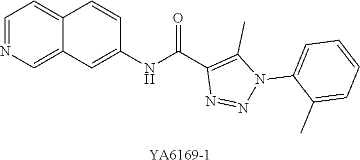 [SMILES: Cc1ccccc1-n1nnc(C(=O)Nc2ccc3ccncc3c2)c1C ; "YA6169-1"] |
| 1020 (YA6162) | 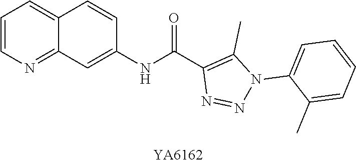 |
| 1021 (YA6055) | 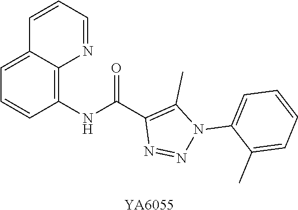 |
| 1022 (YA6169-3) | 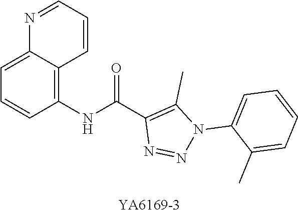 [SMILES: Cc1ccccc1-n1nnc(C(=O)Nc2cccc3ncccc23)c1C ; "YA6169-3"] |
| 1023 (YA6061) | 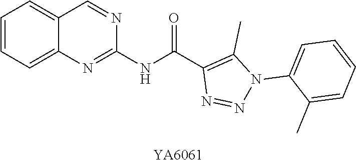 |
| 1024 (YA6045) | 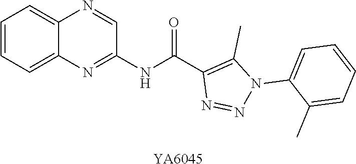 |
| 1025 (YA6147) | 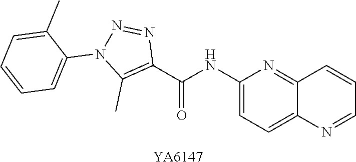 |
| 1026 (YA6047) | 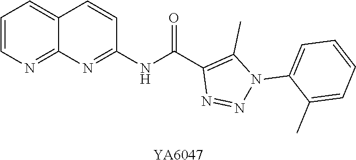 |
| 1027 (YA7013-1) | 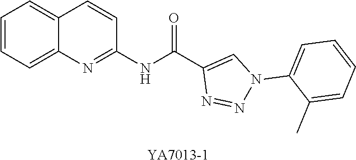 [SMILES: Cc1ccccc1-n1cc(C(=O)Nc2ccc3ccccc3n2)nn1 ; "YA7013-1"] |
| 1028 (YA4114) | 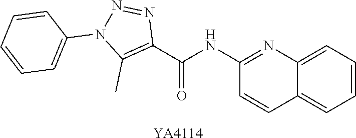 |
| 1029 (YA1144) | 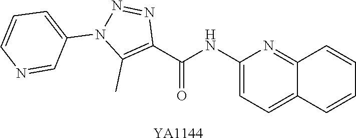 |
| 1030 (YA4112) | 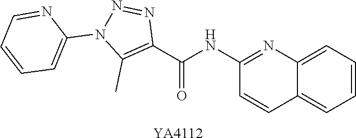 |
| 1031 (YA1144-1) | 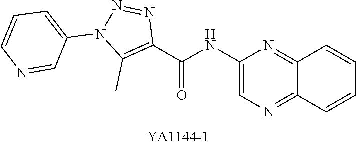 [SMILES: Cc1c(C(=O)Nc2cnc3ccccc3n2)nnn1-c1cccnc1 ; "YA1144-1"] |
| 1032 (YA6136) | 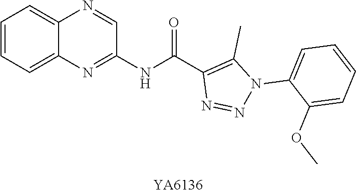 |
| 1033 (YA6137) | 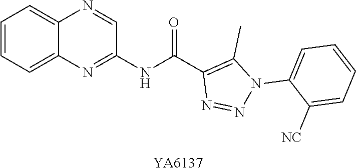 |
| 1034 (YA6138) | 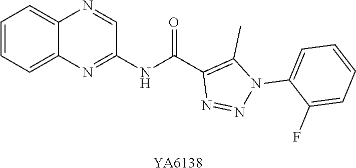 |
| 1035 (YA6139) | 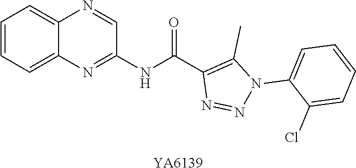 |
| 1036 (YA6141) | 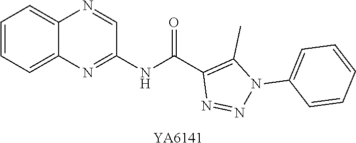 |
| 1037 (YA70031) | 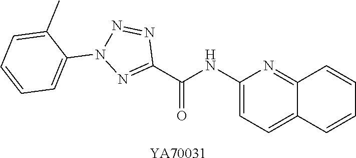 |
| 1038 (YA6171) | 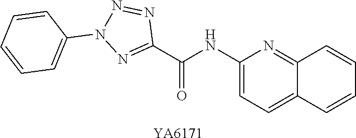 |
| 1039 (YA70032) | 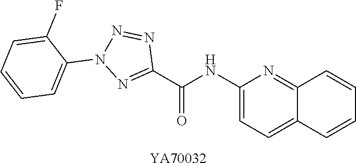 |
| 1040 (YA70033) | 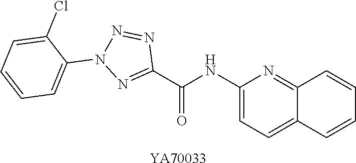 |
| 1041 (YA4184) | 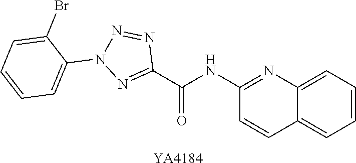 |
| 1042 (YA70034) | 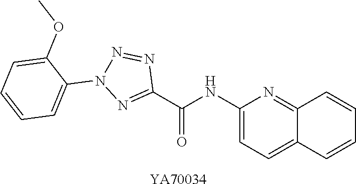 |
| 1043 (YA70035) | 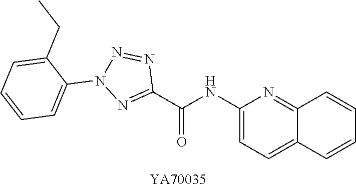 |
| 1044 (YA70036) | 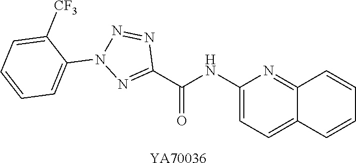 |
| 1045 (YA70037) | 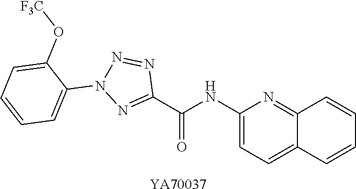 |
| 1046 (YA70038) | 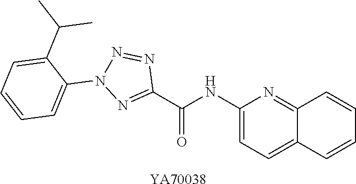 |
| 1047 (YA70041) | 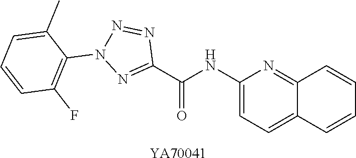 |
| 1048 (YA70042) | 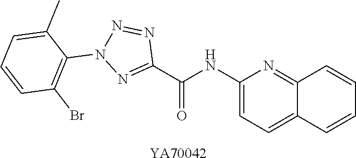 |
| 1049 (YA70043) | 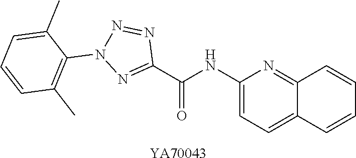 |
| 1050 (YA70044) | 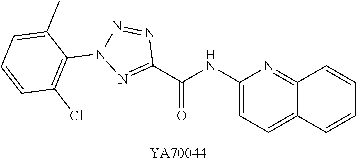 YA70044 |
| 1051 (YA70045) | 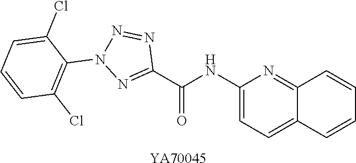 YA70045 |
| 1052 (YA70046) | 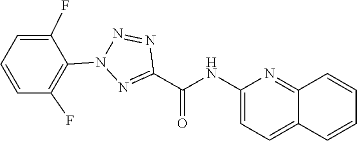 [SMILES: O=C(Nc1ccc2ccccc2n1)c1nnn(-c2c(F)cccc2F)n1] YA70046 |
| 1053 (YA70047) | 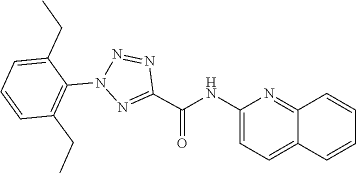 [SMILES: CCc1cccc(CC)c1-n1nnc(C(=O)Nc2ccc3ccccc3n2)n1] YA70047 |
| 1054 (YA6177) | 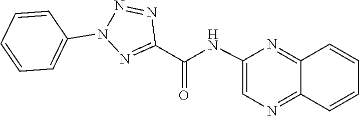 [SMILES: O=C(Nc1cnc2ccccc2n1)c1nnn(-c2ccccc2)n1] YA6177 |
| 1055 (YA7083) | 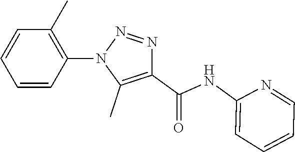 [SMILES: Cc1ccccc1-n1nnc(C(=O)Nc2ccccn2)c1C] YA7083 |
| 1056 (YA7084) | 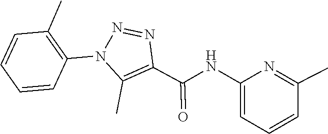 [SMILES: Cc1cccc(NC(=O)c2nnn(-c3ccccc3C)c2C)n1] YA7084 |
| 1057 (YA4117) | 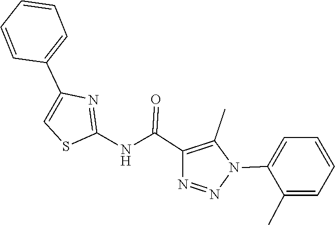 [SMILES: Cc1ccccc1-n1nnc(C(=O)Nc2nc(-c3ccccc3)cs2)c1C] YA4117 |
| 1058 (YA4085) | 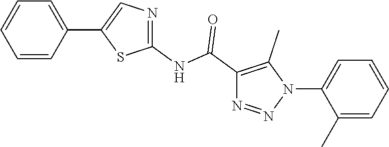 [SMILES: Cc1ccccc1-n1nnc(C(=O)Nc2ncc(-c3ccccc3)s2)c1C] YA7085 |
| 1059 (YA4133) | 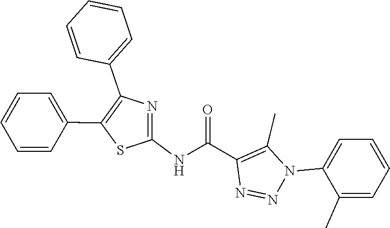 [SMILES: Cc1ccccc1-n1nnc(C(=O)Nc2nc(-c3ccccc3)c(-c3ccccc3)s2)c1C] YA4133 |
| 1060 (YA4105) | 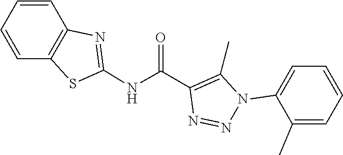 [SMILES: Cc1ccccc1-n1nnc(C(=O)Nc2nc3ccccc3s2)c1C] YA4105 |
| 1061 (YA4061) | 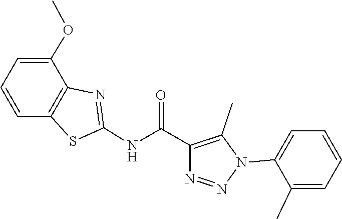 [SMILES: COc1cccc2sc(NC(=O)c3nnn(-c4ccccc4C)c3C)nc12] YA4061 |
| 1062 (YA4045) | 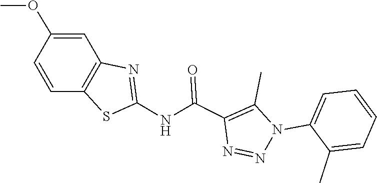 [SMILES: COc1ccc2sc(NC(=O)c3nnn(-c4ccccc4C)c3C)nc2c1] YA4045 |
| 1063 (YA4091) | 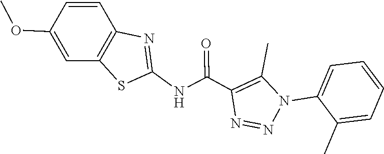 [SMILES: COc1ccc2nc(NC(=O)c3nnn(-c4ccccc4C)c3C)sc2c1] YA4091 |
| 1064 (YA4093) | 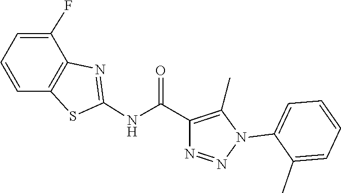 [SMILES: Cc1ccccc1-n1nnc(C(=O)Nc2nc3c(F)cccc3s2)c1C] YA4093 |
| 1065 (YA4101) | 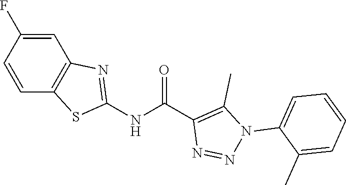 [SMILES: Cc1ccccc1-n1nnc(C(=O)Nc2nc3cc(F)ccc3s2)c1C] YA4101 |
| 1066 (YA4103) | 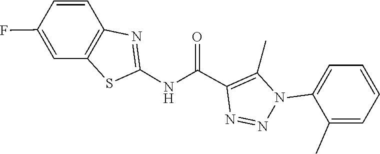 [SMILES: Cc1ccccc1-n1nnc(C(=O)Nc2nc3ccc(F)cc3s2)c1C] YA4103 |
| 1067 (YA4095) | 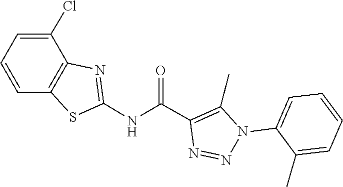 [SMILES: Cc1ccccc1-n1nnc(C(=O)Nc2nc3c(Cl)cccc3s2)c1C] YA4095 |
| 1068 (YA4013) | 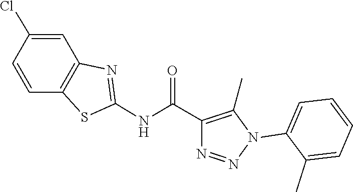 [SMILES: Cc1ccccc1-n1nnc(C(=O)Nc2nc3cc(Cl)ccc3s2)c1C] YA4013 |
| 1069 (YA4071) | 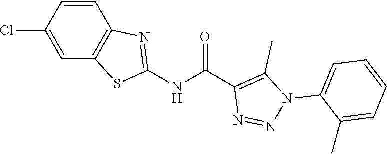 [SMILES: Cc1ccccc1-n1nnc(C(=O)Nc2nc3ccc(Cl)cc3s2)c1C] YA4071 |
| 1070 (YA4097) | 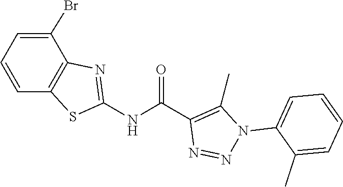 [SMILES: Cc1ccccc1-n1nnc(C(=O)Nc2nc3c(Br)cccc3s2)c1C] YA4097 |
| 1071 (YA4047) | 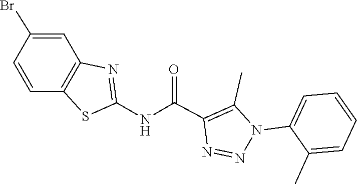 [SMILES: Cc1ccccc1-n1nnc(C(=O)Nc2nc3cc(Br)ccc3s2)c1C] YA4047 |
| 1072 (YA4059) | 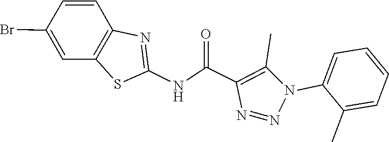 [SMILES: Cc1ccccc1-n1nnc(C(=O)Nc2nc3ccc(Br)cc3s2)c1C] YA4059 |
| 1073 (YA4069) | 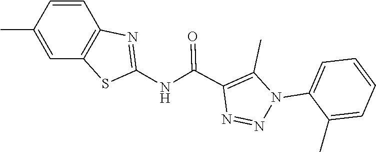 [SMILES: Cc1ccc2nc(NC(=O)c3nnn(-c4ccccc4C)c3C)sc2c1] YA4069 |
| 1074 (YA4065) | 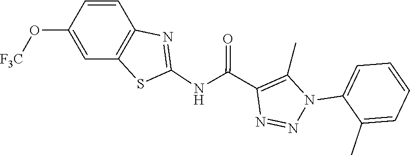 [SMILES: Cc1ccccc1-n1nnc(C(=O)Nc2nc3ccc(OC(F)(F)F)cc3s2)c1C] YA4065 |
| 1075 (YA4063) | 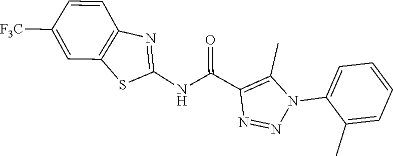 [SMILES: Cc1ccccc1-n1nnc(C(=O)Nc2nc3ccc(C(F)(F)F)cc3s2)c1C] YA4063 |
| 1076 (YA4057) | 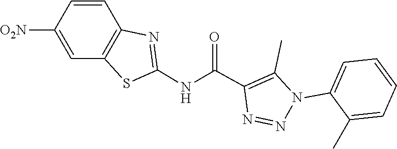 [SMILES: Cc1ccccc1-n1nnc(C(=O)Nc2nc3ccc([N+](=O)[O-])cc3s2)c1C] YA4057 |
| 1077 (YA6029) | 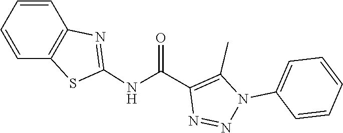 [SMILES: Cc1c(C(=O)Nc2nc3ccccc3s2)nnn1-c1ccccc1] YA6029 |
| 1078 (YA4029) | 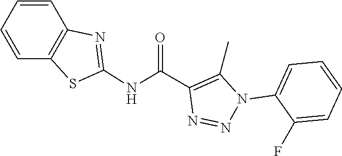 [SMILES: Cc1c(C(=O)Nc2nc3ccccc3s2)nnn1-c1ccccc1F] YA4029 |
| 1079 (YA1072) | 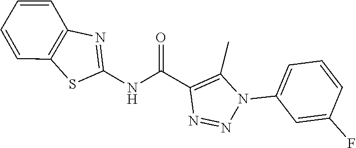 [SMILES: Cc1c(C(=O)Nc2nc3ccccc3s2)nnn1-c1cccc(F)c1] YA1072 |
| 1080 (YA1076) | 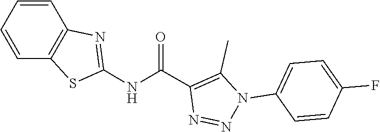 [SMILES: Cc1c(C(=O)Nc2nc3ccccc3s2)nnn1-c1ccc(F)cc1] YA1076 |
| 1081 (YA6027) | 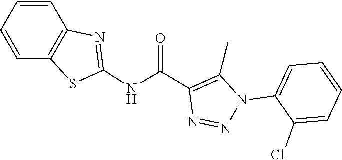 [SMILES: Cc1c(C(=O)Nc2nc3ccccc3s2)nnn1-c1ccccc1Cl] YA6027 |
| 1082 (YA4055) | 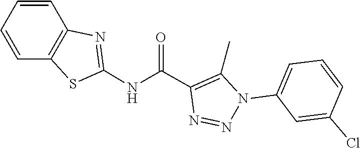 [SMILES: Cc1c(C(=O)Nc2nc3ccccc3s2)nnn1-c1cccc(Cl)c1] YA4055 |
| 1083 (YA1074) | 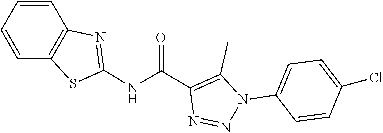 [SMILES: Cc1c(C(=O)Nc2nc3ccccc3s2)nnn1-c1ccc(Cl)cc1] YA1074 |
| 1084 (YA4003) | 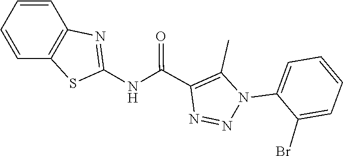 [SMILES: Cc1c(C(=O)Nc2nc3ccccc3s2)nnn1-c1ccccc1Br] YA4003 |
| 1085 (YA1050) | 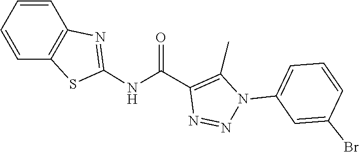 [SMILES: Cc1c(C(=O)Nc2nc3ccccc3s2)nnn1-c1cccc(Br)c1] YA1050 |
| 1086 (YA1086) | 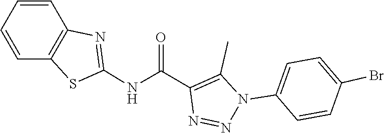 [SMILES: Cc1c(C(=O)Nc2nc3ccccc3s2)nnn1-c1ccc(Br)cc1] YA1086 |
| 1087 (YA6089) | 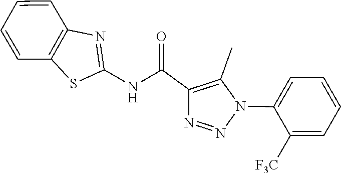 [SMILES: Cc1c(C(=O)Nc2nc3ccccc3s2)nnn1-c1ccccc1C(F)(F)F] YA6089 |
| 1088 (YA1038) | 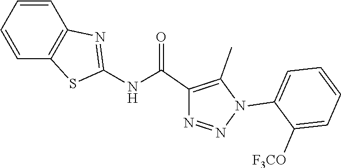 [SMILES: Cc1c(C(=O)Nc2nc3ccccc3s2)nnn1-c1ccccc1OC(F)(F)F] YA1038 |
| 1089 (YA1046) | 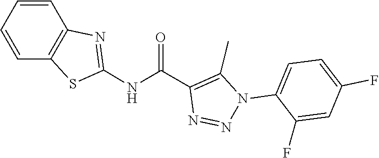 [SMILES: Cc1c(C(=O)Nc2nc3ccccc3s2)nnn1-c1ccc(F)cc1F] YA10146 |
| 1090 (YA1084) | 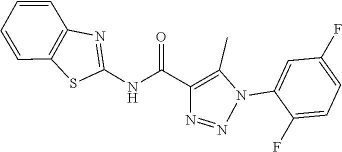 YA1084 |
| 1091 (YA6090) | 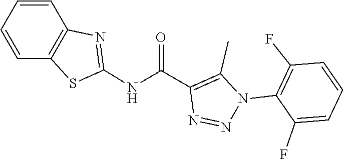 [SMILES: Cc1c(C(=O)Nc2nc3ccccc3s2)nnn1-c1c(F)cccc1F] YA6090 |
| 1092 (YA1066) | 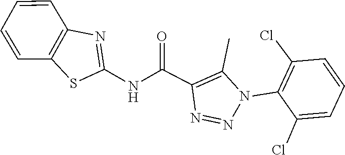 [SMILES: Cc1c(C(=O)Nc2nc3ccccc3s2)nnn1-c1c(Cl)cccc1Cl] YA1066 |
| 1093 (YA1042) | 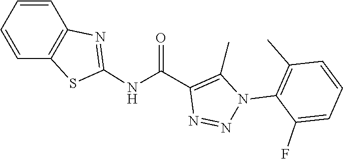 [SMILES: Cc1cccc(F)c1-n1nnc(C(=O)Nc2nc3ccccc3s2)c1C] YA1042 |
| 1094 (YA1068) | 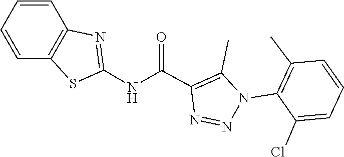 [SMILES: Cc1cccc(Cl)c1-n1nnc(C(=O)Nc2nc3ccccc3s2)c1C] YA1068 |
| 1095 (YA1064) | 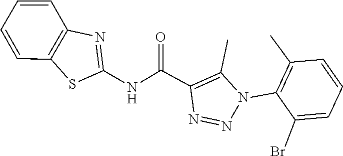 [SMILES: Cc1cccc(Br)c1-n1nnc(C(=O)Nc2nc3ccccc3s2)c1C] YA1064 |
| 1096 (YA4001) | 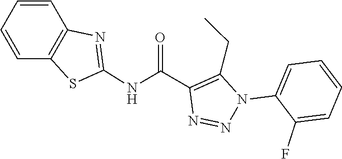 [SMILES: CCc1c(C(=O)Nc2nc3ccccc3s2)nnn1-c1ccccc1F] YA4001 |
| 1097 (YA4005) | 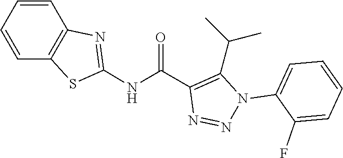 [SMILES: CC(C)c1c(C(=O)Nc2nc3ccccc3s2)nnn1-c1ccccc1F] YA4005 |
| 1098 (YA6176) | 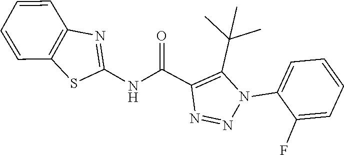 [SMILES: CC(C)(C)c1c(C(=O)Nc2nc3ccccc3s2)nnn1-c1ccccc1F] YA6176 |
| 1099 (YA6168) | 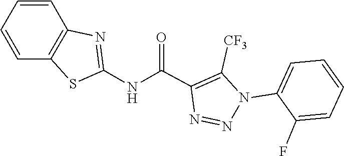 [SMILES: O=C(Nc1nc2ccccc2s1)c1nnn(-c2ccccc2F)c1C(F)(F)F] YA6168 |
| 1100 (YA4155) | 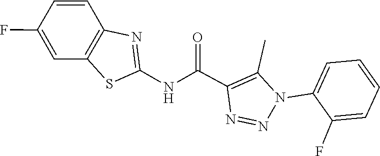 [SMILES: Cc1c(C(=O)Nc2nc3ccc(F)cc3s2)nnn1-c1ccccc1F] YA4155 |
| 1101 (YA4165) | 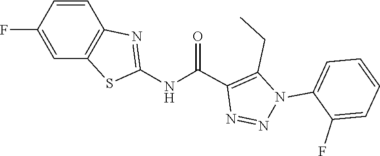 [SMILES: CCc1c(C(=O)Nc2nc3ccc(F)cc3s2)nnn1-c1ccccc1F] YA4165 |
| 1102 (YA6172) | 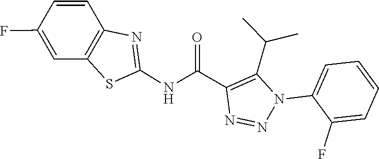 [SMILES: CC(C)c1c(C(=O)Nc2nc3ccc(F)cc3s2)nnn1-c1ccccc1F] YA6172 |
| 1103 (YA6174) | 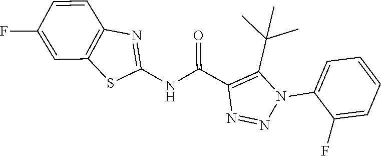 [SMILES: CC(C)(C)c1c(C(=O)Nc2nc3ccc(F)cc3s2)nnn1-c1ccccc1F] YA6174 |
| 1104 (YA6175) | 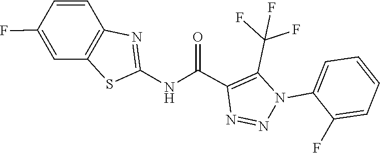 [SMILES: O=C(Nc1nc2ccc(F)cc2s1)c1nnn(-c2ccccc2F)c1C(F)(F)F] YA6175 |
| 1105 (YA1124) | 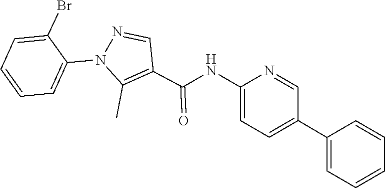 [SMILES: Cc1c(C(=O)Nc2ccc(-c3ccccc3)cn2)cnn1-c1ccccc1Br] YA1124 |
| 1106 (YA1122) | 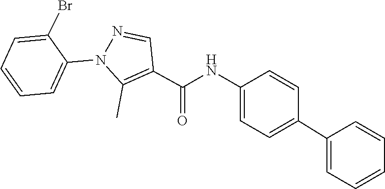 [SMILES: Cc1c(C(=O)Nc2ccc(-c3ccccc3)cc2)cnn1-c1ccccc1Br] YA1122 |
| 1107 (YA6152) | 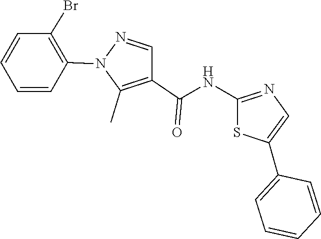 [SMILES: Cc1c(C(=O)Nc2ncc(-c3ccccc3)s2)cnn1-c1ccccc1Br] YA6152 |
| 1108 (YA4087) | 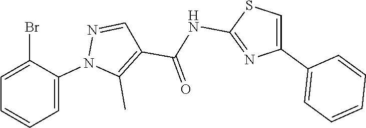 [SMILES: Cc1c(C(=O)Nc2nc(-c3ccccc3)cs2)cnn1-c1ccccc1Br] YA4087 |
| 1109 (YA4033) | 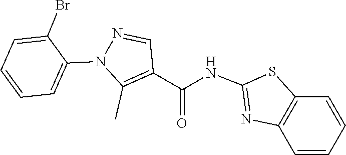 [SMILES: Cc1c(C(=O)Nc2nc3ccccc3s2)cnn1-c1ccccc1Br] YA4033 |
| 1110 (YA7018) | 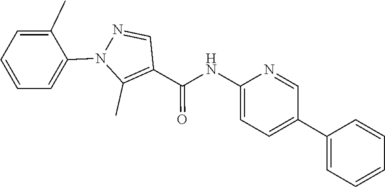 [SMILES: Cc1ccccc1-n1ncc(C(=O)Nc2ccc(-c3ccccc3)cn2)c1C] YA7018 |
| 1111 (YA7016) | 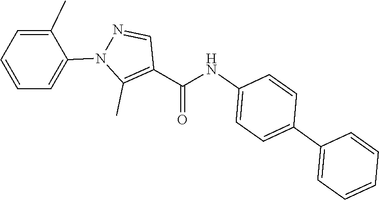 [SMILES: Cc1ccccc1-n1ncc(C(=O)Nc2ccc(-c3ccccc3)cc2)c1C] YA7016 |
| 1112 (YA7019) | 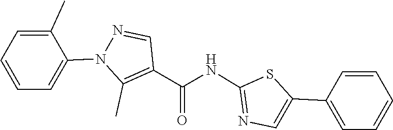 [SMILES: Cc1ccccc1-n1ncc(C(=O)Nc2ncc(-c3ccccc3)s2)c1C] YA7019 |
| 1113 (YA7015) | 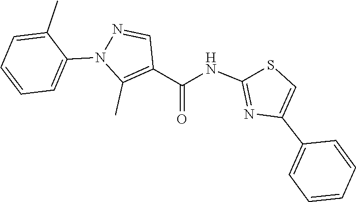 [SMILES: Cc1ccccc1-n1ncc(C(=O)Nc2nc(-c3ccccc3)cs2)c1C] YA7015 |
| 1114 (YA6179) | 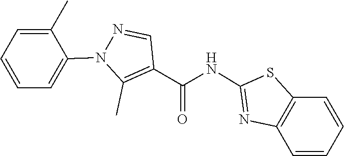 [SMILES: Cc1ccccc1-n1ncc(C(=O)Nc2nc3ccccc3s2)c1C] YA6179 |
| 1115 (YA4011) | 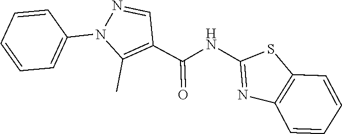 [SMILES: Cc1c(C(=O)Nc2nc3ccccc3s2)cnn1-c1ccccc1] YA4011 |
| 1116 (YA6149) | 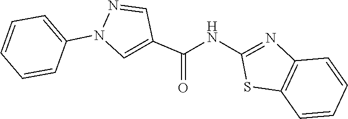 [SMILES: O=C(Nc1nc2ccccc2s1)c1cnn(-c2ccccc2)c1] YA6149 |
| 1117 (YA6143) | 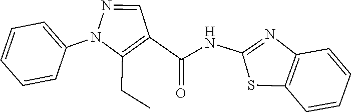 [SMILES: CCc1c(C(=O)Nc2nc3ccccc3s2)cnn1-c1ccccc1] YA6143 |
| 1118 (YA6144) | 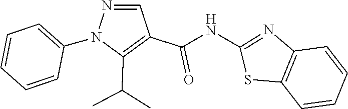 [SMILES: CC(C)c1c(C(=O)Nc2nc3ccccc3s2)cnn1-c1ccccc1] YA6144 |
| 1119 (YA4009) | 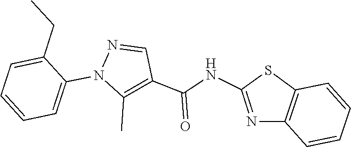 [SMILES: CCc1ccccc1-n1ncc(C(=O)Nc2nc3ccccc3s2)c1C] YA4009 |
| 1120 (YA7020) | 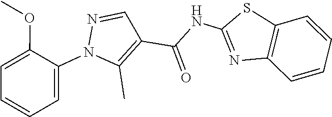 [SMILES: COc1ccccc1-n1ncc(C(=O)Nc2nc3ccccc3s2)c1C] YA7020 |
| 1121 (YA4007) | 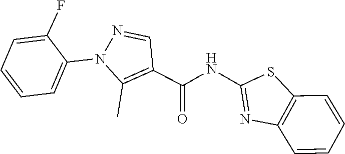 [SMILES: Cc1c(C(=O)Nc2nc3ccccc3s2)cnn1-c1ccccc1F] YA4007 |
| 1122 (YA4025) | 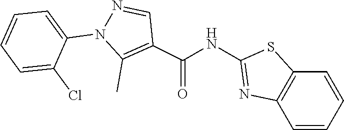 [SMILES: Cc1c(C(=O)Nc2nc3ccccc3s2)cnn1-c1ccccc1Cl] YA4025 |
| 1123 (YA4021) | 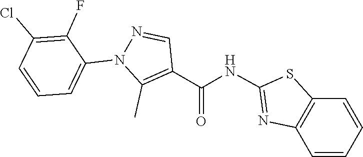 [SMILES: Cc1c(C(=O)Nc2nc3ccccc3s2)cnn1-c1cccc(Cl)c1F] YA4021 |
| 1124 (YA4019) | 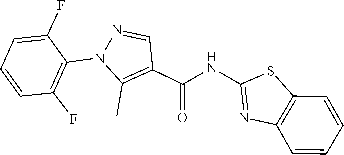 [SMILES: Cc1c(C(=O)Nc2nc3ccccc3s2)cnn1-c1c(F)cccc1F] YA4019 |
| 1125 (YA4017) | 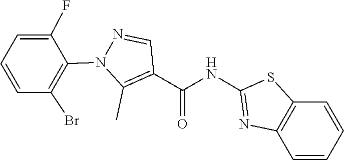 [SMILES: Cc1c(C(=O)Nc2nc3ccccc3s2)cnn1-c1c(F)cccc1Br] YA4017 |
| 1126 (YA4023) | 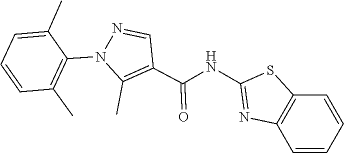 [SMILES: Cc1cccc(C)c1-n1ncc(C(=O)Nc2nc3ccccc3s2)c1C] YA4023 |

In some embodiments, the compound is a compound of any one of formula 1001, 1002, 1024, 1032-1036, 1078, 1096, 1100, 1101, 1114, 1121, 1122, or 1124, or a pharmaceutically acceptable salt thereof. In some embodiments, the compound is a compound of formula 1001.

In one aspect, the disclosure provides compounds of formula (II):

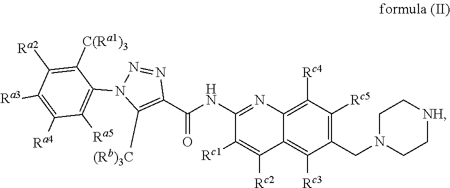

wherein R^{a1}, R^{a2}, R^{a3}, R^{a4}, R^{a5}, R^{b}, R^{c1}, R^{c2}, R^{c3}, R^{c4}, and R^{c5} at each occurrence is independently selected from H, halo, alkyl, alkoxy, and aryl,
with the proviso that one or more R^{a1}, R^{a2}, R^{a3}, R^{a4}, R^{a5}, R^{b}, R^{c1}, R^{c2}, R^{c3}, R^{c4}, and R^{c5} is fluoro;
or a pharmaceutically acceptable salt thereof.

In some embodiments, one or more R^{a1} is fluoro. In some embodiments, each occurrence of R^{a1} is fluoro.

In some embodiments, R^{a3} is fluoro.

In some embodiments, one or more R^{b} is fluoro. In some embodiments, each occurrence of R^{b} is fluoro.

In some embodiments, R^{c2} is fluoro.

In some embodiments, R^{c5} is fluoro.

In some embodiments, the compound comprises only one fluoro group. In some embodiments, the only one fluoro group is at R^{a2}, R^{a3}, R^{a4}, R^{a5}, R^{c1}, R^{c2}, R^{c3}, R^{c4}, or R^{c5}. In some embodiments, the remaining R^{a2}, R^{a3}, R^{a4}, R^{a5}, R^{c1}, R^{c2}, R^{c3}, R^{c4}, and R^{c5} are hydrogen, and each occurrence of Rai is hydrogen, and each occurrence of R^{b} is hydrogen. In some embodiments, the compound comprises exactly three fluoro groups, wherein the three fluoro groups are at each occurrence of Rai or at each occurrence of R^{b}. In some embodiments, the remaining R^{a1} or R^{b} are at each occurrence hydrogen, and R^{a2}, R^{a3}, R^{a4}, R^{a5}, R^{c1}, R^{c2}, R^{c3}, R^{c4}, or R^{c5} are each hydrogen.

In some embodiments, the compound of formula (II) is a compound of any one of formula 2001-2031, or a pharmaceutically acceptable salt thereof:

| Formula No. | Structure |
|---|---|
| 2001 | 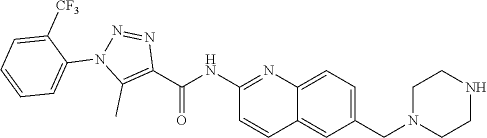 [SMILES: Cc1c(C(=O)Nc2ccc3cc(CN4CCNCC4)ccc3n2)nnn1-c1ccccc1C(F)(F)F] |
| 2002 | 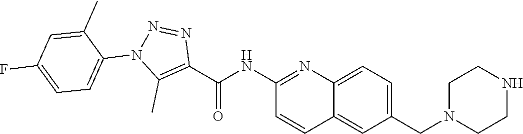 [SMILES: Cc1cc(F)ccc1-n1nnc(C(=O)Nc2ccc3cc(CN4CCNCC4)ccc3n2)c1C] |
| 2003 | 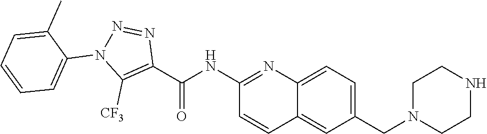 [SMILES: Cc1ccccc1-n1nnc(C(=O)Nc2ccc3cc(CN4CCNCC4)ccc3n2)c1C(F)(F)F] |
| 2004 | 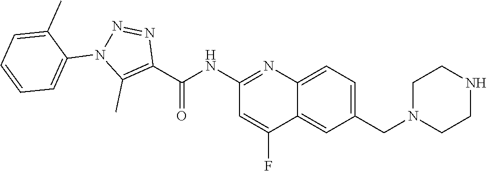 [SMILES: Cc1ccccc1-n1nnc(C(=O)Nc2cc(F)c3cc(CN4CCNCC4)ccc3n2)c1C] |
| 2005 | 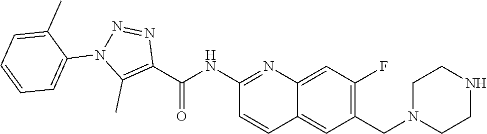 [SMILES: Cc1ccccc1-n1nnc(C(=O)Nc2ccc3cc(CN4CCNCC4)c(F)cc3n2)c1C] |
| 2006 | 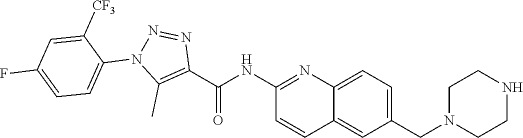 [SMILES: Cc1c(C(=O)Nc2ccc3cc(CN4CCNCC4)ccc3n2)nnn1-c1ccc(F)cc1C(F)(F)F] |
| 2007 | 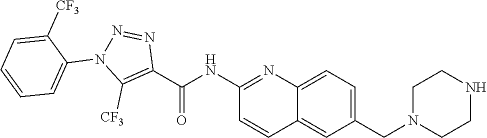 [SMILES: O=C(Nc1ccc2cc(CN3CCNCC3)ccc2n1)c1nnn(-c2ccccc2C(F)(F)F)c1C(F)(F)F] |
| 2008 | 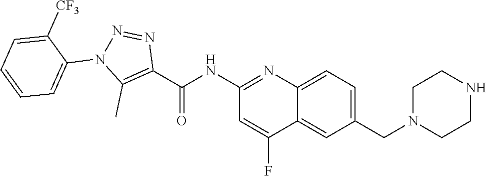 [SMILES: Cc1c(C(=O)Nc2cc(F)c3cc(CN4CCNCC4)ccc3n2)nnn1-c1ccccc1C(F)(F)F] |
| 2009 | 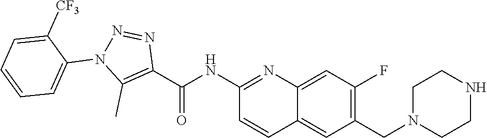 [SMILES: Cc1c(C(=O)Nc2ccc3cc(CN4CCNCC4)c(F)cc3n2)nnn1-c1ccccc1C(F)(F)F] |
| 2010 | 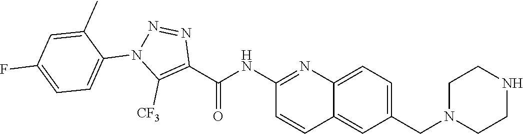 [SMILES: Cc1cc(F)ccc1-n1nnc(C(=O)Nc2ccc3cc(CN4CCNCC4)ccc3n2)c1C(F)(F)F] |
| 2011 | 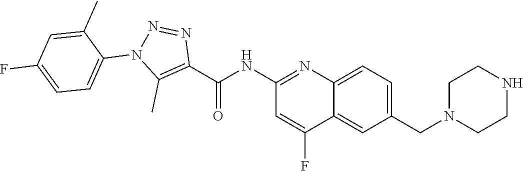 [SMILES: Cc1cc(F)ccc1-n1nnc(C(=O)Nc2cc(F)c3cc(CN4CCNCC4)ccc3n2)c1C] |
| 2012 | 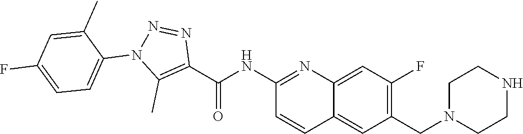 [SMILES: Cc1cc(F)ccc1-n1nnc(C(=O)Nc2ccc3cc(CN4CCNCC4)c(F)cc3n2)c1C] |
| 2013 | 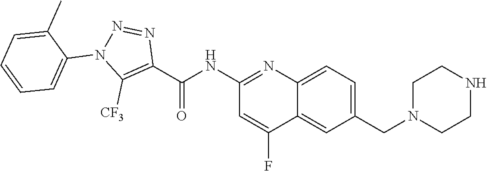 [SMILES: Cc1ccccc1-n1nnc(C(=O)Nc2cc(F)c3cc(CN4CCNCC4)ccc3n2)c1C(F)(F)F] |
| 2014 | 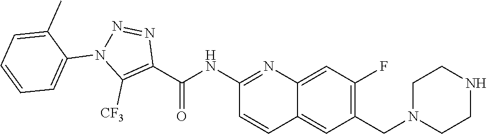 [SMILES: Cc1ccccc1-n1nnc(C(=O)Nc2ccc3cc(CN4CCNCC4)c(F)cc3n2)c1C(F)(F)F] |
| 2015 | 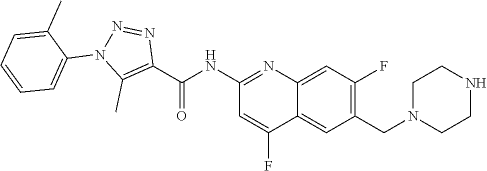 [SMILES: Cc1ccccc1-n1nnc(C(=O)Nc2cc(F)c3cc(CN4CCNCC4)c(F)cc3n2)c1C] |
| 2016 | 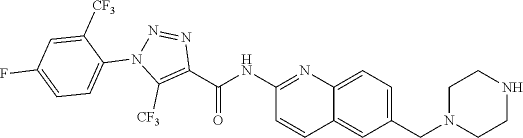 [SMILES: O=C(Nc1ccc2cc(CN3CCNCC3)ccc2n1)c1nnn(-c2ccc(F)cc2C(F)(F)F)c1C(F)(F)F] |
| 2017 | 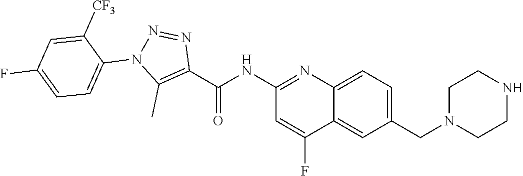 [SMILES: Cc1c(C(=O)Nc2cc(F)c3cc(CN4CCNCC4)ccc3n2)nnn1-c1ccc(F)cc1C(F)(F)F] |
| 2018 | 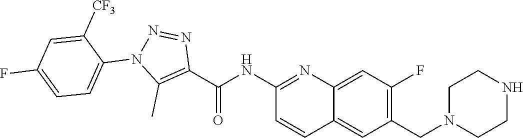 [SMILES: Cc1c(C(=O)Nc2ccc3cc(CN4CCNCC4)c(F)cc3n2)nnn1-c1ccc(F)cc1C(F)(F)F] |
| 2019 | 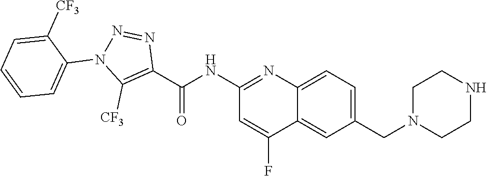 [SMILES: O=C(Nc1cc(F)c2cc(CN3CCNCC3)ccc2n1)c1nnn(-c2ccccc2C(F)(F)F)c1C(F)(F)F] |
| 2020 | 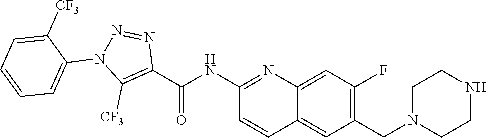 [SMILES: O=C(Nc1ccc2cc(CN3CCNCC3)c(F)cc2n1)c1nnn(-c2ccccc2C(F)(F)F)c1C(F)(F)F] |
| 2021 | 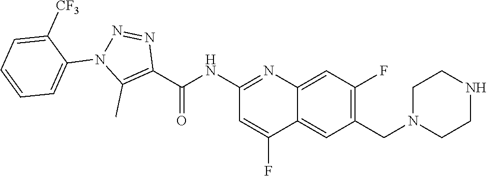 [SMILES: Cc1c(C(=O)Nc2cc(F)c3cc(CN4CCNCC4)c(F)cc3n2)nnn1-c1ccccc1C(F)(F)F] |
| 2022 | 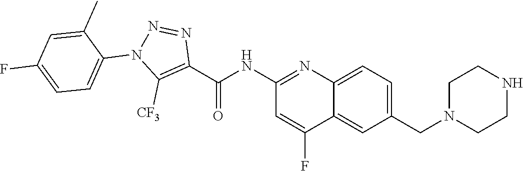 [SMILES: Cc1cc(F)ccc1-n1nnc(C(=O)Nc2cc(F)c3cc(CN4CCNCC4)ccc3n2)c1C(F)(F)F] |
| 2023 | 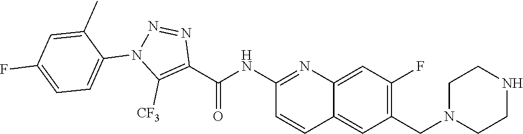 [SMILES: Cc1cc(F)ccc1-n1nnc(C(=O)Nc2ccc3cc(CN4CCNCC4)c(F)cc3n2)c1C(F)(F)F] |
| 2024 | 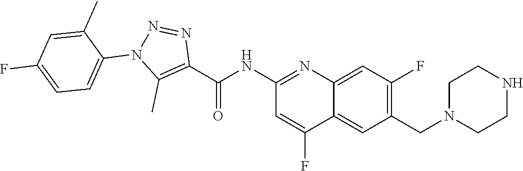 [SMILES: Cc1cc(F)ccc1-n1nnc(C(=O)Nc2cc(F)c3cc(CN4CCNCC4)c(F)cc3n2)c1C] |
| 2025 | 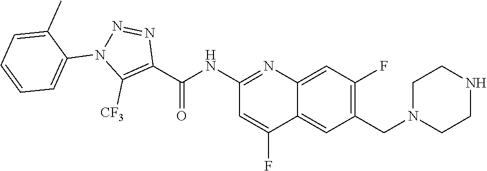 [SMILES: Cc1ccccc1-n1nnc(C(=O)Nc2cc(F)c3cc(CN4CCNCC4)c(F)cc3n2)c1C(F)(F)F] |
| 2026 | 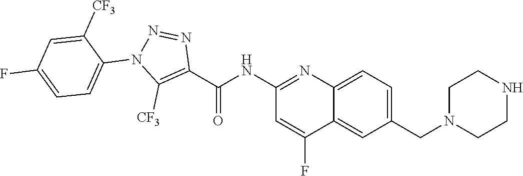 [SMILES: O=C(Nc1cc(F)c2cc(CN3CCNCC3)ccc2n1)c1nnn(-c2ccc(F)cc2C(F)(F)F)c1C(F)(F)F] |
| 2027 | 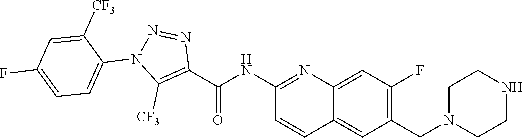 [SMILES: O=C(Nc1ccc2cc(CN3CCNCC3)c(F)cc2n1)c1nnn(-c2ccc(F)cc2C(F)(F)F)c1C(F)(F)F] |
| 2028 | 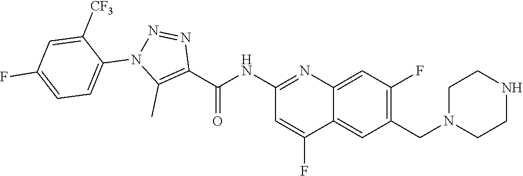 [SMILES: Cc1c(C(=O)Nc2cc(F)c3cc(CN4CCNCC4)c(F)cc3n2)nnn1-c1ccc(F)cc1C(F)(F)F] |
| 2029 | 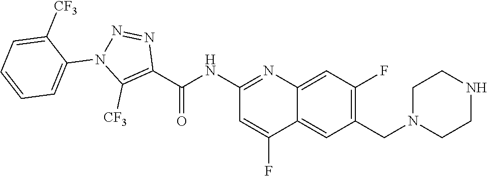 [SMILES: O=C(Nc1cc(F)c2cc(CN3CCNCC3)c(F)cc2n1)c1nnn(-c2ccccc2C(F)(F)F)c1C(F)(F)F] |
| 2030 | 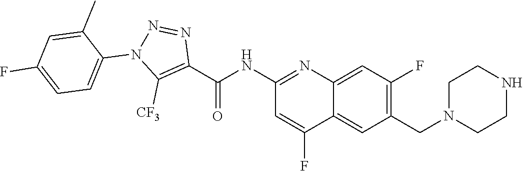 [SMILES: Cc1cc(F)ccc1-n1nnc(C(=O)Nc2cc(F)c3cc(CN4CCNCC4)c(F)cc3n2)c1C(F)(F)F] |
| 2031 | 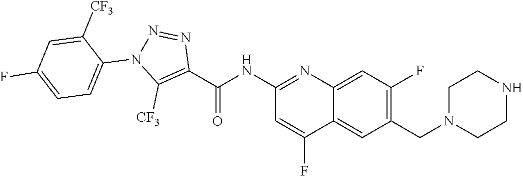 [SMILES: O=C(Nc1cc(F)c2cc(CN3CCNCC3)c(F)cc2n1)c1nnn(-c2ccc(F)cc2C(F)(F)F)c1C(F)(F)F] |

In some embodiments, the compound of formula (II) is a compound of any one of formula 2001-2005, or a pharmaceutically acceptable salt thereof.

In one aspect, the disclosure provides compounds of formula (III):

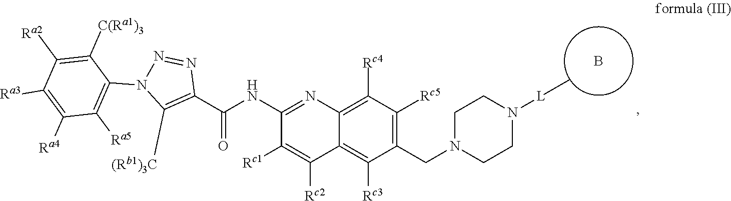

wherein R^{a1}, R^{a2}, R^{a3}, R^{a4}, R^{a5}, R^{b1}, R^{c1}, R^{c2}, R^{c3}, R^{c4}, and R^{c5} at each occurrence is independently selected from hydrogen, halo, alkyl, alkoxy, and aryl;
L is a linking group; and
B is a targeting moiety,
or a pharmaceutically acceptable salt thereof.

In some embodiments, R^{a1}, R^{a2}, R^{a3}, R^{a4}, R^{a5}, R^{b}, R^{c1}, R^{c2}, R^{c3}, R^{c4}, and R^{c5} are at each occurrence hydrogen.

In some embodiments, L comprises one or more linking groups selected from optionally substituted —C₁₋₁₀ alkyl-, —O—C₁₋₁₀ alkyl-, —C₁₋₁₀ alkenyl-, —O—C₁₋₁₀ alkenyl-, —C₁₋₁₀ cycloalkenyl-, —O—C₁₋₁₀ cycloalkenyl-, —C₁₋₁₀ alkynyl-, —O—C₁₋₁₀ alkynyl-, —C₁₋₁₀ aryl-, —O—C₁₋₁₀—, -aryl-, —O—, —S—, —S—S—, —S(O)_{w}—, —C(O)—, —C(O)O—, —OC(O)—, —C(O)S—, —SC(O)—, —OC(O)O—, —N(R^{b})—, —C(O)N(R^{b})—, —N(R^{b})C(O)—, —OC(O)N(R^{b})—, —N(R^{b})C(O)O—, —SC(O)N(R^{b})—, —N(R^{b})C(O)S—, —N(R^{b})C(O)N(R^{b})—, —N(R^{b})C(NR^{b})N(R^{b})—, —N(R^{b})S(O)_{w}—, —S(O)_{w}N(R^{b})—, —S(O)_{w}O—, —OS(O)_{w}—, —OS(O)_{w}O—, —O(O)P(OR)O—, (O)P(O—)₃, —O(S)P(OR^{b})O—, and (S)P(O—)₃, wherein w is 1 or 2, and R^{b} is independently hydrogen, optionally substituted alkyl, or optionally substituted aryl. In some embodiments, L comprises one or more linking groups selected from —C₁₋₁₀ alkyl-N(R^{b})—, —O—C₁₋₁₀ alkyl-, —O—, —N(R^{b})—, and —S—S—. In some embodiments, L comprises one or more linking group selected from

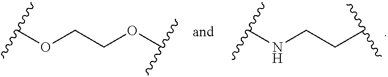 [SMILES: *OCCO* ; *CCN*]

In some embodiments, L comprises

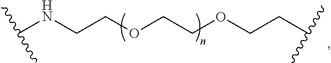

wherein n is an integer selected from 0-5. In some embodiments, L comprises

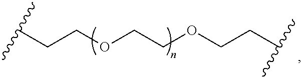

wherein n is an integer selected from 0-5.

In some embodiments, L is a single bond.

In some embodiment, the linker is a cleavable linker. In a non-limiting example, a cleavable linker is a linker that can selectively release the drug moiety (e.g. an YA6060 moiety or analogue thereof) from the targeting moiety under certain conditions, such as at certain pH levels or sensitivity to proteases or esterases. In some embodiments, the linker is a non-cleavable linker. In a non-limiting example, a non-cleavable linker is a linker that does not have a drug release mechanism, so the drug moiety (e.g. an YA6060 moiety or analogue thereof) stays tethered to the targeting moiety.

In some embodiments, the targeting moiety is a moiety that binds to or is a substrate for receptors that are selectively expressed and/or overexpressed in tumor cells and/or tissue. In some embodiments, the targeting moiety targets the folate receptor (FR) and/or organic cation transports (OCTs), and/or organic anion transporting polypeptide 1B1 (OATP1B1). In some embodiments, the targeting moiety comprises one or more moieties selected from biotin, folic acid, biguanide, and carboxylic acid.

In some embodiments, the targeting moiety is selected from:

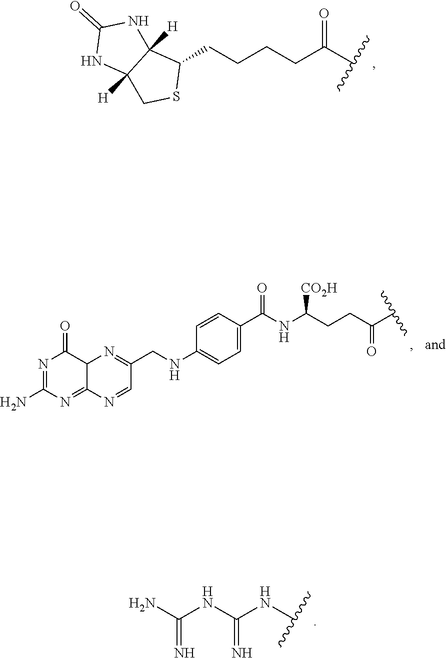 [SMILES: *C(=O)CCCC[C@@H]1SC[C@@H]2NC(=O)N[C@@H]21 ; *C(=O)CC[C@@H](NC(=O)c1ccc(NCC2=NC3C(=O)N=C(N)N=C3N=C2)cc1)C(=O)O ; *NC(=N)NC(=N)N]

In some embodiments, the compound of formula (III) is a compound of any one of formula 3001-3018, or a pharmaceutically acceptable salt thereof:

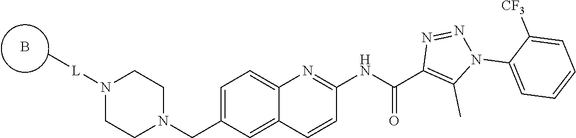

| Formula | | | |
|---|---|---|---|
| No. | B | L | n |
| 3001 | 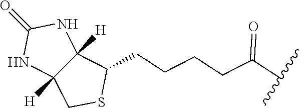 [SMILES: *C(=O)CCCC[C@@H]1SC[C@@H]2NC(=O)N[C@@H]21] | 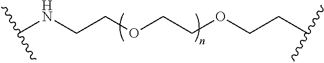 | 0 |
| 3002 | 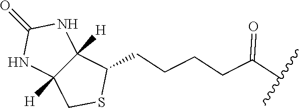 [SMILES: *C(=O)CCCC[C@@H]1SC[C@@H]2NC(=O)N[C@@H]21] | 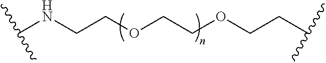 | 1 |
| 3003 | 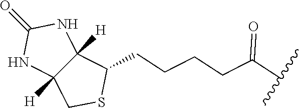 [SMILES: *C(=O)CCCC[C@@H]1SC[C@@H]2NC(=O)N[C@@H]21] | 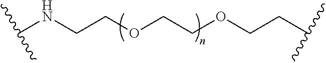 | 2 |
| 3004 | 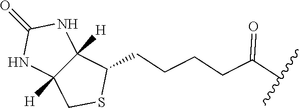 [SMILES: *C(=O)CCCC[C@@H]1SC[C@@H]2NC(=O)N[C@@H]21] | 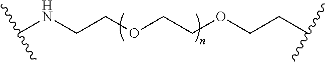 | 3 |
| 3005 | 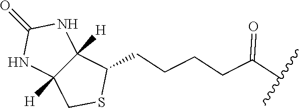 [SMILES: *C(=O)CCCC[C@@H]1SC[C@@H]2NC(=O)N[C@@H]21] | 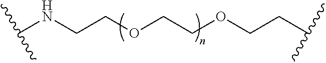 | 4 |
| 3006 | 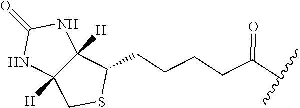 [SMILES: *C(=O)CCCC[C@@H]1SC[C@@H]2NC(=O)N[C@@H]21] | 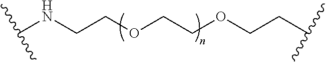 | 5 |
| 3007 | 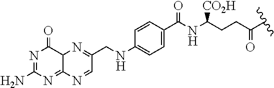 [SMILES: *C(=O)CC[C@@H](NC(=O)c1ccc(NCC2=NC3C(=O)N=C(N)N=C3N=C2)cc1)C(=O)O] | 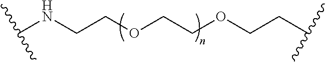 | 0 |
| 3008 | 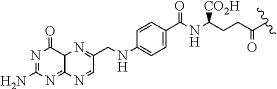 [SMILES: *C(=O)CC[C@@H](NC(=O)c1ccc(NCC2=NC3C(=O)N=C(N)N=C3N=C2)cc1)C(=O)O] | 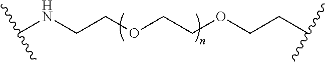 | 1 |
| 3009 | 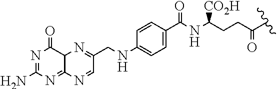 [SMILES: *C(=O)CC[C@@H](NC(=O)c1ccc(NCC2=NC3C(=O)N=C(N)N=C3N=C2)cc1)C(=O)O] | 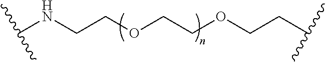 | 2 |
| 3010 | 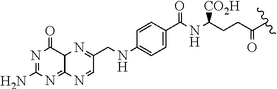 [SMILES: *C(=O)CC[C@@H](NC(=O)c1ccc(NCC2=NC3C(=O)N=C(N)N=C3N=C2)cc1)C(=O)O] | 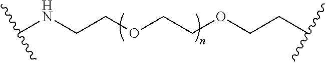 | 3 |
| 3011 | 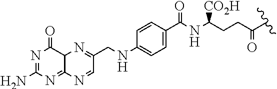 [SMILES: *C(=O)CC[C@@H](NC(=O)c1ccc(NCC2=NC3C(=O)N=C(N)N=C3N=C2)cc1)C(=O)O] | 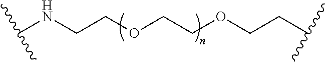 | 4 |
| 3012 | 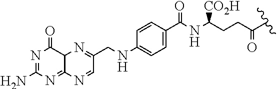 [SMILES: *C(=O)CC[C@@H](NC(=O)c1ccc(NCC2=NC3C(=O)N=C(N)N=C3N=C2)cc1)C(=O)O] | 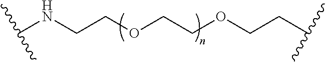 | 5 |
| 3013 | 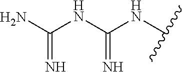 [SMILES: *NC(=N)NC(=N)N] | 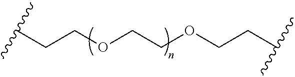 | 0 |
| 3014 | 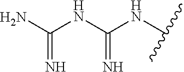 [SMILES: *NC(=N)NC(=N)N] | 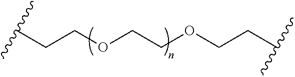 | 1 |
| 3015 | 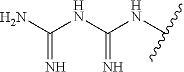 [SMILES: *NC(=N)NC(=N)N] | 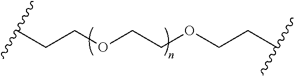 | 2 |
| 3016 | 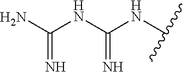 [SMILES: *NC(=N)NC(=N)N] | 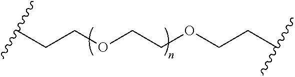 | 3 |
| 3017 | 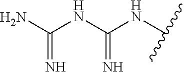 [SMILES: *NC(=N)NC(=N)N] | 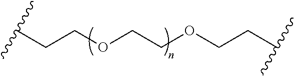 | 4 |
| 3018 | 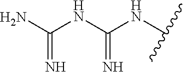 [SMILES: *NC(=N)NC(=N)N] | 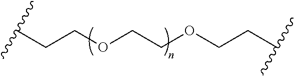 | 5 |

As used herein, the term “alkyl” denotes branched or unbranched hydrocarbon chains, having about 1 to 10 carbons, such as, methyl, ethyl, n-propyl, iso-propyl, n-butyl, sec-butyl, iso-butyl, tert-butyl, 2-methylpentyl, pentyl, hexyl, isohexyl, heptyl, 4,4-dimethyl pentyl, octyl, 2,2,4-trimethylpentyl, and the like. “Substituted alkyl” includes an alkyl group optionally substituted with one or more functional groups which are attached commonly to such chains, such as, hydroxy, halogen, mercapto or thio, cyano, alkylthio, carboxy, nitro, alkoxy, or optionally substituted, alkyl, amino, alkenyl, carboxamido, carbalkoxy, alkynyl, heterocyclyl, aryl, heteroaryl, and the like to form alkyl groups such as trifluoromethyl, 3-hydroxyhexyl, 2-carboxypropyl, 2-fluoroethyl, carboxymethyl, cyanobutyl, phenethyl, benzyl, and the like.

The term “halogen” or “halo” as used herein alone or as part of another group refers to chloro, bromo, fluoro, or iodo.

The term “alkoxy” refers to alkyl-O—, in which alkyl is as defined above.

The term “alkylthio” refers to alkyl-S—, in which alkyl is as defined above.

The term “alkylamino” refers to —NR′R″, in which R′ and R″ each may independently represent H, alkyl, or aryl, all as defined herein.

The term “alkylcarbonyl” refers to —C(═O)-alkyl, in which alkyl is as defined above.

The term “carboxy” refers to the moiety —C(═O)OH.

The term “carbalkoxy” refers to the moiety —C(═O)—O-alkyl, in which alkyl is as defined above.

The term “carboxamido” refers to the moiety —C(═O)—NR′R″, in which R′ and R″, each may independently represent H, alkyl, or aryl, all as defined herein.

The term “alkylsulfonyl” refers to the moiety —S(═O)₂-alkyl, in which alkyl is as defined above.

The term “arylsulfonyl” refers to the moiety —S(═O)₂-aryl, in which aryl is as defined herein. For example, arylsulfonyl may be —S(═O)₂-phenyl.

The term “arylsulfonyloxy” refers to the moiety —OS(═O)₂-alkyl, wherein alkyl is as defined above.

The term “amino(monoalkylamino-, dialkylamino-)sulfinyl” refers to the moiety —S(═O)NR′R″, in which R′ and R″ each may independently represent H, alkyl, or aryl, all as defined herein.

The term “amino(monoalkylamino-, dialkylamino-)sulfonyl” refers to the moiety —S(═O)₂NR′R″, in which R′ and R″ each may independently represent H, alkyl, or aryl, all as defined herein.

The term “alkylsulfonylamino” refers to the moiety —NHS(═O)₂-alkyl, in which alkyl is as previously defined.

The term “hydroxysulfonyloxy” refers to the moiety —OS(═O)₂OH.

The term “alkoxysulfonyloxy” refers to the moiety —OS(═O)₂O-alkyl, in which alkyl is as defined above.

The term “alkylsulfonyloxy” refers to the moiety —OS(═O)₂-alkyl, in which alkyl is as previously defined.

The term “hydroxysulfonyl” refers to the moiety —S(═O)₂OH.

The term “alkoxysulfonyl” refers to the moiety —S(═O)₂O-alkyl, wherein alkyl is as previously defined.

The term “alkylsulfonylalkyl” refers to the moiety -alkyl-S(═O)₂-alkyl, wherein each alkyl may be as previously defined.

The term “amino(monoalkylamino-, dialkylamino-)sulfonylakyl” refers to the moiety -alkyl-S(═O)₂—NR′R″, wherein alkyl is as previously defined, and R′ and R″ each may independently represent H, alkyl, or aryl, all as defined herein.

The term “amino(monoalkylamino-, dialkylamino-)sulfinylalkyl” refer to the moieties -alkyl-S(═O)—NR′R″, wherein alkyl is as previously defined, and R′ and R″ each may independently represent H, alkyl, or aryl, all as defined herein.

Unless otherwise indicated, the term “cycloalkyl” as employed herein alone or as part of another group includes saturated or partially unsaturated (containing 1 or more double bonds) cyclic hydrocarbon groups containing 1 to 3 rings, including monocyclicalkyl, bicyclicalkyl and tricyclicalkyl, containing a total of 3 to 20 carbons forming the rings, preferably 3 to 10 carbons, forming the ring and which may be fused to 1 or 2 aromatic rings as described for aryl, which include cyclopropyl, cyclobutyl, cyclopentyl, cyclohexyl, cycloheptyl, cyclooctyl, cyclodecyl, cyclododecyl, and cyclohexenyl. “Substituted cycloalkyl” includes a cycloalkyl group optionally substituted with 1 or more substituents such as halogen, alkyl, substituted alkyl, alkoxy, hydroxy, aryl, substituted aryl, aryloxy, cycloalkyl, alkylamido, alkanoylamino, oxo, acyl, arylcarbonylamino, amino, nitro, cyano, thiol and/or alkylthio and/or any of the substituents included in the definition of “substituted alkyl.”

Unless otherwise indicated, the term “alkenyl” as used herein by itself or as part of another group refers to straight or branched chain of 2 to 20 carbons, preferably 2 to 12 carbons, and more preferably 2 to 8 carbons in the normal chain, which include one or more double bonds in the normal chain, such as vinyl, 2-propenyl, 3-butenyl, 2-butenyl, 4-pentenyl, 3-pentenyl, 2-hexenyl, 3-hexenyl, 2-heptenyl, 3-heptenyl, 4-heptenyl, 3-octenyl, 3-nonenyl, 4-decenyl, 3-undecenyl, 4-dodecenyl, 4,8,12-tetradecatrienyl, and the like. “Substituted alkenyl” includes an alkenyl group optionally substituted with one or more substituents, such as the substituents included above in the definition of “substituted alkyl” and “substituted cycloalkyl.”

Unless otherwise indicated, the term “alkynyl” as used herein by itself or as part of another group refers to straight or branched chain of 2 to 20 carbons, preferably 2 to 12 carbons and more preferably 2 to 8 carbons in the normal chain, which include one or more triple bonds in the normal chain, such as 2-propynyl, 3-butynyl, 2-butynyl, 4-pentynyl, 3-pentynyl, 2-hexynyl, 3-hexynyl, 2-heptynyl, 3-heptynyl, 4-heptynyl, 3-octynyl, 3-nonynyl, 4-decynyl, 3-undecynyl, 4-dodecynyl and the like. “Substituted alkynyl” includes an alkynyl group optionally substituted with one or more substituents, such as the substituents included above in the definition of “substituted alkyl” and “substituted cycloalkyl.”

Unless otherwise indicated, the term “aryl” or “Ar” as employed herein alone or as part of another group refers to monocyclic, bicyclic, and/or polycyclic aromatic groups containing 6 to 10 carbons in the ring portion (such as phenyl or naphthyl including 1-naphthyl and 2-naphthyl) and may optionally include one to three additional rings fused to a carbocyclic ring or a heterocyclic ring, such as aryl, cycloalkyl, heteroaryl, or cycloheteroalkyl rings or substituted forms thereof.

“Substituted aryl” includes an aryl group optionally substituted with one or more functional groups, such as halo, alkyl, haloalkyl (e.g., trifluoromethyl), alkoxy, haloalkoxy (e.g., difluoromethoxy), alkenyl, alkynyl, cycloalkyl-alkyl, cycloheteroalkyl, cycloheteroalkylalkyl, aryl, heteroaryl, arylalkyl, aryloxy, aryloxyalkyl, arylalkoxy, alkoxycarbonyl, alkylcarbonyl, arylcarbonyl, arylalkenyl, aminocarbonylaryl, arylthio, arylsulfinyl, arylazo, heteroarylalkyl, heteroarylalkenyl, heteroarylheteroaryl, heteroaryloxy, hydroxy, nitro, cyano, amino, substituted amino wherein the amino includes 1 or 2 substituents (which are optionally substituted alkyl, aryl or any of the other substituents recited herein), thiol, alkylthio, arylthio, heteroarylthio, arylthioalkyl, alkoxyarylthio, alkylaminocarbonyl, arylaminocarbonyl, aminocarbonyl, alkylcarbonyloxy, arylcarbonyloxy, alkylcarbonylamino, arylcarbonylamino, arylsulfinyl, arylsulfinylalkyl, arylsulfonylamino, or arylsulfonaminocarbonyl and/or any of the alkyl substituents recited herein.

Unless otherwise indicated, the term “heteroaryl” as used herein alone or as part of another group refers to a 5- to 7-membered aromatic ring which includes 1, 2, 3 or 4 hetero atoms such as nitrogen, oxygen or sulfur and such rings fused to an aryl, cycloalkyl, heteroaryl or heterocycloalkyl ring (e.g. benzothiophene, indole, quinoline, thiazole, isooxazole, benzothiazole, benzimidizole, isoquinoline, pyridine, pyrimidine, benzopyrone, oxazole, thiazole, pyrazine), and includes possible N-oxides. “Substituted heteroaryl” includes a heteroaryl group optionally substituted with 1 to 4 substituents, such as the substituents included above in the definition of “substituted alkyl” and “substituted cycloalkyl.” Substituted heteroaryl also includes fused heteroaryl groups which include, for example, quinoline, isoquinoline, indole, isoindole, carbazole, acridine, benzopyrene, benzopyrone, benzimidazole, benzofuran, isobenzofuran, phenanthroline, purine, and the like.

Moreover, the terms “heterocyclo,” “heterocycle,” or “heterocyclic ring,” as used herein, refer to an unsubstituted or substituted stable 5- to 7-membered monocyclic ring system which may be saturated or unsaturated, and which consists of carbon atoms and from one to four heteroatoms selected from N, O or S, and wherein the nitrogen and sulfur heteroatoms may optionally be oxidized, and the nitrogen heteroatom may optionally be quaternized. The heterocyclic ring may be attached at any heteroatom or carbon atom which results in the creation of a stable structure. Examples of such heterocyclic groups include, but are not limited to, piperidinyl, piperazinyl, oxopiperazinyl, oxopiperidinyl, oxopyrrolidinyl, oxoazepinyl, azepinyl, pyrrolyl, pyrrolidinyl, benzothiophene, chromone, benzopyrene, benzopyrone, furanyl, thienyl, pyrazolyl, pyrazolidinyl, imidazolyl, imidazolinyl, imidazolidinyl, pyridyl, pyrazinyl, pyrimidinyl, pyridazinyl, oxazolyl, oxazolidinyl, isooxazolyl, isoxazolidinyl, morpholinyl, thiazolyl, thiazolidinyl, isothiazolyl, thiadiazolyl, tetrahydropyranyl, thiamorpholinyl, thiamorpholinylsulfoxide, thiamorpholinylsulfone, and oxadiazolyl.

As used herein, the terms “optionally substituted” or “substituted” may indicate that a chemical moiety referred to, for example, alkyl, aryl, and heteroaryl, may be unsubstituted or substituted with one or more groups including, without limitation, alkyl, substituted alkyl, alkenyl, substituted alkenyl, alkynyl, substituted alkynyl, cycloalkyl, substituted cycloalkyl, arylalkyl, substituted arylalkyl, aryl, substituted aryl, heterocycle, substituted heterocycle, heteroaryl, substituted heteroaryl, hydroxyl, amino, substituted amino, alkoxy, substituted alkoxy, halogen, carboxy, nitro, carbalkoxy, substituted carbalkoxy, carboxamido, substituted carboxamido, alkylamino, substituted alkyl amino, monoalkylaminosulfinyl, substituted, monoalkylaminosulfinyl, dialkylaminosulfinyl, substituted dialkylaminosulfinyl, monoalkylaminosulfonyl, substituted monoalkylaminosulfonyl, dialkylaminosulfonyl, substituted dialkylaminosulfonyl, alkylsulfonylamino, substituted alkylsulfonylamino, hydroxysulfonyloxy, alkoxysulfonyloxy, substituted alkoxysulfonyloxy, alkylsulfonyloxy, substituted alkylsulfonyloxy, hydroxysulfonyl, alkoxysulfonyl, substituted alkoxysulfonyl, alkylsulfonylalkyl, substituted alkylsulfonylalkyl, monoalkylaminosulfonylalkyl, substituted monoalkylaminosulfonylalkyl, dialkylaminosulfonylalkyl, substituted dialkylaminosulfonylalkyl, monoalkylaminosulfinylalkyl, substituted monoalkylaminosulfinylalkyl, dialkylaminosulfinylalkyl, substituted dialkylaminosulfinylalkyl, and the like. The chemical moieties of formula (I), formula (10)-(15), formula (20)-(29), formula (31)-(32), formula 1001-1126, formula (II), formula 2001-2031, formula (III), formula 3001-3018, or a pharmaceutically acceptable salt thereof, above, that may be optionally substituted include alkyl, alkenyl, alkynyl, cycloalkyl, arylalkyl, aryl, heterocycle, and heteroaryl, as described herein. For example, optionally substituted alkyl may include both propyl and 2-chloro-propyl. Additionally, “optionally substituted” is also inclusive of embodiments where the named substituent or substituents have multiple substituents rather than simply a single substituent. For example, optionally substituted aryl may include both phenyl and 3-ethyl-5-methyl-6-bromo-phenyl.

The compounds of the disclosure may be administered as salts, which are also within the scope of this disclosure. Pharmaceutically acceptable (i.e., non-toxic, physiologically compatible) salts are preferred. If the compounds of the disclosure have, for example, at least one basic center, they can form acid addition salts. These are formed, for example, with strong inorganic acids, such as mineral acids, for example sulfuric acid, phosphoric acid or a hydrohalic acid, with strong organic carboxylic acids, such as alkane carboxylic acids of 1 to 4 carbon atoms which are unsubstituted or substituted, for example, by halogen, for example acetic acid, such as saturated or unsaturated dicarboxylic acids, for example oxalic, malonic, succinic, maleic, fumaric, phthalic or terephthalic acid, such as hydroxycarboxylic acids, for example ascorbic, glycolic, lactic, malic, tartaric or citric acid, such as amino acids, (for example aspartic or glutamic acid or lysine or arginine), or benzoic acid, or with organic sulfonic acids, such as (C₁-C₄) alkyl or arylsulfonic acids which are unsubstituted or substituted, for example by halogen, for example methyl- or paratoluene-sulfonic acid. Corresponding acid addition salts can also be formed having plural basic centers, if desired. In some embodiments, the pharmaceutically acceptable salt is selected from valproic acid, maleic acid, tartaric acid, oxalic acid, pamoic acid, phosphonic acid, benzoic acid, citric acid, salicylic acid, succinic acid, methanesulfonic acid, malic acid, and p-toluenesulfonic acid. In some embodiments, the pharmaceutically acceptable salt is selected from valproic acid, maleic acid, tartaric acid, oxalic acid, and pamoic acid.

The compounds of the disclosure having at least one acid group (e.g., carboxylic acid) can also form salts with suitable bases. Representative examples of such salts include metal salts, such as alkali metal or alkaline earth metal salts, for example sodium, potassium or magnesium salts, or salts with ammonia or an organic amine, such as morpholine, thiomorpholine, piperidine, pyrrolidine, a mono, di or trihydroxy lower alkylamine, for example ethyl, tert-butyl, diethyl, diisopropyl, triethyl, tributyl or dimethyl-propylamine, or a mono, di or trihydroxy lower alkylamine, for example mono, di or triethanolamine. Corresponding internal salts may also be formed.

For example, certain salts of the compounds described herein which contain a basic group include monohydrochloride, hydrogensulfate, methanesulfonate, phosphate or nitrate. Moreover, certain salts of the compounds described herein which contain an acid group include sodium, potassium and magnesium salts and pharmaceutically acceptable organic amines.

All stereoisomers of the compounds of the disclosure, either in a mixture or in pure or substantially pure form, are considered to be within the scope of this disclosure. The compounds of the disclosure may have asymmetric centers at any of the carbon atoms including any one of the substituents. Consequently, compounds of the disclosure may exist in enantiomeric or diastereomeric forms or in mixtures thereof. Furthermore, where a stereocenter existing in a compound of the disclosure is represented as a racemate, it is understood that the stereocenter may encompass the racemic mixture of R and S isomers, the S isomers, and the R isomers. The processes for preparation of such compounds can utilize racemates, enantiomers, or diastereomers as starting materials. When diastereomeric or enantiomeric products are prepared, they can be separated by conventional methods including, chromatographic, chiral HPLC, fractional crystallization, or distillation. Some compounds of the present disclosure have groups including alkenyls, iminyls, and the like, which may exist as entgegen (E) or zusammen (Z) conformations, in which case all geometric forms thereof, both E and Z, cis and trans, and mixtures thereof, are within the scope of the present disclosure. Accordingly, when such geometric isomeric products are prepared, they can be separated by conventional methods for example, chromatographic, HPLC, distillation or crystallization.

### Methods of Treatment

The compounds of the invention may be used as part of a therapy or methodology in treating a variety of diseases or conditions that implicate the Wnt/β-catenin pathway and/or the AMPK pathway.

In some embodiments, the compounds of the invention are used for treating, preventing, or delaying the progression of a disorder or disease alleviated by inhibiting the Wnt/β-catenin pathway, in a patient in need of such treatment or prevention, by administering a therapeutically effective amount of one or more compounds of formula (I), formula (10)-(15), formula (20)-(29), formula (31)-(32), formula 1001-1126, formula (II), formula 2001-2031, formula (III), formula 3001-3018, 1001-1126, or a pharmaceutically acceptable salt thereof.

In some embodiments, the compounds of the invention are used for treating, preventing, or delaying the progression of a disorder or disease alleviated by activating adenosine monophosphate-activated kinase (AMPK) signaling, in a patient in need of such treatment or prevention, by administering a therapeutically effective amount of one or more compounds of formula (I), formula (10)-(15), formula (20)-(29), formula (31)-(32), formula 1001-1126, formula (II), formula 2001-2031, formula (III), formula 3001-3018, or a pharmaceutically acceptable salt thereof.

In some embodiments, the compounds of the invention are used for treating, preventing, or delaying the progression of a disorder or disease alleviated by both inhibiting Wnt/β-catenin signaling and activating adenosine monophosphate-activated kinase (AMPK) signaling, in a patient in need of such treatment or prevention, by administering a therapeutically effective amount of one or more compounds of formula (I), formula (10)-(15), formula (20)-(29), formula (31)-(32), formula 1001-1126, formula (II), formula 2001-2031, formula (III), formula 3001-3018, or a pharmaceutically acceptable salt thereof. In some embodiments, the inhibiting of Wnt/β-catenin signaling and activating of adenosine monophosphate-activated kinase (AMPK) signaling is associated with and/or proceeds by the mechanism of Axin stabilization.

In some embodiments, the compounds and compositions provided herein may be used as inhibitors of one or more members of the Wnt pathway, including one or more Wnt proteins, and/or activators of the AMPK pathway, including one or more AMPK proteins, and thus can be used to treat a variety of disorders and diseases, such as cancer and other diseases associated with abnormal angiogenesis, cellular proliferation, and cell cycling. In non-limiting examples, the compounds and compositions provided herein can be used to treat cancer, to reduce or inhibit angiogenesis, to reduce or inhibit cellular proliferation and correct a genetic disorder due to mutations in Wnt signaling components.

In some embodiments, the cancer may be one or more of adrenocortical cancer, hepatocellular cancer, hepatoblastoma, malignant melanoma, ovarian cancer, Wilm's tumor, Barrett's esophageal cancer, prostate cancer, colon cancer, colorectal cancer, rectal cancer, pancreatic cancer, bladder cancer, breast cancer, gastric cancer, head & neck cancer, lung cancer, mesothelioma, cervical cancer, uterine cancer, myeloid leukemia cancer, lymphoid leukemia cancer, pilometricoma cancer, medulloblastoma cancer, glioblastoma, and familial adenomatous polyposis. In some embodiments, the cancer is colon cancer. In some embodiments, the cancer is colorectal cancer. In some embodiments, the cancer is breast cancer. In some embodiments, the cancer is triple negative breast cancer.

Non-limiting examples of diseases which may be treated with the compounds and compositions provided herein include a variety of cancers, diabetic retinopathy, neovascular glaucoma, rheumatoid arthritis, psoriasis, mycotic and viral infections, osteochondrodysplasia, Alzheimer's disease, osteoarthritis, polyposis coli, osteoporosis-pseudoglioma syndrome, familial exudative vitreoretinopathy, retinal angiogenesis, early coronary disease, tetra-amelia syndrome, MGllerian-duct regression and virilization, SERKAL syndrome, Fuhrmann syndrome, Al-Awadi/Raas-Rothschild/Schinzel phocomelia syndrome, odonto-onycho-dermal dysplasia, split-hand/foot malformation, caudal duplication syndrome, tooth agenesis, Wilms tumor, skeletal dysplasia, focal dermal hypoplasia, autosomal recessive anonychia, neural tube defects, alpha-thalassemia (ATRX) syndrome, fragile X syndrome, ICF syndrome, Angelman syndrome, Prader-Willi syndrome, Beckwith-Wiedemann Syndrome and Rett syndrome.

In some embodiments, the methods of the disclosure may be used in the treatment of metabolic disease, including, without limitation, type 2 diabetes, obesity, hyperlipidemia, or fatty liver disease. In certain embodiments, the methods of the disclosure may be used in the treatment of type 2 diabetes. In some embodiments, fatty liver disease may include alcoholic fatty liver disease (ALD) or non-alcoholic fatty liver disease (NAFLD). In some embodiments, NAFLD may include one or more of simple fatty liver disease (steatosis), non-alcoholic steatohepatitis (NASH), and liver cirrhosis. In certain embodiments, the methods of the disclosure may be used in the treatment of NASH.

In one aspect, the disclosure includes a method of treating or preventing a disease alleviated by inhibiting Wnt/β-catenin signaling in a patient in need of said treatment or prevention, the method comprising administering a therapeutically effective amount of one or more compounds of formula (I), formula (10)-(15), formula (20)-(29), formula (31)-(32), formula 1001-1126, formula (II), formula 2001-2031, formula (III), formula 3001-3018, or a pharmaceutically acceptable salt thereof.

In one aspect, the disclosure includes a method of treating or preventing a disease alleviated by activating adenosine monophosphate-activated kinase (AMPK) signaling in a patient in need of said treatment or prevention, the method comprising administering a therapeutically effective amount of one or more compounds of formula (I), formula (10)-(15), formula (20)-(29), formula (31)-(32), formula 1001-1126, formula (II), formula 2001-2031, formula (III), formula 3001-3018, or a pharmaceutically acceptable salt thereof.

In one aspect, the disclosure includes a method of treating or preventing a disease alleviated by both inhibiting Wnt/β-catenin signaling and activating adenosine monophosphate-activated kinase (AMPK) signaling in a patient in need of said treatment or prevention, the method comprising administering a therapeutically effective amount of one or more compounds of formula (I), formula (10)-(15), formula (20)-(29), formula (31)-(32), formula 1001-1126, formula (II), formula 2001-2031, formula (III), formula 3001-3018, or a pharmaceutically acceptable salt thereof.

As used herein, the terms “administer,” “administration” or “administering” refer to (1) providing, giving, dosing, and/or prescribing by either a health practitioner or his authorized agent or under his or her direction according to the disclosure; and/or (2) putting into, taking or consuming by the mammal, according to the disclosure.

As used herein, the terms “treat,” “treatment,” and/or “treating” may refer to the management of a disease, disorder, or pathological condition (e.g., cancer or metabolic disease) with the intent to cure, ameliorate, stabilize, and/or control the disease, disorder, or pathological condition. Regarding control of the disease, disorder, or pathological condition more specifically, “control” may include the absence of disease progression, as assessed by the response to the methods recited herein, where such response may be complete (e.g., placing the disease in remission) or partial (e.g., lessening or ameliorating any symptoms associated with the disease). As used herein, the terms “prevent,” “preventing,” and/or “prevention” may refer to reducing the risk of developing a disease, disorder, or pathological condition (e.g., cancer or metabolic disorder).

In some embodiments, the methods of the disclosure may include the modulation of protein activity, regulation, and/or expression. As used herein, the terms “modulate” and “modulation” refer to a change in biological activity for a biological molecule (e.g., a protein, gene, peptide, antibody, and the like), where such change may relate to an increase in biological activity (e.g., increased activity, activation, expression, upregulation, and/or increased expression) or decrease in biological activity (e.g., decreased activity, suppression, deactivation, downregulation, and/or decreased expression) for the biological molecule.

As described herein, in some embodiments, the compounds used in the methods of the disclosure inhibit Wnt signaling, which may result in a reduction of β-catenin. In some embodiments, the compounds used in the methods of the disclosure inhibit Wnt signaling by downregulating β-catenin levels. In some embodiments, the compounds used in the methods of the disclosure inhibit Wnt signaling by downregulating β-catenin levels. In some embodiments, the compounds used in the methods of the disclosure activate phosphorylation of AMPK and its downstream target Acetyl-CoA carboxylase (ACC) is enhanced. In some embodiments, the compounds of the disclosure upregulate Axin protein expression. In some embodiments, the compounds of the disclosure downregulate c-Myc. In some embodiments, the compounds of the disclosure modulate the activity of one or more of casein kinase 1 alpha (CK1α), protein kinase B (Akt/PKB), and glycogen synthase kinase 3 (GSK3). In some embodiments, the compounds of the disclosure upregulate the activity of casein kinase 1 alpha (CK1α). In some embodiments, the compounds of the disclosure downregulate the activity of 182 kDa tankyrase-1-binding protein (TAB182). In some embodiments, the compounds of the disclosure downregulate the activity of one or more of Akt/PKB and GSK3. In some embodiments, the compounds of the disclosure suppress the expression of glucose 6-phosphatase (G6P). In some embodiments, the compounds of the disclosure increase phosphorylation of 5′ adenosine monophosphate-activated protein kinase (AMP kinase or AMPK).

In some embodiments of the disclosure, the methods described herein may include the treatment of certain symptoms of diseases that implicate the Wnt/β-catenin signaling pathway and/or the AMPK pathway.

For example, the methods of the disclosure may include treatments for symptoms of cancer or metabolic diseases. In some embodiments, the methods of the disclosure may include treatments for symptoms of type 2 diabetes. In some embodiments, the methods of the disclosure include the treatment of a fatty liver disease. In some embodiments, the fatty liver disease comprises a nonalcoholic fatty liver disease (NAFLD) or nonalcoholic steatohepatitis (NASH).

In some embodiments, the compounds of the disclosure may suppress glucose production. Furthermore, in some embodiments, the compounds of the disclosure may improve glucose tolerance in a patient in need thereof. In some embodiments, the compounds of the disclosure may reduce fasting glucose levels in a patient in need thereof. In some embodiments, the compounds of the disclosure may suppress gluconeogenesis in a patient in need thereof. In some embodiments, the compounds of the disclosure may reverse obesity and/or decrease weight gain in a patient in need thereof. In some embodiments, the compounds of the disclosure may increase insulin sensitivity in a patient in need thereof.

In some embodiments, the methods may include the co-administration of a compound of the disclosure with an additional therapeutic agent. The term “co-administering” as used herein means a process whereby the combination of a compound of the disclosure and at least one additional therapeutic agent is administered to the same patient. The compound of the disclosure and additional therapeutic may be administered simultaneously, at essentially the same time, or sequentially. If administration takes place sequentially, the compound of the invention may be administered before or after a given additional therapeutic agent or treatment. The compound of the disclosure and additional therapeutic agent or treatment need not be administered by means of the same vehicle or physiologically compatible carrier medium. The compound of the disclosure and the additional therapeutic agent may be administered one or more times and the number of administrations of each component of the combination may be the same or different. In addition, the compound of the disclosure and additional therapeutic agent or treatment need not be administered at the same site.

In some embodiments, the methods of the disclosure may include administering (1) a therapeutically effective amount of one or more of a compound of formula (I), formula (10)-(15), formula (20)-(29), formula (31)-(32), formula 1001-1126, formula (II), formula 2001-2031, formula (III), formula 3001-3018, or a pharmaceutically acceptable salt or a prodrug thereof; and (2) a therapeutically effective amount of an additional therapeutic agent.

In some embodiments, the additional therapeutic agent may include one or more of a RAF inhibitor, an MEK inhibitor, an ERK inhibitor, a VEGFR inhibitor, and an EGFR inhibitor. In some embodiments, the VEGFR inhibitor may include one or more of Bevacizumab (AVASTIN), Aflibercept (ZALTRAP), and Regorafenib (STIVARGA). In some embodiments, the EGFR inhibitor may include one or more of Cetuximab (ERBITUX), Panitumumab (VECTIBIX), and Gefitinib. In some embodiments, the additional therapeutic agent may include pyrvinium.

In some embodiments, the methods of the disclosure include co-administration of an additional therapeutic agent useful for the treatment of fatty liver disease, including, but not limited to, a nonalcoholic fatty liver disease (NAFLD) or nonalcoholic steatohepatitis (NASH). In some embodiments, the additional therapeutic agent is selected from a hyperlipidemic drug, an antihypertensive, an antidiabetic, and a farnesoid X receptor (FXR) ligand. In some embodiments, the additional therapeutic agent is selected from angiotensin II receptor antagonists, angiotensin converting enzyme (ACE) inhibitors, caspase inhibitors, cathepsin B inhibitors, CCR2 chemokine antagonists, CCR5 chemokine antagonists, chloride channel stimulators, cholesterol solubilizers, diacylglycerol O-acyltransferase 1 (DGAT1) inhibitors, dipeptidyl peptidase IV (DPPIV) inhibitors, farnesoid X receptor (FXR) agonists, FXR/TGR5 dual agonists, galectin-3 inhibitors, glucagon-like peptide 1 (GLPl) agonists, glutathione precursors, hepatitis C virus NS3 protease inhibitors, HMG CoA reductase inhibitors, 1 Iβ-hydroxy steroid dehydrogenase (I Iβ-HSDl) inhibitors, IL-Iβ antagonists, IL-6 antagonists, IL-10 agonists, IL-17 antagonists, ileal sodium bile acid cotransporter inhibitors, leptin analogs, 5-lipoxygenase inhibitors, LPL gene stimulators, lysyl oxidase homolog 2 (LOXL2) inhibitors, PDE3 inhibitors, PDE4 inhibitors, phospholipase C (PLC) inhibitors, PPARa agonists, PPARy agonists, PPAR5 agonists, Rho associated protein kinase 2 (ROCK2) inhibitors, sodium glucose transporter-2 (SGLT2) inhibitors, stearoyl CoA desaturase-1 inhibitors, thyroid hormone receptor β agonists, tumor necrosis factor a (TNFa) ligand inhibitors, transglutaminase inhibitors, transglutaminase inhibitor precursors, PTPlb inhibitors, and ASK1 inhibitors.

Non-limiting examples of an additional therapeutic agent include therapeutic agents for hyperlipidemia, antihypertensives, antidiabetics, antioxidants, blood flow improving agents, and bile acid derivatives.

Non-limiting examples of therapeutic agents for hyperlipidemia include polyenephosphatidylcholine, unsaponifiable soybean oil (soy sterol), gamma-oryzanol, riboflavin butyrate, dextran sulfate sodium sulfur 18, pantethine, and elastase; statins such as pravastatin, simvastatin, atorvastatin, fluvastatin, pitavastatin, rosuvastatin, and cerivastatin; fibrates such as simfibrate, clofibrate, clinofibrate, bezafibrate, and fenofibrate; lipolytic enzyme inhibitors such as orlistat and cetilistat; resins such as colestyramine and colestimide; and ezetimibe.

Non-limiting examples of antihypertensives include angiotensin II receptor blockers such as irbesartan, olmesartan medoxomil, candesartan cilexetil, telmisartan, valsartan, and losartan potassium; angiotensin-converting enzyme inhibitors such as alacepril, imidapril hydrochloride, enalapril maleate, captopril, quinapril hydrochloride, cilazapril hydrate, temocapril hydrochloride, delapril hydrochloride, trandolapril, benazepril hydrochloride, perindopril, and lisinopril hydrate; calcium antagonists such as azelnidipine, amlodipine besylate, aranidipine, efonidipine hydrochloride, cilnidipine, nicardipine hydrochloride, nifedipine, nimodipine, nitrendipine, nilvadipine, barnidipine hydrochloride, felodipine, benidipine, and manidipine; [alpha] receptor blocker such as tolazoline, and phentolamine; [beta] receptor blockers such as atenolol, metoprolol, acebutolol, propranolol, pindolol, carvedilol, and labetalol hydrochloride; a receptors stimulant such as clonidine and methyldopa; and diuretics such as eplerenone, hydrochlorothiazide, and furosemide.

Non-limiting examples of antidiabetics include [alpha]-glucosidase inhibitors such as acarbose, voglibose, and miglitol; sulfonyl urea hypoglycemics such as gliclazide, glibenclamide, glimepiride, and tolbutamide; fast-acting insulin secretagogues such as nateglinide and mitiglinide; biguanide hypoglycemics such as metformin hydrochloride and buformin hydrochloride; dipeptidyl phosphatase 4 inhibitors such as sitagliptin, vildagliptin, alogliptin, linagliptin and saxagliptin; thiazolidine reagents such as pioglitazone hydrochloride and rosiglitazone maleate; and glucagon-like peptide 1 derivative reagents such as exenatide, lixisenatide and liraglutide.

Non-limiting examples of antioxidants include vitamins such as ascorbic acid (vitamin C), tocopherol (vitamin E), and tocopherol nicotinate, and N-acetylcysteine, probucol.

Non-limiting examples of blood flow improving agents include cilostazol, ticlopidine hydrochloride, alprostadil, limaprost, beraprost sodium, sarpogrelate hydrochloride, argatroban, naftidrofuryl, isoxsuprine hydrochloride, batroxobin, dihydroergotoxine mesilate, tolazoline hydrochloride, hepronicate, and shimotsu-to extract.

Non-limiting examples of bile acid derivatives include ursodeoxycholic acid, chenodeoxycholic acid, bile powder, deoxycholic acid, cholic acid, bile extract, bear bile, oriental bezoar, and dehydrocholic acid. Preferable examples also include biotin (vitamin B7), cyanocobalamin (vitamin B12), pantothenic acid (vitamin B5), folic acid (vitamin B9), thiamine (vitamin B1), vitamin A, vitamin D, vitamin K, tyrosine, pyridoxine (vitamin B6), branched chain amino acids such as leucine, isoleucine, and valine, calcium, iron, zinc, copper, and magnesium. Other non-limiting examples include components used in designated health foods and functional nutritional foods such as soy protein, chitosan, low molecular weight sodium alginate, dietary fiber from psyllium seed coat, soy peptide with bound phospholipids, phytosterol ester, plant stanol ester, diacylglycerol, globin digest, and tea catechin.

In some embodiments, the methods of the disclosure include co-administration of an additional therapeutic agent useful for the treatment of colorectal cancer (CRC). Non-limiting examples of additional therapeutic agent useful for the treatment of colorectal cancer (CRC) include capecitabine; cetuximab; bevacizumab; a MEK inhibitor such as N-[(R)-2,3-dihydroxy-propoxy]-3,4-difluoro-2-(2-fluoro-4-iodo-phenylamino)-benzamide, or a pharmaceutically acceptable salt thereof; a FOLFOX4 combination including oxaliplatin, 5-fluorouracil and leucovorin; and a FOLFIRI combination include irinotecan, 5-fluorouracil and leucovorin and the like.

Furthermore, the described methods of treatment may normally include medical follow-up to determine the therapeutic or prophylactic effect brought about in the subject undergoing treatment with the compound(s) and/or composition(s) described herein.

Molecular modeling and computer-based modeling may be used in accordance with the disclosure to both understand the protein targets of the therapeutic agents described herein or to direct drug design in the preparation of analogs. Data reflecting the effect of compounds of the disclosure on protein binding, for example, or other resulting in vitro or in vivo activity data, may be used to develop a pharmacophore and pharmacophore model. As used herein, the term “pharmacophore” refers to the ensemble of steric and electronic features that are necessary to ensure the optimal supramolecular interactions with a specific biological target structure and to trigger, activate, block, inhibit or modulate the biological target's biological activity, as the case may be. See, IUPAC, Pure and Applied Chemistry (1998) 70: 1129-1143.

As used herein, the term “pharmacophore model” refers to a representation of points in a defined coordinate system wherein a point corresponds to a position or other characteristic of an atom or chemical moiety in a bound conformation of a ligand and/or an interacting polypeptide, protein, or ordered water. An ordered water is an observable water in a model derived from structural determination of a polypeptide or protein. A pharmacophore model can include, for example, atoms of a bound conformation of a ligand, or portion thereof. A pharmacophore model can include both the bound conformations of a ligand, or portion thereof, and one or more atoms that interact with the ligand and are from a bound polypeptide or protein. Thus, in addition to geometric characteristics of a bound conformation of a ligand, a pharmacophore model can indicate other characteristics including, for example, charge or hydrophobicity of an atom or chemical moiety. A pharmacophore model can incorporate internal interactions within the bound conformation of a ligand or interactions between a bound conformation of a ligand and a polypeptide, protein, or other receptor including, for example, van der Waals interactions, hydrogen bonds ionic bonds, and hydrophobic interactions. A pharmacophore model can be derived from 2 or more bound conformations of a ligand.

Turning to the administration of therapeutics, the compounds of the disclosure may be administered as described herein, or in a form from which the active agent can be derived, such as a prodrug. A “prodrug” is a derivative of a compound described herein, the pharmacologic action of which results from the conversion by chemical or metabolic processes in vivo to the active compound. Prodrugs include compounds wherein an amino acid residue, or a polypeptide chain of two or more (e.g., two, three or four) amino acid residues is covalently joined through an amide or ester bond to a free amino, hydroxyl or carboxylic acid group of formula (I), formula (10)-(15), formula (20)-(29), formula (31)-(32), formula 1001-1126, formula (II), formula 2001-2031, formula (III), formula 3001-3018, or a pharmaceutically acceptable salt thereof. The amino acid residues include but are not limited to the 20 naturally occurring amino acids commonly designated by one or three letter symbols but also include, for example, 4-hydroxyproline, hydroxylysine, desmosine, isodesmosine, 3-methylhistidine, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters. Prodrug esters as employed herein includes esters and carbonates formed by reacting one or more hydroxyls of compounds of the method of the disclosure with alkyl, alkoxy, or aryl substituted acylating agents employing procedures known to those skilled in the art to generate acetates, pivalates, methylcarbonates, benzoates and the like. As further examples, free hydroxyl groups may be derivatized using groups including but not limited to hemisuccinates, phosphate esters, dimethylaminoacetates, and phosphoryloxymethyloxycarbonyls, as outlined in Advanced Drug Delivery Reviews, 1996, 19, 115. Carbamate prodrugs of hydroxyl and amino groups are also included, as are carbonate prodrugs, sulfonate prodrugs, sulfonate esters and sulfate esters of hydroxyl groups. Free amines can also be derivatized to amides, sulfonamides or phosphonamides. All of the stated prodrug moieties may incorporate groups including but not limited to ether, amine and carboxylic acid functionalities. Moreover, any compound that can be converted in vivo to provide the bioactive agent (e.g., a compound of formula (I), formula (10)-(15), formula (20)-(29), formula (31)-(32), formula 1001-1126, formula (II), formula 2001-2031, formula (III), formula 3001-3018, or a pharmaceutically acceptable salt thereof) is a prodrug within the scope of the disclosure. Various forms of prodrugs are well known in the art. A comprehensive description of pro drugs and prodrug derivatives are described in: (a) The Practice of Medicinal Chemistry, Camille G. Wermuth et al., (Academic Press, 1996); (b) Design of Prodrugs, edited by H. Bundgaard, (Elsevier, 1985); (c) A Textbook of Drug Design and Development, P. Krogsgaard-Larson and H. Bundgaard, eds., (Harwood Academic Publishers, 1991).

In general, prodrugs may be designed to improve the penetration of a drug across biological membranes in order to obtain improved drug absorption, to prolong duration of action of a drug (slow release of the parent drug from a prodrug, decreased first-pass metabolism of the drug), to target the drug action (e.g. organ or tumor-targeting, lymphocyte targeting), to modify or improve aqueous solubility of a drug (e.g., i.v. preparations and eyedrops), to improve topical drug delivery (e.g. dermal and ocular drug delivery), to improve the chemical/enzymatic stability of a drug, or to decrease off-target drug effects, and more generally in order to improve the therapeutic efficacy of the compounds utilized in the disclosure.

A compound used in practicing any method of the disclosure may be administered in an amount sufficient to induce the desired therapeutic effect in the recipient thereof. Thus the term “therapeutically effective amount” as used herein refers to an amount of a compound of the disclosure that is sufficient to treat a disease in accordance with the disclosure by administration of one or more of the compounds of formula (I), formula (10)-(15), formula (20)-(29), formula (31)-(32), formula 1001-1126, formula (II), formula 2001-2031, formula (III), formula 3001-3018, or a pharmaceutically acceptable salt or a prodrug thereof. Preferably, the therapeutically effective amount refers to the amount appropriate to inhibit the Wnt/β-catenin pathway. In addition, the term therapeutically effective amount may include the amount of a compound necessary, for example, to bring about a detectable therapeutic, preventative, or ameliorative effect in a patient having a disease as set forth herein. The effect may include, for example, the reduction, prevention, amelioration, or stabilization of symptoms or conditions associated with a disease as described herein.

The compound(s) described herein may also be administered at a dose in range from about 0.01 mg to about 200 mg/kg of body weight per day. A dose of from about 0.1 to 100 mg/kg, or from about 1 to 50 mg/kg per day in one or more applications per day may be effective to produce the desired result. By way of example, a suitable dose for oral administration may be in the range of 1-50 mg/kg of body weight per day, whereas a dose for intravenous administration may be in the range of 1-10 mg/kg of body weight per day.

Of course, as those skilled in the art will appreciate, the dosage actually administered will depend upon the condition being treated, the age, health and weight of the recipient, the type of concurrent treatment, if any, and the frequency of treatment. Moreover, the effective dosage amount may be determined by one skilled in the art on the basis of routine empirical activity testing to measure the bioactivity of the compound(s) in a bioassay, and thus establish the appropriate dosage to be administered.

The compounds used in certain methods of the disclosure may typically be administered from 1-4 times a day, so as to deliver the above-mentioned daily dosage. However, the exact regimen for administration of the compounds described herein will necessarily be dependent on the needs of the individual subject being treated, the type of treatment administered and the judgment of the attending medical specialist. As used herein, the term “subject” or “patient” includes both humans and animals.

In general, the compounds used in the methods of the disclosure can be administered in pure form or, as described herein, with physiologically compatible and/or acceptable carrier mediums, using any acceptable route known in the art, either alone or in combination with one or more other therapeutic agents. Thus, the compound(s) and/or composition(s) of the disclosure can be administered orally, parenterally, such as by intravenous or intraarterial infusion, intramuscular, intraperitoneal, intrathecal or subcutaneous injection, by liposome-mediated delivery, rectally, vaginally, by inhalation or insufflation, transdermally or by otic delivery. In some embodiments, the compound is administered orally.

The orally administered dosage unit may be in the form of tablets, caplets, dragees, pills, semisolids, soft or hard gelatin capsules, aqueous or oily solutions, emulsions, suspensions or syrups. Suitable dosage forms for parenteral administration include injectable solutions or suspensions, suppositories, powder formulations, such as microcrystals or aerosol spray. The active agents of the disclosure may also be incorporated into a conventional transdermal delivery system.

As used herein, the expression “physiologically compatible carrier medium” (or “physiologically acceptable carrier medium” and the like) includes any and all solvents, diluents, or other liquid vehicles, dispersions or suspension aids, surface agent agents, isotonic agents, thickening or emulsifying agents, preservatives, solid binders, lubricants, fillers and the like as suited for the particular dosage form desired. Remington: The Science and Practice of Pharmacy, 20th edition, A. R. Genaro et al., Part 5, Pharmaceutical Manufacturing, pp. 669-1015 (Lippincott Williams & Wilkins, Baltimore, MD/Philadelphia, PA) (2000) discloses various carriers used in formulating pharmaceutical compositions and known techniques for the preparation thereof. Except insofar as any conventional pharmaceutical carrier medium is incompatible with the compounds of the present disclosure, such as by producing an undesirable biological effect or otherwise interacting in an deleterious manner with any other component(s) of a formulation comprising such compounds or agents, its use is contemplated to be within the scope of this disclosure.

For the production of solid dosage forms, including hard and soft capsules, the agents of the disclosure may be mixed with pharmaceutically inert, inorganic or organic excipients, such as lactose, sucrose, glucose, gelatine, malt, silica gel, starch or derivatives thereof, talc, stearic acid or its salts, dried skim milk, vegetable, petroleum, animal or synthetic oils, wax, fat, polyols, and the like. For the production of liquid solutions, emulsions or suspensions or syrups one may use excipients such as water, alcohols, aqueous saline, aqueous dextrose, polyols, glycerine, lipids, phospholipids, cyclodextrins, vegetable, petroleum, animal or synthetic oils. For suppositories one may use excipients, such as vegetable, petroleum, animal or synthetic oils, wax, fat and polyols. For aerosol formulations, one may use compressed gases suitable for this purpose, such as oxygen, nitrogen and carbon dioxide. Pharmaceutical compositions or formulations may also contain one or more additives including, without limitation, preservatives, stabilizers, e.g., UV stabilizers, emulsifiers, sweeteners, salts to adjust the osmotic pressure, buffers, coating materials and antioxidants.

The disclosure further includes controlled-release, sustained release, or extended-release therapeutic dosage forms for administration of the compounds of the disclosure, which involves incorporation of the compounds into a suitable delivery system in the formation of certain compositions. This dosage form controls release of the compound(s) in such a manner that an effective concentration of the compound(s) in the bloodstream may be maintained over an extended period of time, with the concentration in the blood remaining relatively constant, to improve therapeutic results and/or minimize side effects. Additionally, a controlled-release system would provide minimum peak to trough fluctuations in blood plasma levels of the compound.

In pharmaceutical compositions used in practicing the methods of the disclosure more particularly, the specified compound(s) may be present in an amount of at least 0.5 and generally not more than 95% by weight, based on the total weight of the composition, including carrier medium and/or supplemental active agent(s), if any. In some embodiments, the proportion of compound(s) varies between about 30-90% by weight of the composition.

In some embodiments, the compositions of the disclosure may include (1) one or more of a compound of formula (I), formula (10)-(15), formula (20)-(29), formula (31)-(32), formula 1001-1126, formula (II), formula 2001-2031, formula (III), formula 3001-3018, or a pharmaceutically acceptable salt thereof; (2) an additional therapeutic agent; and a physiologically compatible carrier medium. In some embodiments, the additional therapeutic agent may include one or more of a RAF inhibitor, an MEK inhibitor, an ERK inhibitor, a VEGFR inhibitor, and an EGFR inhibitor. In some embodiments, the VEGFR inhibitor may include one or more of Bevacizumab (AVASTIN), Aflibercept (ZALTRAP), and Regorafenib (STIVARGA). In some embodiments, the EGFR inhibitor may include one or more of Cetuximab (ERBITUX), Panitumumab (VECTIBIX) Gefitinib. In some embodiments, the additional therapeutic agent may include pyrvinium. In some embodiments, the additional therapeutic agent may include one or more of angiotensin II receptor antagonists, angiotensin converting enzyme (ACE) inhibitors, caspase inhibitors, cathepsin B inhibitors, CCR2 chemokine antagonists, CCR5 chemokine antagonists, chloride channel stimulators, cholesterol solubilizers, diacylglycerol O-acyltransferase 1 (DGAT1) inhibitors, dipeptidyl peptidase IV (DPPIV) inhibitors, farnesoid X receptor (FXR) agonists, FXR/TGR5 dual agonists, galectin-3 inhibitors, glucagon-like peptide 1 (GLPl) agonists, glutathione precursors, hepatitis C virus NS3 protease inhibitors, HMG CoA reductase inhibitors, 1 Iβ-hydroxy steroid dehydrogenase (I Iβ-HSDl) inhibitors, IL-Iβ antagonists, IL-6 antagonists, IL-10 agonists, IL-17 antagonists, ileal sodium bile acid cotransporter inhibitors, leptin analogs, 5-lipoxygenase inhibitors, LPL gene stimulators, lysyl oxidase homolog 2 (LOXL2) inhibitors, PDE3 inhibitors, PDE4 inhibitors, phospholipase C (PLC) inhibitors, PPARa agonists, PPARy agonists, PPAR5 agonists, Rho associated protein kinase 2 (ROCK2) inhibitors, sodium glucose transporter-2 (SGLT2) inhibitors, stearoyl CoA desaturase-1 inhibitors, thyroid hormone receptor β agonists, tumor necrosis factor a (TNFa) ligand inhibitors, transglutaminase inhibitors, transglutaminase inhibitor precursors, PTPlb inhibitors, and ASK1 inhibitors. In some embodiments, the additional therapeutic agent may include one or more of capecitabine; cetuximab; bevacizumab; a MEK inhibitor such as N-[(R)-2,3-dihydroxy-propoxy]-3,4-difluoro-2-(2-fluoro-4-iodo-phenylamino)-benzamide, or a pharmaceutically acceptable salt thereof; a FOLFOX4 combination including oxaliplatin, 5-fluorouracil and leucovorin; and a FOLFIRI combination include irinotecan, 5-fluorouracil and leucovorin and the like.

In some embodiments, the disclosure provides a pharmaceutical composition comprising one or more of a compound of formula (I), formula (10)-(15), formula (20)-(29), formula (31)-(32), formula 1001-1126, formula (II), formula 2001-2031, formula (III), formula 3001-3018, or a pharmaceutically acceptable salt thereof, and a physiologically compatible carrier medium.

In some embodiments, the disclosure provides a pharmaceutical composition for treating or preventing a disease alleviated by inhibiting Wnt/β-catenin signaling and/or activating AMPK signaling comprising one or more of a compound of formula (I), formula (10)-(15), formula (20)-(29), formula (31)-(32), formula 1001-1126, formula (II), formula 2001-2031, formula (III), formula 3001-3018, or a pharmaceutically acceptable salt thereof, and a physiologically compatible carrier medium.

In some embodiments, the disclosure provides a pharmaceutical composition treating liver fibrosis comprising one or more of a compound of formula (I), formula (10)-(15), formula (20)-(29), formula (31)-(32), formula 1001-1126, formula (II), formula 2001-2031, formula (III), formula 3001-3018, or a pharmaceutically acceptable salt thereof, and a physiologically compatible carrier medium.

In some embodiments, the disclosure provides a pharmaceutical composition treating colorectal cancer (CRC) comprising one or more of a compound of formula (I), formula (10)-(15), formula (20)-(29), formula (31)-(32), formula 1001-1126, formula (II), formula 2001-2031, formula (III), formula 3001-3018, or a pharmaceutically acceptable salt thereof, and a physiologically compatible carrier medium.

In some embodiments, the disclosure provides a pharmaceutical composition treating alcoholic fatty liver disease (ALD) or non-alcoholic fatty liver disease (NAFLD) comprising one or more of a compound of formula (I), formula (10)-(15), formula (20)-(29), formula (31)-(32), formula 1001-1126, formula (II), formula 2001-2031, formula (III), formula 3001-3018, or a pharmaceutically acceptable salt thereof, and a physiologically compatible carrier medium. In some embodiments, the non-alcoholic fatty liver disease is selected from the group consisting of simple fatty liver (steatosis), non-alcoholic steatohepatitis (NASH), and liver cirrhosis.

The following examples describe the disclosure in further detail. These examples are provided for illustrative purposes only, and should in no way be considered as limiting the disclosure.

### EXAMPLES

### Example 1: Wnt/β-Catenin Signaling Pathway Inhibitors with Improved Physiochemical Properties

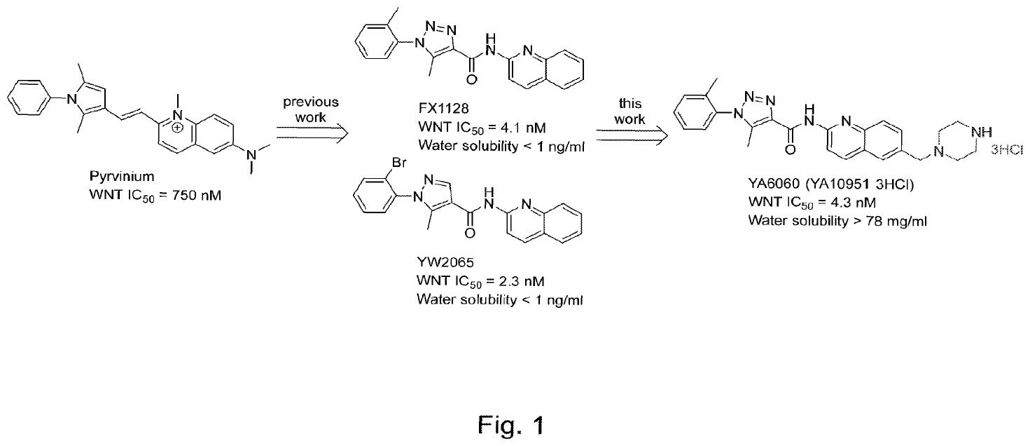

Two potent inhibitors of the Wnt/i-catenin signaling pathway, FX1128 and FX2065 were previously identified. These Wnt signaling inhibitors exhibited single digit nM inhibitory potency in cell and intriguing efficacies in mice models of fatty liver disorder and colorectal cancer, respectively. However, compounds FX1128 and FX2065 indicated suboptimal druglike profiles highlighted by their poor aqueous solubilities, which could adversely impact the ADME and bioavailability of these compounds for their future therapeutic applications. This Example describes a class of new Wnt/β-catenin signaling pathway inhibitors with dramatically enhanced aqueous solubility. As a highlight, the hydrochloride salt form of the 1-methylpiperazine analog YA6060 demonstrated a remarkable aqueous solubility of >78 mg/mL, which accounts for an over 10,000-fold increase from both compounds FX1128 and FX2065 (FIG. 1). Importantly, the new inhibitor YA6060 remains highly potent for the Wnt/i-catenin signaling pathway with a cellular IC₅₀ value of 4.3 nM.

### Chemistry Synthesis

The synthesis of compounds YA10951 and YA6060 began with o-toluidine 1 (Scheme 1). Diazotization of the amino group of compound 1 using sodium nitrite (NaNO₂) and aqueous HCl, followed by the treatment of the intermediate with sodium azide (NaN₃) provided azides, which underwent cyclization with β-ketoester in EtONa/EtOH yielded trizaole-3-carboxylic acids 2. Next, chlorodipyrrolidinocarbenium (PyCIU)-mediated coupling of compounds 2 with 6-bromoquinolin-2-amine in dichloroethane (DCE) provided compound 3 in moderate to good yields. Further, Pd-catalyzed cross-coupling of compound 3 with potassium trifluoroborate derivatives yielded products 4 in good yields. The Boc group of 4 was then removed by trifluoroacetic acid (TFA) to yield amino-containing compound YA10951 in good yields. Alternatively, compound 4 was treated with 2 M HCl in Et₂O to provide the compound YA6060 in good yields.

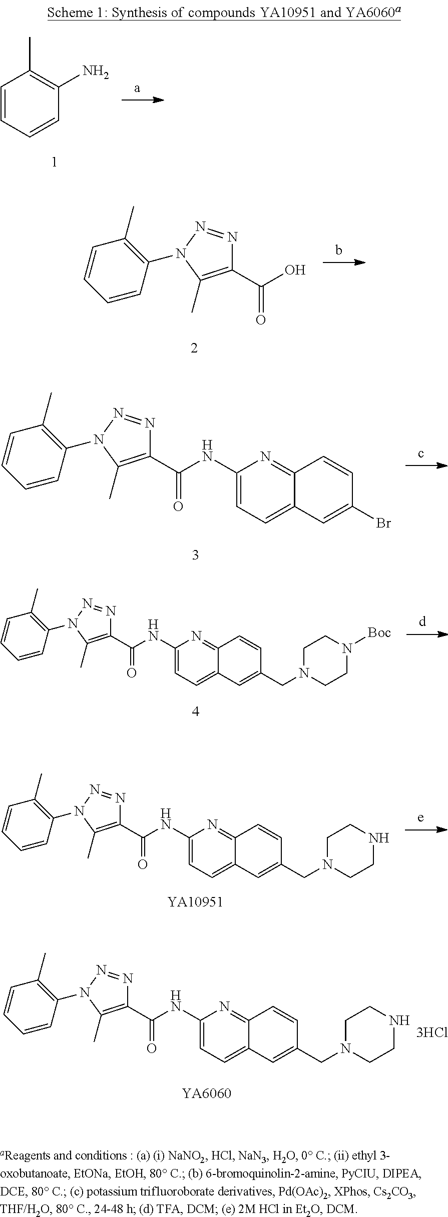

The syntheses of compounds YA6063 and YA6079 is shown in Scheme 2. Suzuki coupling of compound 3 with bis-(pinacolato)diboron in the presence of Pd(dppf)Cl₂ and KOAc yielded the boronic ester YA6063, which was subsequently converted to the hydroxy compound YA6079 in the presence of hydrogen peroxide (H₂O₂).

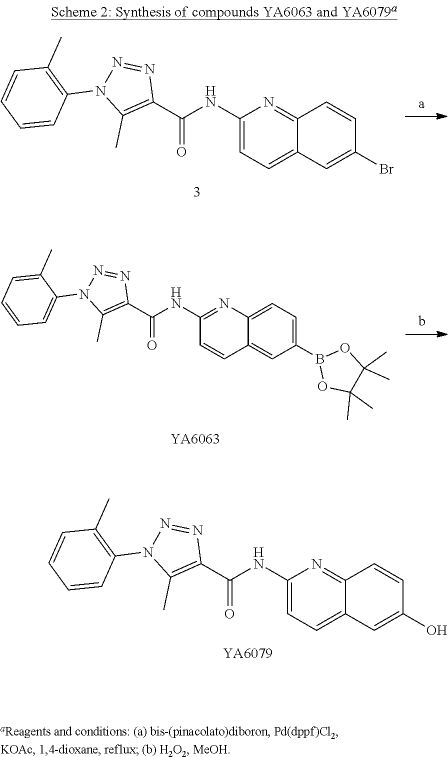

The synthesis of compounds YA6150, YA6167, YA1126, YA6146, YA6159 and YA6160 are shown in Scheme 3. Diazotization of the amino group of o-toluidine 1 using sodium nitrite (NaNO₂) and aqueous HCl, followed by the treatment of the intermediate with sodium azide (NaN₃) provided azides, which underwent cyclization with β-ketoester in EtONa/EtOH yielded trizaole-3-carboxylic acids 2. Next, PyCIU-mediated coupling of compound 2 with various bromoquinolin-2-amines in dichloroethane (DCE) provided compounds YA6150, YA6167, YA1126, and YA6146 in moderate to good yields. Finally, Pd-catalyzed cross-coupling of compounds YA6150 and YA6146 with potassium trifluoroborate derivatives yielded products YA6159 and YA6160 in good yields.

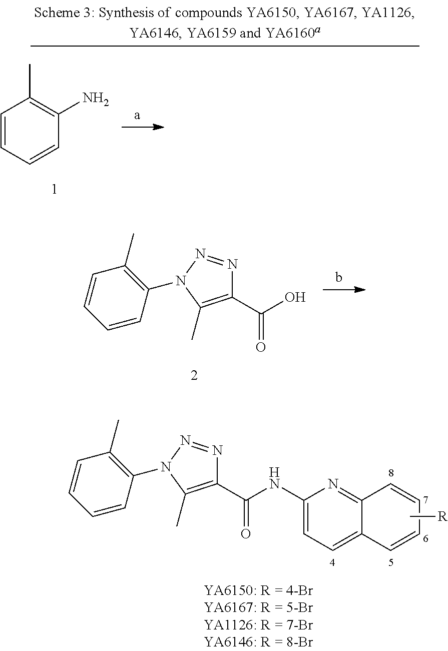

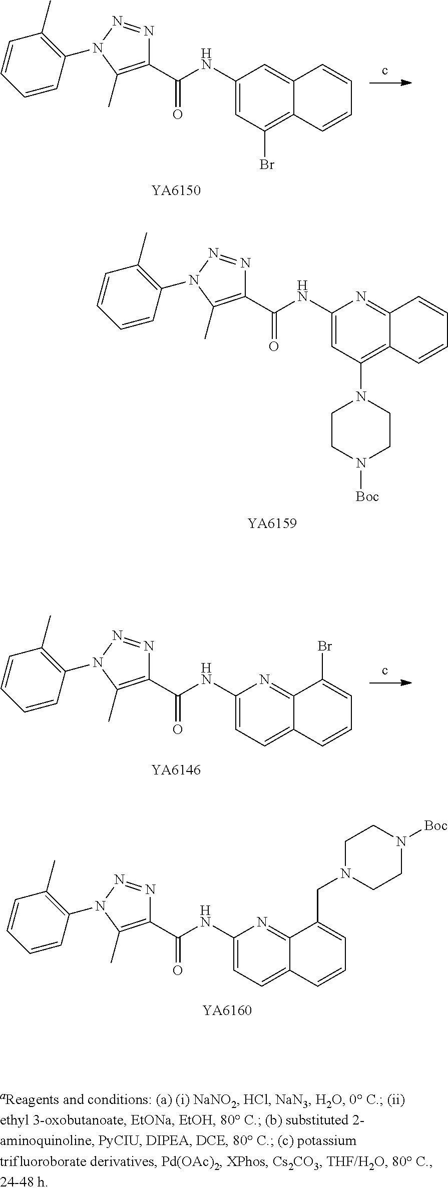

The synthesis of compounds YA4182, YA4179, YA1130 and YA1128 began with o-toluidines (Scheme 4). Diazotization of the amino group of aniline 1 using sodium nitrite (NaNO₂) and aqueous HCl, followed by the treatment of the intermediate with sodium azide (NaN₃) provided azides, which underwent cyclization with β-ketoester in EtONa/EtOH to yield trizaole-3-carboxylic acids 2. Next, PyCIU-mediated coupling of compounds 2 with various aromatic amines in dichloroethane (DCE) provided compounds YA4182, YA4179, YA1130 and YA1128 in moderate to good yields.

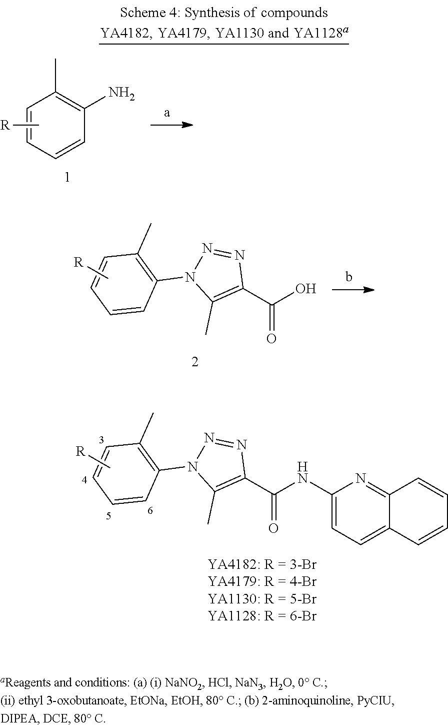

Table 1 shows experimental data showing the inhibition of the Wnt/β-catenin signaling pathway by compounds disclosed herein. Compound FX1128 is included as a comparison.

TABLE 1
Inhibition of the Wnt/β-Catenin Signaling Pathway by Compounds

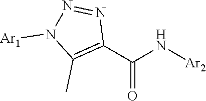

| Compound | cLogP | Ar₁ | Ar₂ | IC₅₀ (nM)^{a} | CC₅₀ (uM)^{b} |
|---|---|---|---|---|---|
| FX1128 | 3.24 | 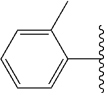 [SMILES: *c1ccccc1C] | 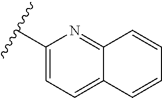 [SMILES: *c1ccc2ccccc2n1] | 4.1 | >92 |
| YA10951 | 2.49 | 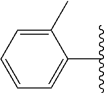 [SMILES: *c1ccccc1C] | 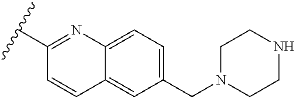 [SMILES: *c1ccc2cc(CN3CCNCC3)ccc2n1] | 4.3 | >92 |
| YA6060 | | 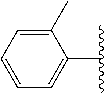 [SMILES: *c1ccccc1C] | 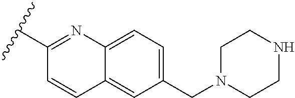 [SMILES: *c1ccc2cc(CN3CCNCC3)ccc2n1] | 4.3 | >92 |
| YA6063 | 4.49 | 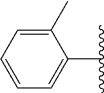 [SMILES: *c1ccccc1C] | 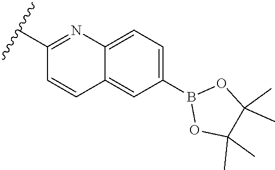 [SMILES: *c1ccc2cc(B3OC(C)(C)C(C)(C)O3)ccc2n1] | 148 | >92 |
| YA6079 | 2.46 | 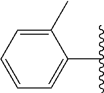 [SMILES: *c1ccccc1C] | 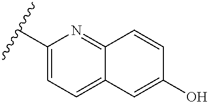 [SMILES: *c1ccc2cc(O)ccc2n1] | >1000 | >92 |
| YA6150 | 3.97 | 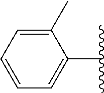 [SMILES: *c1ccccc1C] | 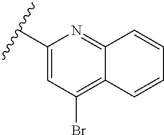 [SMILES: *c1cc(Br)c2ccccc2n1] | >1000 | >92 |
| YA6167 | 3.98 | 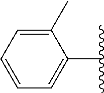 [SMILES: *c1ccccc1C] | 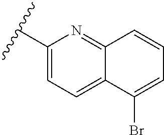 [SMILES: *c1ccc2c(Br)cccc2n1] | >1000 | >92 |
| YA1126 | 3.97 | 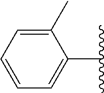 [SMILES: *c1ccccc1C] | 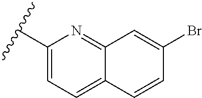 [SMILES: *c1ccc2ccc(Br)cc2n1] | >1000 | >92 |
| YA6146 | 3.96 | 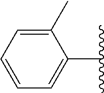 [SMILES: *c1ccccc1C] | 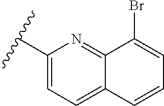 [SMILES: *c1ccc2cccc(Br)c2n1] | >1000 | >92 |
| YA6159 | 3.73 | 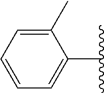 [SMILES: *c1ccccc1C] | 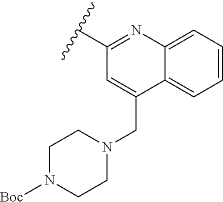 [SMILES: *c1cc(CN2CCN(C(=O)OC(C)(C)C)CC2)c2ccccc2n1] | >1000 | >92 |
| YA6160 | 3.75 | 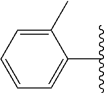 [SMILES: *c1ccccc1C] | 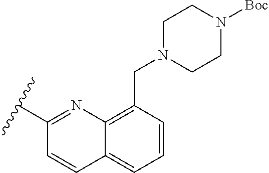 [SMILES: *c1ccc2cccc(CN3CCN(C(=O)OC(C)(C)C)CC3)c2n1] | >1000 | >92 |
| YA4182 | 3.96 | 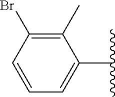 [SMILES: *c1cccc(Br)c1C] | 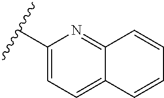 [SMILES: *c1ccc2ccccc2n1] | nt | nt |
| YA4179 | 3.99 | 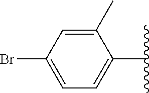 [SMILES: *c1ccc(Br)cc1C] | 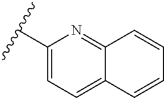 [SMILES: *c1ccc2ccccc2n1] | nt | nt |
| YA1130 | 3.96 | 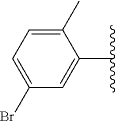 [SMILES: *c1cc(Br)ccc1C] | 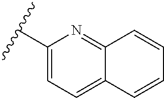 [SMILES: *c1ccc2ccccc2n1] | nt | nt |
| YA1128 | 3.94 | 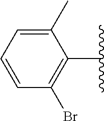 [SMILES: *c1c(C)cccc1Br] | 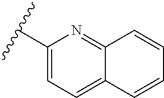 [SMILES: *c1ccc2ccccc2n1] | nt | nt |

^{a}The values of IC₅₀ for each compound to inhibit the Wnt signaling activity, as determined from theluciferase reporter gene assay, were calculated, and data are expressed as mean IC₅₀ (nM) of eachcompound from three independent experiments.
^{b}Note that compounds did not present any apparent cytotoxicity during the short treatment duration used for the luciferase gene reporter assay.

The synthesis of biotinylated compounds YA1103 and YA6023 began with compound YA10951 (Scheme 5). HATU-mediated amide bond formation between compound YA10951 and either D-biotin or biotin-dpeg(4)-CO₂H in the presence of DIPEA generated compounds YA1103 and YA6023 in modest yields.

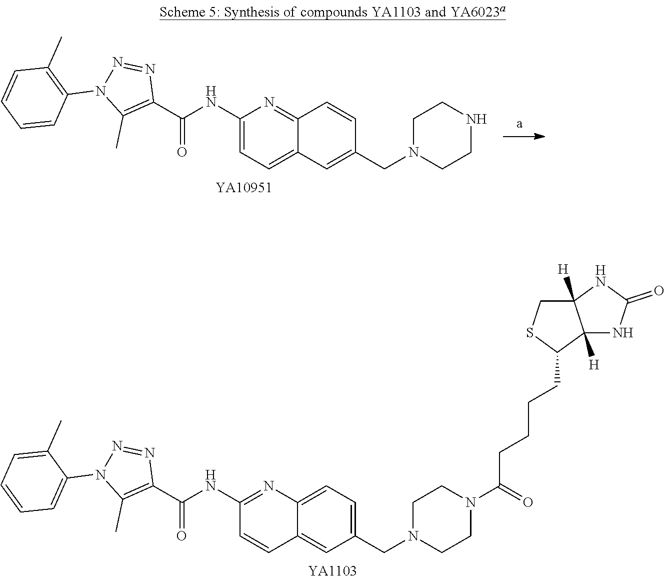

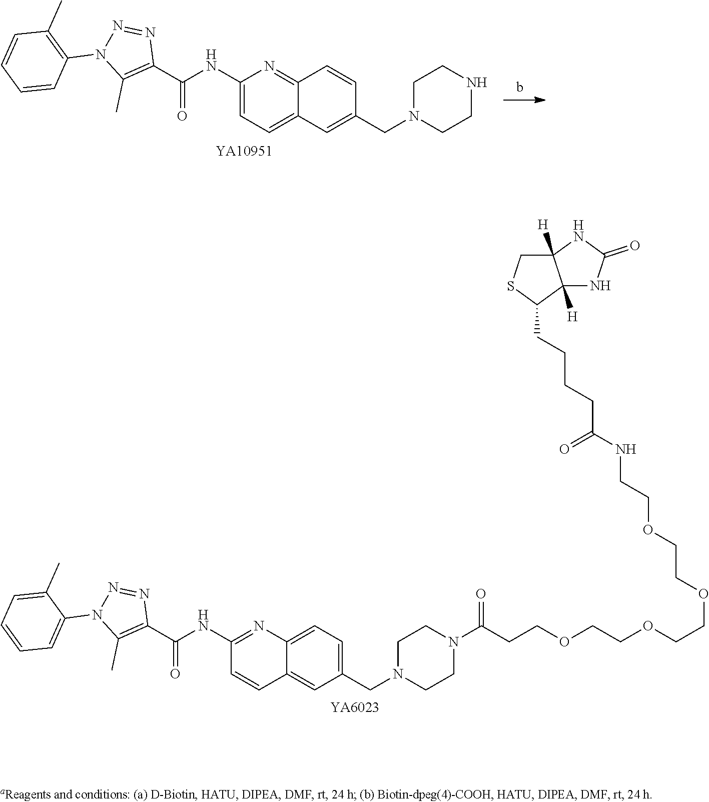

Table 2 shows experimental data showing the inhibition of the Wnt/β-catenin signaling pathway by compounds disclosed herein. Compound FX1128 is included as a comparison.

TABLE 2
Inhibition of the Wnt/β-Catenin signaling pathway by compounds

| | | | IC₅₀ | CC₅₀ |
|---|---|---|---|---|
| Compound | cLogP | structure | (nM)^{a} | (μM)^{b} |
| FX1128 | 3.24 | 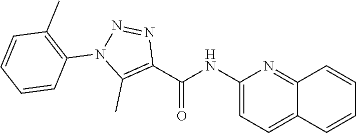 [SMILES: Cc1ccccc1-n1nnc(C(=O)Nc2ccc3ccccc3n2)c1C] | 4.1 | >92 |
| YA1103 | 3.11 | 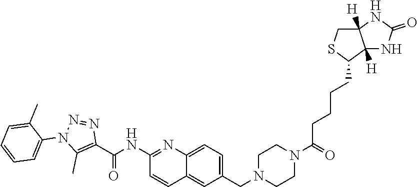 [SMILES: Cc1ccccc1-n1nnc(C(=O)Nc2ccc3cc(CN4CCN(C(=O)CCCC[C@@H]5SC[C@@H]6NC(=O)N[C@@H]65)CC4)ccc3n2)c1C] | nt | nt |
| YA6023 | 3.15 | 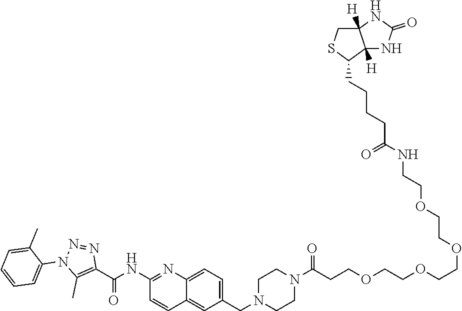 [SMILES: Cc1ccccc1-n1nnc(C(=O)Nc2ccc3cc(CN4CCN(C(=O)CCOCCOCCOCCOCCNC(=O)CCCC[C@@H]5SC[C@@H]6NC(=O)N[C@@H]65)CC4)ccc3n2)c1C] | nt | nt |

^{a}The values of IC₅₀ for each compound to inhibit the Wnt signaling activity, as determined from the luciferase reporter gene assay, were calculated, and data are expressed as mean IC₅₀ (nM) of each compound from three independent experiments.
^{b}Note that compounds did not present any apparent cytotoxicity during the short treatment duration used for the luciferase gene reporter assay.

The synthesis of tetrazole-containing compounds is detailed in Scheme 6. Condensation of ethyl 2-oxoacetate 5 and benzenesulfonohydrazide 6 generated intermediate 7, which was reacted with various aromatic diazo derivatives 9 to provide tetrazoles 10. Hydrolysis of the ethylester using LiOH generated the carboxylates 11, which was coupled to different amino compounds to provide the final products.

The synthesis of triazole-containing compounds began with o-toluidines (Scheme 6). Diazotization of the amino group of anilines 1 was carried out using sodium nitrite (NaNO₂) and aqueous HCl, followed by the treatment of the intermediate with sodium azide (NaN₃) to provide azides, which underwent cyclization with β-ketoester in EtONa/EtOH to yield trizaole-3-carboxylic acids 2. Next, PyCIU-mediated coupling of compounds 2 with various aromatic amines in dichloroethane (DCE) provided triazole-containing compounds in moderate to good yields.

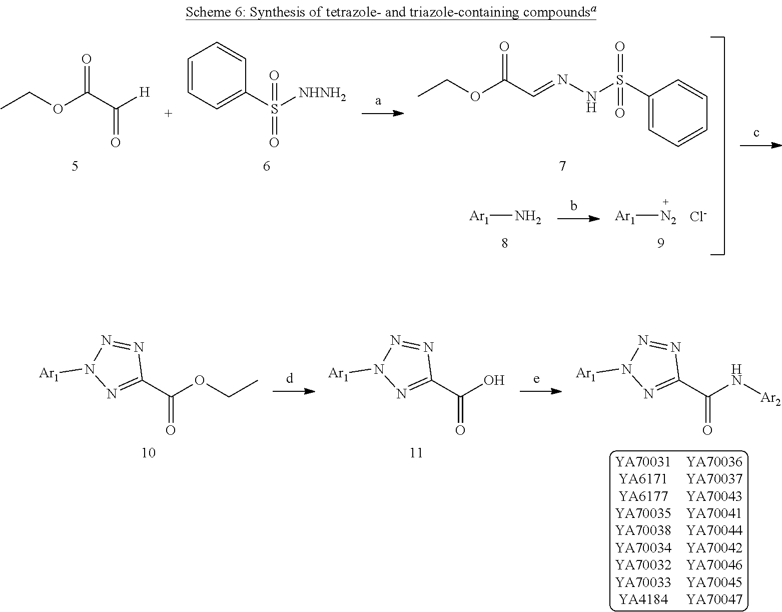

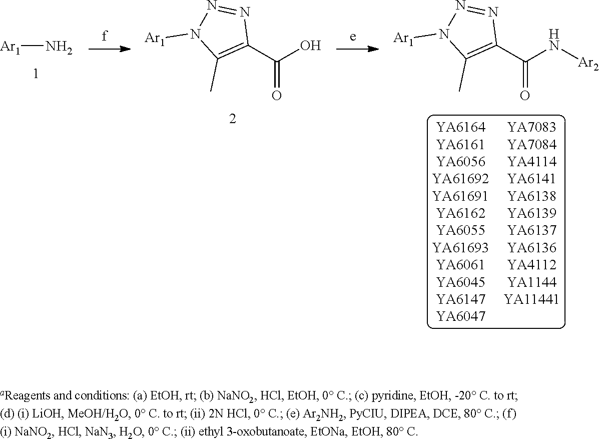

Table 3 shows experimental data showing the inhibition of the Wnt/P3-catenin signaling pathway by compounds disclosed herein. Compound FX1128 is included as a comparison.

TABLE 3
Inhibition of the Wnt/B-Catenin signaling pathway by compounds

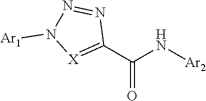

| | | | | | IC₅₀ | CC₅₀ |
|---|---|---|---|---|---|---|
| Cmpd | cLogP | Ar₁ | X | Ar₂ | (nM)ª | (μM) |
| FX1128 | 3.24 | 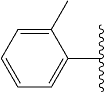 [SMILES: *c1ccccc1C] | CCH₃ | 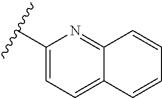 [SMILES: *c1ccc2ccccc2n1] | 4.1 | >92 |
| YA6164 | 2.91 | 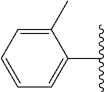 [SMILES: *c1ccccc1C] | CCH₃ | 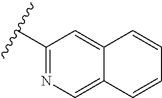 [SMILES: *c1cc2ccccc2cn1] | >1000 | >92 |
| YA6161 | 2.95 | 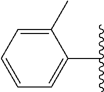 [SMILES: *c1ccccc1C] | CCH₃ | 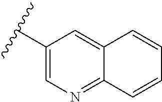 [SMILES: *c1cnc2ccccc2c1] | >1000 | >92 |
| YA6056 | 3.04 | 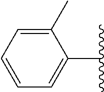 [SMILES: *c1ccccc1C] | CCH₃ | 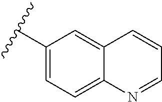 [SMILES: *c1ccc2ncccc2c1] | >1000 | >92 |
| YA61692 | 2.70 | 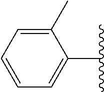 [SMILES: *c1ccccc1C] | CCH₃ | 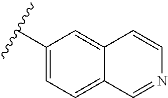 [SMILES: *c1ccc2cnccc2c1] | >1000 | >92 |
| YA61691 | 2.71 | 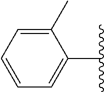 [SMILES: *c1ccccc1C] | CCH₃ | 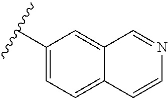 [SMILES: *c1ccc2ccncc2c1] | >1000 | >92 |
| YA6162 | 3.03 | 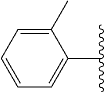 [SMILES: *c1ccccc1C] | CCH₃ | 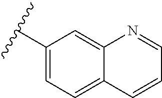 [SMILES: *c1ccc2cccnc2c1] | >1000 | >92 |
| YA6055 | 2.90 | 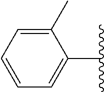 [SMILES: *c1ccccc1C] | CCH₃ | 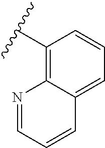 [SMILES: *c1cccc2cccnc12] | >1000 | >92 |
| YA61693 | 2.99 | 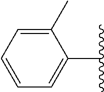 [SMILES: *c1ccccc1C] | CCH₃ | 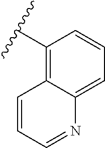 [SMILES: *c1cccc2ncccc12] | >1000 | >92 |
| YA6061 | 2.36 | 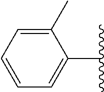 [SMILES: *c1ccccc1C] | CCH₃ | 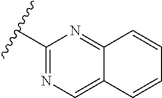 [SMILES: *c1ncc2ccccc2n1] | >1000 | >92 |
| YA6045 | 2.29 | 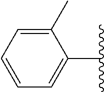 [SMILES: *c1ccccc1C] | CCH₃ | 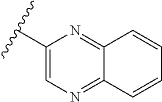 [SMILES: *c1cnc2ccccc2n1] | 1.8 | >92 |
| YA6147 | 2.30 | 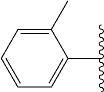 [SMILES: *c1ccccc1C] | CCH₃ | 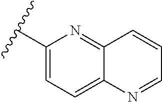 [SMILES: *c1ccc2ncccc2n1] | >1000 | >92 |
| YA6047 | 2.33 | 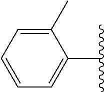 [SMILES: *c1ccccc1C] | CCH₃ | 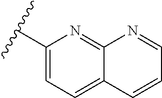 [SMILES: *c1ccc2cccnc2n1] | >1000 | >92 |
| YA7083 | 2.43 | 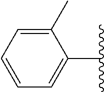 [SMILES: *c1ccccc1C] | CCH₃ | 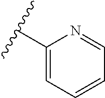 [SMILES: *c1ccccn1] | nt | nt |
| YA7084 | 1.78 | 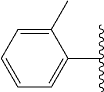 [SMILES: *c1ccccc1C] | CCH₃ | 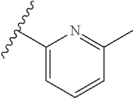 [SMILES: *c1cccc(C)n1] | nt | nt |
| YA4114 | 2.76 | 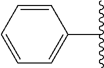 [SMILES: *c1ccccc1] | CCH₃ | 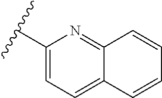 [SMILES: *c1ccc2ccccc2n1] | nt | nt |
| YA6141 | 2.17 | 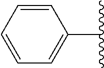 [SMILES: *c1ccccc1] | CCH₃ | 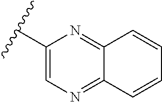 [SMILES: *c1cnc2ccccc2n1] | 1.7 | >92 |
| YA6138 | 2.52 | 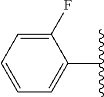 [SMILES: *c1ccccc1F] | CCH₃ | 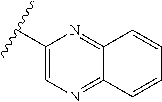 [SMILES: *c1cnc2ccccc2n1] | 0.15 | >92 |
| YA6139 | 2.83 | 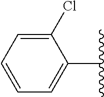 [SMILES: *c1ccccc1Cl] | CCH₃ | 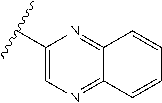 [SMILES: *c1cnc2ccccc2n1] | 0.11 | >92 |
| YA6137 | 1.94 | 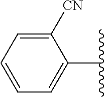 [SMILES: *c1ccccc1C#N] | CCH₃ | 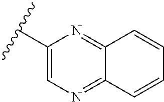 [SMILES: *c1cnc2ccccc2n1] | 6 | >92 |
| YA6136 | 2.20 | 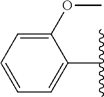 [SMILES: *c1ccccc1OC] | CCH₃ | 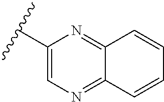 [SMILES: *c1cnc2ccccc2n1] | 1.8 | >92 |
| YA4112 | 2.14 | 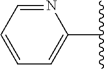 [SMILES: *c1ccccn1] | CCH₃ | 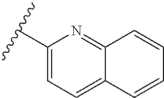 [SMILES: *c1ccc2ccccc2n1] | nt | nt |
| YA1144 | 1.98 | 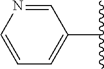 [SMILES: *c1cccnc1] | CCH₃ | 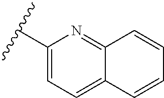 [SMILES: *c1ccc2ccccc2n1] | nt | nt |
| YA11441 | 1.10 | 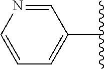 [SMILES: *c1cccnc1] | CCH₃ | 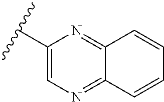 [SMILES: *c1cnc2ccccc2n1] | nt | nt |
| YA70131 | 2.85 | 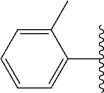 [SMILES: *c1ccccc1C] | CH | 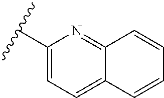 [SMILES: *c1ccc2ccccc2n1] | >1000 | >92 |
| YA70031 | 2.67 | 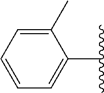 [SMILES: *c1ccccc1C] | N | 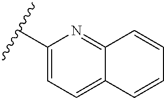 [SMILES: *c1ccc2ccccc2n1] | >1000 | >92 |
| YA6171 | 2.47 | 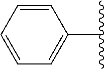 [SMILES: *c1ccccc1] | N | 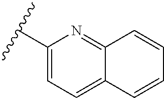 [SMILES: *c1ccc2ccccc2n1] | >1000 | >92 |
| YA6177 | 1.85 | 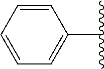 [SMILES: *c1ccccc1] | N | 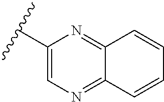 [SMILES: *c1cnc2ccccc2n1] | >1000 | >92 |
| YA70035 | 3.23 | 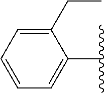 [SMILES: *c1ccccc1CC] | N | 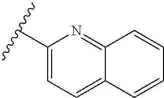 [SMILES: *c1ccc2ccccc2n1] | nt | nt |
| YA70038 | 3.51 | 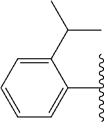 [SMILES: *c1ccccc1C(C)C] | N | 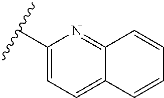 [SMILES: *c1ccc2ccccc2n1] | nt | nt |
| YA70034 | 2.57 | 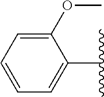 [SMILES: *c1ccccc1OC] | N | 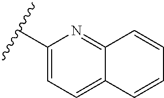 [SMILES: *c1ccc2ccccc2n1] | >1000 | >92 |
| YA70032 | 2.97 | 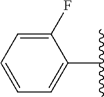 [SMILES: *c1ccccc1F] | N | 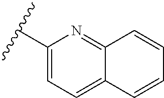 [SMILES: *c1ccc2ccccc2n1] | >1000 | >92 |
| YA70033 | 3.10 | 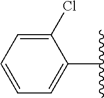 [SMILES: *c1ccccc1Cl] | N | 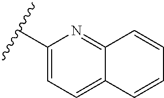 [SMILES: *c1ccc2ccccc2n1] | >1000 | >92 |
| YA4184 | 3.38 | 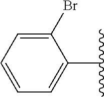 [SMILES: *c1ccccc1Br] | N | 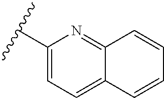 [SMILES: *c1ccc2ccccc2n1] | nt | nt |
| YA70036 | 3.85 | 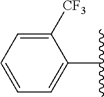 [SMILES: *c1ccccc1C(F)(F)F] | N | 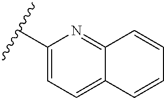 [SMILES: *c1ccc2ccccc2n1] | nt | nt |
| YA70037 | 3.75 | 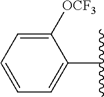 [SMILES: *c1ccccc1OC(F)(F)F] | N | 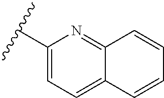 [SMILES: *c1ccc2ccccc2n1] | nt | nt |
| YA70043 | 2.79 | 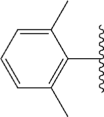 [SMILES: *c1c(C)cccc1C] | N | 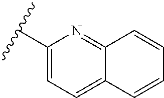 [SMILES: *c1ccc2ccccc2n1] | nt | nt |
| YA70041 | 3.04 | 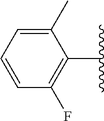 [SMILES: *c1c(C)cccc1F] | N | 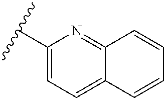 [SMILES: *c1ccc2ccccc2n1] | nt | nt |
| YA70044 | 3.23 | 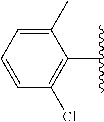 [SMILES: *c1c(C)cccc1Cl] | N | 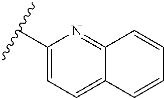 [SMILES: *c1ccc2ccccc2n1] | nt | nt |
| YA70042 | 3.50 | 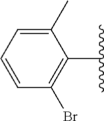 [SMILES: *c1c(C)cccc1Br] | N | 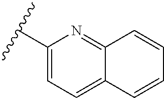 [SMILES: *c1ccc2ccccc2n1] | nt | nt |
| YA70046 | 3.39 | 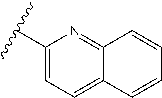 [SMILES: *c1ccc2ccccc2n1] | N | 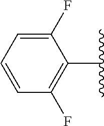 [SMILES: *c1c(F)cccc1F] | nt | nt |
| YA70045 | 3.74 | 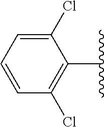 [SMILES: *c1c(Cl)cccc1Cl] | N | 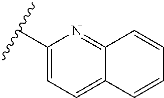 [SMILES: *c1ccc2ccccc2n1] | nt | nt |
| YA70047 | 3.83 | 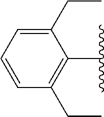 [SMILES: *c1c(CC)cccc1CC] | N | 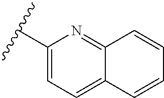 [SMILES: *c1ccc2ccccc2n1] | nt | nt |

^{a}The values of IC₅₀ for each compound to inhibit the Wnt signaling activity, as determined from the luciferase reporter gene assay, were calculated, and data are expressed as mean IC₅₀ (nM) of each compound from three independent experiments.
^{b}Note that compounds did not present any apparent cytotoxicity during the short treatment duration used for the luciferase gene reporter assay.

Table 4 shows experimental data showing the inhibition of the Wnt/β-catenin signaling pathway by compounds disclosed herein. Compound FX1128 is included as a comparison.

TABLE 4
Inhibition of the Wnt/β-Catenin signaling pathway by compounds

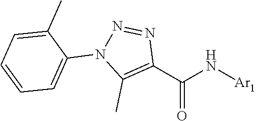

| | | | IC₅₀ | CC₅₀ |
|---|---|---|---|---|
| Cmpd | Ar₁ | Inhibition (%, 1 μM) | (nM)ª | (μM)^{b} |
| FX1128 | 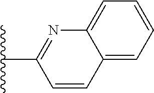 [SMILES: *c1ccc2ccccc2n1] | | 4.1 | >92 |
| YA4085 | 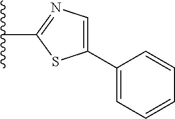 [SMILES: *c1ncc(-c2ccccc2)s1] | 37 | >1000 | >92 |
| YA4171 | 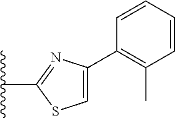 [SMILES: *c1nc(-c2ccccc2C)cs1] | | >1000 | >92 |
| YA4141 | 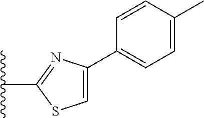 [SMILES: *c1nc(-c2ccc(C)cc2)cs1] | | >1000 | >92 |
| YA4169 | 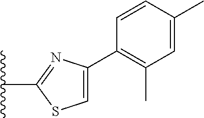 [SMILES: *c1nc(-c2ccc(C)cc2C)cs1] | | >1000 | >92 |
| YA4143 | 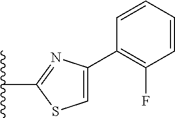 [SMILES: *c1nc(-c2ccccc2F)cs1] | | >1000 | >92 |
| YA4145 | 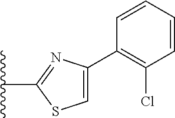 [SMILES: *c1nc(-c2ccccc2Cl)cs1] | | >1000 | >92 |
| YA4135 | 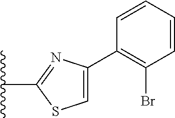 [SMILES: *c1nc(-c2ccccc2Br)cs1] | | >1000 | >92 |
| YA4173 | 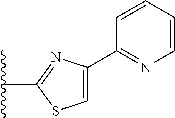 [SMILES: *c1nc(-c2ccccn2)cs1] | 7 | >1000 | >92 |
| YA4133 | 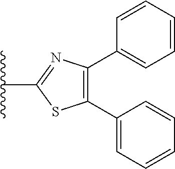 [SMILES: *c1nc(-c2ccccc2)c(-c2ccccc2)s1] | 49 | >1000 | >92 |
| YA4067 | 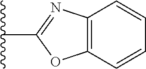 [SMILES: *c1nc2ccccc2o1] | 14 | >1000 | >92 |
| YA4061 | 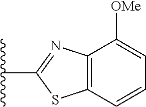 [SMILES: *c1nc2c(OC)cccc2s1] | 27 | >1000 | >92 |
| YA4045 | 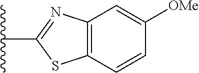 [SMILES: *c1nc2cc(OC)ccc2s1] | ND | >1000 | >92 |
| YA4091 | 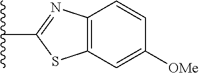 [SMILES: *c1nc2ccc(OC)cc2s1] | 12 | >1000 | >92 |
| YA4093 | 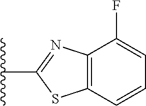 [SMILES: *c1nc2c(F)cccc2s1] | 37 | >1000 | >92 |
| YA4101 | 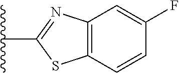 [SMILES: *c1nc2cc(F)ccc2s1] | | 16 | >92 |
| YA4103 | 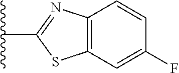 [SMILES: *c1nc2ccc(F)cc2s1] | | 4 | >92 |
| YA4095 | 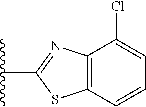 [SMILES: *c1nc2c(Cl)cccc2s1] | 8 | >1000 | >92 |
| YA4013 | 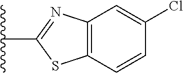 [SMILES: *c1nc2cc(Cl)ccc2s1] | 61 | 202 | >92 |
| YA4071 | 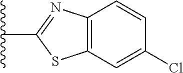 [SMILES: *c1nc2ccc(Cl)cc2s1] | | 67 | >92 |
| YA4097 | 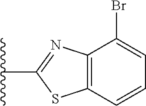 [SMILES: *c1nc2c(Br)cccc2s1] | 11 | >1000 | >92 |
| YA4047 | 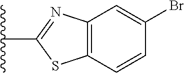 [SMILES: *c1nc2cc(Br)ccc2s1] | 14 | >1000 | >92 |
| YA4059 | 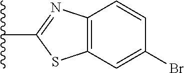 [SMILES: *c1nc2ccc(Br)cc2s1] | 31 | >1000 | >92 |
| YA4069 | 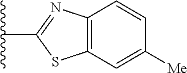 [SMILES: *c1nc2ccc(C)cc2s1] | | 15 | >92 |
| YA4065 | 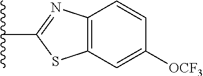 [SMILES: *c1nc2ccc(OC(F)(F)F)cc2s1] | 16 | >1000 | >92 |
| YA4063 | 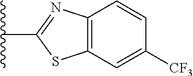 [SMILES: *c1nc2ccc(C(F)(F)F)cc2s1] | 24 | >1000 | >92 |
| YA4057 | 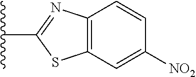 [SMILES: *c1nc2ccc([N+](=O)[O-])cc2s1] | ND | >1000 | >92 |
| Pyrvinium | 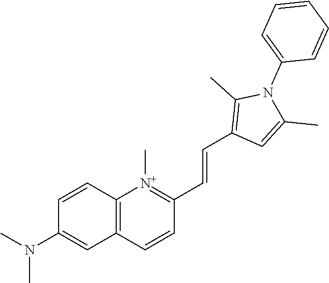 | 69 | 599 | >92 |
| XAV939 | 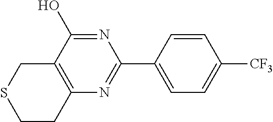 | 74 | 166 | >92 |

^{a}The values of IC₅₀ for each compound to inhibit the Wnt signaling activity, as determined from the luciferase reporter gene assay, were calculated, and data are expressed as mean IC₅₀ (nM) of each compound from three independent experiments.
^{b}Note that compounds did not present any apparent cytotoxicity during the short treatment duration used for the luciferase gene reporter assay.

Table 5 shows experimental data showing the inhibition of the Wnt/β-catenin signaling pathway by compounds disclosed herein. Compound FX1128 is included as a comparison.

TABLE 5
Inhibition of Wnt/β-Catenin Signaling Pathway by Compounds

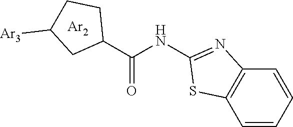

| Cmpd | Ar₂ | Ar₃ | Inhibition (%, 1 μM) | IC₅₀ (nM)^{a} | CC₅₀ (μM)^{b} |
|---|---|---|---|---|---|
| FX1128 | 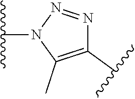 [SMILES: *c1nnn(*)c1C] | 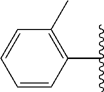 [SMILES: *c1ccccc1C] | | 4.1 | >92 |
| YA4138 | 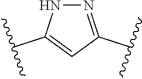 [SMILES: *c1cc(*)[nH]n1] | 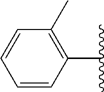 [SMILES: *c1ccccc1C] | | >1000 | >92 |
| YA6148 | 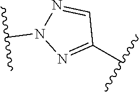 [SMILES: *c1cnn(*)n1] | 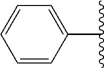 [SMILES: *c1ccccc1] | 33 | >1000 | >92 |
| YA4136 | 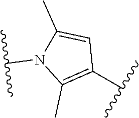 [SMILES: *c1cc(C)n(*)c1C] | 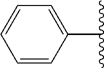 [SMILES: *c1ccccc1] | | >1000 | >92 |
| YA6082 | 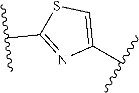 [SMILES: *c1csc(*)n1] | 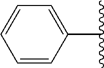 [SMILES: *c1ccccc1] | 70 | 470 | >92 |

^{a}The values of IC₅₀ for each compound to inhibit the Wnt signaling activity, as determined from the luciferase reporter gene assay, were calculated, and data are expressed as mean IC₅₀ (nM) of each compound from three independent experiments.
^{b}Note that compounds did not present any apparent cytotoxicity during the short treatment duration used for the luciferase gene reporter assay.

Table 6 shows experimental data showing the inhibition of the Wnt/β-catenin signaling pathway by compounds disclosed herein. Compound FX1128 is included as a comparison.

TABLE 6
Inhibition of Wnt/β-Catenin Signaling Pathway by Compounds

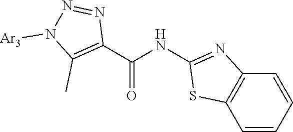

| Cmpd | Ar₃ | Inhibition (%, 1 μM) | IC₅₀ (nM)^{a} | CC₅₀ (μM)^{b} |
|---|---|---|---|---|
| FX1128 | | | 4.1 | >92 |
| YA6029 | 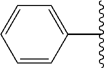 [SMILES: *c1ccccc1] | 81 | 7.82 | >92 |
| YA4029 | 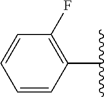 [SMILES: *c1ccccc1F] | 100 | 0.46 | >92 |
| YA1072 | 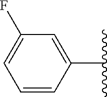 [SMILES: *c1cccc(F)c1] | 42 | >1000 | >92 |
| YA1076 | 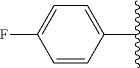 [SMILES: *c1ccc(F)cc1] | 15 | >1000 | >92 |
| YA6027 | 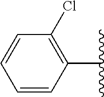 [SMILES: *c1ccccc1Cl] | 98 | 0.8 | >92 |
| YA4055 | 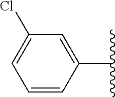 [SMILES: *c1cccc(Cl)c1] | ND | ND | >92 |
| YA1074 | 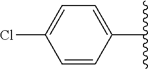 [SMILES: *c1ccc(Cl)cc1] | 18 | >1000 | >92 |
| YA4003 | 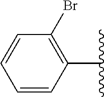 [SMILES: *c1ccccc1Br] | 100 | 1.8 | >92 |
| YA1050 | 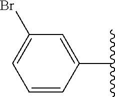 [SMILES: *c1cccc(Br)c1] | 7 | >1000 | >92 |
| YA1086 | 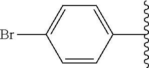 [SMILES: *c1ccc(Br)cc1] | 32 | >1000 | >92 |
| YA6089 | 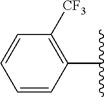 [SMILES: *c1ccccc1C(F)(F)F] | 89 | 17 | >92 |
| YA1038 | 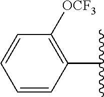 [SMILES: *c1ccccc1OC(F)(F)F] | 37 | >1000 | >92 |
| YA1046 | 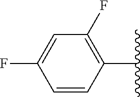 [SMILES: *c1ccc(F)cc1F] | 40 | >1000 | >92 |
| YA1084 | 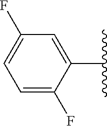 [SMILES: *c1cc(F)ccc1F] | 88 | 4.7 | >92 |
| YA6090 | 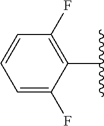 [SMILES: *c1c(F)cccc1F] | 97 | 2.5 | >92 |
| YA1066 | 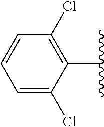 [SMILES: *c1c(Cl)cccc1Cl] | 88 | 19 | >92 |
| YA1042 | 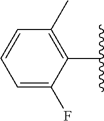 [SMILES: *c1c(C)cccc1F] | 92 | 3.3 | >92 |
| YA1068 | 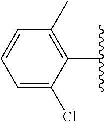 [SMILES: *c1c(C)cccc1Cl] | 80 | 35 | >92 |
| YA1064 | 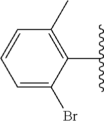 [SMILES: *c1c(C)cccc1Br] | 72 | 126 | >92 |
| YA4037 | 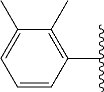 [SMILES: *c1cccc(C)c1C] | 83 | 18 | >92 |
| YA4039 | 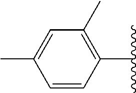 [SMILES: *c1ccc(C)cc1C] | 88 | 17 | >92 |
| YA6009 | 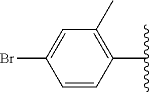 [SMILES: *c1ccc(Br)cc1C] | 15 | >1000 | >92 |
| Pyrvinium | 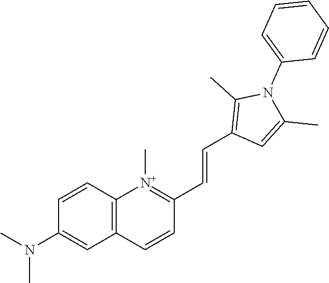 | 69 | 599 | >92 |
| XAV939 | 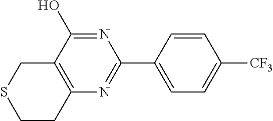 | 74 | 166 | >92 |

^{a}The values of IC₅₀ for each compound to inhibit the Wnt signaling activity, as determined from the luciferase reporter gene assay, were calculated, and data are expressed as mean IC₅₀ (nM) of each compound from three independent experiments.
^{b}Note that compounds did not present any apparent cytotoxicity during the short treatment duration used for the luciferase gene reporter assay.

Table 7 shows experimental data showing the inhibition of the Wnt/β-catenin signaling pathway by compounds disclosed herein.

TABLE 7
Inhibition of Wnt/β-Catenin Signaling Pathway by Compounds

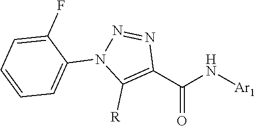

| Cmpd | Ar₁ | R | Inhibition (%, 1 μM) | IC₅₀ (nM)^{a} | CC₅₀ (μM)^{b} |
|---|---|---|---|---|---|
| YA4029 | 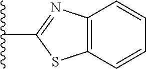 [SMILES: *c1nc2ccccc2s1] | Me | | 0.46 | >92 |
| YA4001 | 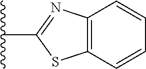 [SMILES: *c1nc2ccccc2s1] | Et | | 0.76 | >92 |
| YA4005 | 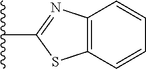 [SMILES: *c1nc2ccccc2s1] | i-Pr | 99 | 8.8 | >92 |
| YA6176 | 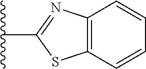 [SMILES: *c1nc2ccccc2s1] | t-Bu | 58 | 251 | >92 |
| YA6168 | 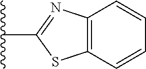 [SMILES: *c1nc2ccccc2s1] | CF | 91 | 3.3 | >92 |
| YA4155 | 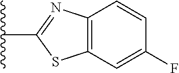 [SMILES: *c1nc2ccc(F)cc2s1] | Me | 96 | 0.4 | >92 |
| YA4165 | 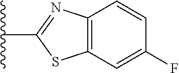 [SMILES: *c1nc2ccc(F)cc2s1] | Et | | 0.5 | >92 |
| YA6172 | 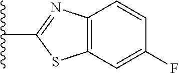 [SMILES: *c1nc2ccc(F)cc2s1] | i-Pr | 98 | 7.6 | >92 |
| YA6174 | 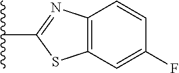 [SMILES: *c1nc2ccc(F)cc2s1] | t-Bu | 40 | >1000 | >92 |
| YA6175 | 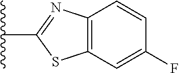 [SMILES: *c1nc2ccc(F)cc2s1] | CF3 | 85 | 17 | >92 |
| Pyrvinium | 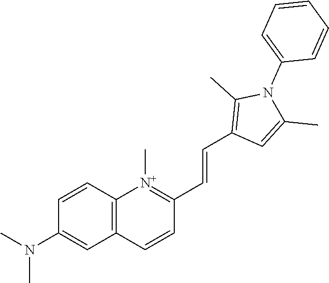 | | 69 | 599 | >92 |
| XAV939 | 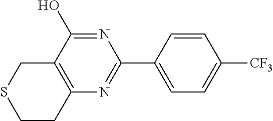 | | 74 | 166 | >92 |

^{a}The values of IC₅₀ for each compound to inhibit the Wnt signaling activity, as determined from the luciferase reporter gene assay, were calculated, and data are expressed as mean IC₅₀ (nM) of each compound from three independent experiments.
^{b}Note that compounds did not present any apparent cytotoxicity during the short treatment duration used for the luciferase gene reporter assay.

The synthesis of thiazole-triazole based compounds began with o-toluidines (Scheme 7). Diazotization of the amino group of anilines 1 was carried out using sodium nitrite (NaNO₂) and aqueous HCl, followed by the treatment of the intermediate with sodium azide (NaN₃) to provide azides, which underwent cyclization with β-ketoester in EtONa/EtOH yielded trizaole-3-carboxylic acids 2. Next, PyCIU-mediated coupling of compounds 2 with various aromatic amines in dichloroethane (DCE) provided thiazole-triazole based compounds in moderate to good yields.

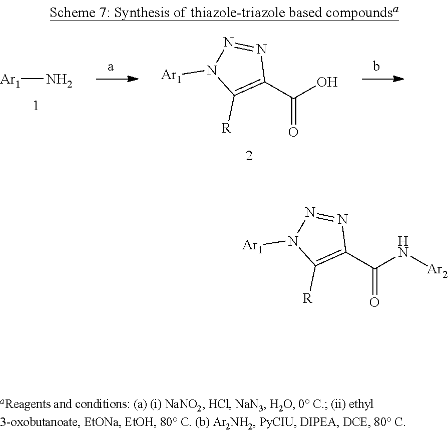

The synthesis of pyrazole compounds is detailed in Scheme 8. Condensation of ethyl acetoacetate 12 with N,N-dimethylformamide dimethyl acetal in EtOH provided compound 13 in good yields. Next, pyrazole formation using compound 13 and a substituted hydrazine in the presence of DIPEA led to the generation of the ester intermediate, which underwent hydrolysis using aqueous NaOH to afford carboxylic acids 14 in good yields. Finally, PyCIU-mediated coupling of acids 14 with various aromatic amines yielded the final products in moderate to good yields.

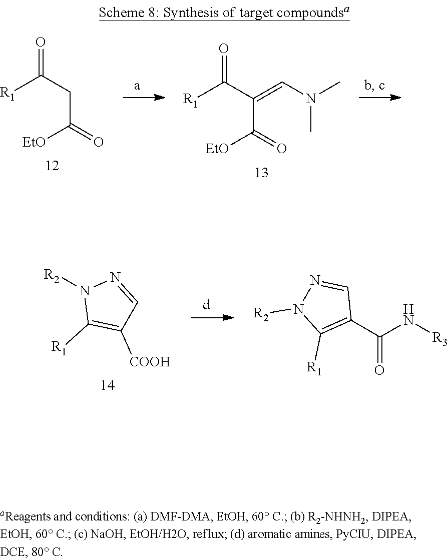

Table 8 shows experimental data showing the inhibition of the Wnt/P3-catenin signaling pathway by compounds disclosed herein.

TABLE 8
Inhibition of the Wnt/β-Catenin signaling pathway by compounds

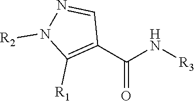

| | | | | IC₅₀ | CC₅₀ |
|---|---|---|---|---|---|
| Cmpd | R₁ | R₂ | R₃ | (nM)ª | (μM)^{b} |
| YA1124 | Me | 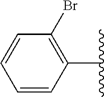 [SMILES: *c1ccccc1Br] | 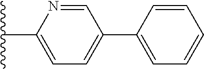 [SMILES: *c1ccc(-c2ccccc2)cn1] | nt | nt |
| YA1122 | Me | 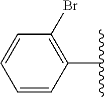 [SMILES: *c1ccccc1Br] | 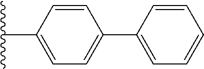 [SMILES: *c1ccc(-c2ccccc2)cc1] | nt | nt |
| YA6152 | Me | 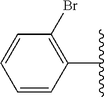 [SMILES: *c1ccccc1Br] | 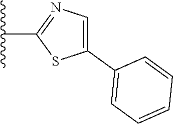 [SMILES: *c1ncc(-c2ccccc2)s1] | 220 | >92 |
| YA4087 | Me | 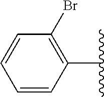 [SMILES: *c1ccccc1Br] | 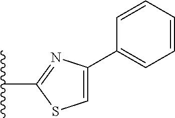 [SMILES: *c1nc(-c2ccccc2)cs1] | >1000 | >92 |
| YA4033 | Me | 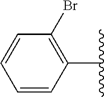 [SMILES: *c1ccccc1Br] | 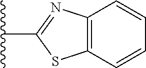 [SMILES: *c1nc2ccccc2s1] | 5.8 | >92 |
| YA7018 | Me | 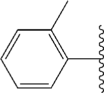 [SMILES: *c1ccccc1C] | 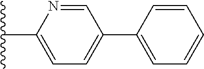 [SMILES: *c1ccc(-c2ccccc2)cn1] | >1000 | >92 |
| YA7016 | Me | 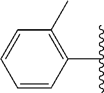 [SMILES: *c1ccccc1C] | 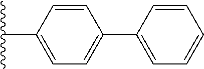 [SMILES: *c1ccc(-c2ccccc2)cc1] | >1000 | >92 |
| YA7019 | Me | 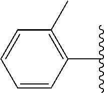 [SMILES: *c1ccccc1C] | 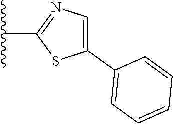 [SMILES: *c1ncc(-c2ccccc2)s1] | >1000 | >92 |
| YA7015 | Me | 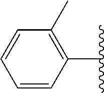 [SMILES: *c1ccccc1C] | 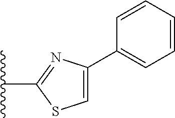 [SMILES: *c1nc(-c2ccccc2)cs1] | >1000 | >92 |
| YA6179 | Me | 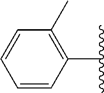 [SMILES: *c1ccccc1C] | 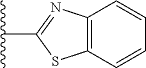 [SMILES: *c1nc2ccccc2s1] | 1.4 | >92 |
| YA4011 | Me | 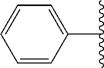 [SMILES: *c1ccccc1] | 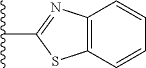 [SMILES: *c1nc2ccccc2s1] | 8.2 | >92 |
| YA6149 | H | 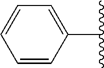 [SMILES: *c1ccccc1] | 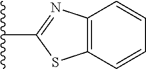 [SMILES: *c1nc2ccccc2s1] | nt | >92 |
| YA6143 | Et | 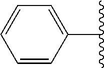 [SMILES: *c1ccccc1] | 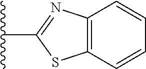 [SMILES: *c1nc2ccccc2s1] | 3.6 | >92 |
| YA6144 | i-Pr | 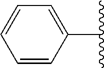 [SMILES: *c1ccccc1] | 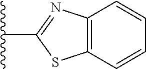 [SMILES: *c1nc2ccccc2s1] | >1000 | >92 |
| YA4009 | Me | 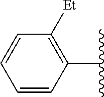 [SMILES: *c1ccccc1CC] | 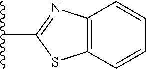 [SMILES: *c1nc2ccccc2s1] | 6.7 | >92 |
| YA7020 | Me | 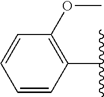 [SMILES: *c1ccccc1OC] | 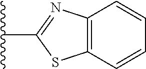 [SMILES: *c1nc2ccccc2s1] | 9.5 | >92 |
| YA4007 | Me | 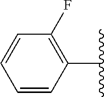 [SMILES: *c1ccccc1F] | 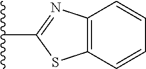 [SMILES: *c1nc2ccccc2s1] | 1.3 | >92 |
| YA4025 | Me | 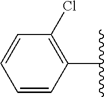 [SMILES: *c1ccccc1Cl] | 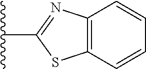 [SMILES: *c1nc2ccccc2s1] | 0.4 | >92 |
| YA4021 | Me | 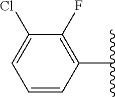 [SMILES: *c1cccc(Cl)c1F] | 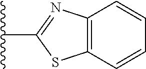 [SMILES: *c1nc2ccccc2s1] | 9.8 | >92 |
| YA4019 | Me | 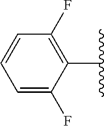 [SMILES: *c1c(F)cccc1F] | 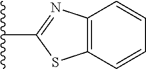 [SMILES: *c1nc2ccccc2s1] | 0.9 | >92 |
| YA4017 | Me | 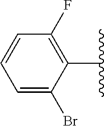 [SMILES: *c1c(F)cccc1Br] | 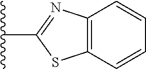 [SMILES: *c1nc2ccccc2s1] | 8.2 | >92 |
| YW2065 | Me | 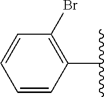 [SMILES: *c1ccccc1Br] | 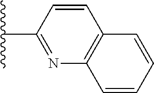 [SMILES: *c1ccc2ccccc2n1] | 2.3 | >92 |

| | | | |
|---|---|---|---|
| XAV939 | 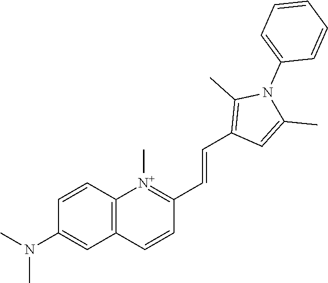 [SMILES: Cc1cc(/C=C/c2ccc3cc(N(C)C)ccc3[n+]2C)c(C)n1-c1ccccc1] | 599 | >92 |
| Pyrvinium | 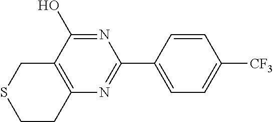 [SMILES: Oc1nc(-c2ccc(C(F)(F)F)cc2)nc2c1CSCC2] | 166 | >92 |

^{a}The values of IC₅₀ for each compound to inhibit the Wnt signaling activity, as determined from the luciferase reporter gene assay, were calculated, and data are expressed as mean IC₅₀ (nM) of each compound from three independent experiments.
^{b}Note that compounds did not present any apparent cytotoxicity during the short treatment duration used for the luciferase gene reporter assay.

### Experimental Section

All chemicals were obtained from commercial suppliers and used without further purification. Analytical thin layer chromatography was visualized by ultraviolet light at 256 nM. ¹H NMR spectra were recorded on a Varian (400 MHz) spectrometer. Data are presented as follows: chemical shift (in ppm on the δ scale relative to δ=0.00 ppm for the protons in tetramethylsilane (TMS)), integration, multiplicity (s=singlet, d=doublet, t=triplet, q=quartet, m=multiplet, br=broad), coupling constant (J/Hz). ¹³C NMR spectra were recorded at 100 MHz, and all chemical shifts values are reported in ppm on the δ scale with an internal reference of δ 77.0 or 39.0 for CDCl₃ or DMSO-d₆, respectively. The purities of title compounds were determined by analytic HPLC, performed on an Agilent 1100 instrument and a reverse-phase column (Waters XTerrra RP18, 5 μM, 4.6×250 mm). All compounds were eluted with 60% acetonitrile/40 water (containing 0.1% TFA) over 20 mins with a detection at 260 nM and a flow rate at 1.0 mL/min. All tested compounds were >95% pure.

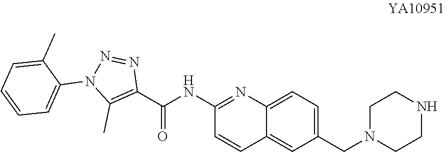 [SMILES: Cc1ccccc1-n1nnc(C(=O)Nc2ccc3cc(CN4CCNCC4)ccc3n2)c1C]

5-Methyl-N-(6-(piperazin-1-ylmethyl)quinolin-2-yl)-1-(o-tolyl)-1H-1,2,3-triazole-4-carboxamide (YA10951). The title compound was synthesized according to General Procedure E (%, a white solid): ¹H-NMR (400 MHz, CDCl₃) δ 8.53 (d, J=9.6 Hz, 1H), 8.16 (d, J=9.6 Hz, 1H), 7.85 (d, J=9.6 Hz, 1H), 7.69-7.67 (m, 2H), 7.49 (t, J=7.2 Hz, 1H), 7.43-7.37 (m, 2H), 7.25 (t, J=8.0 Hz, 1H), 3.64 (s, 2H), 2.94 (s, 4H), 2.50 (s, 7H), 2.06 (s, 3H); ¹³C-NMR (100 MHz, CDCl₃) δ 160.0, 150.4, 146.4, 138.8, 138.2, 137.8, 135.5, 135.1, 134.2, 131.5, 130.9, 127.7, 127.2, 126.1, 114.2, 63.3, 54.0, 45.7, 17.2, 9.3;

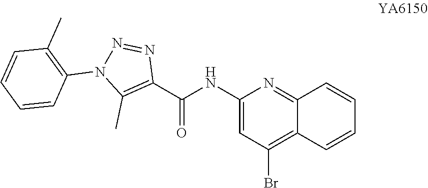 [SMILES: Cc1ccccc1-n1nnc(C(=O)Nc2cc(Br)c3ccccc3n2)c1C]

N-(4-bromoquinolin-2-yl)-5-methyl-1-(o-tolyl)-1H-1,2,3-triazole-4-carboxamide (YA6150). The title compound was synthesized according to General Procedure E (%, a white solid): ¹H-NMR (400 MHz, CDCl₃) δ 9.91 (s, 1H), 8.97 (s, 1H), 8.14 (d, J=8.0 Hz, 1H), 7.90 (d, J=8.0 Hz, 1H), 7.72 (t, J=8.0 Hz, 1H), 7.56-7.49 (m, 2H), 7.45-7.39 (m, 2H), 7.28 (d, J=8.0 Hz, 1H), 2.54 (s, 3H), 2.09 (s, 3H); ¹³C-NMR (100 MHz, CDCl₃) δ 159.9, 150.0, 147.1, 139.0, 137.5, 135.5, 135.4, 134.2, 131.5, 130.9, 130.8, 128.2, 127.1, 126.7, 126.3, 125.9, 117.9, 17.3, 9.3;

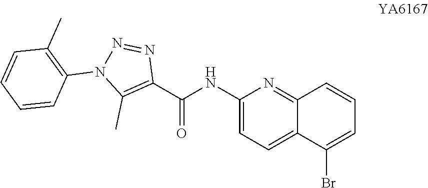 [SMILES: Cc1ccccc1-n1nnc(C(=O)Nc2ccc3c(Br)cccc3n2)c1C]

N-(5-Bromoquinolin-2-yl)-5-methyl-1-(o-tolyl)-1H-1,2,3-triazole-4-carboxamide (YA6167). The title compound was synthesized according to General Procedure E (%, a white solid): ¹H-NMR (400 MHz, CDCl₃) δ 9.20 (s, 1H), 8.10 (d, J=6.4 Hz, 1H), 7.58 (d, J=13.6 Hz, 1H), 7.53-7.49 (m, 2H), 7.46-7.40 (m, 2H), 7.31-7.25 (m, 2H), 5.97 (d, J=13.2 Hz, 1H), 2.48 (s, 3H), 2.09 (s, 3H); ¹³C-NMR (100 MHz, CDCl₃) δ 159.6, 147.1, 139.0, 137.7, 136.3, 135.6, 134.4, 131.7, 131.4, 131.2, 130.0, 127.4, 127.3, 127.2, 124.0, 122.9, 117.1, 105.1, 17.5, 9.4;

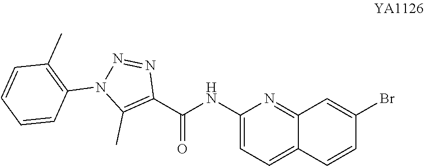 [SMILES: Cc1ccccc1-n1nnc(C(=O)Nc2ccc3ccc(Br)cc3n2)c1C]

N-(7-Bromoquinolin-2-yl)-5-methyl-1-(o-tolyl)-1H-1,2,3-triazole-4-carboxamide (YA1126). The title compound was synthesized according to General Procedure E (%, a white solid): ¹H-NMR (400 MHz, CDCl₃) δ 9.92 (s, 1H), 8.56 (d, J=8.0 Hz, 1H), 8.16 (d, J=8.0 Hz, 1H), 8.08 (s, 1H), 7.65 (d, J=8.0 Hz, 1H), 7.54-7.38 (m, 4H), 7.27-7.25 (m, 1H), 2.51 (s, 3H), 2.07 (s, 3H); ¹³C-NMR (100 MHz, CDCl₃) δ 160.0, 151.3, 147.6, 139.0, 138.3, 137.8, 135.5, 134.2, 131.5, 131.0, 130.2, 128.7, 128.6, 127.2, 124.9, 124.1, 114.4, 17.3, 9.3;

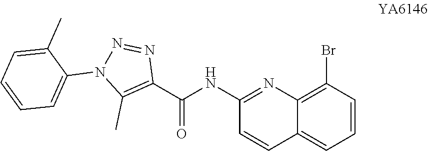 [SMILES: Cc1ccccc1-n1nnc(C(=O)Nc2ccc3cccc(Br)c3n2)c1C]

N-(8-Bromoquinolin-2-yl)-5-methyl-1-(o-tolyl)-1H-1,2,3-triazole-4-carboxamide (YA6146). The title compound was synthesized according to General Procedure E (%, a white solid): ¹H-NMR (400 MHz, CDCl₃) δ 10.02 (s, 1H), 8.55 (d, J=8.0 Hz, 1H), 8.13 (d, J=8.0 Hz, 1H), 7.93 (d, J=6.8 Hz, 1H), 7.68 (d, J=8.0 Hz, 1H), 7.44 (t, J=8.0, 7.2 Hz, 1H), 7.37-7.32 (m, 2H), 7.23-7.20 (m, 2H), 2.46 (s, 3H), 2.01 (s, 3H); ¹³C-NMR (100 MHz, CDCl₃) δ 160.0, 151.3, 144.0, 139.1, 137.6, 135.5, 134.2, 133.6, 131.5, 130.9, 127.4, 127.3, 127.1, 125.5, 122.9, 115.0, 17.2, 9.3;

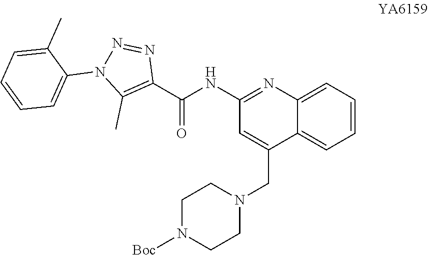 [SMILES: Cc1ccccc1-n1nnc(C(=O)Nc2cc(CN3CCN(C(=O)OC(C)(C)C)CC3)c3ccccc3n2)c1C]

tert-Butyl 4-((2-(5-methyl-1-(o-tolyl)-1H-1,2,3-triazole-4-carboxamido)quinolin-4-yl)methyl)piperazine-1-carboxylate (YA6159). ¹H-NMR (400 MHz, CDCl₃) δ 9.90 (s, 1H), 8.56 (s, 1H), 8.22 (d, J=8.0 Hz, 1H), 7.92 (d, J=8.0 Hz, 1H), 7.68 (t, J=6.8, 8.0 Hz, 1H), 7.52-7.38 (m, 4H), 7.26 (d, J=8.0 Hz, 1H), 3.96 (s, 2H), 3.46 (s, 4H), 2.52 (s, 7H), 2.07 (s, 3H), 1.45 (s, 9H); ¹³C-NMR (100 MHz, CDCl₃) δ 160.0, 154.8, 150.3, 147.3, 146.0, 138.8, 137.8, 135.5, 134.3, 131.5, 130.9, 129.7, 128.3, 127.2, 127.1, 125.9, 124.9, 124.4, 114.7, 79.6, 60.3, 53.2, 44.1, 43.2, 28.4, 17.2, 9.3;

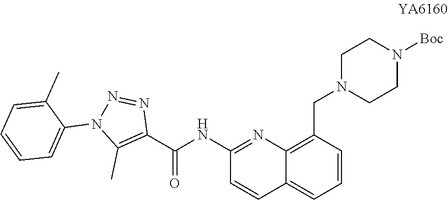 [SMILES: Cc1ccccc1-n1nnc(C(=O)Nc2ccc3cccc(CN4CCN(C(=O)OC(C)(C)C)CC4)c3n2)c1C]

tert-Butyl 4-((2-(5-methyl-1-(o-tolyl)-1H-1,2,3-triazole-4-carboxamido)quinolin-8-yl)methyl)piperazine-1-carboxylate (YA6160). ¹H-NMR (400 MHz, CDCl₃) δ 9.91 (s, 1H), 8.54 (d, J=8.0 Hz, 1H), 8.19 (d, J=8.0 Hz, 1H), 7.79 (d, J=6.0 Hz, 1H), 7.70 (d, J=7.6 Hz, 1H), 7.50 (t, J=7.6 Hz, 1H), 7.46-7.38 (m, 4H), 7.27 (d, J=8.0 Hz, 1H), 4.20 (s, 2H), 3.49 (s, 4H), 2.58 (s, 4H), 2.52 (s, 3H), 2.08 (s, 3H), 1.46 (s, 9H); ¹³C-NMR (100 MHz, CDCl₃) δ 159.9, 154.8, 149.7, 145.5, 138.9, 138.7, 137.8, 135.5, 134.6, 134.2, 131.5, 130.9, 129.9, 127.2, 126.5, 126.4, 124.8, 113.9, 79.5, 56.8, 53.2, 44.2, 43.3, 28.4, 17.2, 9.3;

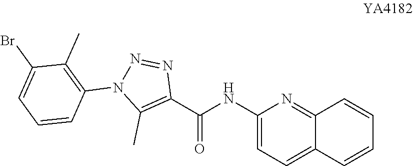 [SMILES: Cc1c(Br)cccc1-n1nnc(C(=O)Nc2ccc3ccccc3n2)c1C]

1-(3-Bromo-2-methylphenyl)-5-methyl-N-(quinolin-2-yl)-1H-1,2,3-triazole-4-carboxamide (YA4182). The title compound was synthesized according to General Procedure E (%, a white solid): ¹H-NMR (400 MHz, CDCl₃) δ 9.91 (s, 1H), 8.54 (d, J=8.8 Hz, 1H), 8.21 (d, J=8.8 Hz, 1H), 7.90 (d, J=8.8 Hz, 1H), 7.79 (d, J=8.0 Hz, 2H), 7.68 (t, J=7.6, 8.0 Hz, 1H), 7.46 (t, J=6.8, 8.0 Hz, 1H), 7.29-7.23 (m, 2H), 2.51 (s, 3H), 2.09 (S, 3H); ¹³C-NMR (100 MHz, CDCl₃) δ 159.8, 150.5, 146.9, 139.0, 138.5, 137.9, 136.1, 135.1, 130.0, 127.9, 127.8, 127.5, 126.6, 126.4, 125.2, 114.2, 18.1, 9.3;

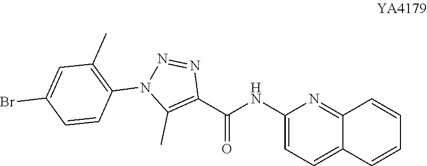 [SMILES: Cc1cc(Br)ccc1-n1nnc(C(=O)Nc2ccc3ccccc3n2)c1C]

1-(4-Bromo-2-methylphenyl)-5-methyl-N-(quinolin-2-yl)-1H-1,2,3-triazole-4-carboxamide (YA4179). The title compound was synthesized according to General Procedure E (%, a white solid): ¹H-NMR (400 MHz, CDCl₃) δ 9.95 (br s, 1H), 8.55 (d, J=8.0 Hz, 1H), 8.23 (d, J=8.0 Hz, 1H), 7.82 (d, J=8.0 Hz, 1H), 7.80 (d, J=8.0 Hz, 1H), 7.70 (t, J=7.2 Hz, 1H), 7.61 (s, 1H), 7.55 (d, J=8.0 Hz, 1H), 7.47 (t, J=7.2, 6.8 Hz, 1H), 7.14 (d, J=7.6 Hz, 1H), 2.52 (s, 3H), 2.06 (s, 3H); ¹³C-NMR (100 MHz, CDCl₃) δ 159.8, 150.5, 138.9, 138.6, 137.7, 134.5, 133.2, 130.4, 130.1, 128.6, 127.6, 127.5, 126.3, 125.3, 125.0, 114.2, 17.2, 9.27;

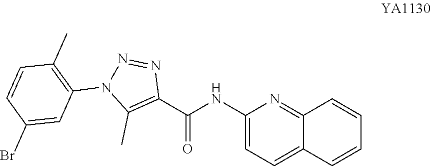 [SMILES: Cc1ccc(Br)cc1-n1nnc(C(=O)Nc2ccc3ccccc3n2)c1C]

1-(5-Bromo-2-methylphenyl)-5-methyl-N-(quinolin-2-yl)-1H-1,2,3-triazole-4-carboxamide (YA1130). The title compound was synthesized according to General Procedure E (%, a white solid): ¹H-NMR (400 MHz, CDCl₃) δ 9.90 (s, 1H), 8.54 (d, J=9.2 Hz, 1H), 8.21 (d, J=9.2 Hz, 1H), 7.90 (d, J=7.6 Hz, 1H), 7.80 (d, J=8.0 Hz, 1H), 7.68 (t, J=7.2, 8.4 Hz, 1H), 7.62 (d, J=7.6 Hz, 1H), 7.48-7.44 (m, 2H), 7.31 (d, J=8.4 Hz, 1H), 2.53 (s, 3H), 2.02 (s, 3H); ¹³C-NMR (100 MHz, CDCl₃) δ 159.8, 150.5, 146.9, 138.9, 138.5, 135.3, 134.7, 134.0, 132.8, 130.2, 130.0, 127.8, 127.5, 126.4, 125.2, 119.9, 114.2, 16.9, 9.3;

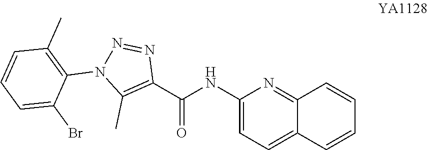 [SMILES: Cc1cccc(Br)c1-n1nnc(C(=O)Nc2ccc3ccccc3n2)c1C]

1-(2-Bromo-6-methylphenyl)-5-methyl-N-(quinolin-2-yl)-1H-1,2,3-triazole-4-carboxamide (YA1128). The title compound was synthesized according to General Procedure E (%, a white solid): ¹H-NMR (400 MHz, CDCl₃) δ 9.94 (s, 1H), 8.55 (d, J=8.0 Hz, 1H), 8.21 (d, J=8.0 Hz, 1H), 7.91 (d, J=8.0 Hz, 1H), 7.80 (d, J=8.0 Hz, 1H), 7.68 (t, J=6.8, 8.0 Hz, 1H), 7.63-7.61 (m, 1H), 7.46 (t, J=7.6, 7.2 Hz, 1H), 7.37 (d, J=6.0 Hz, 2H), 2.50 (s, 3H), 2.06 (s, 3H); ¹³C-NMR (100 MHz, CDCl₃) δ 159.9, 150.6, 147.0, 138.7, 138.4, 137.9, 133.6, 132.1, 131.2, 130.3, 130.0, 127.8, 127.5, 126.4, 125.2, 122.3, 114.2, 18.0, 8.9;

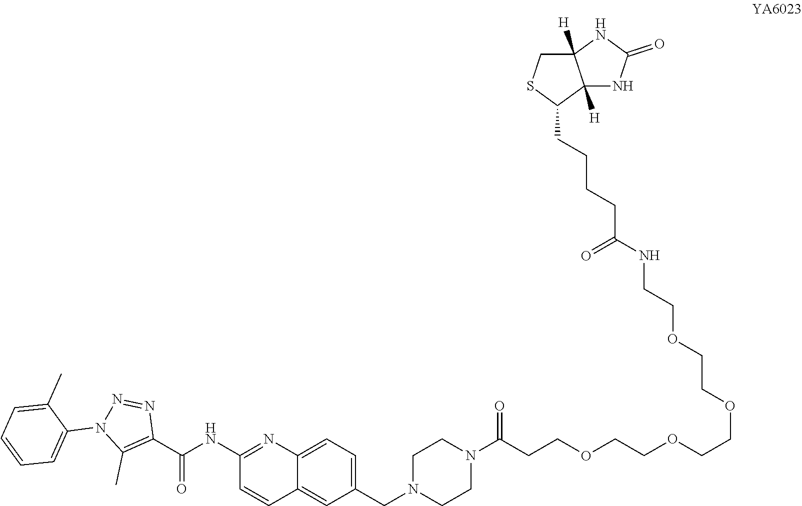 [SMILES: Cc1ccccc1-n1nnc(C(=O)Nc2ccc3cc(CN4CCN(C(=O)CCOCCOCCOCCOCCNC(=O)CCCC[C@@H]5SC[C@@H]6NC(=O)N[C@@H]65)CC4)ccc3n2)c1C]

### General Procedure A: Synthesis of Compound YA6023

To a solution of 6-bromoquinolin-2-amine (0.5 mmol) and 5-methyl-1-(o-tolyl)-1H-1,2,3-triazole-4-carboxylic acid (0.675 mmol) in DCE (2 mL) was added PyCIU (0.775 mmol) and DIPEA (2.33 mmol). The mixture was stirred at 80° C. overnight, then cooled and concentrated. To the resulting residue was added ethyl acetate (30 mL), and the mixture was washed with brine, dried over Na₂SO₄ and concentrated. The crude material was purified by column chromatography (hexane/AcOEt, v/v=4/1 to 2/1) to give product YA1061 (60%).

In a conical shaped microwave vial was added YA1061 (0.169 mmol), potassium ((4-(tert-butoxycarbonyl)piperazin-1-yl)methyl)trifluoroborate (0.338 mmol), 2-dicyclohexylphosphino-2′,4′,6′-triisopropylbiphenyl (XPhos, 0.034 mmol), cesium carbonate (0.507 mmol), and palladium (II) acetate (0.017 mmol), THF (0.5 mL) and water (0.05 mL). The reaction mixture was sealed and stirred at room temperature for 10 min. Once a clear solution was obtained, the vial was heated to 80° C. for 15 min followed by 145° C. for 45 min. After cooling, the reaction mixture was diluted with DCM and dried over Na₂SO₄. The solution was filtered, concentrated and purified by column chromatography (5-15% MeOH in DCM) to give compound YA1095 (70%).

A mixture of YA1095 (2 mmol) and TFA (15 mmol) in anhydrous DCM (5 mL) was stirred at room temperature for 0.5 h. The reaction mixture was then evaporated under reduced pressure. The resulting crude product was used in the next without further purification.

To a solution of Biotin-dpeg(4)-COOH in dry DMF (5 mL), the above products (3.0 mmol), HATU (6 mmol), and DIPEA (0.3 mmol) were added, and the mixture was stirred at room temperature for 24 h. The reaction solution was diluted with water and extracted with EA. The organic layer was washed with brine and then dried with sodium sulfate, filtered, and evaporated and purified by column chromatography (10% MeOH in DCM) to give final product YA6023 (50%). ¹H-NMR (400 MHz, CDCl₃) δ 9.87 (s, 1H), 8.49 (d, J=8.0 Hz, 1H), 8.13 (d, J=8.0 Hz, 1H), 7.82 (d, J=8.0 Hz, 1H), 7.65-7.63 (m, 2H), 7.45-7.35 (m, 3H), 7.21 (d, J=6.8 Hz, 2H), 6.90 (s, 1H), 6.42 (s, 1H), 5.46 (s, 1H), 4.43 (s, 1H), 4.25 (s, 1H), 3.74 (t, J=6.4, 7.2 Hz, 2H), 3.63-3.37 (m, 22H), 3.08-3.07 (m, 2H), 2.58 (t, J=6.4 Hz, 2H), 2.46-2.43 (m, 7H), 2.17 (t, J=7.2 Hz, 2H), 2.02 (s, 3H), 1.66-1.59 (m, 4H), 1.39-1.38 (m, 2H); ¹³C-NMR (100 MHz, CDCl₃) δ 173.3, 169.3, 163.8, 160.0, 150.5, 146.4, 138.8, 138.2, 137.7, 135.5, 134.7, 134.2, 131.5, 131.3, 130.9, 127.8, 127.1, 126.1, 114.3, 70.4, 70.3, 70.0, 69.9, 67.3, 62.6, 61.7, 60.1, 55.5, 53.1, 52.8, 45.6, 41.5, 40.5, 39.1, 35.9, 33.4, 28.1, 28.0, 25.6, 18.6, 17.2, 9.3; HRMS (ESI): calcd. for C₄₆H₆₃N₁₀O₈S [M+H]⁺ 915.4551, found 915.4546.

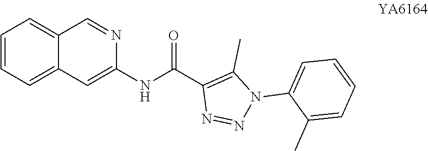 [SMILES: Cc1ccccc1-n1nnc(C(=O)Nc2cc3ccccc3cn2)c1C]

N-(Isoquinolin-3-yl)-5-methyl-1-(o-tolyl)-1H-1,2,3-triazole-4-carboxamide (YA6164). The title compound was synthesized according to General Procedure A (50%, a white solid): ¹H-NMR (400 MHz, CDCl₃) δ 9.83 (s, 1H), 9.07 (s, 1H), 8.72 (s, 1H), 7.93 (d, J=8.0 Hz, 1H), 7.85 (d, J=8.0 Hz, 1H), 7.66 (t, J=6.8, 8.0 Hz, 1H), 7.52-7.49 (m, 2H), 7.44-7.39 (m, 2H), 7.27 (d, J=7.2 Hz, 1H), 2.54 (s, 3H), 2.09 (s, 3H); ¹³C-NMR (100 MHz, CDCl₃) δ 159.6, 151.4, 146.2, 138.5, 137.9, 137.8, 135.5, 134.3, 131.5, 130.8, 130.7, 127.5, 127.2, 127.1, 126.8, 126.6, 125.8, 107.8, 17.2, 9.3;

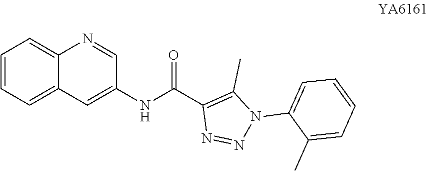 [SMILES: Cc1ccccc1-n1nnc(C(=O)Nc2cnc3ccccc3c2)c1C]

5-Methyl-N-(quinolin-3-yl)-1-(o-tolyl)-1H-1,2,3-triazole-4-carboxamide (YA6161). The title compound was synthesized according to General Procedure A (50%, a white solid): ¹H-NMR (400 MHz, CDCl₃) δ 9.43 (s, 1H), 8.99 (s, 1H), 8.92 (s, 1H), 8.08 (d, J=8.0 Hz, 1H), 7.84 (d, J=8.0 Hz, 1H), 7.66 (t, J=6.8, 8.0 Hz, 1H), 7.57-7.50 (m, 2H), 7.45-7.39 (m, 2H), 7.27 (d, J=8.0 Hz, 1H), 2.53 (s, 3H), 2.08 (s, 3H); ¹³C-NMR (100 MHz, CDCl₃) δ 159.8, 145.3, 144.1, 138.6, 137.7, 135.4, 134.2, 131.5, 131.3, 131.0, 129.1, 128.3, 127.7, 127.3, 127.2, 127.1, 123.5, 17.2, 9.2;

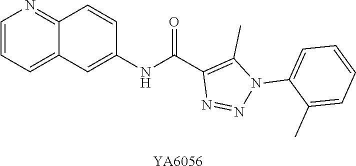

5-Methyl-N-(quinolin-6-yl)-1-(o-tolyl)-1H-1,2,3-triazole-4-carboxamide (YA6056). The title compound was synthesized according to General Procedure A (50%, a white solid): ¹H-NMR (400 MHz, CDCl₃) δ 9.32 (s, 1H), 8.82 (s, 1H), 8.51 (s, 1H), 8.14-8.07 (m, 2H), 7.76 (d, J=8.0 Hz, 1H), 7.48-7.46 (m, 1H), 7.42-7.36 (m, 3H), 7.24 (d, J=6.4 Hz, 1H), 2.49 (s, 3H), 2.05 (s, 3H); ¹³C-NMR (100 MHz, CDCl₃) δ 159.5, 149.5, 145.6, 138.5, 137.9, 135.8, 135.6, 135.4, 134.2, 131.5, 130.9, 130.4, 128.9, 127.1, 124.4, 123.2, 121.7, 115.9, 17.2, 9.2;

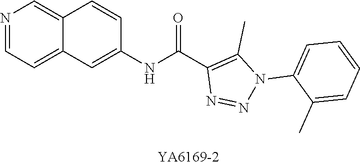 [SMILES: Cc1ccccc1-n1nnc(C(=O)Nc2ccc3cnccc3c2)c1C ; "YA6169-2"]

N-(Isoquinolin-6-yl)-5-methyl-1-(o-tolyl)-1H-1,2,3-triazole-4-carboxamide (YA6169-2). The title compound was synthesized according to General Procedure A (50%, a white solid): ¹H-NMR (400 MHz, CDCl₃) δ 9.39 (s, 1H), 9.17 (s, 1H), 8.49 (s, 2H), 7.97 (d, J=8.4 Hz, 1H), 7.71 (d, J=8.8 Hz, 1H), 7.63 (d, J=5.2 Hz, 1H), 7.51 (t, J=7.2, 6.8 Hz, 1H), 7.44-7.38 (m, 2H), 7.25 (d, J=6.4 Hz, 1H), 2.52 (s, 3H), 2.07 (s, 3H); ¹³C-NMR (100 MHz, CDCl₃) δ 159.6, 151.8, 143.6, 139.1, 138.7, 137.8, 136.9, 135.4, 134.2, 131.5, 131.0, 128.9, 127.2, 127.1, 125.8, 120.9, 120.4, 114.2, 17.2, 9.2;

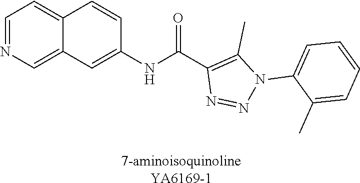 [SMILES: Cc1ccccc1-n1nnc(C(=O)Nc2ccc3ccncc3c2)c1C ; "7-aminoisoquinolineYA6169-1"]

N-(Isoquinolin-7-yl)-5-methyl-1-(o-tolyl)-1H-1,2,3-triazole-4-carboxamide (YA6169-1). The title compound was synthesized according to General Procedure A (50%, a white solid): ¹H-NMR (400 MHz, CDCl₃) δ 9.60 (s, 1H), 8.95 (d, J=3.2 Hz, 1H), 8.46 (d, J=8.0 Hz, 1H), 8.18 (d, J=7.6 Hz, 1H), 8.00 (d, J=8.0 Hz, 1H), 7.76 (d, J=8.0 Hz, 1H), 7.50-7.38 (m, 4H), 7.26 (d, J=8.0 Hz, 1H), 2.50 (s, 3H), 2.09 (s, 3H); ¹³C-NMR (100 MHz, CDCl₃) δ 159.8, 150.5, 148.7, 138.6, 138.0, 135.5, 134.3, 132.1, 131.5, 130.9, 129.8, 129.4, 127.2, 127.1, 127.0, 122.4, 121.1, 120.5, 17.3, 9.2;

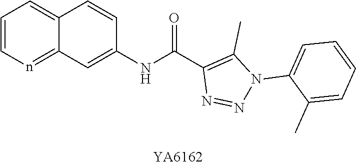

5-Methyl-N-(quinolin-7-yl)-1-(o-tolyl)-1H-1,2,3-triazole-4-carboxamide (YA6162). The title compound was synthesized according to General Procedure A (50%, a white solid): ¹H-NMR (400 MHz, CDCl₃) δ 9.37 (s, 1H), 8.90 (d, J=4.0 Hz, 1H), 8.38 (s, 1H), 8.11 (d, J=7.6 Hz, 1H), 8.04 (d, J=8.0 Hz, 1H), 7.83 (d, J=8.0 Hz, 1H), 7.49 (d, J=7.6, 7.2 Hz, 1H), 7.43-7.37 (m, 2H), 7.34-7.26 (m, 1H), 7.25 (d, J=8.0 Hz, 1H), 2.51 (s, 3H), 2.06 (s, 3H); ¹³C-NMR (100 MHz, CDCl₃) δ 159.4, 151.1, 148.9, 138.7, 138.0, 135.6, 135.5, 134.3, 131.5, 130.9, 128.7, 127.1, 125.4, 120.6, 120.2, 117.6, 17.2, 9.2;

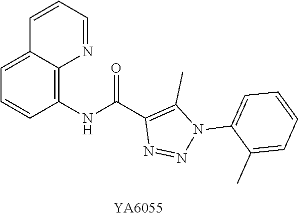

5-Methyl-N-(quinolin-8-yl)-1-(o-tolyl)-1H-1,2,3-triazole-4-carboxamide (YA6055). The title compound was synthesized according to General Procedure A (50%, a white solid): ¹H-NMR (400 MHz, CDCl₃) δ 11.61 (s, 1H), 8.94-8.89 (m, 2H), 8.16 (d, J=8.0 Hz, 1H), 7.57-7.53 (m, 2H), 7.47 (d, J=7.2 Hz, 2H), 7.43-7.36 (m, 2H), 7.26 (d, J=6.8 Hz, 1H), 2.53 (s, 3H), 2.08 (s, 3H); ¹³C-NMR (100 MHz, CDCl₃) δ 159.8, 148.7, 138.9, 138.7, 138.2, 136.1, 135.6, 134.6, 134.5, 131.4, 130.7, 128.0, 127.2, 127.1, 127.0, 121.8, 121.7, 116.4, 17.2, 9.2; HRMS (ESI): calcd. for C₂₀H₁₈N₅O [M+H]⁺ 344.1511, found 344.1508.

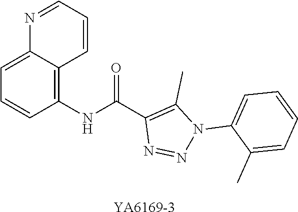 [SMILES: Cc1ccccc1-n1nnc(C(=O)Nc2cccc3ncccc23)c1C ; "YA6169-3"]

5-Methyl-N-(quinolin-5-yl)-1-(o-tolyl)-1H-1,2,3-triazole-4-carboxamide (YA6169-3). The title compound was synthesized according to General Procedure A (50%, a white solid): ¹H-NMR (400 MHz, CDCl₃) δ 9.80 (s, 1H), 9.14 (s, 1H), 8.64 (d, J=8.8 Hz, 1H), 8.37 (d, J=7.6 Hz, 1H), 8.18 (d, J=8.4 Hz, 1H), 7.94 (t, J=8.0 Hz, 1H), 7.70-7.56 (m, 4H), 7.44 (d, J=8.0 Hz, 1H), 2.68 (s, 3H), 2.27 (s, 3H); ¹³C-NMR (100 MHz, CDCl₃) δ 159.8, 150.5, 148.6, 138.6, 138.0, 135.4, 134.3, 132.1, 131.5, 130.9, 129.8, 129.4, 127.2, 127.1, 127.0, 122.4, 121.1, 120.6, 17.2, 9.2;

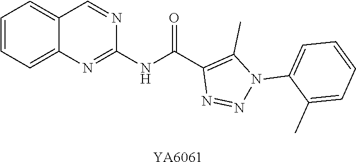

5-Methyl-N-(quinazolin-2-yl)-1-(o-tolyl)-1H-1,2,3-triazole-4-carboxamide (YA6061). The title compound was synthesized according to General Procedure A (50%, a white solid): ¹H-NMR (400 MHz, CDCl₃) δ 10.01 (s, 1H), 9.37 (s, 1H), 8.02 (d, J=8.0 Hz, 1H), 7.89 (t, J=7.6, 8.0 Hz, 2H), 7.58-7.49 (m, 2H), 7.45-7.39 (m, 2H), 7.29 (d, J=6.0 Hz, 1H), 2.54 (s, 3H), 2.08 (s, 3H); ¹³C-NMR (100 MHz, CDCl₃) δ 162.2, 158.7, 154.2, 151.2, 139.4, 135.5, 134.6, 134.3, 131.5, 130.8, 127.7, 127.3, 127.1, 127.0, 126.2, 122.5, 17.2, 9.2;

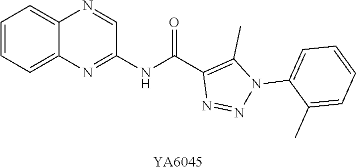

5-Methyl-N-(quinoxalin-2-yl)-1-(o-tolyl)-1H-1,2,3-triazole-4-carboxamide (YA6045). The title compound was synthesized according to General Procedure A (50%, a white solid): ¹H-NMR (400 MHz, CDCl₃) δ 9.99 (s, 1H), 9.91 (s, 1H), 8.10 (d, J=7.6 Hz, 1H), 7.91 (d, J=8.0 Hz, 1H), 7.73 (t, J=6.8, 8.0 Hz, 1H), 7.67 (t, J=7.2 Hz, 1H), 7.50 (t, J=7.2 Hz, 1H), 7.44-7.38 (m, 2H), 7.26 (d, J=6.4 Hz, 1H), 2.53 (s, 3H), 2.08 (s, 3H); ¹³C-NMR (100 MHz, CDCl₃) δ 159.7, 146.3, 141.0, 140.5, 139.4, 139.2, 137.3, 135.4, 134.1, 131.5, 131.0, 130.5, 129.1, 128.1, 127.8, 127.2, 127.1, 17.2, 9.3; HRMS (ESI): calcd. for C₁₉H₁₇N₆O [M+H]⁺ 345.1464, found 345.1469.

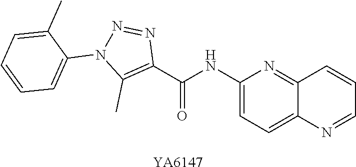

5-Methyl-N-(1,5-naphthyridin-2-yl)-1-(o-tolyl)-1H-1,2,3-triazole-4-carboxamide (YA6147). The title compound was synthesized according to General Procedure A (50%, a white solid): ¹H-NMR (400 MHz, CDCl₃) δ 9.98 (s, 1H), 8.86 (d, J=4.0 Hz, 1H), 8.78 (d, J=8.0 Hz, 1H), 8.42 (d, J=8.0 Hz, 1H), 8.19 (d, J=8.0 Hz, 1H), 7.60-7.57 (m, 1H), 7.49 (t, J=7.6, 7.2 Hz, 1H), 7.43-7.37 (m, 2H), 7.24 (d, J=8.0 Hz, 1H), 2.51 (s, 3H), 2.06 (s, 3H); ¹³C-NMR (100 MHz, CDCl₃) δ 159.9, 150.9, 149.5, 142.6, 142.3, 139.6, 139.0, 137.6, 135.6, 135.4, 134.2, 131.5, 130.9, 127.1, 124.7, 117.5, 17.2, 9.3;

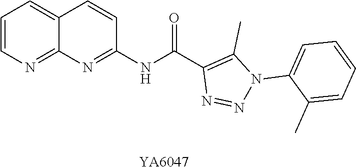

5-Methyl-N-(1,8-naphthyridin-2-yl)-1-(o-tolyl)-1H-1,2,3-triazole-4-carboxamide (YA6047). The title compound was synthesized according to General Procedure A (50%, a white solid): ¹H-NMR (400 MHz, CDCl₃) δ 10.13 (s, 1H), 9.04 (s, 1H), 8.69 (d, J=8.0 Hz, 1H), 8.23 (d, J=8.0 Hz, 1H), 8.15 (d, J=8.0 Hz, 1H), 7.49 (t, J=7.6 Hz, 1H), 7.42-7.36 (m, 3H), 7.24 (d, J=8.0 Hz, 1H), 2.50 (s, 3H, CH₃), 2.06 (s, 3H, CH₃); ¹³C-NMR (100 MHz, CDCl₃) δ 160.2, 155.1, 153.9, 153.3, 139.4, 139.0, 137.5, 136.5, 135.4, 134.2, 131.5, 130.9, 127.1, 120.8, 120.7, 115.2, 17.3, 9.3; HRMS (ESI): calcd. for C₁₉H₁₇N₆O [M+H]⁺ 345.1464, found 345.1463.

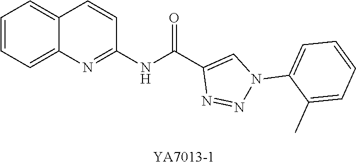 [SMILES: Cc1ccccc1-n1cc(C(=O)Nc2ccc3ccccc3n2)nn1 ; "YA7013-1"]

N-(Quinolin-2-yl)-1-(o-tolyl)-1H-1,2,3-triazole-4-carboxamide (YA7013-1). The title compound was synthesized according to General Procedure A (50%, a white solid):

¹H-NMR (400 MHz, CDCl₃) δ 9.87 (s, 1H), 8.54 (d, J=8.4 Hz, 1H), 8.41 (s, 1H), 8.21 (d, J=8.8 Hz, 1H), 7.91 (d, J=8.4 Hz, 1H), 7.80 (d, J=8.0 Hz, 1H), 7.68 (t, J=8.0, 7.6 Hz, 1H), 7.48-7.38 (m, 5H), 2.25 (s, 3H); ¹³C-NMR (100 MHz, CDCl₃) δ 158.5, 150.3, 146.9, 142.9, 138.5, 135.8, 133.6, 131.7, 130.5, 130.0, 127.8, 127.7, 127.5, 127.1, 126.5, 125.9, 125.3, 114.3, 17.9;

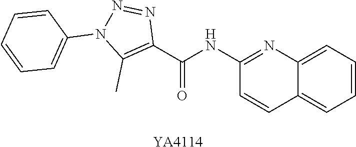

5-Methyl-1-phenyl-N-(quinolin-2-yl)-1H-1,2,3-triazole-4-carboxamide (YA4114). The title compound was synthesized according to General Procedure A (50%, a white solid): ¹H-NMR (400 MHz, CDCl₃) δ 9.91 (s, 1H), 8.54 (d, J=9.2 Hz, 1H), 8.19 (d, J=9.6 Hz, 1H), 7.90 (d, J=8.4 Hz, 1H), 7.78 (d, J=8.0 Hz, 1H), 7.67 (t, J=8.0, 7.6 Hz, 1H), 7.59-7.57 (m, 3H), 7.49-7.43 (m, 3H), 2.69 (s, 1H); ¹³C-NMR (100 MHz, CDCl₃) δ 160.0, 150.6, 146.9, 138.4, 138.2, 137.9, 135.4, 130.1, 130.0, 129.7, 127.8, 127.5, 126.4, 125.3, 125.2, 114.2, 9.9;

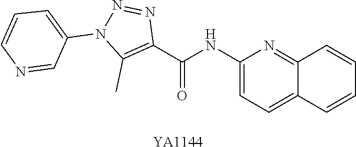

5-Methyl-1-(pyridin-3-yl)-N-(quinolin-2-yl)-1H-1,2,3-triazole-4-carboxamide (YA1144). The title compound was synthesized according to General Procedure A (50%, a white solid): ¹H-NMR (400 MHz, CDCl₃) δ 9.88 (s, 1H), 8.82 (d, J=5.6 Hz, 2H), 8.52 (d, J=8.0 Hz, 1H), 8.19 (d, J=8.0 Hz, 1H), 7.89 (d, J=8.0 Hz, 2H), 7.78 (d, J=7.6 Hz, 1H), 7.67 (t, J=8.0, 7.2 Hz, 1H), 7.58-7.54 (m, 1H), 7.45 (t, J=7.6, 8.0 Hz, 1H), 2.73 (s, 3H); ¹³C-NMR (100 MHz, CDCl₃) δ 159.6, 151.2, 150.5, 146.9, 145.8, 138.6, 138.5, 138.2, 132.7, 132.3, 130.0, 127.7, 127.5, 126.4, 125.3, 124.2, 114.2, 9.9;

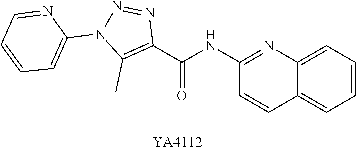

5-Methyl-1-(pyridin-2-yl)-N-(quinolin-2-yl)-1H-1,2,3-triazole-4-carboxamide (YA4112). The title compound was synthesized according to General Procedure A (50%, a white solid): ¹H-NMR (400 MHz, CDCl₃) δ 9.90 (s, 1H), 8.59-8.54 (m, 2H), 8.20 (d, J=9.6 Hz, 1H), 8.00-7.93 (m, 2H), 7.90 (d, J=7.6 Hz, 1H), 7.78 (d, J=8.0 Hz, 1H), 7.67 (t, J=8.0, 7.6 Hz, 1H), 7.46-7.41 (m, 2H), 3.02 (s, 3H); ¹³C-NMR (100 MHz, CDCl₃) δ 159.9, 150.6, 150.2, 148.5, 146.9, 139.1, 138.9, 138.7, 138.4, 130.0, 127.7, 127.5, 126.4, 125.2, 124.2, 118.2, 114.3, 11.0;

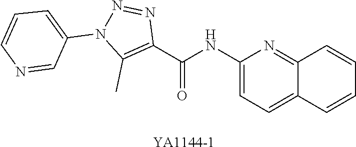 [SMILES: Cc1c(C(=O)Nc2ccc3ccccc3n2)nnn1-c1cccnc1 ; "YA1144-1"]

5-Methyl-1-(pyridin-3-yl)-N-(quinoxalin-2-yl)-1H-1,2,3-triazole-4-carboxamide (YA1144-1). The title compound was synthesized according to General Procedure A (50%, a white solid): ¹H-NMR (400 MHz, CDCl₃) δ 9.96 (s, 1H), 9.88 (s, 1H), 8.84-8.82 (m, 2H), 8.09 (d, J=8.0 Hz, 1H), 7.90 (d, J=8.0 Hz, 2H), 7.73 (t, J=8.0 Hz, 1H), 7.66 (t, J=8.0 Hz, 1H), 7.59 (t, J=6.4 Hz, 1H), 2.75 (s, 3H); ¹³C-NMR (100 MHz, CDCl₃) δ 159.4, 151.4, 146.2, 145.9, 140.9, 140.5, 139.3, 138.6, 138.1, 132.7, 132.2, 130.6, 129.1, 128.3, 127.8, 124.3, 9.9;

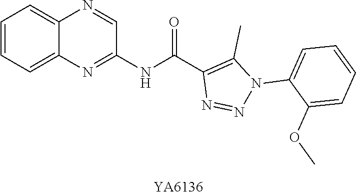

1-(2-Methoxyphenyl)-5-methyl-N-(quinoxalin-2-yl)-1H-1,2,3-triazole-4-carboxamide (YA6136). The title compound was synthesized according to General Procedure A (50%, a white solid): ¹H-NMR (400 MHz, CDCl₃) δ 9.99 (s, 1H), 9.90 (s, 1H), 8.09 (d, J=8.0 Hz, 1H), 7.89 (d, J=8.0 Hz, 1H), 7.72 (t, J=6.8, 8.0 Hz, 1H), 7.65 (t, J=8.0, 6.8 Hz, 1H), 7.55 (t, J=8.0, 7.6 Hz, 1H), 7.39 (d, J=8.0 Hz, 1H), 7.15-7.09 (m, 2H), 3.82 (s, 3H), 2.53 (s, 3H); ¹³C-NMR (100 MHz, CDCl₃) δ 159.9, 153.9, 146.4, 141.0, 140.0, 139.4, 137.0, 132.3, 130.5, 129.1, 128.4, 128.1, 127.8, 123.9, 121.1, 112.2, 55.9, 9.3;

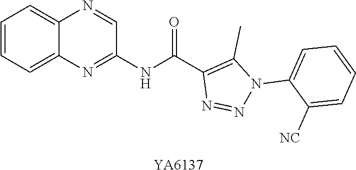

1-(2-Cyanophenyl)-5-methyl-N-(quinoxalin-2-yl)-1H-1,2,3-triazole-4-carboxamide (YA6137). The title compound was synthesized according to General Procedure A (50%, a white solid): ¹H-NMR (400 MHz, CDCl₃) δ 9.99 (s, 1H), 9.89 (s, 1H), 8.12 (d, J=8.0 Hz, 1H), 7.97-7.88 (m, 3H), 7.80-7.74 (m, 2H), 7.69 (t, J=8.0, 6.8 Hz, 1H), 7.62 (d, J=8.0 Hz, 1H), 2.71 (s, 3H); ¹³C-NMR (100 MHz, CDCl₃) δ 159.2, 146.2, 140.9, 140.6, 139.4, 136.9, 134.3, 134.2, 131.4, 130.6, 129.1, 128.3, 127.8, 114.6, 111.4, 9.6;

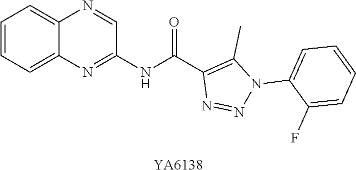

1-(2-Fluorophenyl)-5-methyl-N-(quinoxalin-2-yl)-1H-1,2,3-triazole-4-carboxamide (YA6138). The title compound was synthesized according to General Procedure A (50%, a white solid): ¹H-NMR (400 MHz, CDCl₃) δ 9.96 (s, 1H), 9.87 (s, 1H), 8.08 (d, J=8.0 Hz, 1H), 7.89 (d, J=8.0 Hz, 1H), 7.72 (t, J=6.8, 8.0 Hz, 1H), 7.67-7.51 (m, 3H), 7.40-7.32 (m, 2H), 2.62 (s, 3H); ¹³C-NMR (100 MHz, CDCl₃) δ 159.5, 156.1 (d, J=252.1 Hz), 146.3, 140.9, 140.5, 140.2, 139.4, 137.4, 132.7 (d, J=7.7 Hz), 130.5, 129.1, 128.6, 128.2, 127.8, 125.3 (d, J=3.9 Hz), 123.1 (d, J=11.6 Hz), 117.1 (d, J=19.3 Hz), 9.2;

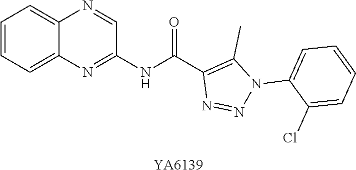

1-(2-Chlorophenyl)-5-methyl-N-(quinoxalin-2-yl)-1H-1,2,3-triazole-4-carboxamide (YA6139). The title compound was synthesized according to General Procedure A (50%, a white solid): ¹H-NMR (400 MHz, CDCl₃) δ 9.98 (s, 1H), 9.90 (s, 1H), 8.10 (d, J=7.6 Hz, 1H), 7.90 (d, J=8.0 Hz, 1H), 7.73 (t, J=7.2, 7.6 Hz, 1H), 7.68-7.63 (m, 2H), 7.58 (t, J=7.6, 8.0 Hz, 1H), 7.53-7.48 (m, 2H), 2.57 (s, 3H); ¹³C-NMR (100 MHz, CDCl₃) δ 159.6, 146.3, 141.0, 140.5, 140.2, 139.4, 137.2, 132.9, 132.3, 131.8, 130.8, 130.5, 129.1, 128.2, 128.1, 127.8, 9.3;

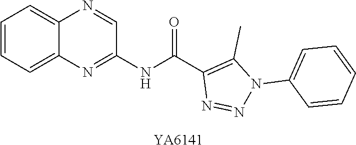

5-Methyl-1-phenyl-N-(quinoxalin-2-yl)-1H-1,2,3-triazole-4-carboxamide (YA6141). The title compound was synthesized according to General Procedure A (50%, a white solid): ¹H-NMR (400 MHz, CDCl₃) δ 9.97 (s, 1H), 9.89 (s, 1H), 8.08 (d, J=8.0 Hz, 1H), 7.89 (d, J=8.0 Hz, 1H), 7.72 (t, J=7.2, 7.6 Hz, 1H), 7.67-7.58 (m, 4H), 7.48 (d, J=6.8 Hz, 2H), 2.71 (s, 3H); ¹³C-NMR (100 MHz, CDCl₃) δ 159.7, 146.3, 140.9, 140.5, 139.4, 138.3, 137.7, 135.3, 130.5, 130.2, 129.8, 129.1, 128.2, 127.8, 125.2, 10.0;

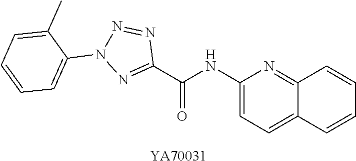

N-(Quinolin-2-yl)-2-(o-tolyl)-2H-tetrazole-5-carboxamide (YA70031). The title compound was synthesized according to General Procedure B (50%, a white solid): ¹H-NMR (400 MHz, CDCl₃) δ 9.77 (s, 1H), 8.58 (d, J=8.0 Hz, 1H), 8.24 (d, J=8.0 Hz, 1H), 7.89 (d, J=8.0 Hz, 1H), 7.80 (d, J=8.0 Hz, 1H), 7.69-7.64 (m, 2H), 7.49-7.42 (m, 4H), 2.41 (s, 3H); ¹³C-NMR (100 MHz, CDCl₃) δ 159.5, 154.9, 149.8, 146.6, 138.9, 135.9, 133.1, 132.1, 131.0, 130.2, 127.7, 127.6, 127.1, 126.7, 125.7, 125.3, 114.4, 18.8;

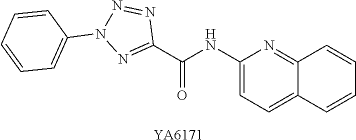

2-Phenyl-N-(quinolin-2-yl)-2H-tetrazole-5-carboxamide (YA6171). The title compound was synthesized according to General Procedure B (50%, a white solid): ¹H-NMR (400 MHz, CDCl₃) δ 9.74 (s, 1H), 8.58 (d, J=8.0 Hz, 1H), 8.26-8.21 (m, 3H), 7.90 (d, J=8.0 Hz, 1H), 7.81 (d, J=8.0 Hz, 1H), 7.69 (t, J=8.0, 6.8 Hz, 1H), 7.61-7.53 (m, 3H), 7.48 (t, J=7.2, 8.0 Hz, 1H); ¹³C-NMR (100 MHz, CDCl₃) δ 159.6, 154.8, 149.8, 146.6, 139.0, 136.4, 130.7, 130.3, 129.9, 127.7, 127.6, 127.0, 125.7, 120.3, 114.4;

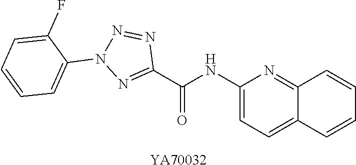

2-(2-Fluorophenyl)-N-(quinolin-2-yl)-2H-tetrazole-5-carboxamide (YA70032). The title compound was synthesized according to General Procedure B (50%, a white solid): ¹H-NMR (400 MHz, CDCl₃) δ 9.75 (s, 1H), 8.58 (d, J=8.0 Hz, 1H), 8.25 (d, J=8.0 Hz, 1H), 7.94-7.89 (m, 2H), 7.81 (d, J=8.0 Hz, 1H), 7.69 (d, J=7.6, 8.0 Hz, 1H), 7.60 (d, J=6.4 Hz, 1H), 7.48 (d, J=7.2, 7.6 Hz, 1H), 7.43-7.39 (m, 2H); ¹³C-NMR (100 MHz, CDCl₃) δ 159.7, 155.9 (d, J_{CF}=258.6 Hz), 154.6, 149.8, 146.6, 139.0, 132.7 (d, J_{CF}=7.7 Hz), 130.3 127.6, 126.7, 125.8, 125.6, 125.1 (d, J_{CF}=3.8 Hz), 124.6, 118.0 (d, J_{CF}=19.3 Hz), 114.4;

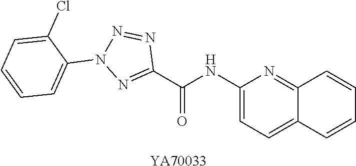

2-(2-Chlorophenyl)-N-(quinolin-2-yl)-2H-tetrazole-5-carboxamide (YA70033). The title compound was synthesized according to General Procedure B (50%, a white solid): ¹H-NMR (400 MHz, CDCl₃) δ 9.75 (s, 1H), 8.58 (d, J=8.0 Hz, 1H), 8.25 (d, J=8.0 Hz, 1H), 7.79 (d, J=8.0 Hz, 1H), 7.81 (d, J=8.0 Hz, 1H), 7.71-7.65 (m, 3H), 7.58 (t, J=8.0 Hz, 1H), 7.53-7.47 (m, 2H); ¹³C-NMR (100 MHz, CDCl₃) δ 159.7, 154.7, 149.8, 146.6, 139.0, 134.4, 132.5, 131.3, 130.3, 129.8, 127.8, 127.7, 127.6, 126.7, 125.7, 114.4;

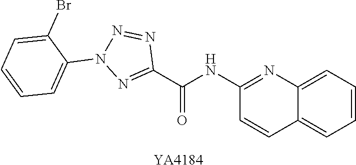

2-(2-Bromophenyl)-N-(quinolin-2-yl)-2H-tetrazole-5-carboxamide (YA4184). The title compound was synthesized according to General Procedure B (50%, a white solid): ¹H-NMR (400 MHz, CDCl₃) δ 9.75 (s, 1H), 8.59 (d, J=8.0 Hz, 1H), 8.26 (d, J=8.0 Hz, 1H), 7.90 (d, J=8.0 Hz, 1H), 7.85-7.82 (m, 2H), 7.71 (t, J=6.8, 8.0 Hz, 1H), 7.65-7.62 (m, 1H), 7.58-7.50 (m, 3H); ¹³C-NMR (100 MHz, CDCl₃) δ 159.7, 154.7, 149.8, 146.7, 139.0, 136.1, 134.4, 132.7, 130.3, 128.5, 128.0, 127.7, 127.6, 126.7, 125.8, 118.9, 114.4;

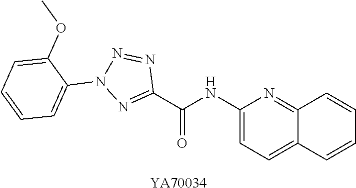

2-(2-Methoxyphenyl)-N-(quinolin-2-yl)-2H-tetrazole-5-carboxamide (YA70034). The title compound was synthesized according to General Procedure B (50%, a white solid): ¹H-NMR (400 MHz, CDCl₃) δ 9.74 (s, 1H), 8.58 (d, J=8.0 Hz, 1H), 8.23 (d, J=8.0 Hz, 1H), 7.88 (d, J=7.6 Hz, 1H), 7.80 (d, J=8.0 Hz, 1H), 7.68 (t, J=7.2, 8.0 Hz, 1H), 7.59-7.54 (m, 2H), 7.47 (t, J=7.6, 7.2 Hz, 1H), 7.12 (t, J=8.0 Hz, 2H), 3.87 (s, 3H); ¹³C-NMR (100 MHz, CDCl₃) δ 159.4, 155.0, 153.5, 149.9, 146.7, 138.9, 132.8, 130.2, 127.7, 127.6, 126.9, 126.7, 125.6, 120.7, 114.4, 112.7, 56.2;

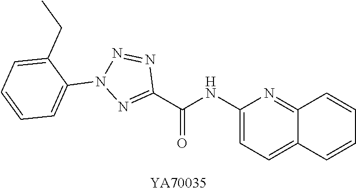

2-(2-Ethylphenyl)-N-(quinolin-2-yl)-2H-tetrazole-5-carboxamide (YA70035). The title compound was synthesized according to General Procedure B (50%, a white solid): ¹H-NMR (400 MHz, CDCl₃) δ 9.76 (s, 1H), 8.60 (d, J=8.0 Hz, 1H), 8.26 (d, J=8.0 Hz, 1H), 7.90 (d, J=8.0 Hz, 1H), 7.82 (d, J=8.0 Hz, 1H), 7.70 (t, J=7.6, 7.2 Hz, 1H), 7.60-7.42 (m, 5H), 2.71 (q, J=7.2 Hz, 2H), 1.17 (t, J=6.8 Hz, 3H); ¹³C-NMR (100 MHz, CDCl₃) δ 159.5, 154.9, 149.8, 146.7, 139.3, 138.9, 135.4, 131.4, 130.4, 130.3, 127.7, 127.6, 127.0, 126.7, 125.8, 125.7, 114.4, 24.8, 14.8;

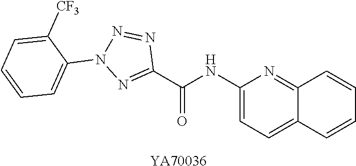

N-(Quinolin-2-yl)-2-(2-(trifluoromethyl)phenyl)-2H-tetrazole-5-carboxamide (YA70036). The title compound was synthesized according to General Procedure B (50%, a white solid): ¹H-NMR (400 MHz, CDCl₃) δ 9.74 (s, 1H), 8.54 (d, J=8.0 Hz, 1H), 8.22 (d, J=8.0 Hz, 1H), 7.92 (d, J=7.2 Hz, 1H), 7.87 (d, J=8.0 Hz, 1H), 7.82-7.78 (m, 3H), 7.71-7.65 (m, 2H), 7.46 (t, J=7.2, 7.6 Hz, 1H); ¹³C-NMR (100 MHz, CDCl₃) δ 159.9, 154.6, 149.7, 146.6, 138.9, 133.7, 133.2, 132.0, 130.3, 128.1, 128.0, 127.9, 127.7, 127.6, 126.7, 125.8, 123.6 (q, J=272.8 Hz), 114.3;

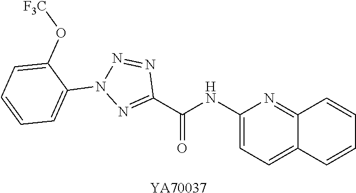

N-(quinolin-2-yl)-2-(2-(trifluoromethoxy)phenyl)-2H-tetrazole-5-carboxamide (YA70037). The title compound was synthesized according to General Procedure B (50%, a white solid): ¹H-NMR (400 MHz, CDCl₃) δ 9.76 (s, 1H), 8.57 (d, J=8.0 Hz, 1H), 8.25 (d, J=8.0 Hz, 1H), 7.89 (t, J=8.0 Hz, 2H), 7.81 (d, J=8.0 Hz, 1H), 7.69-7.66 (m, 2H), 7.58-7.54 (m, 2H), 7.48 (t, J=7.2, 7.6 Hz, 1H); ¹³C-NMR (100 MHz, CDCl₃) δ 159.7, 154.6, 149.7, 146.6, 142.1, 139.0, 132.6, 130.3, 129.3, 127.8, 127.7, 127.6, 126.8, 126.7, 125.8, 122.6, 121.5 (q, J=259.8 Hz), 114.4;

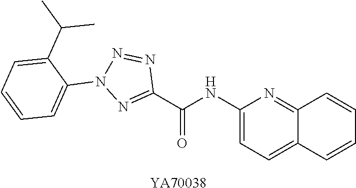

2-(2-Isopropylphenyl)-N-(quinolin-2-yl)-2H-tetrazole-5-carboxamide (YA70038). The title compound was synthesized according to General Procedure B (50%, a white solid): ¹H-NMR (400 MHz, CDCl₃) δ 9.76 (s, 1H), 8.59 (d, J=8.0 Hz, 1H), 8.25 (d, J=8.0 Hz, 1H), 7.89 (d, J=7.6 Hz, 1H), 7.81 (d, J=7.6 Hz, 1H), 7.69 (t, J=8.0, 7.6 Hz, 1H), 7.61-7.54 (m, 2H), 7.50-7.47 (m, 2H), 7.39 (t, J=6.8, 7.2 Hz, 1H), 2.92-2.89 (m, 1H), 1.22 (d, J=6.8 Hz, 6H); ¹³C-NMR (100 MHz, CDCl₃) δ 159.6, 154.9, 149.8, 146.7, 144.2, 138.9, 134.8, 131.7, 130.2, 127.7, 127.6, 127.2, 126.8, 126.7, 126.2, 125.7, 114.4, 28.2, 23.5;

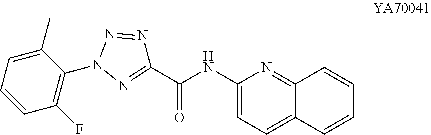 [SMILES: Cc1cccc(F)c1-n1nnc(C(=O)Nc2ccc3ccccc3n2)n1]

2-(2-Fluoro-6-methylphenyl)-N-(quinolin-2-yl)-2H-tetrazole-5-carboxamide (YA70041). The title compound was synthesized according to General Procedure B (50%, a white solid): ¹H-NMR (400 MHz, CDCl₃) δ 9.76 (s, 1H), 8.56 (d, J=8.0 Hz, 1H), 8.23 (d, J=8.0 Hz, 1H), 7.87 (d, J=8.0 Hz, 1H), 7.79 (d, J=8.0 Hz, 1H), 7.67 (t, J=8.0, 7.6 Hz, 1H), 7.52-7.44 (m, 2H), 7.20-7.14 (m, 2H), 2.15 (s, 3H); ¹³C-NMR (100 MHz, CDCl₃) δ 159.9, 158.3 (d, J=254.8 Hz), 154.7, 149.8, 146.6, 138.9, 137.5, 132.8 (d, J=9.0 Hz), 130.2, 127.7 (d, J=9.0 Hz), 126.7, 126.5 (d, J=2.6 Hz), 125.7, 124.5 (d, J=12.9 Hz), 114.4 (d, J=19.2 Hz), 17.3;

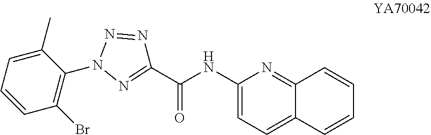 [SMILES: Cc1cccc(Br)c1-n1nnc(C(=O)Nc2ccc3ccccc3n2)n1]

2-(2-Bromo-6-methylphenyl)-N-(quinolin-2-yl)-2H-tetrazole-5-carboxamide (YA70042). The title compound was synthesized according to General Procedure B (50%, a white solid): ¹H-NMR (400 MHz, CDCl₃) δ 9.78 (s, 1H), 8.59 (d, J=8.0 Hz, 1H), 8.26 (d, J=8.0 Hz, 1H), 7.90 (d, J=8.0 Hz, 1H), 7.82 (d, J=7.6 Hz, 1H), 7.70 (t, J=7.2, 7.6 Hz, 1H), 7.63 (d, J=8.0 Hz, 1H), 7.49 (t, J=7.6, 7.2 Hz, 1H), 7.43-7.38 (m, 2H), 2.08 (s, 3H); ¹³C-NMR (100 MHz, CDCl₃) δ 159.9, 154.7, 149.8, 146.7, 139.0, 138.1, 135.6, 132.7, 131.2, 130.3, 130.1, 127.7, 127.6, 126.7, 125.8, 121.1, 114.4, 17.8;

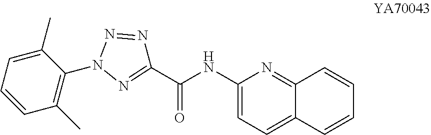 [SMILES: Cc1cccc(C)c1-n1nnc(C(=O)Nc2ccc3ccccc3n2)n1]

2-(2,6-Dimethylphenyl)-N-(quinolin-2-yl)-2H-tetrazole-5-carboxamide (YA70043). The title compound was synthesized according to General Procedure B (50%, a white solid): ¹H-NMR (400 MHz, CDCl₃) δ 9.77 (s, 1H), 8.59 (d, J=8.0 Hz, 1H), 8.26 (d, J=8.0 Hz, 1H), 7.90 (d, J=8.0 Hz, 1H), 7.82 (d, J=7.6 Hz, 1H), 7.70 (t, J=7.2, 7.6 Hz, 1H), 7.49 (t, J=6.8, 8.0 Hz, 1H), 7.41 (t, J=7.6, 7.2 Hz, 1H), 7.23 (d, J=7.6 Hz, 2H), 2.02 (s, 6H); ¹³C-NMR (100 MHz, CDCl₃) δ 159.8, 154.9, 149.8, 146.7, 138.9, 135.7, 135.2, 131.3, 130.2, 128.7, 127.8, 127.6, 126.7, 125.7, 114.4, 17.4;

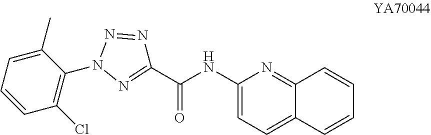 [SMILES: Cc1cccc(Cl)c1-n1nnc(C(=O)Nc2ccc3ccccc3n2)n1]

2-(2-Chloro-6-methylphenyl)-N-(quinolin-2-yl)-2H-tetrazole-5-carboxamide (YA70044). The title compound was synthesized according to General Procedure B (50%, a white solid): ¹H-NMR (400 MHz, CDCl₃) δ 9.77 (s, 1H), 8.60 (d, J=8.0 Hz, 1H), 8.28 (d, J=8.0 Hz, 1H), 7.91 (d, J=7.6 Hz, 1H), 7.84 (d, J=8.0 Hz, 1H), 7.72 (t, J=6.8, 8.0 Hz, 1H), 7.52-7.48 (m, 3H), 7.35 (d, J=6.4 Hz, 1H), 2.10 (s, 3H); ¹³C-NMR (100 MHz, CDCl₃) δ 159.9, 154.7, 149.8, 146.6, 138.9, 137.9, 134.0, 132.4, 131.9, 130.3, 129.5, 128.0, 127.7, 127.6, 126.7, 125.7, 114.4, 17.6;

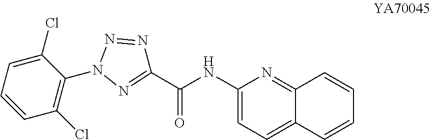 [SMILES: O=C(Nc1ccc2ccccc2n1)c1nnn(-c2c(Cl)cccc2Cl)n1]

2-(2,6-Dichlorophenyl)-N-(quinolin-2-yl)-2H-tetrazole-5-carboxamide (YA70045). The title compound was synthesized according to General Procedure B (50%, a white solid): ¹H-NMR (400 MHz, CDCl₃) δ 9.75 (s, 1H), 8.58 (d, J=8.0 Hz, 1H), 8.26 (d, J=8.0 Hz, 1H), 7.89 (d, J=8.0 Hz, 1H), 7.82 (d, J=7.6 Hz, 1H), 7.69 (t, J=7.2, 8.0 Hz, 1H), 7.56 (s, 3H), 7.49 (t, J=6.8, 8.0 Hz, 1H); ¹³C-NMR (100 MHz, CDCl₃) δ 160.0, 154.5, 149.7, 146.6, 139.0, 133.8, 133.3, 132.8, 130.3, 129.0, 127.7, 127.6, 126.7, 125.8, 114.4;

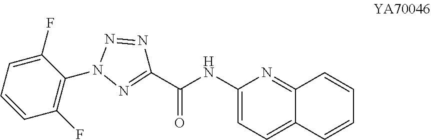 [SMILES: O=C(Nc1ccc2ccccc2n1)c1nnn(-c2c(F)cccc2F)n1]

2-(2,6-Difluorophenyl)-N-(quinolin-2-yl)-2H-tetrazole-5-carboxamide (YA70046). The title compound was synthesized according to General Procedure B (50%, a white solid): ¹H-NMR (400 MHz, CDCl₃) δ 9.73 (s, 1H), 8.57 (d, J=8.0 Hz, 1H), 8.26 (d, J=8.0 Hz, 1H), 7.90 (d, J=8.0 Hz, 1H), 7.82 (d, J=8.0 Hz, 1H), 7.71-7.61 (m, 2H), 7.49 (t, J=6.8, 8.0 Hz, 1H), 7.24-7.20 (m, 2H); ¹³C-NMR (100 MHz, CDCl₃) δ 160.1, 158.3 (d, J=258.6 Hz), 154.4, 149.7, 146.6, 139.0, 133.6 (d, J=9.0 Hz), 133.5 (d, J=9.0 Hz), 130.3, 127.7, 127.6, 126.7, 125.8, 114.4, 112.8 (d, J=2.6 Hz), 112.6 (d, J=2.6 Hz);

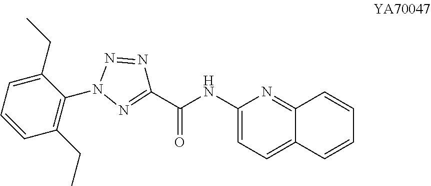 [SMILES: CCc1cccc(CC)c1-n1nnc(C(=O)Nc2ccc3ccccc3n2)n1]

2-(2,6-Diethylphenyl)-N-(quinolin-2-yl)-2H-tetrazole-5-carboxamide (YA70047). The title compound was synthesized according to General Procedure B (50%, a white solid): ¹H-NMR (400 MHz, CDCl₃) δ 9.75 (s, 1H), 8.54 (d, J=8.0 Hz, 1H), 8.19 (d, J=8.0 Hz, 1H), 7.84 (d, J=8.0 Hz, 1H), 7.76 (d, J=8.0 Hz, 1H), 7.64 (t, J=7.6, 8.0 Hz, 1H), 7.47-7.41 (m, 2H), 7.22 (d, J=7.6 Hz, 2H), 2.19 (q, J=8.0 Hz, 4H), 1.03 (t, J=8.0 Hz, 6H); ¹³C-NMR (100 MHz, CDCl₃) δ 159.6, 154.9, 149.8, 146.7, 141.1, 138.9, 134.6, 131.8, 130.2, 127.8, 127.6, 127.0, 126.7, 125.8, 114.4, 24.3, 14.7;

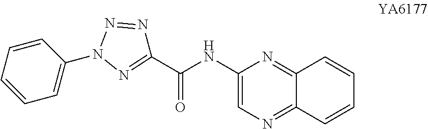 [SMILES: O=C(Nc1cnc2ccccc2n1)c1nnn(-c2ccccc2)n1]

2-Phenyl-N-(quinoxalin-2-yl)-2H-tetrazole-5-carboxamide (YA6177). The title compound was synthesized according to General Procedure B (50%, a white solid): ¹H-NMR (400 MHz, DMSO-d₆) δ 11.87 (s, 1H), 9.61 (s, 1H), 8.20 (d, J=7.6 Hz, 2H), 8.09 (d, J=8.0 Hz, 1H), 7.97 (d, J=7.6 Hz, 1H), 7.87-7.77 (m, 2H), 7.74-7.66 (m, 3H); ¹³C-NMR (100 MHz, DMSO-d₆) δ 159.7, 156.4, 146.8, 141.0, 140.7, 140.2, 136.4, 131.4, 130.7, 129.4, 129.3, 128.1, 120.8;

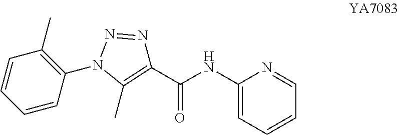 [SMILES: Cc1ccccc1-n1nnc(C(=O)Nc2ccccn2)c1C]

5-Methyl-N-(pyridin-2-yl)-1-(o-tolyl)-1H-1,2,3-triazole-4-carboxamide (YA7083).

¹H-NMR (400 MHz, CDCl₃) δ 9.67 (s, 1H), 8.35-8.31 (m, 2H), 7.72 (t, J=7.6, 8.0 Hz, 1H), 7.47 (t, J=7.2, 7.6 Hz, 1H), 7.42-7.35 (m, 2H), 7.24 (d, J=7.6 Hz, 1H), 7.05 (t, J=5.6, 6.0 Hz, 1H), 2.47 (s, 3H), 2.04 (s, 3H); ¹³C-NMR (100 MHz, CDCl₃) δ 159.7, 151.2, 148.3, 138.6, 138.2, 137.8, 135.5, 134.3, 131.5, 130.8, 127.1, 127.1, 119.8, 113.9, 17.2, 9.2;

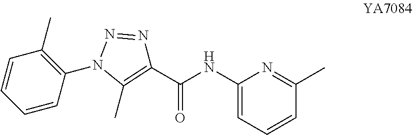 [SMILES: Cc1cccc(NC(=O)c2nnn(-c3ccccc3C)c2C)n1]

5-Methyl-N-(6-methylpyridin-2-yl)-1-(o-tolyl)-1H-1,2,3-triazole-4-carboxamide (YA7084).

¹H-NMR (400 MHz, CDCl₃) δ 9.61 (s, 1H), 8.12 (d, J=8.0 Hz, 1H), 7.61 (t, J=8.0, 7.6 Hz, 1H), 7.47 (t, J=8.0, 7.6 Hz, 1H), 7.41-7.35 (m, 2H), 7.23 (d, J=6.8 Hz, 1H), 6.91 (d, J=7.6 Hz, 1H), 2.48 (s, 3H), 2.04 (s, 3H); ¹³C-NMR (100 MHz, CDCl₃) δ 159.6, 157.2, 150.4, 138.5, 138.4, 137.8, 135.5, 134.3, 131.5, 130.8, 127.2, 127.1, 126.9, 119.3, 110.7, 24.1, 17.2, 9.2;

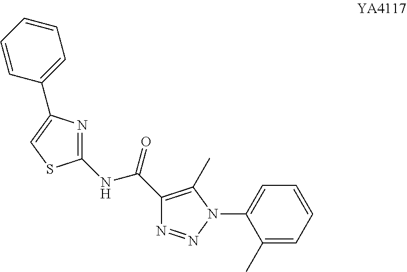 [SMILES: Cc1ccccc1-n1nnc(C(=O)Nc2nc(-c3ccccc3)cs2)c1C]

5-Methyl-N-(4-phenylthiazol-2-yl)-1-(o-tolyl)-1H-1,2,3-triazole-4-carboxamide (YA4117). The title compound was synthesized according to General Procedure B (%, a white solid): ¹H-NMR (400 MHz, CDCl₃) δ 10.47 (s, 1H), 7.79 (d, J=7.2 Hz, 2H), 7.39-7.22 (m, 6H), 7.15-7.09 (m, 2H), 2.40 (s, 3H), 1.95 (s, 3H); ¹³C-NMR (100 MHz, CDCl₃) δ 158.7, 156.7, 150.3, 139.3, 136.5, 135.4, 134.3, 134.0, 131.5, 131.0, 128.7, 128.0, 127.2, 127.1, 126.1, 107.6, 17.2, 9.2; HRMS (ESI): calcd. for C₂₀H₁₈N₅OS [M+H]⁺ 376.1232, found 376.1239.

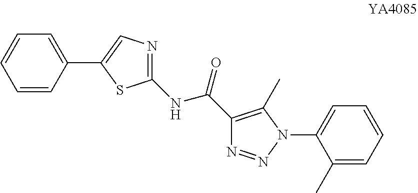 [SMILES: Cc1ccccc1-n1nnc(C(=O)Nc2ncc(-c3ccccc3)s2)c1C]

5-Methyl-N-(5-phenylthiazol-2-yl)-1-(o-tolyl)-1H-1,2,3-triazole-4-carboxamide (YA4085). The title compound was synthesized according to General Procedure B (%, a white solid): ¹H-NMR (400 MHz, CDCl₃) δ 10.79 (s, 1H), 7.54 (s, 1H), 7.30-7.28 (m, 2H), 7.20 (t, J=7.2 Hz, 1H), 7.14-7.07 (m, 4H), 7.00-6.94 (m, 2H), 2.22 (s, 3H), 1.76 (s, 3H); ¹³C-NMR (100 MHz, CDCl₃) δ 158.7, 156.5, 139.4, 136.6, 135.4, 134.1, 133.9, 132.9, 131.8, 131.5, 131.0, 129.0, 127.7, 127.2, 127.1, 126.2, 17.2, 9.3; HRMS (ESI): calcd. for C₂₀H₁₈N₅OS [M+H]⁺ 376.1232, found 376.1235.

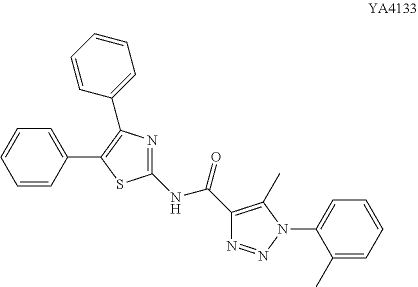 [SMILES: Cc1ccccc1-n1nnc(C(=O)Nc2nc(-c3ccccc3)c(-c3ccccc3)s2)c1C]

N-(4,5-Diphenylthiazol-2-yl)-5-methyl-1-(o-tolyl)-1H-1,2,3-triazole-4-carboxamide (YA4133). The title compound was synthesized according to General Procedure B (%, a white solid): ¹H-NMR (400 MHz, CDCl₃) δ 10.54 (s, 1H), 7.56-7.25 (m, 14H), 2.53 (s, 3H), 2.09 (s, 3H); ¹³C-NMR (100 MHz, CDCl₃) δ 153.9, 149.9, 140.0, 134.6, 131.7, 130.7, 130.1, 129.3, 127.4, 126.8, 126.2, 124.8, 124.1, 124.0, 123.6, 123.1, 123.0, 122.4, 122.4, 12.5, 4.5; HRMS (ESI): calcd. for C₂₆H₂₂N₅OS [M+H]⁺ 452.1545, found 452.1555.

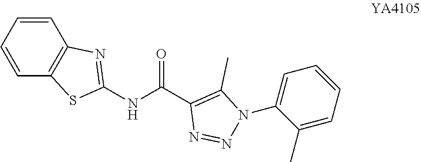 [SMILES: Cc1ccccc1-n1nnc(C(=O)Nc2nc3ccccc3s2)c1C]

N-(Benzo[d]thiazol-2-yl)-5-methyl-1-(o-tolyl)-1H-1,2,3-triazole-4-carboxamide (YA4105). The title compound was synthesized according to General Procedure B (%, a white solid): ¹H-NMR (400 MHz, CDCl₃) δ 10.53 (s, 1H), 7.80-7.78 (m, 2H), 7.45-7.27 (m, 5H), 7.18 (d, J=6.8 Hz, 1H), 2.46 (s, 3H), 2.00 (s, 3H); ¹³C-NMR (100 MHz, CDCl₃) δ 159.1, 156.8, 148.7, 139.6, 136.4, 135.4, 134.0, 132.3, 131.6, 131.0, 127.2, 127.1, 126.2, 124.0, 121.3, 17.2, 9.2; HRMS (ESI): calcd. for C₁₈H₁₆N₅OS [M+H]⁺ 350.1076, found 350.1076.

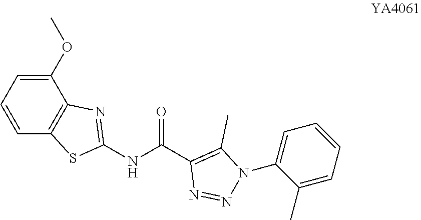 [SMILES: COc1cccc2sc(NC(=O)c3nnn(-c4ccccc4C)c3C)nc12]

N-(4-Methoxybenzo[d]thiazol-2-yl)-5-methyl-1-(o-tolyl)-1H-1,2,3-triazole-4-carboxamide (YA4061). The title compound was synthesized according to General Procedure B (%, a white solid): ¹H-NMR (400 MHz, CDCl₃) δ 10.97 (s, 1H), 7.84-7.81 (m, 1H), 7.78-7.70 (m, 3H), 7.64-7.56 (m, 2H), 7.25 (d, J=8.0 Hz, 1H), 4.38 (s, 3H), 2.85 (s, 3H), 2.39 (s, 3H); ¹³C-NMR (100 MHz, CDCl₃) δ 159.0, 155.6, 152.2, 139.4, 138.5, 136.3, 135.3, 134.0, 133.6, 131.5, 131.0, 127.1, 127.0, 124.9, 113.3, 106.8, 55.9, 17.2, 9.2; HRMS (ESI): calcd. for C₁₉H₁₈N₅O₂S [M+H]⁺ 380.1181, found 380.1191.

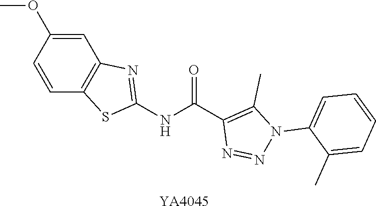

N-(5-Methoxybenzo[d]thiazol-2-yl)-5-methyl-1-(o-tolyl)-1H-1,2,3-triazole-4-carboxamide (YA4045). The title compound was synthesized according to General Procedure B (%, a white solid): ¹H-NMR (400 MHz, CDCl₃) δ 10.50 (s, 1H), 7.67 (d, J=8.8 Hz, 1H), 7.49 (t, J=6.4, 7.2 Hz, 1H), 7.43-7.34 (m, 3H), 7.24 (d, J=7.6 Hz, 1H), 6.95 (d, J=8.4 Hz, 1H), 3.86 (s, 3H), 2.50 (s, 3H), 2.05 (s, 3H); ¹³C-NMR (100 MHz, CDCl₃) δ 159.1, 158.9, 158.0, 149.9, 139.6, 136.4, 135.4, 134.0, 131.5, 131.0, 127.2, 127.1, 124.0, 121.6, 113.6, 104.3, 55.6, 17.2, 9.2; HRMS (ESI): calcd. for C₁₉H₁₈N₅O₂S [M+H]⁺ 380.1181, found 380.1183.

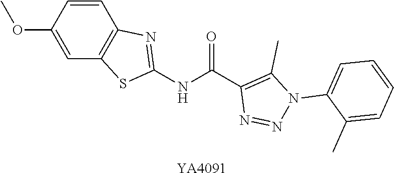

N-(6-Methoxybenzo[d]thiazol-2-yl)-5-methyl-1-(o-tolyl)-1H-1,2,3-triazole-4-carboxamide (YA4091). The title compound was synthesized according to General Procedure B (%, a white solid): ¹H-NMR (400 MHz, CDCl₃) δ 10.67 (s, 1H), 7.91 (d, J=8.0 Hz, 1H), 7.69 (t, J=8.0, 6.8 Hz, 1H), 7.63-7.56 (m, 2H), 7.50 (s, 1H), 7.43 (d, J=8.0 Hz, 1H), 7.24 (d, J=8.8 Hz, 1H), 4.06 (s, 3H), 2.70 (s, 3H), 2.25 (s, 3H); ¹³C-NMR (100 MHz, CDCl₃) δ 158.9, 156.9, 154.7, 143.0, 139.4, 136.4, 135.4, 134.0, 133.5, 131.5, 131.0, 127.2, 127.1, 121.8, 115.2, 104.2, 55.8, 17.2, 9.2; HRMS (ESI): calcd. for C₁₉H₁₈N₅O₂S [M+H]⁺ 380.1181, found 380.1176.

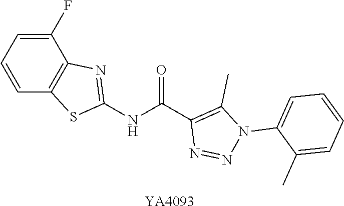

N-(4-Fluorobenzo[d]thiazol-2-yl)-5-methyl-1-(o-tolyl)-1H-1,2,3-triazole-4-carboxamide (YA4093). The title compound was synthesized according to General Procedure B (%, a white solid): ¹H-NMR (400 MHz, CDCl₃) δ 10.50 (s, 1H), 7.59 (d, J=8.0 Hz, 1H), 7.49 (t, J=7.2 Hz, 1H), 7.42-7.38 (m, 2H), 7.24-7.22 (m, 2H), 7.13 (t, J=8.0 Hz, 1H), 2.51 (s, 3H), 2.05 (s, 3H); ¹³C-NMR (100 MHz, CDCl₃) δ 159.2, 157.2, 154.8 (d, J_{CF}=252.1 Hz), 139.7, 137.5 (d, J_{CF}=12.9 Hz), 136.2, 135.3, 135.0, 133.9, 131.5, 131.0, 127.2 (d, J_{CF}=14.1 Hz), 124.6 (d, J_{CF}=7.7 Hz), 117.0 (d, J_{CF}=2.6 Hz), 112.1 (d, J_{CF}=18.0 Hz), 17.2, 9.2; HRMS (ESI): calcd. for C₁₈H₁₅FN₅OS [M+H]⁺ 368.0981, found 368.0979.

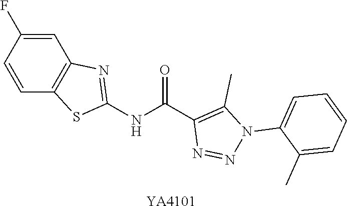

N-(5-Fluorobenzo[d]thiazol-2-yl)-5-methyl-1-(o-tolyl)-1H-1,2,3-triazole-4-carboxamide (YA4101). The title compound was synthesized according to General Procedure B (%, a white solid): ¹H-NMR (400 MHz, CDCl₃) δ 10.53 (s, 1H), 7.78-7.74 (m, 1H), 7.53-7.50 (m, 2H), 7.45-7.38 (m, 2H), 7.24 (d, J=7.6 Hz, 1H), 7.11-7.06 (m, 1H), 2.52 (s, 3H), 2.07 (s, 3H); ¹³C-NMR (100 MHz, CDCl₃) δ 161.9 (d, J=241.9 Hz), 159.1, 159.0, 149.9 (d, J=12.9 Hz) 139.7, 136.2, 135.4, 134.0, 131.5, 131.0, 127.6, 127.2 (d, J=11.5 Hz), 122.0 (d, J=9.0 Hz), 112.3 (d, J=24.5 Hz), 107.7 (d, J=23.2 Hz) 17.2, 9.2; HRMS (ESI): calcd. for C₁₈H₁₅FN₅OS [M+H]⁺ 368.0981, found 368.0974.

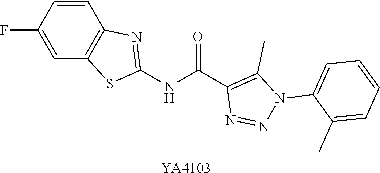

N-(6-Fluorobenzo[d]thiazol-2-yl)-5-methyl-1-(o-tolyl)-1H-1,2,3-triazole-4-carboxamide (YA4103). The title compound was synthesized according to General Procedure B (%, a white solid): ¹H-NMR (400 MHz, CDCl₃) δ 10.69 (s, 1H), 7.72-7.71 (m, 1H), 7.49-7.44 (m, 2H), 7.40-7.34 (m, 2H), 7.21 (d, J=7.6 Hz, 1H), 7.11 (t, J=7.6 Hz, 1H), 2.48 (s, 3H), 2.02 (s, 3H); ¹³C-NMR (100 MHz, CDCl₃) δ 160.8, 158.4, 157.8 (d, J_{CF}=256.0 Hz), 145.2, 139.7, 136.2, 135.4, 134.0, 133.3 (d, J_{CF}=10.3 Hz), 131.5, 131.0, 127.1 (d, J_{CF}=11.6 Hz), 122.2 (d, J_{CF}=9.1 Hz), 114.5 (d, J_{CF}=24.4 Hz), 107.6 (d, J_{CF}=25.7 Hz), 17.2, 9.2; HRMS (ESI): calcd. for C₁₈H₁₅FN₅OS [M+H]⁺ 368.0981, found 368.0979.

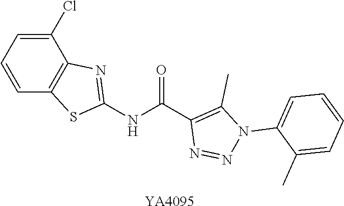

N-(4-Chlorobenzo[d]thiazol-2-yl)-5-methyl-1-(o-tolyl)-1H-1,2,3-triazole-4-carboxamide (YA4095). The title compound was synthesized according to General Procedure B (%, a white solid): ¹H-NMR (400 MHz, CDCl₃) δ 10.71 (s, 1H), 7.73 (d, J=8.0 Hz, 1H), 7.51-7.37 (m, 4H), 7.26-7.22 (m, 2H), 2.51 (s, 3H), 2.05 (s, 3H); ¹³C-NMR (100 MHz, CDCl₃) δ 159.2, 157.5, 145.8, 139.7, 136.2, 135.3, 133.9, 133.6, 131.5, 131.0, 127.2, 127.0, 126.5, 126.0, 124.5, 119.9, 17.2, 9.2; HRMS (ESI): calcd. for C₁₈H₁₅ClN₅OS [M+H]⁺ 384.0686, found 384.0694.

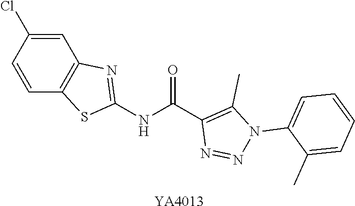

N-(5-Chlorobenzo[d]thiazol-2-yl)-5-methyl-1-(o-tolyl)-1H-1,2,3-triazole-4-carboxamide (YA4013). The title compound was synthesized according to General Procedure B (31%, a yellow solid): ¹H-NMR (400 MHz, CDCl₃) δ 7.83 (s, 1H), 7.76 (d, J=8.8 Hz, 1H), 7.53-7.51 (m, 1H), 7.46-7.40 (m, 2H), 7.31 (d, J=8.4 Hz, 1H), 7.26-7.24 (m, 1H), 2.53 (s, 3H), 2.08 (s, 3H); ¹³C-NMR (100 MHz, CDCl₃) δ 159.1, 158.3, 149.8, 139.7, 136.2, 135.4, 134.0, 132.1, 131.6, 131.1, 130.6, 127.2, 127.1, 124.3, 122.1, 121.2, 17.2, 9.2; HRMS (ESI): calcd. for C₁₈H₁₅ClN₅OS [M+H]⁺ 384.0686, found 384.0683.

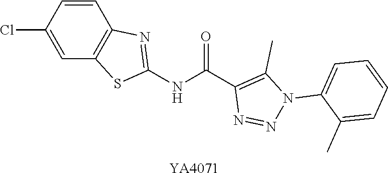

N-(6-Chlorobenzo[d]thiazol-2-yl)-5-methyl-1-(o-tolyl)-1H-1,2,3-triazole-4-carboxamide (YA4071). The title compound was synthesized according to General Procedure B (%, a white solid): ¹H-NMR (400 MHz, CDCl₃) δ 10.77 (s, 1H), 7.89 (s, 1H), 7.81 (d, J=8.4 Hz, 1H), 7.59 (t, J=8.0, 6.8 Hz, 1H), 7.53-7.46 (m, 3H), 7.33 (d, J=8.0 Hz, 1H), 2.61 (s, 3H), 2.15 (s, 3H); ¹³C-NMR (100 MHz, CDCl₃) δ 159.1, 157.2, 147.3, 139.7, 136.2, 135.4, 133.9, 133.5, 131.6, 131.0, 129.4, 127.2, 127.1, 126.9, 122.1, 120.9, 17.2, 9.2; HRMS (ESI): calcd. for C₁₈H₁₅ClN₅OS [M+H]⁺ 384.0686, found 384.0692.

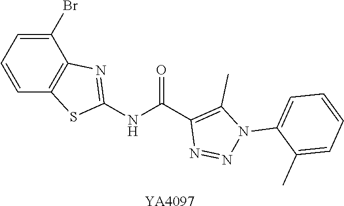

N-(4-Bromobenzo[d]thiazol-2-yl)-5-methyl-1-(o-tolyl)-1H-1,2,3-triazole-4-carboxamide (YA4097). The title compound was synthesized according to General Procedure B (%, a white solid): ¹H-NMR (400 MHz, CDCl₃) δ 10.70 (s, 1H), 7.77 (d, J=8.0 Hz, 1H), 7.64 (d, J=7.2 Hz, 1H), 7.50 (t, J=7.2 Hz, 1H), 7.43-7.39 (m, 2H), 7.24-7.16 (m, 2H), 2.51 (s, 3H), 2.05 (s, 3H); ¹³C-NMR (100 MHz, CDCl₃) δ 159.2, 157.3, 147.1, 139.8, 136.2, 135.4, 133.9, 133.1, 131.6, 131.0, 129.7, 127.2, 127.1, 124.9, 120.5, 114.7, 17.2, 9.2; HRMS (ESI): calcd. for C₁₈H₁₅BrN₅OS [M+H]⁺ 428.0181, found 428.0185.

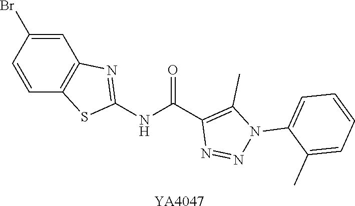

N-(5-Bromobenzo[d]thiazol-2-yl)-5-methyl-1-(o-tolyl)-1H-1,2,3-triazole-4-carboxamide (YA4047). The title compound was synthesized according to General Procedure B (%, a white solid): ¹H-NMR (400 MHz, CDCl₃) δ 10.61 (s, 1H), 7.99 (s, 1H), 7.70 (d, J=8.4 Hz, 1H), 7.52 (t, J=8.0 Hz, 1H), 7.45-7.39 (m, 3H), 7.25 (d, J=8.0 Hz, 1H), 2.53 (s, 3H), 2.07 (s, 3H); ¹³C-NMR (100 MHz, CDCl₃) δ 159.1, 158.0, 150.1, 139.7, 136.2, 135.4, 133.9, 131.6, 131.1, 127.2, 127.1, 127.0, 124.2, 122.4, 119.8, 17.2, 9.2; HRMS (ESI): calcd. for C₁₁H₁₅BrN₅OS [M+H]⁺ 428.0181, found 428.0187.

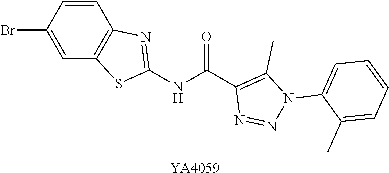

N-(6-Bromobenzo[d]thiazol-2-yl)-5-methyl-1-(o-tolyl)-1H-1,2,3-triazole-4-carboxamide (YA4059). The title compound was synthesized according to General Procedure B (%, a white solid): ¹H-NMR (400 MHz, CDCl₃) δ 10.47 (s, 1H), 8.10 (s, 1H), 7.82 (d, J=8.4 Hz, 1H), 7.68-7.63 (m, 2H), 7.58-7.52 (m, 2H), 7.38 (d, J=7.2 Hz, 1H), 2.66 (s, 3H), 2.20 (s, 3H); ¹³C-NMR (100 MHz, CDCl₃) δ 159.1, 157.1, 147.7, 139.7, 136.2, 135.4, 134.0, 133.9, 131.6, 131.1, 129.6, 127.2, 127.1, 123.8, 122.5, 116.9, 17.2, 9.2; HRMS (ESI): calcd. for C₁₁H₁₅BrN₅OS [M+H]⁺ 428.0181, found 428.0191.

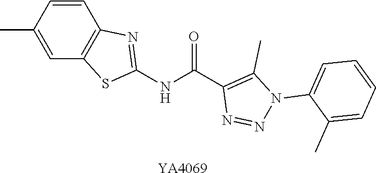

5-Methyl-N-(6-methylbenzo[d]thiazol-2-yl)-1-(o-tolyl)-1H-1,2,3-triazole-4-carboxamide (YA4069). The title compound was synthesized according to General Procedure B (%, a white solid): ¹H-NMR (400 MHz, CDCl₃) δ 10.76 (s, 1H), 7.83 (d, J=8.8 Hz, 1H), 7.74 (s, 1H), 7.61 (t, J=7.2 Hz, 1H), 7.54-7.48 (m, 2H), 7.38-7.34 (m, 2H), 2.63 (s, 3H), 2.59 (s, 3H), 2.17 (s, 3H); ¹³C-NMR (100 MHz, CDCl₃) δ 159.0, 156.0 146.7, 139.5, 136.4, 135.4, 134.0, 133.9, 132.4, 131.5, 131.0, 127.7, 127.2, 127.1, 121.1, 120.8, 21.5, 17.2, 9.2; HRMS (ESI): calcd. for C₁₉H₁₈N₅OS [M+H]⁺ 364.1232, found 364.1237.

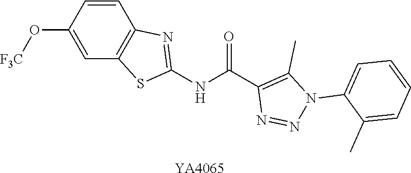

5-Methyl-1-(o-tolyl)-N-(6-(trifluoromethoxy)benzo[d]thiazol-2-yl)-1H-1,2,3-triazole-4-carboxamide (YA4065). The title compound was synthesized according to General Procedure B (%, a white solid): ¹H-NMR (400 MHz, CDCl₃) δ 10.47 (s, 1H), 7.48 (d, J=8.4 Hz, 1H), 7.38 (s, 1H), 7.18 (t, J=8.0, 7.2 Hz, 1H), 7.11-7.05 (m, 2H), 6.98 (d, J=8.4 Hz, 1H), 6.92 (d, J=7.2 Hz, 1H), 2.20 (s, 3H), 1.73 (s, 3H); ¹³C-NMR (100 MHz, CDCl₃) δ 159.2, 157.8, 147.4, 145.4, 139.8, 136.2, 135.4, 133.9, 133.1, 131.5, 131.0, 127.2, 127.0, 124.4 (q, J=256.0 Hz, CF₃), 122.0, 120.0, 114.1, 17.2, 9.2; HRMS (ESI): calcd. for C₁₉H₁₅F₃N₅O₂S [M+H]⁺ 434.0899, found 434.0889.

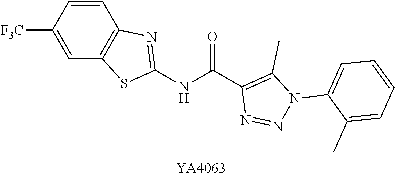

5-Methyl-1-(o-tolyl)-N-(6-(trifluoromethyl)benzo[d]thiazol-2-yl)-1H-1,2,3-triazole-4-carboxamide (YA4063). The title compound was synthesized according to General Procedure B (%, a white solid): ¹H-NMR (400 MHz, CDCl₃) δ 10.59 (s, 1H), 7.90 (s, 1H), 7.67 (d, J=8.8 Hz, 1H), 7.44 (d, J=8.4 Hz, 1H) 7.28 (t, J=8.0, 6.8 Hz, 1H), 7.22-7.15 (m, 2H), 7.01 (d, J=8.0 Hz, 1H), 2.30 (s, 3H), 1.84 (s, 3H); ¹³C-NMR (100 MHz, CDCl₃) δ 159.3, 151.1, 139.9, 136.1, 135.4, 133.9, 132.4, 131.6, 131.1, 128.4, 127.2, 127.0, 126.3 (q, J=32.2 Hz) 123.3, 123.0 (q, J=271.5 Hz, CF₃), 121.5, 119.0, 17.2, 9.2; HRMS (ESI): calcd. for C₁₉H₁₅F₃N₅O [M+H]⁺ 418.0949, found 418.0953.

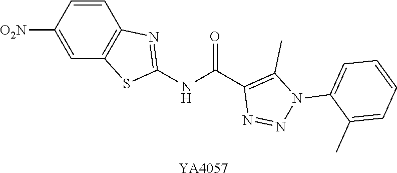

5-Methyl-N-(6-nitrobenzo[d]thiazol-2-yl)-1-(o-tolyl)-1H-1,2,3-triazole-4-carboxamide (YA4057). The title compound was synthesized according to General Procedure B (%, a white solid): ¹H-NMR (400 MHz, CDCl₃) δ 10.75 (s, 1H), 8.76 (s, 1H), 8.32 (d, J=9.2 Hz, 1H), 7.87 (d, J=9.2 Hz, 1H), 7.51 (t, J=8.0, 6.8 Hz, 1H), 7.45-7.38 (m, 2H), 7.24 (s, 1H), 2.52 (s, 3H), 2.06 (s, 3H); ¹³C-NMR (100 MHz, CDCl₃) δ 161.4, 159.3, 153.3, 143.9, 140.2, 135.9, 135.3, 133.8, 132.7, 131.6, 131.2, 127.3, 127.0, 122.0, 121.3, 118.1, 17.2, 9.3; HRMS (ESI): calcd. for C₁₈H₁₅N₆O₃S [M+H]⁺ 395.0926, found 395.0932.

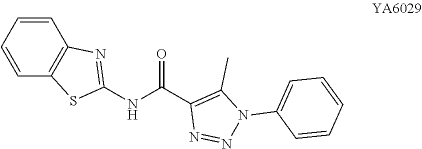 [SMILES: Cc1c(C(=O)Nc2nc3ccccc3s2)nnn1-c1ccccc1]

N-(Benzo[d]thiazol-2-yl)-5-methyl-1-phenyl-1H-1,2,3-triazole-4-carboxamide (YA6029). The title compound was synthesized according to General Procedure B (50%, a white solid): ¹H-NMR (400 MHz, CDCl₃) δ 10.54 (s, 1H), 7.85 (d, J=8.0 Hz, 2H), 7.60-7.61 (m, 3H), 7.50-7.44 (m, 3H), 7.33 (t, J=7.6, 7.2 Hz, 1H), 2.72 (s, 3H); ¹³C-NMR (100 MHz, CDCl₃) δ 159.0, 156.8, 148.7, 138.6, 136.8, 135.2, 132.2, 130.3, 129.8, 126.3, 125.2, 124.0, 121.3, 121.3, 9.9; HRMS (ESI): calcd. for C₁₇H₁₄N₅OS [M+H]⁺ 336.0919, found 336.0911.

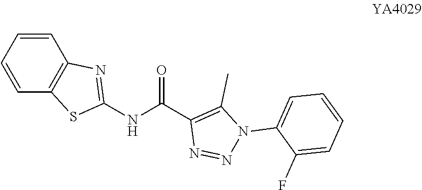 [SMILES: Cc1c(C(=O)Nc2nc3ccccc3s2)nnn1-c1ccccc1F]

N-(benzo[d]thiazol-2-yl)-1-(2-fluorophenyl)-5-methyl-1H-1,2,3-triazole-4-carboxamide (YA4029). The title compound was synthesized according to General Procedure B (%, a white solid): ¹H-NMR (400 MHz, CDCl₃) δ 10.50 (s, 1H), 7.85 (d, J=8.4 Hz, 2H), 7.65-7.60 (m, 1H), 7.54 (t, J=7.6 Hz, 1H), 7.47 (t, J=8.0 Hz, 1H), 7.43-7.32 (m, 3H), 2.64 (s, 3H); ¹³C-NMR (100 MHz, CDCl₃) δ 158.8, 156.8, 156.0 (d, J_{CF}=252.2 Hz), 148.6, 140.5, 136.5, 132.7 (d, J_{CF}=7.8 Hz), 132.2, 128.5, 126.2, 125.4 (d, J_{CF}=3.8 Hz), 124.0, 123.0 (d, J_{CF}=12.8 Hz), 121.3, 117.1 (d, J_{CF}=19.3 Hz), 9.2; HRMS (ESI): calcd. for C₁₇H₁₃FN₅OS [M+H]⁺ 354.0825, found 354.0823.

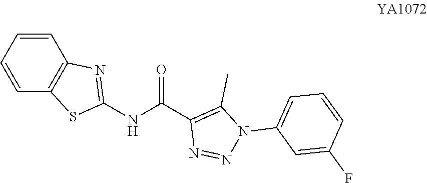 [SMILES: Cc1c(C(=O)Nc2nc3ccccc3s2)nnn1-c1cccc(F)c1]

N-(Benzo[d]thiazol-2-yl)-1-(3-fluorophenyl)-5-methyl-1H-1,2,3-triazole-4-carboxamide (YA1072). The title compound was synthesized according to General Procedure B (%, a white solid): ¹H-NMR (400 MHz, CDCl₃) δ 10.48 (s, 1H), 7.74 (d, J=8.0 Hz, 2H), 7.51-7.46 (m, 1H), 7.36 (t, J=7.6, 8.0 Hz, 1H), 7.25-7.16 (m, 4H), 2.64 (s, 3H); ¹³C-NMR (100 MHz, CDCl₃) δ 164.0, 161.5 (d, J=170.1 Hz), 156.7, 148.7, 138.6, 137.0, 136.3 (d, J=9.0 Hz), 132.3, 131.3 (d, J=9.0 Hz), 126.3, 124.0, 121.3, 120.8 (d, J=3.9 Hz), 117.6 (d, J=21.9 Hz), 113.1 (d, J=24.5 Hz), 9.9; HRMS (ESI): calcd. for C₁₇H₁₃FN₅OS [M+H]⁺ 354.0825, found 354.0826.

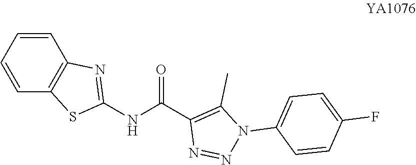 [SMILES: Cc1c(C(=O)Nc2nc3ccccc3s2)nnn1-c1ccc(F)cc1]

N-(Benzo[d]thiazol-2-yl)-1-(4-fluorophenyl)-5-methyl-1H-1,2,3-triazole-4-carboxamide (YA1076). The title compound was synthesized according to General Procedure B (%, a white solid): ¹H-NMR (400 MHz, DMSO-d₆) δ 12.71 (s, 1H), 8.03 (d, J=8.0 Hz, 1H), 7.82-7.76 (m, 3H), 7.56-7.46 (m, 3H), 7.35 (t, J=7.2 Hz, 1H), 2.60 (s, 3H); ¹³C-NMR (100 MHz, DMSO-d₆) δ 164.3, 161.8, 160.7 (d, J=254.8 Hz), 148.7, 139.9, 137.2, 131.9, 128.5 (d, J=9.1 Hz), 127.4, 126.7, 124.2, 122.2, 121.0, 117.3 (d, J=23.1 Hz), 115.6, 10.0; HRMS (ESI): calcd. for C₁₇H₁₃FN₅OS [M+H]⁺ 354.0825, found 354.0831.

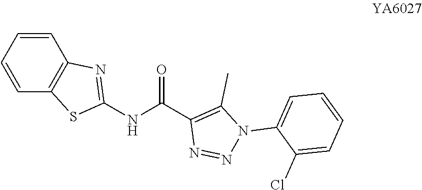 [SMILES: Cc1c(C(=O)Nc2nc3ccccc3s2)nnn1-c1ccccc1Cl]

N-(Benzo[d]thiazol-2-yl)-1-(2-chlorophenyl)-5-methyl-1H-1,2,3-triazole-4-carboxamide (YA6027). The title compound was synthesized according to General Procedure B (50%, a white solid): ¹H-NMR (400 MHz, CDCl₃) δ 10.61 (s, 1H), 7.83 (d, J=7.2 Hz, 2H), 7.63 (d, J=7.6 Hz, 1H), 7.57 (t, J=6.8, 8.0 Hz, 1H), 7.52-7.45 (m, 3H), 7.32 (t, J=8.0, 6.8 Hz, 1H), 2.56 (s, 3H); ¹³C-NMR (100 MHz, CDCl₃) δ 158.9, 156.8, 148.7, 140.5, 136.4, 132.7, 132.4, 132.2, 131.6, 130.8, 129.1, 128.2, 126.3, 124.0, 121.3, 9.27; HRMS (ESI): calcd. for C₁₇H₁₃ClN₅OS [M+H]⁺ 370.0529, found 370.0527.

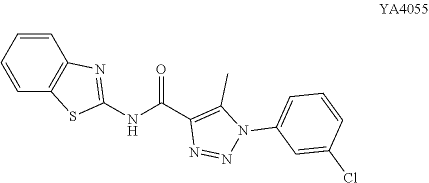 [SMILES: Cc1c(C(=O)Nc2nc3ccccc3s2)nnn1-c1cccc(Cl)c1]

N-(Benzo[d]thiazol-2-yl)-1-(3-chlorophenyl)-5-methyl-1H-1,2,3-triazole-4-carboxamide (YA4055). The title compound was synthesized according to General Procedure B (%, a white solid): ¹H-NMR (400 MHz, CDCl₃) δ 10.50 (s, 1H), 7.85 (d, J=8.0 Hz, 2H), 7.57-7.54 (m, 3H), 7.47 (t, J=8.0 Hz, 1H), 7.40 (d, J=7.2 Hz, 1H), 7.34 (t, J=8.0 Hz, 1H), 2.74 (s, 3H); ¹³C-NMR (100 MHz, CDCl₃) δ 158.8, 156.7, 148.6, 138.7, 137.0, 136.1, 135.6, 132.2, 130.8, 130.5, 126.3, 125.5, 124.1, 123.2, 121.3, 121.2, 9.9; HRMS (ESI): calcd. for C₁₇H₁₃ClN₅OS [M+H]⁺ 370.0529, found 370.0535.

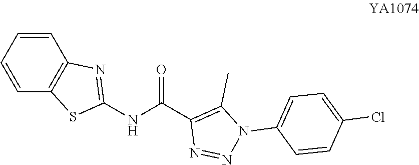 [SMILES: Cc1c(C(=O)Nc2nc3ccccc3s2)nnn1-c1ccc(Cl)cc1]

N-(Benzo[d]thiazol-2-yl)-1-(4-chlorophenyl)-5-methyl-1H-1,2,3-triazole-4-carboxamide (YA1074). The title compound was synthesized according to General Procedure B (%, a white solid):

¹H-NMR (400 MHz, DMSO-d₆) δ 12.71 (s, 1H), 8.02 (d, J=7.6 Hz, 1H), 7.81-7.75 (m, 5H), 7.47 (t, J=7.2, 7.6 Hz, 1H), 7.35 (t, J=8.0, 7.2 Hz, 1H), 2.62 (s, 3H); ¹³C-NMR (100 MHz, DMSO-d₆) δ 139.9, 137.3, 135.3, 134.4, 132.0, 130.3, 127.8, 126.7, 124.2, 122.2, 121.0, 10.1; HRMS (ESI): calcd. for C₁₇H₁₃ClN₅OS [M+H]⁺ 370.0529, found 370.0529.

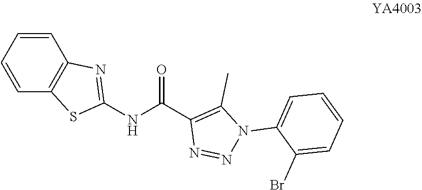 [SMILES: Cc1c(C(=O)Nc2nc3ccccc3s2)nnn1-c1ccccc1Br]

N-(Benzo[d]thiazol-2-yl)-1-(2-bromophenyl)-5-methyl-1H-1,2,3-triazole-4-carboxamide (YA4003). The title compound was synthesized according to General Procedure B (31%, a yellow solid): ¹H-NMR (400 MHz, CDCl₃) δ 10.5 (br s, 1H), 7.77-7.72 (m, 3H), 7.49-7.34 (m, 4H), 7.24 (t, J=8.0, 7.2 Hz, 1H), 2.48 (s, 3H); ¹³C-NMR (100 MHz, CDCl₃) δ 158.9, 156.8, 148.7, 140.3, 136.4, 134.4, 133.9, 132.5, 132.3, 129.2, 128.8, 126.3, 124.0, 121.4, 121.3, 9.4; HRMS (ESI): calcd. for C₁₇H₁₃BrN₅OS [M+H]⁺ 414.0024, found 414.0023.

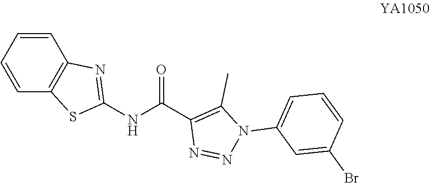 [SMILES: Cc1c(C(=O)Nc2nc3ccccc3s2)nnn1-c1cccc(Br)c1]

N-(Benzo[d]thiazol-2-yl)-1-(3-bromophenyl)-5-methyl-1H-1,2,3-triazole-4-carboxamide (YA1050). The title compound was synthesized according to General Procedure B (%, a white solid): ¹H-NMR (400 MHz, CDCl₃) δ 10.53 (s, 1H), 7.83 (d, J=8.0 Hz, 2H), 7.72-7.68 (m, 2H), 7.49-7.42 (m, 3H), 7.32 (t, J=6.8, 8.0 Hz, 1H), 2.72 (s, 3H); ¹³C-NMR (100 MHz, CDCl₃) δ 158.8, 156.7, 148.7, 138.7, 137.0, 136.2, 133.5, 132.3, 131.0, 128.3, 126.3, 124.0, 123.7, 123.3, 121.3, 9.9; HRMS (ESI): calcd. for C₁₇H₁₃BrN₅OS [M+H]⁺ 414.0024, found 414.0024.

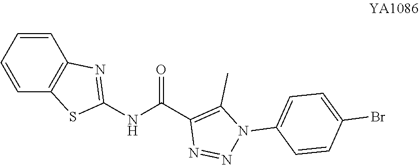 [SMILES: Cc1c(C(=O)Nc2nc3ccccc3s2)nnn1-c1ccc(Br)cc1]

N-(Benzo[d]thiazol-2-yl)-1-(4-bromophenyl)-5-methyl-1H-1,2,3-triazole-4-carboxamide (YA1086). The title compound was synthesized according to General Procedure B (%, a white solid): ¹H-NMR (400 MHz, CDCl₃) δ 12.71 (s, 1H), 8.02 (d, J=7.6 Hz, 1H), 7.89 (d, J=7.6 Hz, 2H), 7.80 (d, J=8.0 Hz, 1H), 7.67 (d, J=8.0 Hz, 2H), 7.47 (t, J=7.2, 7.6 Hz, 1H), 7.34 (t, J=8.0, 6.8 Hz, 1H), 2.62 (s, 3H); ¹³C-NMR (100 MHz, CDCl₃) δ 134.8, 133.2, 128.0, 126.7, 124.2, 123.9, 122.2, 121.1, 10.1; HRMS (ESI): calcd. for C₁₇H₁₃BrN₅OS [M+H]⁺ 414.0024, found 414.0033.

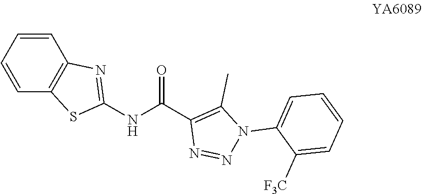 [SMILES: Cc1c(C(=O)Nc2nc3ccccc3s2)nnn1-c1ccccc1C(F)(F)F]

N-(Benzo[d]thiazol-2-yl)-5-methyl-1-(2-(trifluoromethyl)phenyl)-1H-1,2,3-triazole-4-carboxamide (YA6089). The title compound was synthesized according to General Procedure B (60%, a white solid): ¹H-NMR (400 MHz, CDCl₃) δ 10.56 (s, 1H), 7.94 (d, J=7.2 Hz, 1H), 7.85-7.76 (m, 4H), 7.47-7.40 (m, 2H), 7.33 (t, J=8.0 Hz, 1H), 2.52 (s, 3H); ¹³C-NMR (100 MHz, CDCl₃) δ 158.8, 156.7, 148.7, 140.9, 136.2, 133.4, 132.3, 131.7, 129.7, 128.2, 127.9, 127.9, 126.3, 124.0, 123.7 (q, J=270 Hz, CF₃), 121.3, 120.9, 9.2; HRMS (ESI): calcd. for C₁₈H₁₃F₃N₅OS [M+H]⁺ 404.0793, found 404.0792.

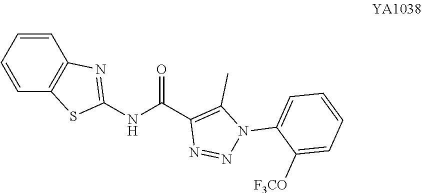 [SMILES: Cc1c(C(=O)Nc2nc3ccccc3s2)nnn1-c1ccccc1OC(F)(F)F]

N-(Benzo[d]thiazol-2-yl)-5-methyl-1-(2-(trifluoromethoxy)phenyl)-1H-1,2,3-triazole-4-carboxamide (YA1038). The title compound was synthesized according to General Procedure B (%, a white solid): ¹H-NMR (400 MHz, CDCl₃) δ 10.93 (s, 1H), 8.07 (t, J=6.4 Hz, 2H), 7.90-7.88 (m, 1H), 7.76-7.75 (m, 3H), 7.67 (t, J=6.8, 8.0 Hz, 1H), 7.55 (t, J=7.2, 7.6 Hz, 1H), 2.82 (s, 3H); ¹³C-NMR (100 MHz, CDCl₃) δ 158.9, 156.8, 148.7, 143.7, 140.5, 136.4, 132.6, 132.3, 129.1, 127.9, 127.5, 126.2, 124.0 (q, J=270.2 Hz), 121.7, 118.7, 9.2; HRMS (ESI): calcd. for C₁₈H₁₃F₃N₅O₂S [M+H]⁺ 420.0742, found 420.0739.

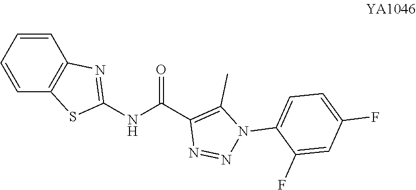 [SMILES: Cc1c(C(=O)Nc2nc3ccccc3s2)nnn1-c1ccc(F)cc1F]

N-(Benzo[d]thiazol-2-yl)-1-(2,4-difluorophenyl)-5-methyl-1H-1,2,3-triazole-4-carboxamide (YA1046). The title compound was synthesized according to General Procedure B (%, a white solid): ¹H-NMR (400 MHz, CDCl₃) δ 10.50 (s, 1H), 7.85-7.83 (m, 2H), 7.56-7.50 (m, 1H), 7.46 (t, J=8.0, 7.6 Hz, 1H), 7.33 (t, J=8.0, 6.8 Hz, 1H), 7.14-7.10 (m, 2H), 2.62 (s, 3H); ¹³C-NMR (100 MHz, CDCl₃) δ 165.4 (d, J=254.8 Hz), 165.3 (d, J=254.7 Hz), 158.7, 156.7, 155.2, 148.7, 140.5, 136.6, 132.3, 129.8 (d, J=10.2 Hz), 126.3, 124.1, 121.3 (d, J=26.0 Hz), 113.0 (d, J=23.2 Hz), 105.7 (t, J=25.7, 23.2 Hz), 9.1; HRMS (ESI): calcd. for C₁₇H₁₂F₂N₅OS [M+H]⁺ 372.0731, found 372.0728.

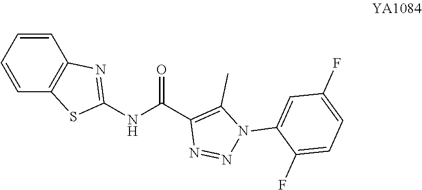 [SMILES: Cc1c(C(=O)Nc2nc3ccccc3s2)nnn1-c1cc(F)ccc1F]

N-(Benzo[d]thiazol-2-yl)-1-(2,5-difluorophenyl)-5-methyl-1H-1,2,3-triazole-4-carboxamide (YA1084). The title compound was synthesized according to General Procedure B (%, a white solid): ¹H-NMR (400 MHz, CDCl₃) δ 10.57 (s, 1H), 7.82 (t, J=6.4 Hz, 2H), 7.43 (t, J=8.0, 7.6 Hz, 1H), 7.33-7.27 (m, 4H), 2.63 (s, 3H); ¹³C-NMR (100 MHz, CDCl₃) δ 159.6 (d, J=245.7 Hz), 158.7, 156.7, 153.5 (d, J=247.0 Hz), 148.7, 140.5, 136.7, 132.3, 126.3, 124.1, 121.3, 119.6 (dd, J=7.7, 23.1 Hz), 118.3 (dd, J=21.9, 9.0 Hz), 115.8 (d, J=27.0 Hz), 9.2; HRMS (ESI): calcd. for C₁₇H₁₂F₂N₅OS [M+H]⁺ 372.0731, found 372.0723.

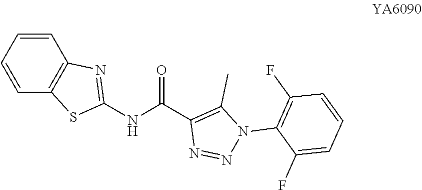 [SMILES: Cc1c(C(=O)Nc2nc3ccccc3s2)nnn1-c1c(F)cccc1F]

N-(Benzo[d]thiazol-2-yl)-1-(2,6-difluorophenyl)-5-methyl-1H-1,2,3-triazole-4-carboxamide (YA6090). The title compound was synthesized according to General Procedure B (60%, a white solid): ¹H-NMR (400 MHz, CDCl₃) δ 10.62 (s, 1H), 7.84-7.81 (m, 2H), 7.62-7.54 (m, 1H), 7.43 (t, J=6.8, 8.0 Hz, 1H), 7.31 (t, J=7.6, 7.2 Hz, 1H), 7.18 (t, J=8.0 Hz, 2H), 2.60 (s, 3H); ¹³C-NMR (100 MHz, CDCl₃) δ 158.9 (d, J_{CF}=214.9 Hz), 158.6, 156.3, 148.7, 141.2, 136.4, 133.0 (t, J_{CF}=9.0, 10.3 Hz), 132.3, 126.2, 124.0, 121.3, 112.8 (dd, J_{CF}=3.8, 19.3 Hz), 8.8; HRMS (ESI): calcd. for C₁₇H₁₂F₂N₅OS [M+H]⁺ 372.0731, found 372.0730.

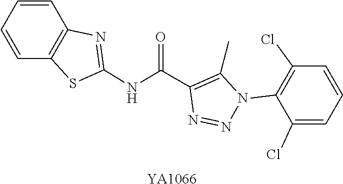

N-(Benzo[d]thiazol-2-yl)-1-(2,6-dichlorophenyl)-5-methyl-1H-1,2,3-triazole-4-carboxamide (YA1066). The title compound was synthesized according to General Procedure B (%, a white solid): ¹H-NMR (400 MHz, CDCl₃) δ 10.63 (s, 1H), 7.84-7.83 (m, 2H), 7.56-7.43 (m, 4H), 7.32 (t, J=8.0 Hz, 1H), 2.54 (s, 3H); ¹³C-NMR (100 MHz, CDCl₃) δ 158.8, 156.7, 148.7, 140.7, 136.4, 134.3, 132.7, 132.3, 130.9, 129.1, 126.3, 124.0, 121.3, 8.7; HRMS (ESI): calcd. for C₁₇H₁₂C₁₂N₅OS [M+H]⁺ 404.0140, found 404.0150.

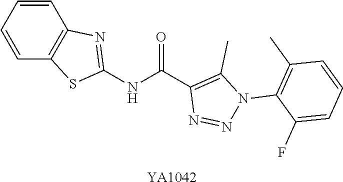

N-(Benzo[d]thiazol-2-yl)-1-(2-fluoro-6-methylphenyl)-5-methyl-1H-1,2,3-triazole-4-carboxamide (YA1042). The title compound was synthesized according to General Procedure B (%, a white solid): ¹H-NMR (400 MHz, CDCl₃) δ 10.63 (s, 1H), 7.83 (d, J=6.4 Hz, 2H), 7.49-7.42 (m, 2H), 7.31 (t, J=7.2, 8.0 Hz, 1H), 7.20 (d, J=7.6 Hz, 1H), 7.14 (t, J=8.0 Hz, 1H), 2.53 (s, 3H), 2.08 (s, 3H); ¹³C-NMR (100 MHz, CDCl₃) δ 158.9, 158.5 (d, J=252.2 Hz), 156.8, 148.7, 140.6, 138.4, 136.4, 132.3 (d, J=9.0 Hz), 126.7, 126.3, 124.0, 122.2 (d, J=14.2 Hz), 121.3, 114.2 (d, J=19.3 Hz), 17.2, 8.8; HRMS (ESI): calcd. for C₁₈H₁₅FN₅OS [M+H]⁺ 368.0981, found 368.0986.

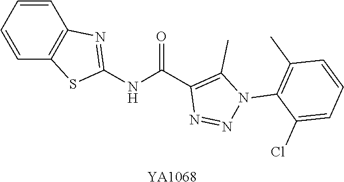

N-(Benzo[d]thiazol-2-yl)-1-(2-chloro-6-methylphenyl)-5-methyl-1H-1,2,3-triazole-4-carboxamide (YA1068). The title compound was synthesized according to General Procedure B (%, a white solid): ¹H-NMR (400 MHz, CDCl₃) δ 10.70 (s, 1H), 7.84-7.81 (m, 2H), 7.44-7.39 (m, 3H), 7.29-7.30 (m, 2H), 2.50 (s, 3H), 2.02 (s, 3H); ¹³C-NMR (100 MHz, CDCl₃) δ 159.0, 156.8, 148.7, 140.3, 138.5, 136.5, 132.3, 131.9, 131.8, 129.7, 128.0, 126.2, 124.0, 121.3, 17.7, 8.8; HRMS (ESI): calcd. for C₁₈H₁₅ClN₅OS [M+H]⁺ 384.0686, found 384.0693.

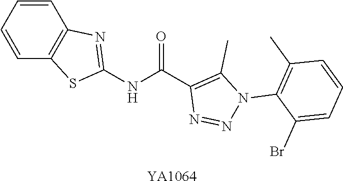

N-(Benzo[d]thiazol-2-yl)-1-(2-bromo-6-methylphenyl)-5-methyl-1H-1,2,3-triazole-4-carboxamide (YA1064). The title compound was synthesized according to General Procedure B (%, a white solid): ¹H-NMR (400 MHz, CDCl₃) δ 10.58 (s, 1H), 7.84 (d, J=8.0 Hz, 2H), 7.62 (t, J=4.4 Hz, 1H), 7.45 (t, J=8.0, 7.6 Hz, 1H), 7.37-7.31 (m, 3H), 2.51 (s, 3H), 2.05 (s, 3H); ¹³C-NMR (100 MHz, CDCl₃) δ 159.0, 156.7, 148.7, 140.1, 138.6, 136.5, 133.4, 132.2, 131.3, 130.3, 126.3, 124.0, 122.1, 121.3, 18.0, 8.9; HRMS (ESI): calcd. for C₁₁H₁₅BrN₅OS [M+H]⁺ 428.0181, found 428.0176.

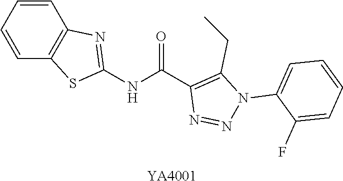

N-(Benzo[d]thiazol-2-yl)-5-ethyl-1-(2-fluorophenyl)-1H-1,2,3-triazole-4-carboxamide (YA4001). The title compound was synthesized according to General Procedure B (31%, a yellow solid): ¹H-NMR (400 MHz, CDCl₃) δ 10.61 (s, 1H), 7.83 (d, J=8.4 Hz, 1H), 7.61-7.59 (m, 1H), 7.48-7.42 (m, 2H), 7.39-7.29 (m, 3H), 3.02 (q, J=6.8, 8.0, 7.2 Hz, 2H), 1.18 (t, J=6.8, 8.0 Hz, 3H); ¹³C-NMR (100 MHz, CDCl₃) δ 158.6, 156.8, 156.5 (d, J_{CF}=253.4 Hz), 148.7, 145.8, 136.0, 132.9 (d, J_{CF}=7.7 Hz), 132.2, 128.8, 126.2, 125.3, 124.0, 123.2 (d, J_{CF}=11.5 Hz), 121.3, 117.2 (d, J_{CF}=19.3 Hz), 16.9, 12.7; HRMS (ESI): calcd. for C₁₈H₁₅FN₅OS [M+H]⁺ 368.0981, found 368.0976.

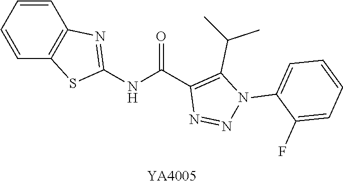

N-(Benzo[d]thiazol-2-yl)-1-(2-fluorophenyl)-5-isopropyl-1H-1,2,3-triazole-4-carboxamide (YA4005). The title compound was synthesized according to General Procedure B (31%, a yellow solid): ¹H-NMR (400 MHz, CDCl₃) δ 10.67 (br s, 1H), 7.83 (d, J=8.0 Hz, 2H), 7.65-7.59 (m, 1H), 7.47-7.44 (m, 2H), 7.40-7.30 (m, 3H), 3.31-3.24 (m, 1H), 1.40 (d, J=5.6 Hz, 6H); ¹³C-NMR (100 MHz, CDCl₃) δ 158.4, 156.9, 156.8 (d, J_{CF}=253.4 Hz), 149.3, 148.7, 135.9, 133.0 (d, J_{CF}=7.7 Hz), 132.2, 129.1, 126.3, 125.2 (d, J_{CF}=2.5 Hz), 124.0, 121.2 (d, J_{CF}=10.3 Hz), 117.1 (d, J_{CF}=18.0 Hz), 25.2, 19.9; HRMS (ESI): calcd. for C₁₉H₁₇FN₅OS [M+H]⁺ 382.1138, found 382.1146.

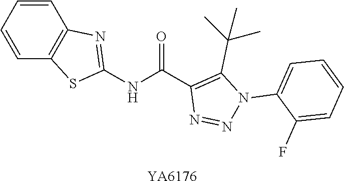

N-(Benzo[d]thiazol-2-yl)-5-(tert-butyl)-1-(2-fluorophenyl)-1H-1,2,3-triazole-4-carboxamide (YA6176). The title compound was synthesized according to General Procedure B (60%, a white solid): ¹H-NMR (400 MHz, CDCl₃) δ 10.89 (s, 1H), 7.84 (dd, J=8.0 Hz, 2H), 7.59-7.58 (m, 1H), 7.45 (t, J=7.6, 7.2 Hz, 2H), 7.35-7.26 (m, 3H), 1.42 (s, 9H); ¹³C-NMR (100 MHz, CDCl₃) δ 158.8, 157.4 (d, J_{CF}=252.2 Hz), 157.2, 151.4, 148.8, 136.6, 132.8 (d, J_{CF}=7.8 Hz), 132.2, 129.3, 126.7 (d, J_{CF}=14.2 Hz), 126.2, 124.8 (d, J_{CF}=3.9 Hz), 123.9, 121.3 (d, J_{CF}=11.6 Hz), 116.7 (d, J_{CF}=18.0 Hz), 33.2, 29.7; HRMS (ESI): calcd. for C₂₀H₁₉FN₅OS [M+H]⁺ 396.1294, found 396.1290.

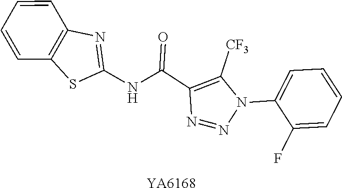

N-(Benzo[d]thiazol-2-yl)-1-(2-fluorophenyl)-5-(trifluoromethyl)-1H-1,2,3-triazole-4-carboxamide (YA6168). The title compound was synthesized according to General Procedure B (60%, a white solid): ¹H-NMR (400 MHz, CDCl₃) δ 10.93 (s, 1H), 7.86-7.81 (m, 2H), 7.67-7.61 (m, 1H), 7.52 (t, J=7.2 Hz, 1H), 7.45 (t, J=6.8, 8.0 Hz, 1H), 7.40-7.32 (m, 3H); ¹³C-NMR (100 MHz, CDCl₃) δ 156.7, 156.2 (d, J_{CF}=254.7 Hz), 155.6, 148.4, 139.5, 133.6 (d, J_{CF}=6.4 Hz), 132.3, 128.1, 126.4, 125.1 (d, J_{CF}=3.9 Hz), 124.4, 123.5 (d, J_{CF}=12.8 Hz), 121.4 (d, J_{CF}=5.2 Hz), 118.5 (q, J=270.1 Hz, CF₃), 116.9 (d, J_{CF}=19.3 Hz); HRMS (ESI): calcd. for C₁₇H₁₀F₄N₅OS [M+H]⁺ 408.0542, found 408.0542.

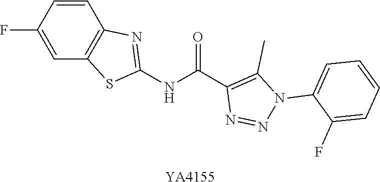

N-(6-Fluorobenzo[d]thiazol-2-yl)-1-(2-fluorophenyl)-5-methyl-1H-1,2,3-triazole-4-carboxamide (YA4155). The title compound was synthesized according to General Procedure B (%, a white solid): ¹H-NMR (400 MHz, DMSO-d₆) δ 12.63 (s, 1H), 7.75 (d, J=8.0 Hz, 1H), 7.65-7.59 (m, 3H), 7.46 (t, J=8.0 Hz, 1H), 7.35 (t, J=6.8, 8.0 Hz, 1H), 7.14 (t, J=8.0 Hz, 1H), 2.34 (s, 3H); ¹³C-NMR (100 MHz, DMSO-d₆) δ 159.2 (d, J_{CF}=238.0 Hz), 156.2 (d, J_{CF}=250.9 Hz), 145.5, 141.0, 136.9, 133.7 (d, J_{CF}=7.7 Hz), 133.3 (d, J_{CF}=11.6 Hz), 129.5, 126.2, 122.9 (d, J_{CF}=11.6 Hz), 122.2 (d, J_{CF}=9.0 Hz), 117.6 (d, J_{CF}=19.3 Hz), 114.8 (d, J_{CF}=24.5 Hz), 108.6 (d, J_{CF}=27.0 Hz), 9.4; HRMS (ESI): calcd. for C₁₇H₁₂F₂N₅OS [M+H]⁺ 372.0731, found 372.0735.

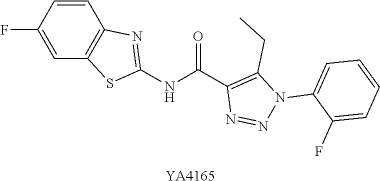

5-Ethyl-N-(6-fluorobenzo[d]thiazol-2-yl)-1-(2-fluorophenyl)-1H-1,2,3-triazole-4-carboxamide (YA4165). The title compound was synthesized according to General Procedure B (%, a white solid): ¹H-NMR (400 MHz, CDCl₃) δ 10.60 (s, 1H), 7.76-7.72 (m, 1H), 7.63-7.58 (m, 1H), 7.51-7.45 (m, 2H), 7.39-7.32 (m, 2H), 7.14 (t, J=8.0 Hz, 1H), 3.02 (m, 2H), 1.17 (t, J=7.2 Hz, 3H); ¹³C-NMR (100 MHz, CDCl₃) δ 159.6 (d, J_{CF}=241.8 Hz), 158.7, 156.5 (d, J_{CF}=253.4 Hz), 156.4, 145.8, 145.2, 135.9, 133.2 (d, J_{CF}=10.3 Hz), 132.9 (d, J_{CF}=7.8 Hz), 128.8, 125.3 (d, J_{CF}=3.8 Hz), 123.1 (d, J_{CF}=12.9 Hz), 122.1 (d, J_{CF}=9.0 Hz), 117.2 (d, J_{CF}=18.0 Hz), 114.5 (d, J_{CF}=24.4 Hz), 107.5 (d, J_{CF}=25.8 Hz), 16.9, 12.6; HRMS (ESI): calcd. for C₁₈H₁₄F₂N₅OS [M+H]⁺ 386.0887, found 386.0888.

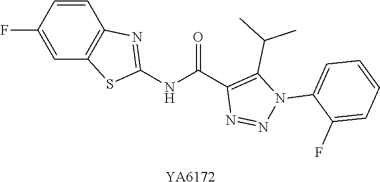

N-(6-Fluorobenzo[d]thiazol-2-yl)-1-(2-fluorophenyl)-5-isopropyl-1H-1,2,3-triazole-4-carboxamide (YA6172). The title compound was synthesized according to General Procedure B (%, a white solid): ¹H-NMR (400 MHz, CDCl₃) δ 10.71 (s, 1H), 7.76-7.73 (m, 1H), 7.64-7.59 (m, 1H), 7.51-7.43 (m, 2H), 7.39-7.32 (m, 2H), 7.18-7.13 (m, 1H), 3.29-3.22 (m, 1H), 1.39 (d, J=6.4 Hz, 6H); ¹³C-NMR (100 MHz, CDCl₃) δ 159.7 (d, J_{CF}=239.3 Hz), 156.8 (d, J_{CF}=253.4 Hz), 156.6, 149.4, 145.2, 135.8, 133.2, 133.0 (d, J_{CF}=7.7 Hz), 129.1, 125.2, 125.1, 123.7 (d, J_{CF}=12.9 Hz), 122.1 (d, J_{CF}=9.0 Hz), 117.1 (d, J_{CF}=19.3 Hz), 114.6 (d, J_{CF}=24.4 Hz), 107.6 (d, J_{CF}=25.8 Hz), 25.2, 19.9; HRMS (ESI): calcd. for C₁₉H₁₆F₂N₅OS [M+H]⁺ 400.1044, found 400.1042.

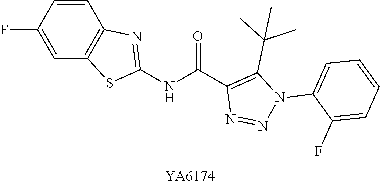

5-(Tert-butyl)-N-(6-fluorobenzo[d]thiazol-2-yl)-1-(2-fluorophenyl)-1H-1,2,3-triazole-4-carboxamide (YA6174). The title compound was synthesized according to General Procedure B (%, a white solid): ¹H-NMR (400 MHz, CDCl₃) δ 10.89 (s, 1H), 7.76-7.73 (m, 1H), 7.61-7.56 (m, 1H), 7.51 (dd, J=7.6 Hz, 1H), 7.45 (t, J=6.8, 7.2 Hz, 1H), 7.35-7.26 (m, 2H), 7.18-7.13 (m, 1H), 1.41 (s, 9H); ¹³C-NMR (100 MHz, CDCl₃) δ 159.6 (d, J_{CF}=241.8 Hz), 158.6, 157.4 (d, J_{CF}=252.2 Hz), 156.9, 151.4, 145.3, 136.4, 133.1 (d, J_{CF}=10.3 Hz), 132.8 (d, J_{CF}=7.7 Hz), 129.3, 126.6 (d, J_{CF}12.9 Hz), 124.7 (d, J_{CF}=3.9 Hz), 122.1 (d, J_{CF}=7.7 Hz), 116.6 (d, J_{CF}=19.3 Hz), 114.5 (d, J_{CF}24.4 Hz), 107.5 (d, J_{CF}=25.7 Hz), 33.2, 29.6; HRMS (ESI): calcd. for C₂₀H₁₈F₂N₅OS [M+H]⁺ 414.1200, found 414.1207.

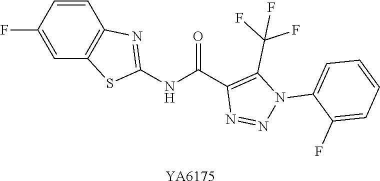

N-(6-Fluorobenzo[d]thiazol-2-yl)-1-(2-fluorophenyl)-5-(trifluoromethyl)-1H-1,2,3-triazole-4-carboxamide (YA6175). The title compound was synthesized according to General Procedure B (%, a white solid): ¹H-NMR (400 MHz, CDCl₃) δ 10.73 (s, 1H), 7.79-7.76 (m, 1H), 7.69-7.63 (m, 1H), 7.55-7.52 (m, 2H), 7.42-7.34 (m, 2H), 7.19 (td, J=8.0 Hz, 1H); ¹³C-NMR (100 MHz, CDCl₃) δ 159.8 (d, J_{CF}=243.1 Hz), 156.3 (d, J_{CF}=254.7 Hz), 156.3, 155.6, 145.0, 139.4, 133.6 (d, J_{CF}=6.4 Hz), 133.3 (d, J_{CF}=10.3 Hz), 128.0, 125.1 (d, J_{CF}=2.6 Hz), 123.4 (d, J_{CF}=14.2 Hz), 122.4 (d, J_{CF}=9.0 Hz), 119.8 (q, J=270.8 Hz, CF₃), 116.9 (d, J_{CF}=18.0 Hz), 114.8 (d, J_{CF}=23.2 Hz), 107.7 (d, J_{CF}=25.7 Hz); HRMS (ESI): calcd. for C₁₇H₉F₅N₅OS [M+H]⁺ 426.0448, found 426.0438.

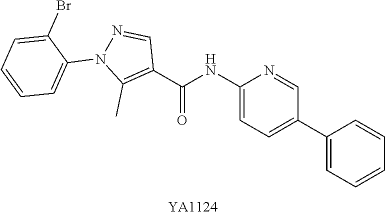

1-(2-Bromophenyl)-5-methyl-N-(5-phenylpyridin-2-yl)-1H-pyrazole-4-carboxamide (YA1124). The title compound was synthesized according to General Procedure E (%, a white solid): ¹H-NMR (400 MHz, CDCl₃) δ 8.56-8.52 (m, 2H), 8.41 (d, J=8.0 Hz, 1H), 8.06 (s, 1H), 7.95 (d, J=8.0 Hz, 1H), 7.75 (d, J=7.6 Hz, 1H), 7.58 (d, J=8.0 Hz, 2H), 7.50-7.36 (m, 6H), 2.49 (s, 3H); ¹³C-NMR (100 MHz, CDCl₃) δ 161.7, 150.7, 146.0, 145.0, 142.5, 138.7, 137.9, 137.4, 136.9, 133.6, 132.8, 131.4, 129.6, 129.5, 129.1, 128.5, 127.8, 126.8, 122.3, 114.9, 113.9, 11.4;

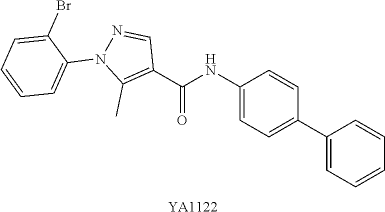

N-([1,1′-Biphenyl]-4-yl)-1-(2-bromophenyl)-5-methyl-1H-pyrazole-4-carboxamide (YA1122). The title compound was synthesized according to General Procedure E (%, a white solid): ¹H-NMR (400 MHz, CDCl₃) δ 8.02 (s, 1H), 7.90 (s, 1H), 7.73 (d, J=8.0 Hz, 1H), 7.68 (d, J=8.0 Hz, 2H), 7.59-7.56 (m, 4H), 7.48-7.31 (m, 6H), 2.46 (m, 3H); ¹³C-NMR (100 MHz, CDCl₃) δ 161.8, 144.8, 140.5, 138.4, 137.9, 137.2, 137.1, 133.6, 131.4, 129.7, 128.8, 128.5, 127.6, 127.1, 126.8, 122.3, 120.6, 115.2, 11.3;

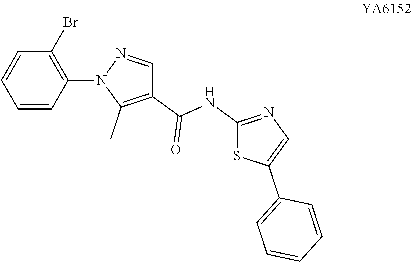 [SMILES: Cc1c(C(=O)Nc2ncc(-c3ccccc3)s2)cnn1-c1ccccc1Br]

1-(2-Bromophenyl)-5-methyl-N-(5-phenylthiazol-2-yl)-1H-pyrazole-4-carboxamide (YA6152). The title compound was synthesized according to General Procedure E (%, a white solid): ¹H-NMR (400 MHz, CDCl₃) δ 12.42 (s, 1H), 8.24 (s, 1H), 7.77 (d, J=7.6 Hz, 1H), 7.55-7.40 (m, 6H), 7.37 (t, J=7.2, 7.6 Hz, 2H), 7.28 (t, J=7.2, 7.6 Hz, 1H), 2.53 (s, 3H); ¹³C-NMR (100 MHz, CDCl₃) δ 161.5, 159.1, 145.7, 139.7, 137.8, 133.7, 132.6, 131.6, 131.5, 129.6, 129.0, 128.6, 127.8, 126.3, 122.3, 113.7, 11.4;

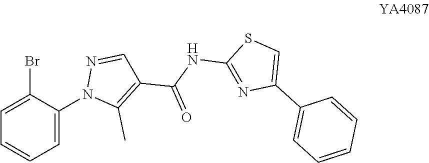 [SMILES: Cc1c(C(=O)Nc2nc(-c3ccccc3)cs2)cnn1-c1ccccc1Br]

1-(2-Bromophenyl)-5-methyl-N-(4-phenylthiazol-2-yl)-1H-pyrazole-4-carboxamide (YA4087). The title compound was synthesized according to General Procedure E (%, a white solid): ¹H-NMR (400 MHz, CDCl₃) δ 10.65 (s, 1H), 8.07 (s, 1H), 8.02 (d, J=7.2 Hz, 2H), 7.93 (d, J=8.0 Hz, 1H), 7.67 (t, J=8.0 Hz, 1H), 7.59-7.57 (m, 3H), 7.54-7.47 (m, 2H), 7.40 (s, 1H), 2.67 (s, 3H); ¹³C-NMR (100 MHz, CDCl₃) δ 160.9, 158.5, 149.9, 145.6, 138.6, 137.7, 134.2, 133.6, 131.4, 129.5, 128.7, 128.4, 128.0, 126.1, 122.2, 113.0, 107.9, 11.4; HRMS (ESI): calcd. for C₂₋H₁₆BrN₄OS [M+H]⁺ 439.0228, found 439.0217.

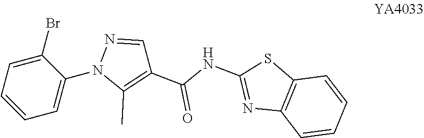 [SMILES: Cc1c(C(=O)Nc2nc3ccccc3s2)cnn1-c1ccccc1Br]

N-(Benzo[d]thiazol-2-yl)-1-(2-bromophenyl)-5-methyl-1H-pyrazole-4-carboxamide (YA4033). The title compound was synthesized according to General Procedure E (%, a white solid): ¹H-NMR (400 MHz, CDCl₃) δ 8.07 (s, 1H), 7.84 (d, J=6.8 Hz, 1H), 7.75 (d, J=8.0 Hz, 1H), 7.66 (d, J=7.6 Hz, 1H), 7.49-7.32 (m, 6H), 2.53 (s, 3H); ¹³C-NMR (100 MHz, CDCl₃) δ 161.7, 159.7, 147.5, 146.0, 139.5, 137.7, 133.6, 131.8, 131.4, 129.6, 128.5, 126.3, 124.0, 122.1, 121.4, 120.7, 113.2, 11.60; HRMS (ESI): calcd. for C₁₁H₁₄BrN₄OS [M+H]⁺ 413.0072, found 413.0074.

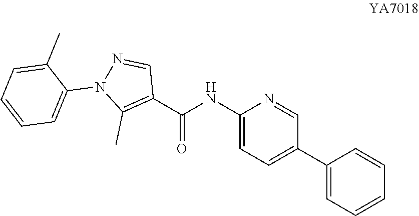 [SMILES: Cc1ccccc1-n1ncc(C(=O)Nc2ccc(-c3ccccc3)cn2)c1C]

5-Methyl-N-(5-phenylpyridin-2-yl)-1-(o-tolyl)-1H-pyrazole-4-carboxamide (YA7018). The title compound was synthesized according to General Procedure E (%, a white solid): ¹H-NMR (400 MHz, CDCl₃) δ 8.72 (s, 1H), 7.98 (d, J=8.0 Hz, 1H), 7.67 (s, 1H), 7.58 (d, J=7.2 Hz, 1H), 7.50-7.29 (m, 5H), 7.20 (d, J=8.0 Hz, 1H), 2.40 (s, 3H), 2.02 (s, 3H); ¹³C-NMR (100 MHz, CDCl₃) δ 167.2, 152.9, 147.7, 146.2, 141.0, 137.3, 136.9, 136.7, 135.9, 135.5, 131.2, 129.9, 129.2, 128.3, 127.5, 127.2, 126.8, 122.0, 115.8, 17.2, 11.5;

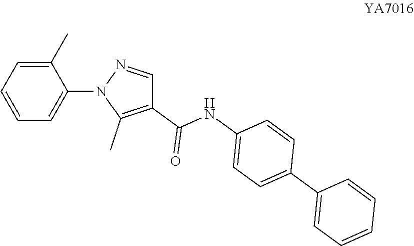 [SMILES: Cc1ccccc1-n1ncc(C(=O)Nc2ccc(-c3ccccc3)cc2)c1C]

N-([1,1′-biphenyl]-4-yl)-5-methyl-1-(o-tolyl)-1H-pyrazole-4-carboxamide (YA7016). The title compound was synthesized according to General Procedure E (%, a white solid): ¹H-NMR (400 MHz, CDCl₃) δ 8.12 (s, 1H), 7.94 (s, 1H), 7.82 (d, J=6.8 Hz, 2H), 7.72 (d, J=7.2 Hz, 4H), 7.58-7.43 (m, 6H), 7.35 (d, J=8.0 Hz, 1H), 2.55 (s, 3H), 2.18 (s, 3H); ¹³C-NMR (100 MHz, CDCl₃) δ 162.0, 144.0, 140.5, 137.9, 137.6, 137.3, 137.1, 136.0, 131.2, 129.9, 128.8, 127.6, 127.1, 126.9, 126.8, 120.5, 114.9, 17.2, 11.2;

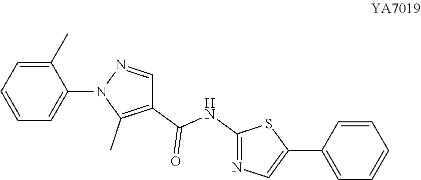 [SMILES: Cc1ccccc1-n1ncc(C(=O)Nc2ncc(-c3ccccc3)s2)c1C]

5-Methyl-N-(5-phenylthiazol-2-yl)-1-(o-tolyl)-1H-pyrazole-4-carboxamide (YA7019). The title compound was synthesized according to General Procedure E (%, a white solid): ¹H-NMR (400 MHz, CDCl₃) δ 12.50 (s, 1H), 8.44 (s, 1H), 7.76-7.75 (m, 3H), 7.66-7.47 (m, 7H), 2.71 (s, 3H), 2.31 (s, 3H); ¹³C-NMR (100 MHz, CDCl₃) δ 161.6, 159.1, 145.1, 139.2, 137.4, 135.9, 132.7, 132.5, 131.6, 131.3, 130.0, 129.1, 127.8, 127.5, 126.9, 126.2, 113.3, 17.3, 11.4;

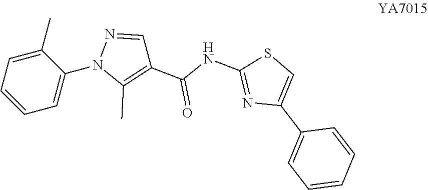 [SMILES: Cc1ccccc1-n1ncc(C(=O)Nc2nc(-c3ccccc3)cs2)c1C]

5-Methyl-N-(4-phenylthiazol-2-yl)-1-(o-tolyl)-1H-pyrazole-4-carboxamide (YA7015). The title compound was synthesized according to General Procedure E (%, a white solid): ¹H-NMR (400 MHz, CDCl₃) δ 10.48 (s, 1H), 7.42 (s, 3H), 7.00-6.89 (m, 6H), 6.81 (s, 1H), 6.75 (d, J=7.6 Hz, 1H), 2.03 (s, 3H), 1.56 (s, 3H); ¹³C-NMR (100 MHz, CDCl₃) δ 161.3, 158.9, 149.8, 144.8, 138.4, 137.3, 135.9, 134.2, 131.1, 129.9, 128.7, 127.9, 127.4, 126.7, 126.1, 112.7, 107.9, 17.1, 11.3;

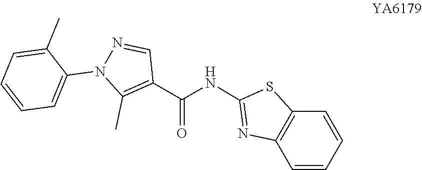 [SMILES: Cc1ccccc1-n1ncc(C(=O)Nc2nc3ccccc3s2)c1C]

N-(Benzo[d]thiazol-2-yl)-5-methyl-1-(o-tolyl)-1H-pyrazole-4-carboxamide (YA6179). The title compound was synthesized according to General Procedure E (%, a white solid): ¹H-NMR (400 MHz, CDCl₃) δ 11.58 (s, 1H), 8.03 (s, 1H), 7.83 (d, J=7.2 Hz, 1H), 7.53 (d, J=8.0 Hz, 1H), 7.40-7.26 (m, 5H), 7.17 (d, J=7.2 Hz, 1H), 2.49 (s, 3H), 2.04 (s, 3H); ¹³C-NMR (100 MHz, CDCl₃) δ 161.7, 159.6, 147.8, 145.2, 139.0, 137.3, 135.8, 131.9, 131.2, 130.0, 127.5, 126.8, 126.2, 123.9, 121.4, 120.6, 112.9, 17.2, 11.5;

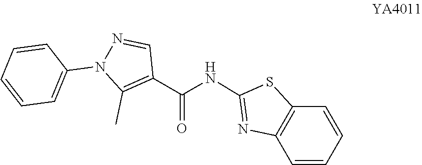 [SMILES: Cc1c(C(=O)Nc2nc3ccccc3s2)cnn1-c1ccccc1]

N-(Benzo[d]thiazol-2-yl)-5-methyl-1-phenyl-1H-pyrazole-4-carboxamide (YA4011). The title compound was synthesized according to General Procedure E (27%, a yellow solid): ¹H-NMR (400 MHz, CDCl₃) δ 8.01 (s, 1H), 7.84 (d, J=8.0 Hz, 1H), 7.57-7.45 (m, 4H), 7.40-7.36 (m, 3H), 7.30 (t, J=7.6 Hz, 1H), 2.69 (s, 3H); ¹³C-NMR (100 MHz, CDCl₃) δ 162.1, 160.0, 148.0, 144.5, 139.7, 138.8, 132.2, 129.7, 129.2, 126.6, 125.8, 124.3, 121.8, 120.8, 114.3, 12.6; HRMS (ESI): calcd. for C₁₈H₁₅N₄OS [M+H]⁺ 335.0967, found 335.0978.

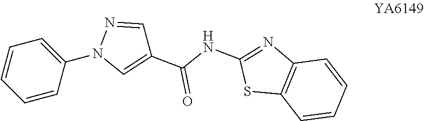 [SMILES: O=C(Nc1nc2ccccc2s1)c1cnn(-c2ccccc2)c1]

N-(Benzo[d]thiazol-2-yl)-1-phenyl-1H-pyrazole-4-carboxamide (YA6149). The title compound was synthesized according to General Procedure E (31%, a yellow solid): ¹H-NMR (400 MHz, DMSO-d₆) δ 12.71 (s, 1H), 9.31 (s, 1H), 8.50 (s, 1H), 8.00 (d, J=7.6 Hz, 1H), 7.88 (d, J=8.0 Hz, 2H), 7.76 (d, J=7.6 Hz, 1H), 7.55 (t, J=7.2, 6.8 Hz, 2H), 7.46-7.37 (m, 2H), 7.31 (t, J=8.0, 6.8 Hz, 1H); ¹³C-NMR (100 MHz, DMSO-d₆) δ 160.8, 158.8, 149.0, 141.7, 139.4, 132.0, 131.2, 130.2, 127.9, 126.6, 124.0, 122.2, 120.8, 119.5, 118.6;

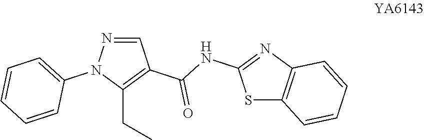 [SMILES: CCc1c(C(=O)Nc2nc3ccccc3s2)cnn1-c1ccccc1]

N-(Benzo[d]thiazol-2-yl)-5-ethyl-1-phenyl-1H-pyrazole-4-carboxamide (YA6143). The title compound was synthesized according to General Procedure E (%, a white solid): ¹H-NMR (400 MHz, CDCl₃) δ 11.07 (s, 1H), 8.00 (s, 1H), 7.83 (d, J=7.6 Hz, 1H), 7.57-7.47 (m, 4H), 7.40-7.35 (m, 3H), 7.31-7.28 (s, 1H), 3.10 (q, J=8.0, 7.6 Hz, 2H), 1.25 (t, J=8.0, 6.8 Hz, 3H); ¹³C-NMR (100 MHz, CDCl₃) δ 161.2, 159.2, 150.3, 147.9, 139.0, 138.6, 132.0, 129.4, 129.2, 126.2, 125.9, 123.9, 121.4, 120.6, 112.9, 18.8, 13.7;

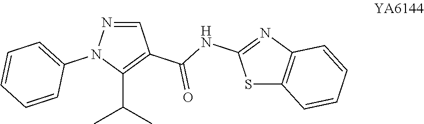 [SMILES: CC(C)c1c(C(=O)Nc2nc3ccccc3s2)cnn1-c1ccccc1]

N-(Benzo[d]thiazol-2-yl)-5-isopropyl-1-phenyl-1H-pyrazole-4-carboxamide (YA6144). The title compound was synthesized according to General Procedure E (31%, a yellow solid): ¹H-NMR (400 MHz, CDCl₃) δ 11.43 (s, 1H), 7.99 (s, 1H), 7.84 (d, J=8.0 Hz, 1H), 7.52-7.27 (m, 8H), 3.43-3.39 (m, 1H), 1.48 (s, 3H), 1.46 (s, 3H); ¹³C-NMR (100 MHz, CDCl₃) δ 161.5, 159.6, 153.3, 147.9, 139.9, 139.3, 131.9, 129.4, 129.3, 126.6, 126.0, 123.8, 121.4, 120.4, 113.1, 26.6, 20.6;

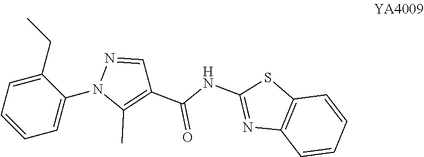 [SMILES: CCc1ccccc1-n1ncc(C(=O)Nc2nc3ccccc3s2)c1C]

N-(Benzo[d]thiazol-2-yl)-1-(2-ethylphenyl)-5-methyl-1H-pyrazole-4-carboxamide (YA4009). The title compound was synthesized according to General Procedure E (27%, a yellow solid): ¹H-NMR (400 MHz, CDCl₃) δ 7.95 (s, 1H), 7.77 (d, J=8.0 Hz, 1H), 7.53 (d, J=7.6 Hz, 1H), 7.41-7.22 (m, 5H), 7.08 (d, J=8.0 Hz, 1H), 2.42 (s, 3H), 2.28 (q, J=7.2 Hz, 2H), 1.01 (t, J=7.2 Hz, 3H); ¹³C-NMR (100 MHz, CDCl₃) δ 161.5, 159.2, 147.7, 145.5, 141.7, 138.7, 136.7, 131.9, 130.2, 129.6, 127.6, 126.7, 126.2, 123.9, 121.4, 120.6, 112.7, 23.9, 14.4, 11.6; HRMS (ESI): calcd. for C₂₀H₁₉N₄OS [M+H]⁺ 363.1280, found 363.1285.

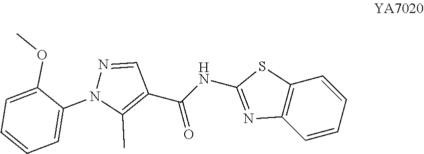 [SMILES: COc1ccccc1-n1ncc(C(=O)Nc2nc3ccccc3s2)c1C]

N-(Benzo[d]thiazol-2-yl)-1-(2-methoxyphenyl)-5-methyl-1H-pyrazole-4-carboxamide (YA7020). The title compound was synthesized according to General Procedure E (27%, a yellow solid): ¹H-NMR (400 MHz, CDCl₃) δ 11.59 (s, 1H), 8.05 (s, 1H), 7.82 (d, J=7.2 Hz, 1H), 7.51 (d, J=8.0 Hz, 1H), 7.44 (t, J=7.6, 8.0 Hz, 1H), 7.35-7.25 (m, 3H), 7.07-7.02 (m, 2H), 3.77 (s, 3H), 2.51 (s, 3H); ¹³C-NMR (100 MHz, CDCl₃) δ 161.9, 159.6, 154.3, 147.8, 146.3, 142.4, 139.3, 131.9, 131.1, 128.8, 127.1, 126.1, 123.8, 121.3, 120.9, 120.6, 112.9, 112.0, 55.8, 11.5;

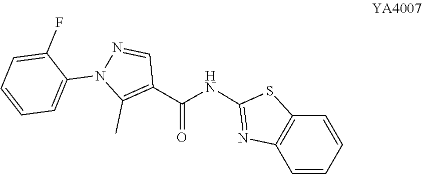 [SMILES: Cc1c(C(=O)Nc2nc3ccccc3s2)cnn1-c1ccccc1F]

N-(Benzo[d]thiazol-2-yl)-1-(2-fluorophenyl)-5-methyl-1H-pyrazole-4-carboxamide (YA4007). The title compound was synthesized according to General Procedure E (31%, a yellow solid): ¹H-NMR (400 MHz, CDCl₃) δ 8.08 (s, 1H), 7.84 (d, J=7.6 Hz, 1H), 7.61 (d, J=7.6 Hz, 1H), 7.51-7.39 (m, 3H), 7.34-7.29 (m, 3H), 2.60 (s, 3H); ¹³C-NMR (100 MHz, CDCl₃) δ 161.5, 159.5, 157.9 (d, J=250.9 Hz), 147.5, 146.2, 139.8, 131.8, 131.4 (d, J=6.4 Hz), 128.9, 126.2, 125.0 (d, J=3.8 Hz), 124.0, 121.4, 120.5, 116.9 (d, J=19.3 Hz), 113.6, 11.4; HRMS (ESI): calcd. for C₁₈H₁₄FN₄OS [M+H]⁺ 353.0872, found 353.0879.

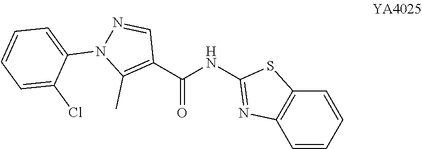 [SMILES: Cc1c(C(=O)Nc2nc3ccccc3s2)cnn1-c1ccccc1Cl]

N-(Benzo[d]thiazol-2-yl)-1-(2-chlorophenyl)-5-methyl-1H-pyrazole-4-carboxamide (YA4025). The title compound was synthesized according to General Procedure E (27%, a yellow solid): ¹H-NMR (400 MHz, CDCl₃) δ 8.08 (s, 1H), 7.84 (d, J=7.6 Hz, 1H), 7.68 (d, J=8.0 Hz, 1H), 7.58 (d, J=8.0 Hz, 1H), 7.51-7.39 (m, 4H), 7.33 (t, J=7.6, 6.4 Hz, 1H), 2.53 (s, 3H); ¹³C-NMR (100 MHz, CDCl₃) δ 161.7, 159.7, 147.6, 146.2, 139.7, 136.1, 132.2, 131.8, 131.2, 130.5, 129.5, 127.8, 126.2, 124.0, 121.4, 120.6, 113.3, 11.5; HRMS (ESI): calcd. for C₁₈H₁₄ClN₄OS [M+H]⁺ 369.0577, found 369.0579.

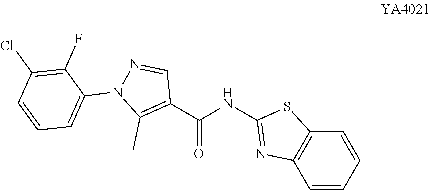 [SMILES: Cc1c(C(=O)Nc2nc3ccccc3s2)cnn1-c1cccc(Cl)c1F]

N-(Benzo[d]thiazol-2-yl)-1-(3-chloro-2-fluorophenyl)-5-methyl-1H-pyrazole-4-carboxamide (YA4021). The title compound was synthesized according to General Procedure E (31%, a yellow solid): ¹H-NMR (400 MHz, CDCl₃) δ 8.07 (s, 1H), 7.84 (d, J=8.0 Hz, 1H), 7.59-7.57 (m, 2H), 7.42-7.28 (m, 4H), 2.61 (s, 3H); ¹³C-NMR (100 MHz, CDCl₃) δ 161.6, 159.8, 154.1 (d, J=253.4 Hz), 147.4, 146.3, 140.3, 131.9, 131.8, 127.6 (d, J=11.6 Hz), 127.3, 126.2, 124.9 (d, J=5.2 Hz), 124.0, 122.7 (d, J=16.7 Hz), 121.4, 120.4, 113.9, 11.4; HRMS (ESI): calcd. for C₁₈H₁₃ClFN₄OS [M+H]⁺ 387.0483, found 387.0472.

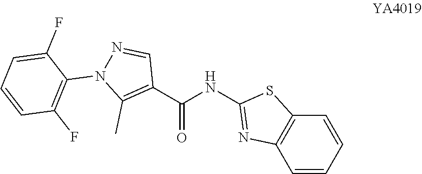 [SMILES: Cc1c(C(=O)Nc2nc3ccccc3s2)cnn1-c1c(F)cccc1F]

N-(benzo[d]thiazol-2-yl)-1-(2,6-difluorophenyl)-5-methyl-1H-pyrazole-4-carboxamide (YA4019). The title compound was synthesized according to General Procedure E (31%, a yellow solid): ¹H-NMR (400 MHz, CDCl₃) δ 8.12 (s, 1H), 7.84 (d, J=7.6 Hz, 1H), 7.63 (d, J=8.0 Hz, 1H), 7.51-7.50 (m, 1H), 7.42-7.40 (m, 1H), 7.34-7.32 (m, 1H), 7.15-7.13 (m, 2H), 2.59 (s, 3H); ¹³C-NMR (100 MHz, CDCl₃) δ 161.4, 159.6 (d, J=256 Hz), 157.1 (d, J=253.4 Hz), 147.5 (d, J=19.3 Hz), 140.4, 131.7 (t, J=9.0, 10.3 Hz), 126.3, 124.0, 121.3, 120.6, 113.6, 112.5 (d, J=21.9 Hz), 11.0; HRMS (ESI): calcd. for C₁₈H₁₃F₂N₄OS [M+H]⁺ 371.0778, found 371.0782.

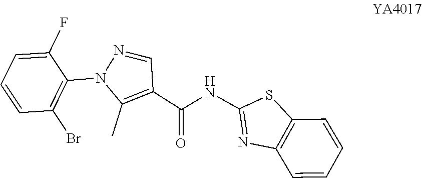 [SMILES: Cc1c(C(=O)Nc2nc3ccccc3s2)cnn1-c1c(F)cccc1Br]

N-(Benzo[d]thiazol-2-yl)-1-(2-bromo-6-fluorophenyl)-5-methyl-1H-pyrazole-4-carboxamide (YA4017). The title compound was synthesized according to General Procedure E (27%, a yellow solid): ¹H-NMR (400 MHz, CDCl₃) δ 8.34 (s, 1H), 8.04 (d, J=7.6 Hz, 1H), 7.83 (d, J=8.0 Hz, 1H), 7.76 (d, J=8.4 Hz, 1H), 7.65-7.58 (m, 2H), 7.54-7.45 (m, 2H), 2.75 (s, 3H); ¹³C-NMR (100 MHz, CDCl₃) δ 161.6, 160.1 (d, J=256 Hz), 159.9, 147.5, 146.9, 140.5, 132.4 (d, J=9.0 Hz), 131.8, 129.0 (d, J=2.6 Hz), 126.5, 126.3, 124.0 (d, J=15.5 Hz), 121.3, 120.7, 116.0 (d, J=19.3 Hz), 113.5, 11.1; HRMS (ESI): calcd. for C₁₁H₁₃BrFN₄OS [M+H]⁺ 430.9977, found 430.9981.

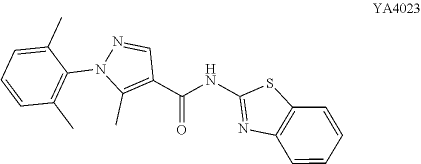 [SMILES: Cc1cccc(C)c1-n1ncc(C(=O)Nc2nc3ccccc3s2)c1C]

N-(Benzo[d]thiazol-2-yl)-1-(2,6-dimethylphenyl)-5-methyl-1H-pyrazole-4-carboxamide (YA4023). The title compound was synthesized according to General Procedure E (27%, a yellow solid): ¹H-NMR (400 MHz, CDCl₃) δ 12.00 (s, 1H), 8.06 (s, 1H), 7.83 (d, J=7.6 Hz, 1H), 7.51 (d, J=6.8 Hz, 1H), 7.28-7.27 (m, 3H), 7.16 (d, J=7.2 Hz, 2H), 2.45 (s, 3H), 1.96 (s, 6H); ¹³C-NMR (100 MHz, CDCl₃) δ 161.9, 159.9, 147.7, 145.3, 139.6, 136.5, 136.1, 131.9, 129.8, 128.4, 126.1, 123.9, 121.4, 120.6, 112.7, 17.2, 11.0;

### Example 2: Novel Axin Stabilizer YA6060 is a Promising Therapy for Nonalcoholic Steatohepatitis (NASH)

This Example describes the synthesis and properties of a novel class of compounds, of which, triazole YA6060 showed dual activities of β-catenin downregulation and AMPK activation via Axin stabilization. YA6060 demonstrated a highly promising drug-like profile and a remarkable efficacy preventing dimethylnitrosamine-induced hepatic fibrosis. This Example also describes the synthesis and several relevant studies including toxicity assessment and anti-NASH efficacy evaluation. Animal models of hepatic metabolic disorders are used to collect critical efficacy data in support of YA6060 development.

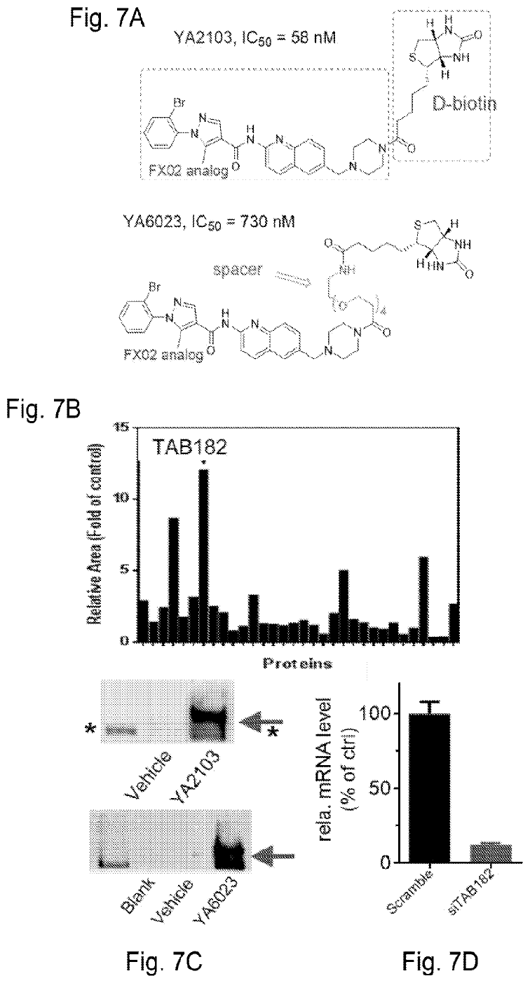

YA6060 (FIG. 2) is a New Chemical Entity (NCE) that holds promise for the difficult-to-treat liver disease of nonalcoholic steatohepatitis (NASH). Although not wishing to be bound by any particular theory, it was hypothesized that increased expression of Axin in the liver could lead to AMPK activation and consequently reduce high fat-induced hepatic steatosis. Simultaneous β-catenin degradation and AMPK activation via Axin stabilization represents an innovative strategy for the treatment of NAFLD/NASH (FIG. 5).

Small molecules that induce the degradation of β-catenin and the activation of AMPK was previously identified, and some of these compounds showed efficacy in a mouse model of hepatic steatosis. Described herein are an improved second generation of compounds, including YA6060 and YA6045

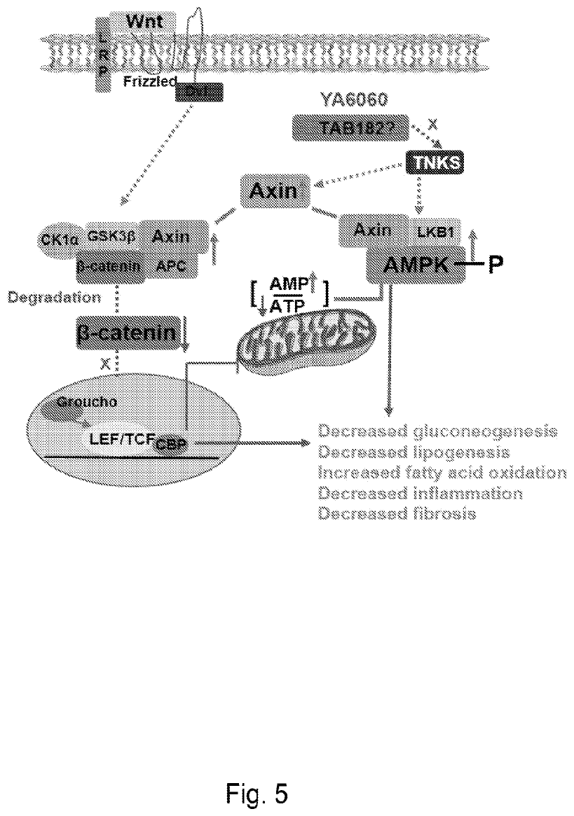

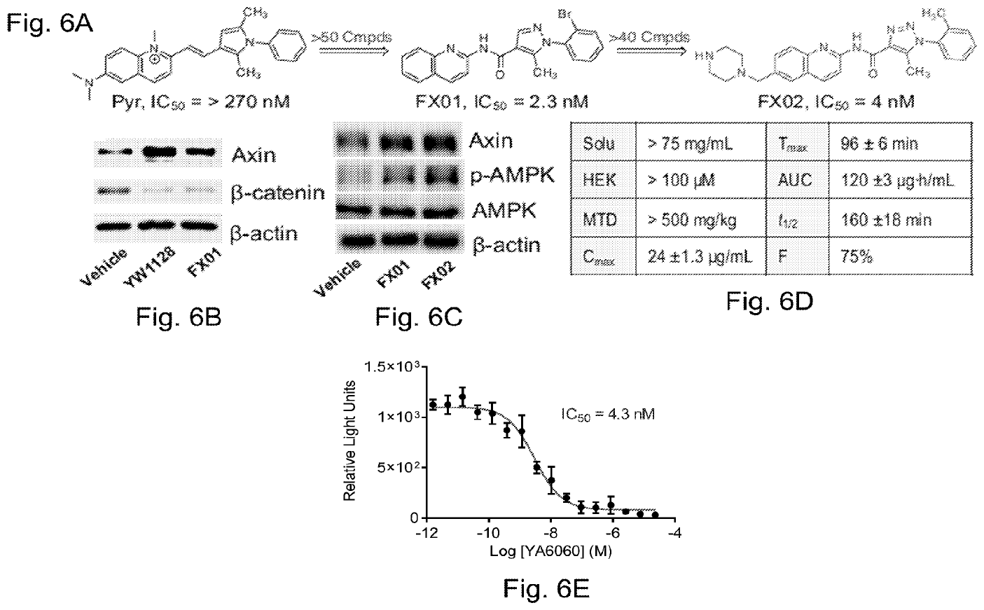

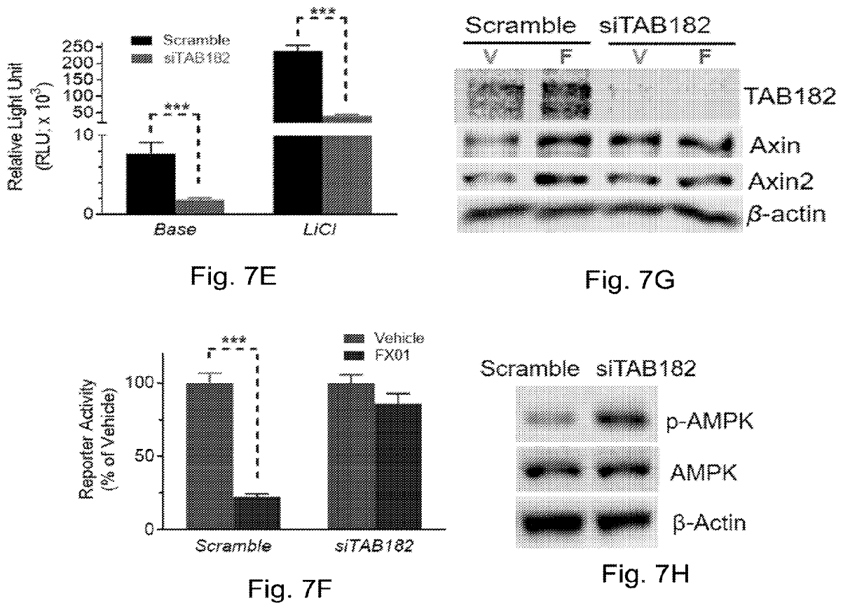

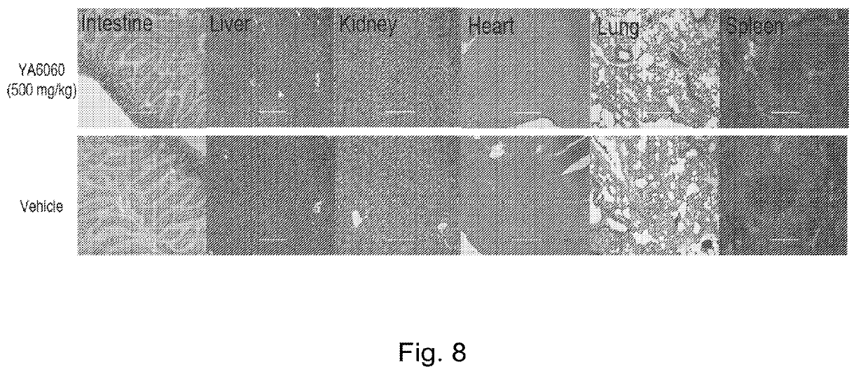

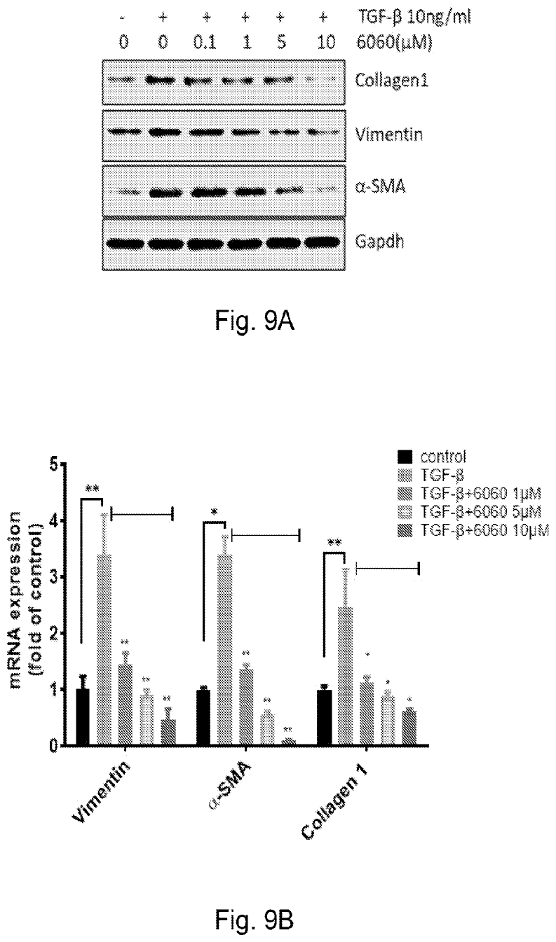

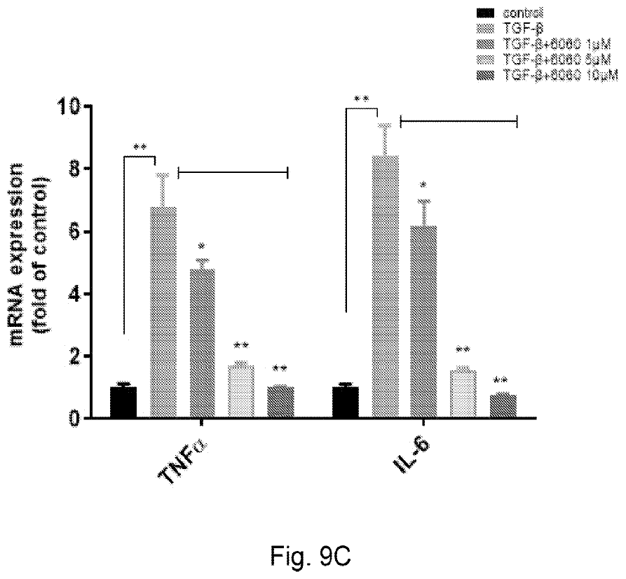

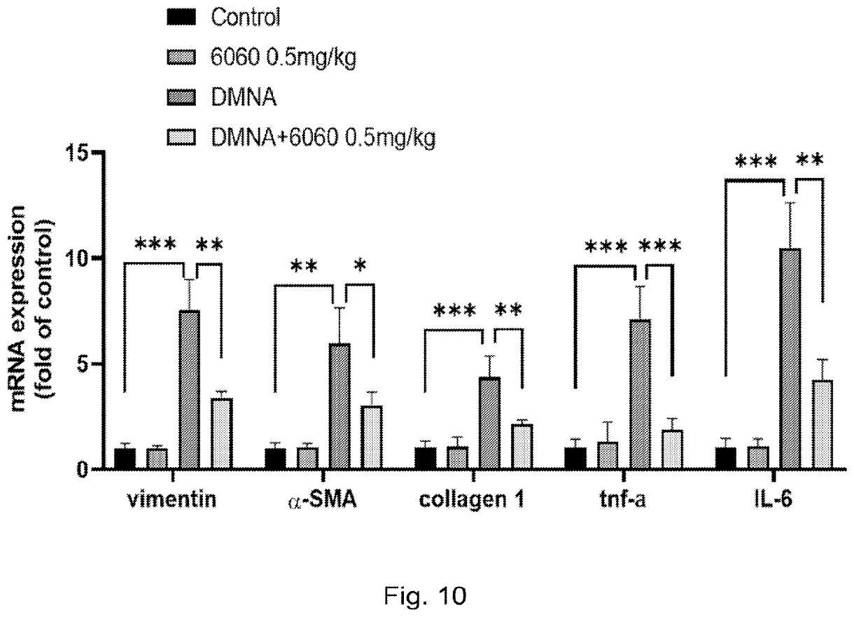

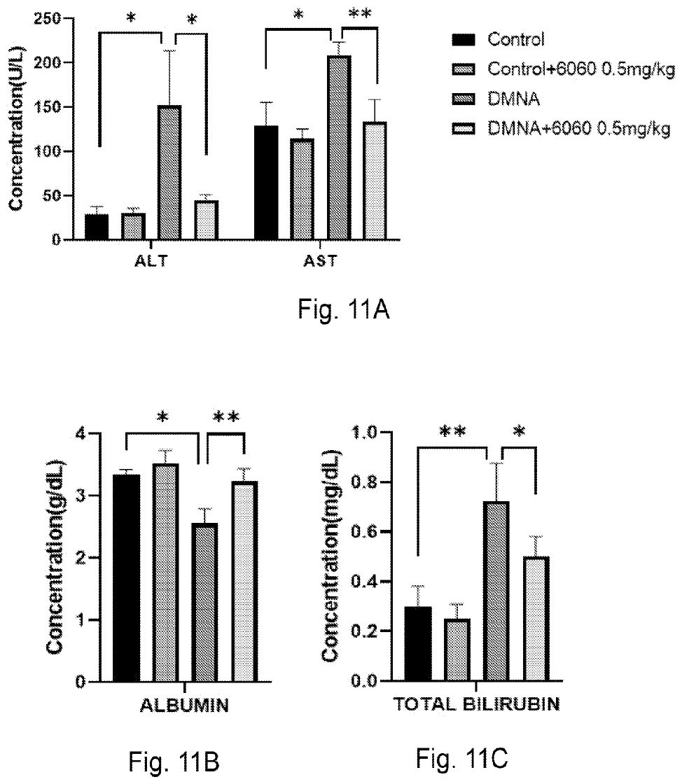

YA6060 is an Axin stabilizer that exhibits the dual activities (FIG. 6A-FIG. 6E). Owning to its activity of β-catenin downregulation, YA6060 is a potent Wnt/β-catenin signaling inhibitor (IC₅₀=4.0 nM). Using biotinylated analogs and proteomic approaches, the tankyrase-binding protein 1 (TAB182) was found to be a direct target of YA6060 analogs (FIG. 5 and FIG. 7A-7H). YA6060 exhibited a high aqueous solubility (>75 mg/mL), little toxicity (mice, single MTD>500 mg/kg), and a good oral bioavailability (75%) (FIG. 3A-FIG. 3B and FIG. 8). Using TGF-β-treated LX-2 cells, it was found that YA6060 significantly reduced the expression of fibrosis markers (vimentin, smooth muscle actin, and Type I collagen) and inflammation markers (TNFα and IL-6) (FIG. 9A-FIG. 9C). YA6060 had strong anti-fibrotic effects in a liver fibrosis mouse model. Non-toxic doses of YA6060 (0.5 or 5 mg/kg/day) reduced dimethylnitrosamine (DMNA)-induced fibrosis in mouse liver (FIG. 4), and led to decreased expression of fibrosis and inflammation markers (FIG. 10). While DMNA caused liver injury, mice receiving YA6060 co-treatment had normal liver function (FIG. 11A-FIG. 11C). Although not wishing to be bound by any particular theory, the data suggest that YA6060 had a protective role in this model system. Function changes in β-catenin, AMPK, and their associated signaling pathways have been implicated in diseases beyond NASH, increasing the therapeutic potential of YA6060 and analogues thereof.

In some embodiments, YA6060 is prescribed by physician to NASH patients. In some embodiments, YA6060 is used by patients of other diseases such as metabolic syndromes and certain cancer.

In preliminary mice studies, YA6060 exhibited no obvious toxicity at a single dose of 500 mg/kg resulting in a >1,000-fold safety margin over an efficacy dose (0.5 mg/kg) for liver fibrosis. If the toxicity evaluation in mice indicated a much narrower therapeutic window, additional compounds of the disclosure can be evaluated and/or lower doses of YA6060 can be combined with other potential therapies. In some embodiments, toxicity and off-target side effects can be addressed with hybrid compounds of which cellular uptake is mediated by the liver-specific OCT1 transporter proteins. In a non-limiting example, extrahepatic side effects of YA6060 can be addressed by synthesizing OCT1-mediated YA6060 analogs or evaluate other related compounds.

Steatosis is the most outstanding feature of NAFLD/NASH, while fibrosis is the hallmark of its progression. Compared to earlier Wnt signaling inhibitors, YA6060 demonstrated a remarkable efficacy for liver fibrosis. In some embodiments, YA6060 is combined with other potential therapies in order to improve fibrosis by at least one stage and/or statistically reduce liver triglyceride content in mouse models. In a non-limiting example, mechanistically YA6060 and metformin could synergize to activate AMPK.

Comprehensive ADME profiling and regulatory toxicity studies. A scale up of the YA6060 synthesis is prepared. Second, although preliminary data indicates an excellent safety profile for YA6060 (FIG. 3, FIG. 8, and FIG. 11A-11C), an accurate estimate of the MTD is needed to determine YA6060's therapeutic index (TI). Lastly and importantly, an ideal drug candidate for NASH reduces key clinical endpoints (i.e., steatosis, hepatic inflammation, and liver cell injury) and have anti-fibrotic effects, while correcting underlying metabolic disorders, such as hepatic insulin resistance and obesity. Although YA6060 has shown promising efficacy for these endpoints, additional data is directly collected from NASH animal models.

Synthesis: 200 mg of YA6060 has been synthesized and purified. YA6060 is scaled up to the 5 g level for use in further studies. Compound YA6060 (and all synthetic intermediates) are characterized using ¹H-NMR, ¹³C-NMR, and high-resolution Mass Spectrometry. Purity (by HPLC) exceeds 95%.

Toxicity evaluation in mice: YA6060 is evaluated for tolerance and general toxicity in mice. First, the single-dose MTD in mice is determined using dose escalation studies. 5 mouse/dose administered YA6060 via oral gavage are assessed for lethal toxicity within 7-21 days. Half log dose intervals re employed. The tolerable daily X5d dosing is determined, starting with 20% of the single-dose MTD as the daily dose in a 5d regimen. The daily dose is increased or decreased, sequentially as necessary, to find the 5d repeat-dose MTD. Animals are evaluated by physical exams, microscopic examination of major organs, serum chemistries, and complete blood counts. MTD>500 mg/kg and in combination with efficacy data, to achieve a TI>1,000.

Anti-NASH efficacy in ob/ob AMLN mice: The genetically obese ob/ob mice treated with AMLN diet of which lipid composition closely reflects a prototypic fast-food diet (i.e., ob/ob AMLN mice) has been widely adopted for testing the anti-NASH efficacy of various compound classes To investigate the anti-NASH efficacy of YA6060 in ob/ob AMLN mice, 8-week old ob/ob mice are fed with AMLN diet (Research Diets, Inc.; D09100310) for 8 weeks and divided into 4 groups (n=15/group). The AMLN mice then receive saline, 0.1, 0.5, or 5 mg/kg YA6060 by oral gavage daily for 4 weeks before euthanasia. Mouse body weight, food intake, and activities are examined for general toxicity response twice per week. The methods for outcome measurement have been established previously. Briefly, liver and plasma triglyceride content is determined. To assess liver injury, fresh-frozen liver sections are prepared to detect reactive oxygen species (ROS) levels and cell death by the fluorescent probe DHE and TUNEL assay, respectively. Liver damage is assessed by serum ALT and AST enzyme levels. H&E staining is used to examine general liver histology, Sirius red staining for fibrosis, and Oil Red O for lipids. The biomarkers (fibrosis and inflammation) are examined by immunoblotting and RT-PCR. To assess general metabolic status, glucose tolerance test and measurement of fasting glucose and insulin levels is performed in subgroups of mice. The primary outcome endpoints are hepatic triglyceride content and fibrosis stage. Overall, YA6060 is expected to decrease hepatic steatosis and fibrosis, enhance hepatic insulin sensitivity, and improve metabolic homeostasis. Improvement in fibrosis by at least 1 Ishak stage with significantly reduced hepatic triglyceride content is expected.

Anti-NASH efficacy in mice administered Western diet with weekly tetrachloride: None of the present models fulfill all requirements for an ideal NASH model. Multiple NASH models should be used for the preclinical characterization of candidate anti-NASH drugs. The mouse model administered a Western diet (WD) with weekly CCl₄ has been reported to closely resemble human NASH. This model is also employed to test YA6060. As described, male C57BL/6J mice are fed WD and a high sugar solution, with weekly I.P. injection of CCl₄, for 12 weeks. The mice are then divided into 4 groups (n=15/group) and receive saline, 0.1, 0.5, or 5 mg/kg YA6060, respectively, by oral gavage daily for 4 weeks before euthanasia. The outcome measurement and data analysis is essentially the same as described above. Improvement in fibrosis by at least 1 stage with significantly reduced hepatic triglyceride content is expected.

### Example 3: Anti-Fibrotic Efficacy of YA6060

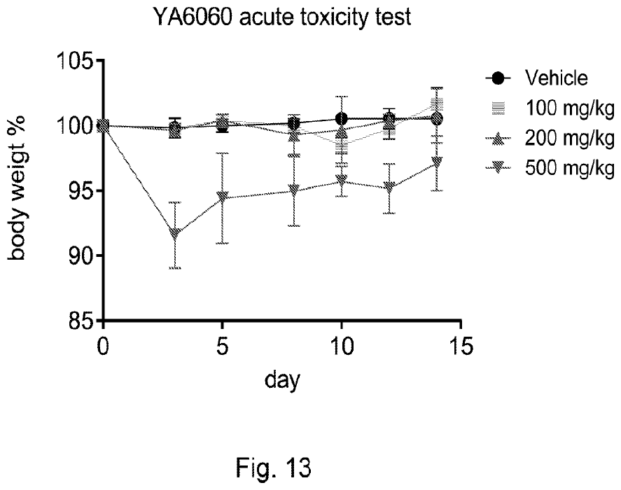

YA6060 was found to have dual activities of Wnt signaling inhibition and AMPK activation (FIG. 6A-FIG. 6E), and YA6060 treatment was found to have no noticeable toxic effects in mouse tissues (FIG. 8). YA6060 did not exhibit acute toxicity in a test in mice (FIG. 13) Body weight change over time in mice received YA6060. 11 weeks C57BL/6 mice were treated for 14 consecutive days by oral garage with YA6060 at the indicated doses once daily and vehicle (saline), respectively.

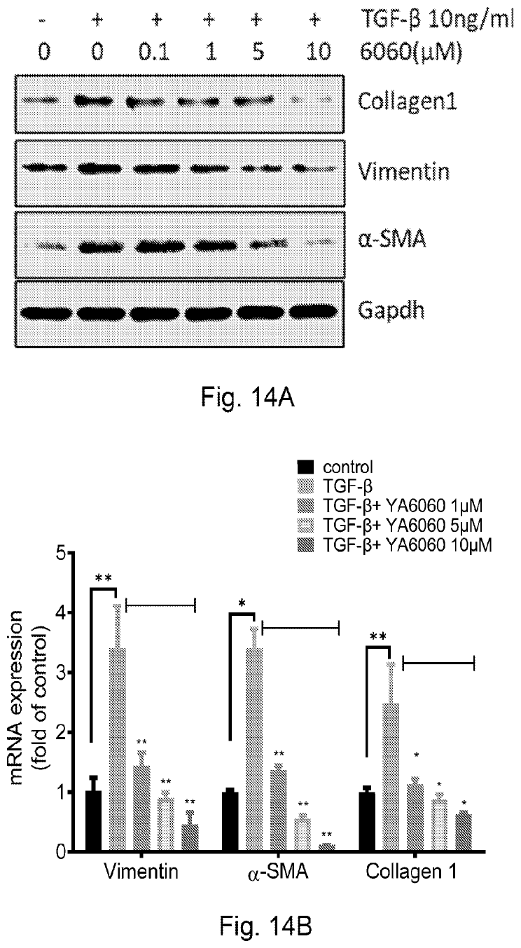

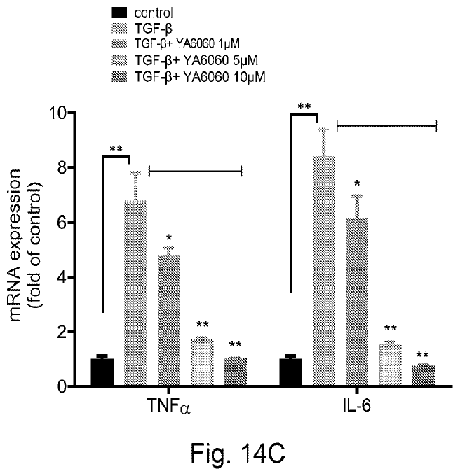

The anti-fibrotic efficacy of YA6060 was demonstrated in LX-2 cells (FIG. 14A-FIG. 14C). The effect of YA6060 treatment on the expression of fibrotic markers in LX-2 cells was examined. LX-2 cells were treated with TGF-beta to model fibrosis. Protein expression of collagen 1, vimentin, and alpha-SMA by Western blotting (FIG. 14A). mRNA expression of collagen 1, vimentin, and alpha-SMA by RT-PCR was examined (FIG. 14B). mRNA expression of genes associated with inflammation by RT-PCR was examined (FIG. 14C). The cells were treated with the indicated concentrations of YA6060 for 24 hours. *P<0.05, **P<0.01. The effect of YA6060 treatment on liver fibrosis in mice was also examined (FIG. 15). C57BL mice were treated with 4 weeks of dimethylnitrosamine (DMNA) (for each week: 10 ug/g/day for 3 days, then no treatment for 4 days) to establish the animal model of liver fibrosis. The mice received either saline control or YA6060 with or without DMNA. The liver tissues were stained by Sirius red (SR) to show fibrosis.

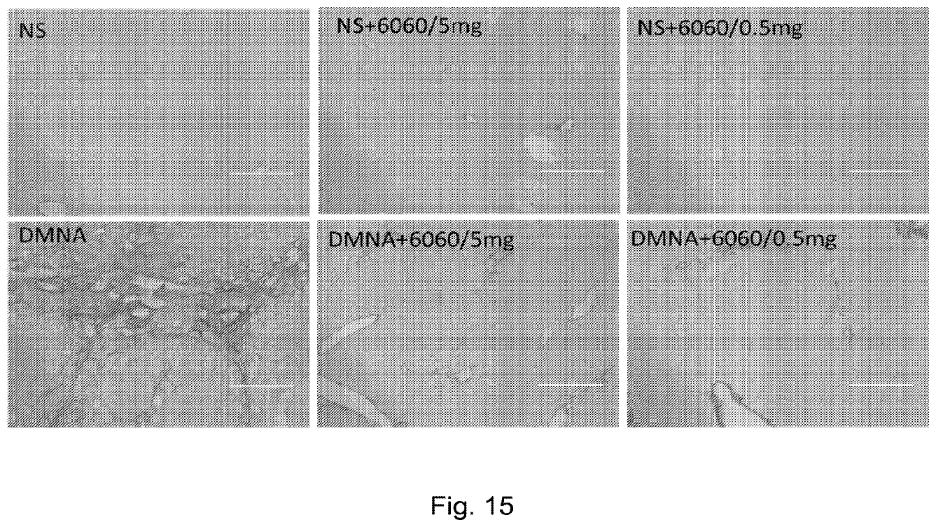

Fibrosis in the liver tissues of the mice that received YA6060 treatment was quantified (FIG. 16). C57BL mice were treated with 4 weeks of dimethylnitrosamine (DMNA) (for each week: 10 ug/g/day for 3 days, then no treatment for 4 days) to establish the animal model of liver fibrosis. The mice received either saline control or YA6060 with or without DMNA. The collagen of liver tissues was stained by Sirius red (SR) and quantitated by Image J software. ***P<0.001.

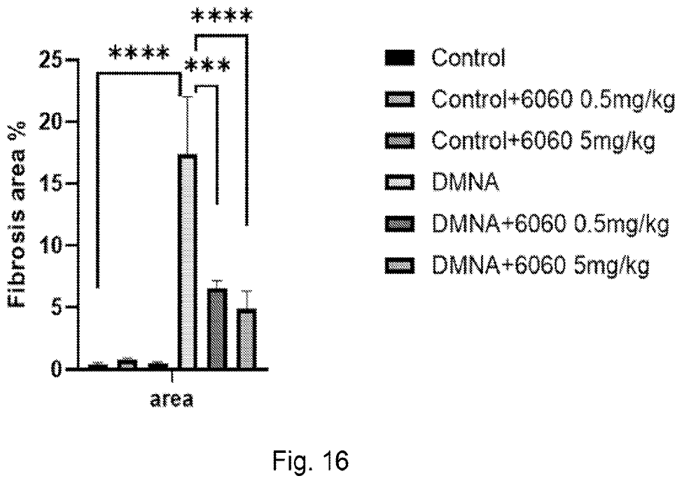

The effect of DMNA and YA6060 treatment on hepatic histology in mice was examined (FIG. 17). C57BL mice were treated with 4 weeks of dimethylnitrosamine (DMNA) (for each week: 10 ug/g/day for 3 days, then no treatment for 4 days) to establish the animal model of liver fibrosis. The mice received either saline control or YA6060 with or without DMNA. The liver tissues were stained by H&E. DMNA treatment led to inflammation infiltration which could be ameliorated by YA6060 treatment.

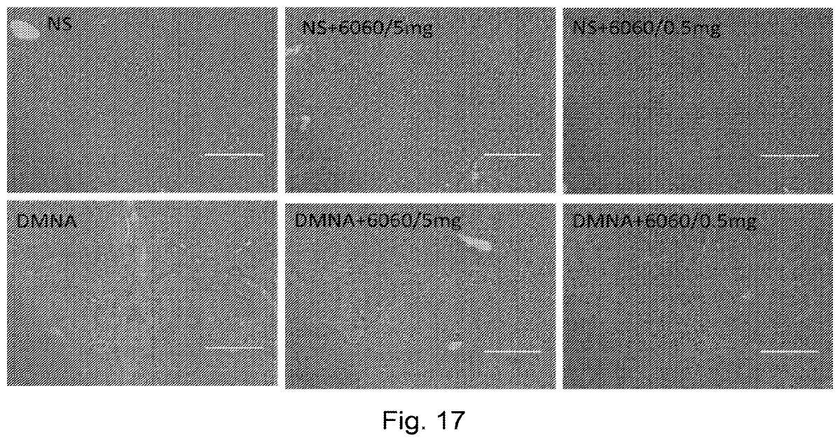

The number of mice in Ishak fibrosis stage was examined (FIG. 18). C57BL mice were treated with 4 weeks of dimethylnitrosamine (DMNA) (for each week: 10 ug/g/day for 3 days, then no treatment for 4 days) to establish the animal model of liver fibrosis. The mice received either saline control or YA6060 with or without DMNA. The histology of liver tissues were scored according to Ishak Fibrosis Stage for each mouse.

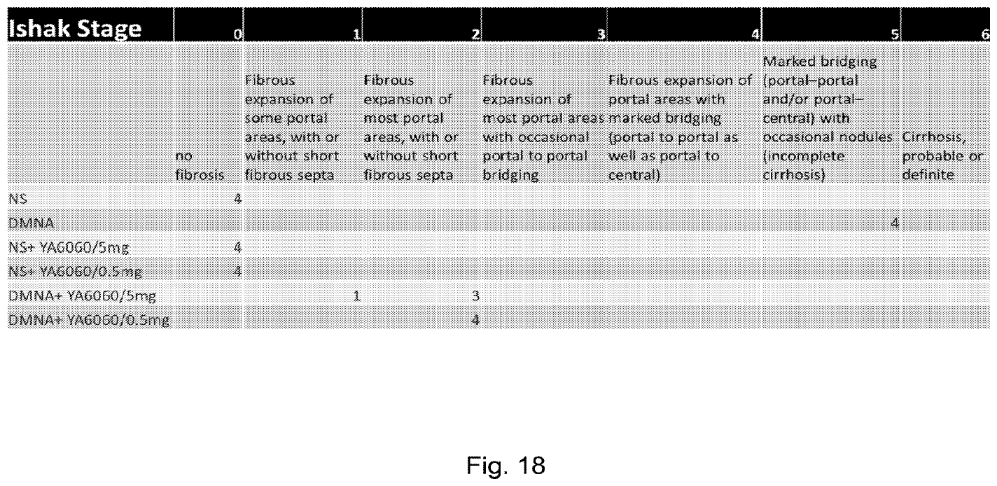

The effect of YA6060 treatment on the hepatic expression of fibrotic and inflammatory markers in mice was examined (FIG. 19). C57BL mice were treated with 4 weeks of dimethylnitrosamine (DMNA) (for each week: 10 ug/g/day for 3 days, then no treatment for 4 days) to establish the animal model of liver fibrosis. The mice received either saline control or YA6060 with or without DMNA. The mRNA expression of fibrotic and inflammatory marker genes was determined by RT-PCR. *P<0.05, **P<0.01, ***P<0.001.

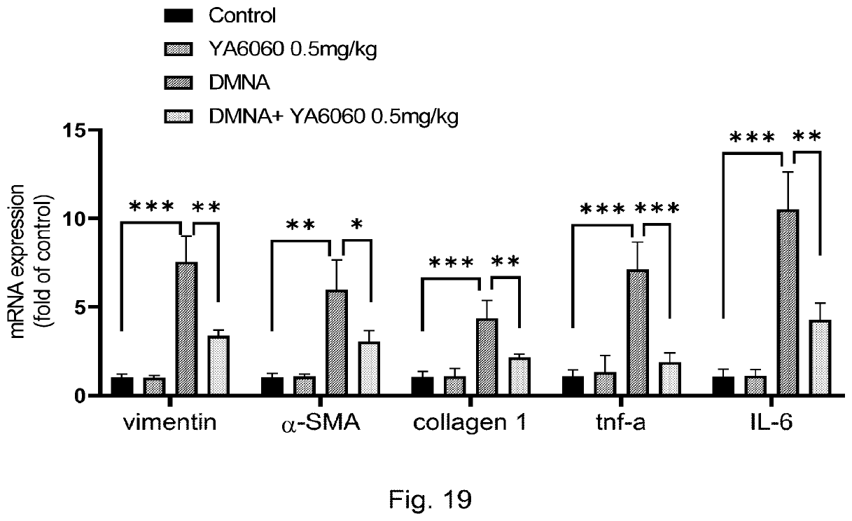

The effect of DMNA and YA6060 treatment on body weight change (FIG. 20A) and liver/body weight (FIG. 20B) in mice were examined. C57BL mice were treated with 4 weeks of dimethylnitrosamine (DMNA) (for each week: 10 ug/g/day for 3 days, then no treatment for 4 days) to establish the animal model of liver fibrosis. The mice received either saline control or YA6060 with or without DMNA. *P<0.05, **P<0.01, ***P<0.001.

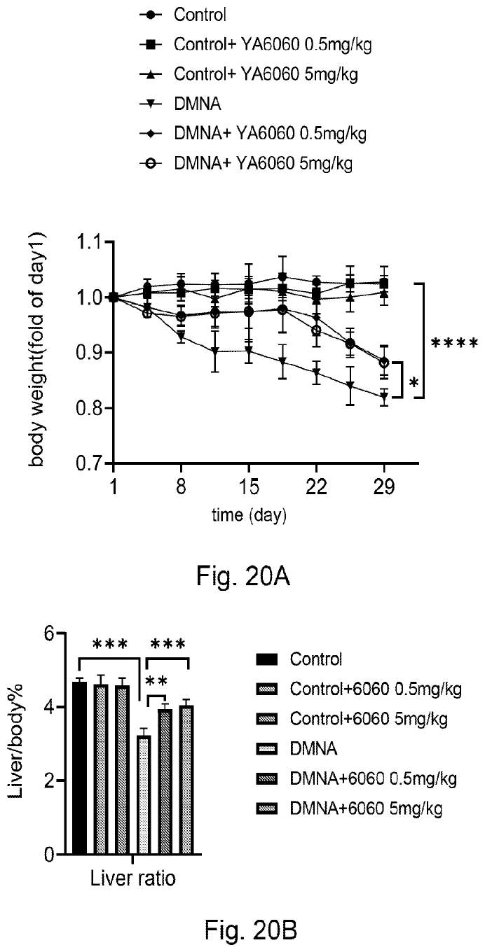

The effect of DMNA and YA6060 treatment on liver function in mice was examined (FIG. 21A-FIG. 21C). C57BL mice were treated with 4 weeks of dimethylnitrosamine (DMNA) (for each week: 10 ug/g/day for 3 days, then no treatment for 4 days) to establish the animal model of liver fibrosis. The mice received either saline control or YA6060 with or without DMNA. The liver function was determined by analyzing the serum biochemistry of albumin (FIG. 21A), total bilirubin (FIG. 21B), and ALT and AST levels (FIG. 21C). *P<0.05, **P<0.01 as compared to the control.

### Example 4: YA6060 is a Dual Activator for Wnt Signaling and AMPK

This Example describes a novel class of compounds, of which, triazole YW2065 showed dual activities of Wnt signaling inhibition and AMPK activation via the mechanism of Axin stabilization. Compound YW2065 selectively kills Wnt-dependent CRC cells, and indicated promising efficacy against CRC cell growth both in vitro and in mice. Based on the chemical structure of YW2065, a new analogue YA6060 was designed and synthesized. While maintaining dual activity of Wnt inhibition and AMPK activation, the new analogue YA6060 demonstrated a highly promising druglike profile highlighted by oral bioavailability (F=75%), half-life (t_{1/2}=160 min), maximum toxic dose (MTD>500 mg/kg) and aqueous solubility (>75 mg/mL). Moreover, using biotinylated analogs and proteomic approaches, it was discovered that the tankyrase-binding protein 1 (TAB182) was the protein target of the new analogues.

Wnt signaling and AMPK pathways are validated targets for anticancer therapies. YW2065 was reported to have dual activities of Wnt inhibition and AMPK activation (FIG. 6A-FIG. 6D). YW2065 was modified to yield a new analogue YA6060, which not only maintained high potency, but also showed excellent physiochemical and PK/Tox properties. YA6060 showed promising anti-CRC effects through direct binding to TAB182.

YA6060 was developed based on previous compound YW2065. Structure modification of known Wnt inhibitor Pyr provided YW2065, and further modification of YW2065 provided new compound YA6060 (FIG. 6A). A dual-luciferase gene reporter assay was used to test inhibitory activities of new compounds on Wnt signaling. The IC₅₀ value of YA6060 was determined to be 4 nM, comparable to that of parent YW2065 (2.3 nM). Both compounds decreased the protein level of β-catenin while stabilizing the level of Axin 1 (FIG. 6B FIG. 6C), the concentration-limiting part of the destruction complex for β-catenin. Axin is also reported to form a complex with AMPK and its upstream kinase LKB1 (FIG. 12A and FIG. 12B) to facilitate AMPK activation. Therefore, the effects of new compounds on AMPK was examined. The levels of phosphorylated AMPK (p-AMPK) were significantly increased by both YW2065 and YA6060 (FIG. 6C), albeit no changes in total AMPK and mRNA transcripts from cell lysates (data not shown). YA6060 and its analogs had dual activities of potent Wnt signaling inhibition and AMPK activation, which were dependent on the levels of scaffolding protein Axin.

YA6060 has an excellent druglike profile. Compared to Pyr and YW2065, YA6060 showed dramatically enhanced druglike properties without any clinical toxicity. The ADMET and PK properties of YA6060 was determined. Compared to Pyr and YW2065 that have poor solubility (<0.01 mg/mL), new lead YA6060 show remarkable solubility of >75 mg/mL in water (FIG. 6D). In PK studies, at an oral dose of 5 mg/kg, the Cₘₐₓ and Tₘₐₓ values of compound YA6060 were determined to be 24 μg/mL and 96 min, respectively. The half-life (t_{1/2}) was 160 min. Moreover, compound YA6060 indicated an excellent oral bioavailability (F) of 75%, which is dramatically enhanced from that of previous lead YW2065 (13%). Importantly, YA6060 was not toxic in normal HEK293 cells (IC₅₀>100 μM), and well tolerated in mice at a dose as high as 500 mg/kg in a 21-day evaluation without any histological evidence of toxicity in the intestine, as well as other major organs including liver, kidney, heart, lung, and spleen (FIG. 8).

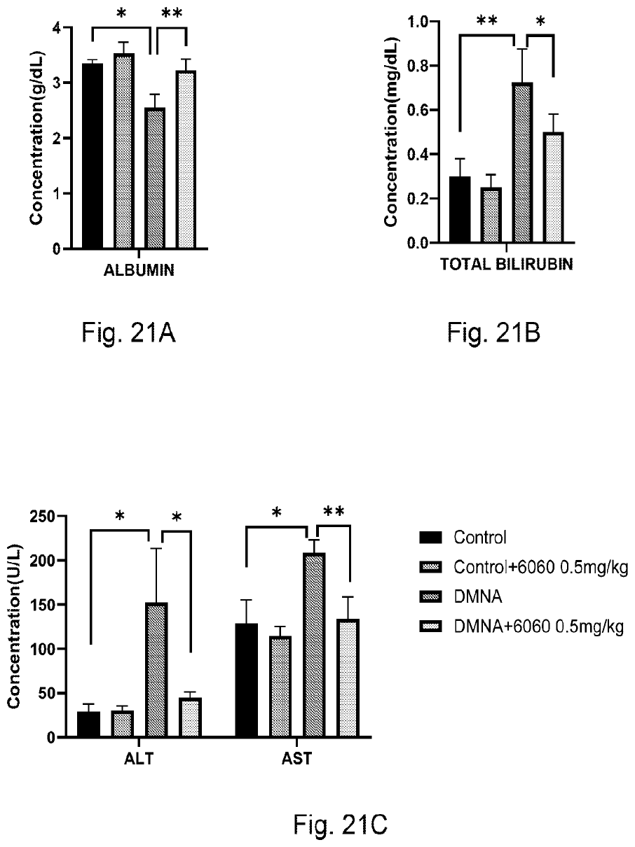

YW2065 and YA6060 selectively kill CRC cells. Inhibition of Wnt signaling by YW2065 was confirmed in human CRC SW480 (IC₅₀=1.2 nM) and SW620 (IC₅₀=0.19 nM) cells using luciferase gene reporter assays (FIG. 22A). Treatment of cells by the compound decreased levels of β-catenin, increased levels of Axin (FIG. 22B), and decreased transcripts of Wnt target genes axin2, cMyc, cyclin D1, MAIP7 and S100A4 (FIG. 22C). The Wnt-dependent cell lines such as SW480, SW620, HCT116 and HT29, which harbor mutations of APC or β-catenin, were more sensitive to YW2065 treatment as compared to RKO, A549 (lung cancer) and normal HEK293 cells, which are known to be independent of Wnt signaling (FIG. 22D). Compared to YW2065, YA6060 has an improved efficacy in killing the CRC cells (e.g., FIG. 22E in HT29 cells, IC₅₀: YA6060 vs YW2065; 8.6 vs 17 μM). Notably, the IC₅₀ values of YW2065 and YA6060 in killing CRC cells were actually comparable to those of 5-FU and oxaliplatin, two current first-line chemotherapeutic agents for CRC, as reported in literature (low μM).

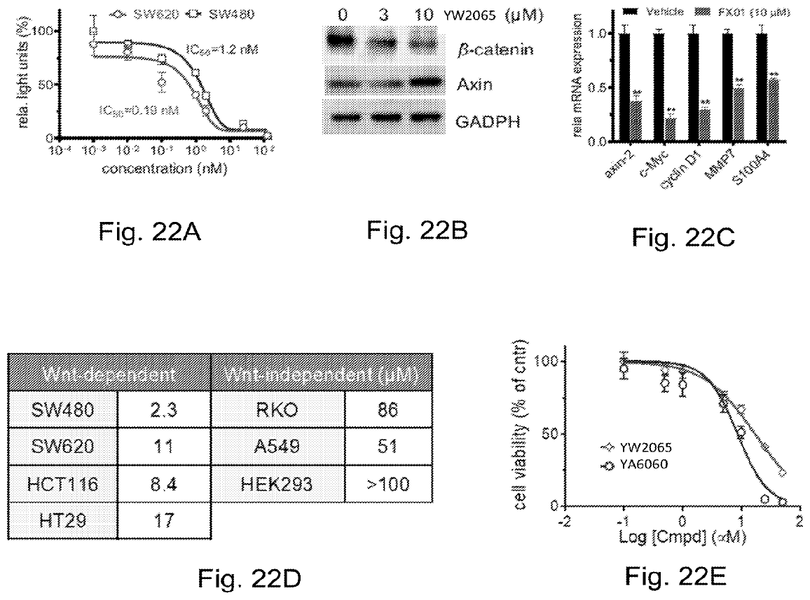

YW2065 functions through Axin stabilization. YW2065 inhibited the colony formation for SW480, SW620, and HT29 cells (FIG. 23A), leading to more G1 phase arrest (data not shown) and apoptosis in SW620 cells (FIG. 23B). Moreover, SW620 cells became resistant to YW2065 treatment when Axin was knocked down or a constitutively active β-catenin mutant was overexpressed (FIG. 23C), consistent with the mechanistic engagement of Axin and Wnt pathway in YW2065 action. While not wishing to be bound by any particular theory, the remaining efficacy by YW2065 in Wnt-independent cells is expected to be at least partially related to AMPK activation

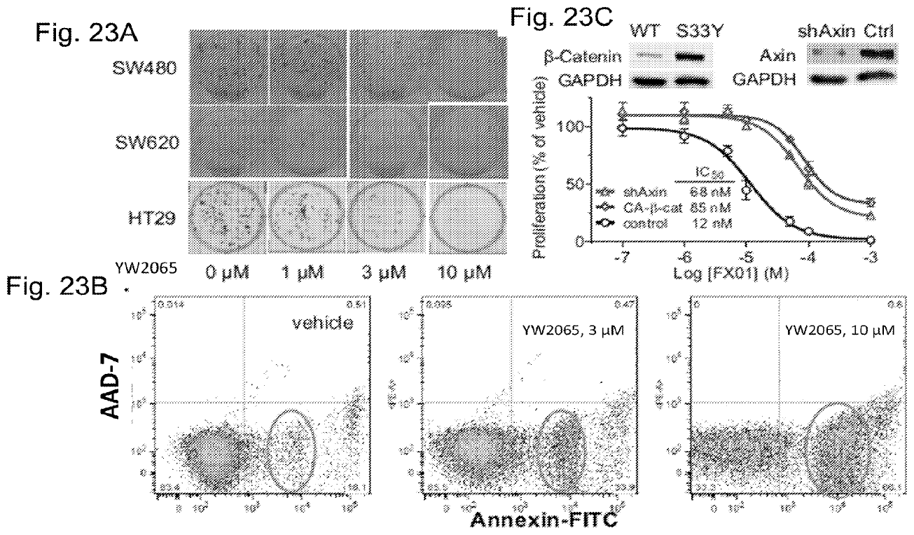

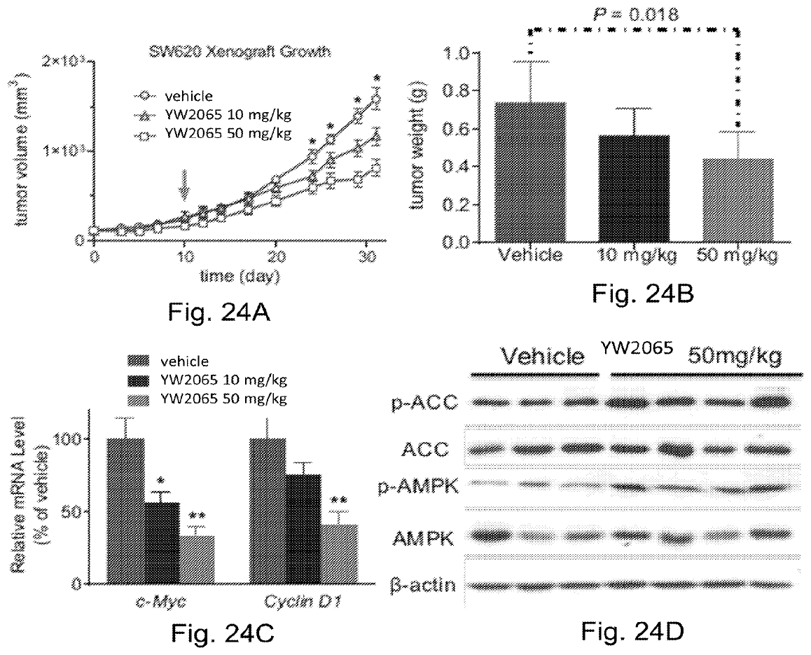

YW2065 suppresses CRC in vivo. The anti-CRC potential of YW2065 was tested using nude mice that received implantation of SW620 cells. YW2065 reduced both tumor growth (FIG. 24A) and tumor weight (FIG. 24B) in a dose-dependent manner, while the body weight gain was not affected. Moreover, the transcripts of Wnt target genes c-Myc and cyclin D1 were reduced by YW2065 (FIG. 24C) while the phosphorylation of AMPK and its downstream ACC was enhanced (FIG. 6D). A murine Orthotopic transplantation model of CRC invasion and dissemination (UMB IACUC #0917017) was established. Briefly, 2×10⁶ murine CRC MC38Luc1 cells were injected S.C. into the flanks of syngeneic C57BL/6 mice. The tumors were resected 3 weeks later and cut into small pieces. The abdomen of anesthetized mice was incised, the cecum exposed and a tumor piece was sutured to the cecal surface of the colon (FIG. 25A). Tumor invasion into the cecum and an average of 1.5 metastatic liver nodules/mouse (n=10) were found after 5 weeks (FIG. 25B). Notably, the metastases were hematogenous; contiguous tumor invasion was not detected. MC38luc1 cells expressed luciferase and their growth in vivo were monitored by real time bioluminescent imaging (BLI) using a Xenogen IVIS-200 Optical Imaging System (FIG. 25C and FIG. 25D).

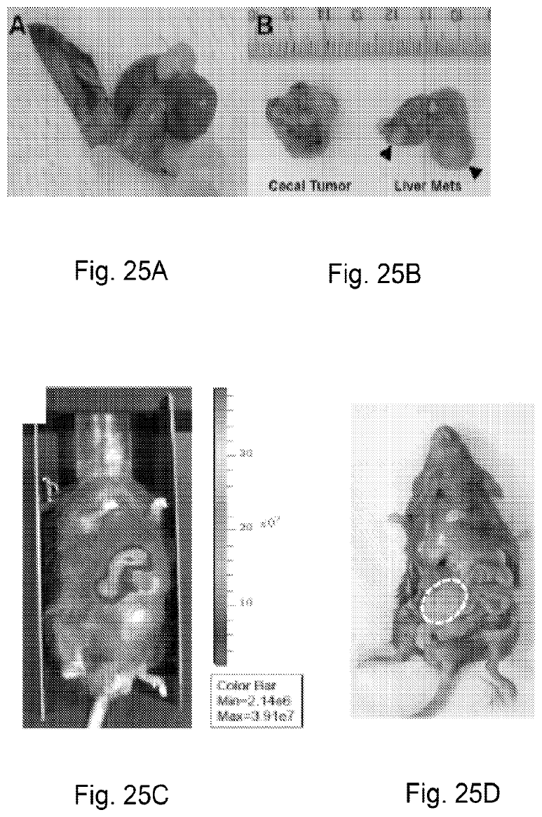

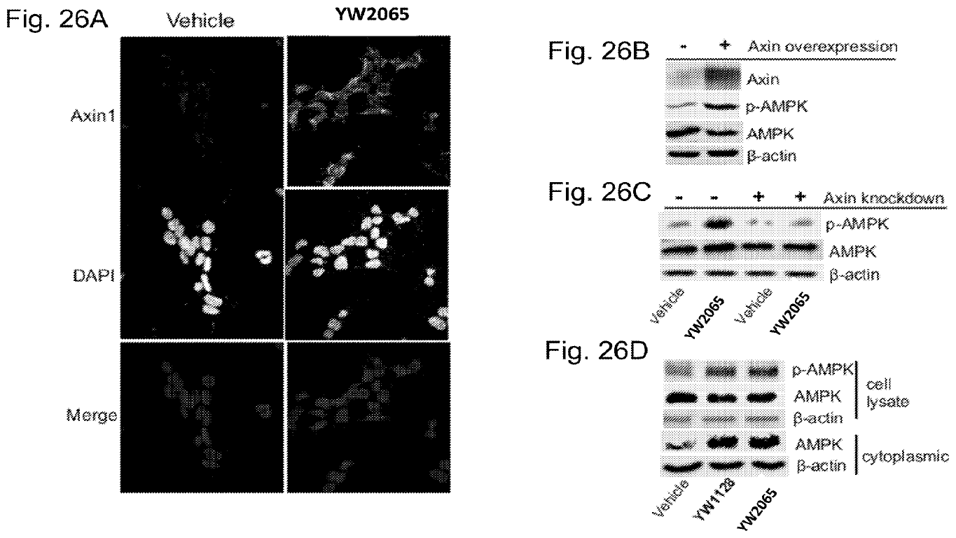

The effects of FX analogs in Axin stabilization and AMPK activation were further studied by genetic manipulation of Axin. Axin overexpression led to enhanced AMPK activation (FIG. 26A and FIG. 26B). In contrast, it has been reported that knockdown of Axin in cells and in mouse liver impairs AMPK activation. While not wishing to be bound by any particular theory, the activation of AMPK by YW2065 seemed to be dependent on Axin expression as Axin knockdown abolished this effect (FIG. 26C). It was also observed that the levels of both p-AMPK and cytosolic AMPK were increased by YW2065 and its analog YW1128, albeit no changes in total AMPK proteins from cell lysates (FIG. 26D). While not wishing to be bound by any particular theory, this result suggests cellular redistribution of AMPK by the YW2065 and its analog. Axin stabilization and AMPK activation occurred as early as three hours after treatment with a potent YW2065 analog (YW2049, FIG. 27). This timeline is consistent with the reports that Axin can form a complex with AMPK. While not wishing to be bound by any particular theory, together these data suggest a mechanism of action that is independent of canonical nuclear β-catenin signaling on gene expression

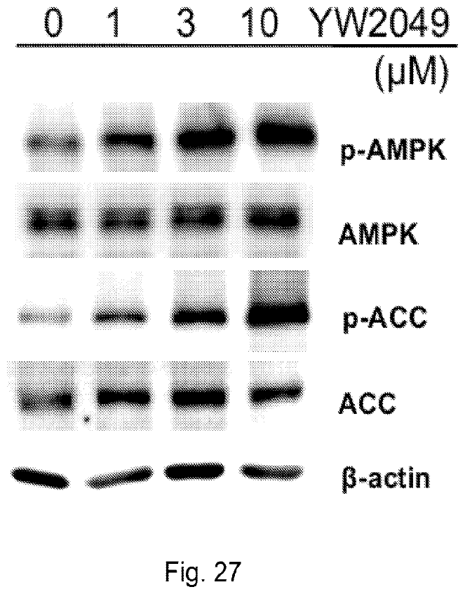

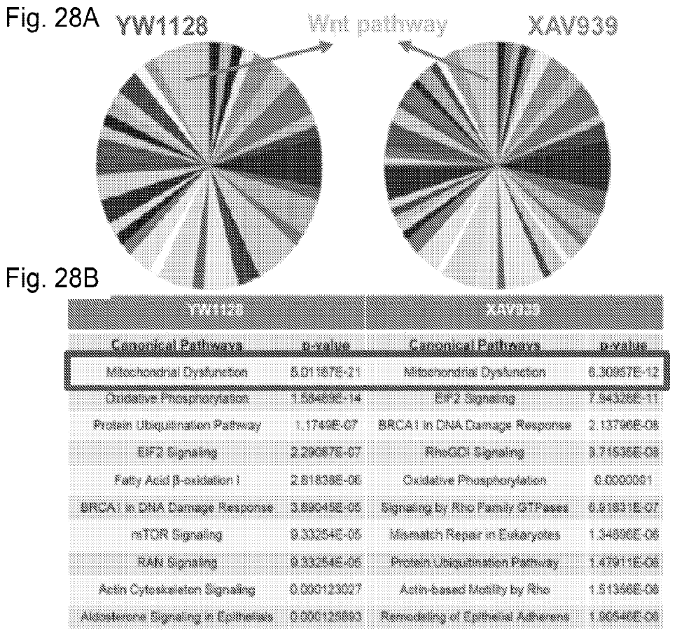

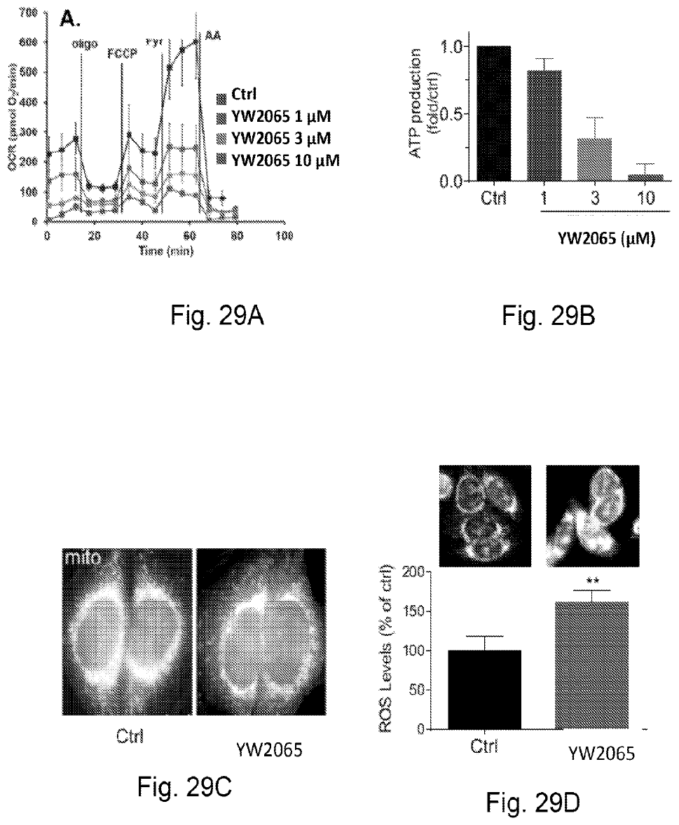

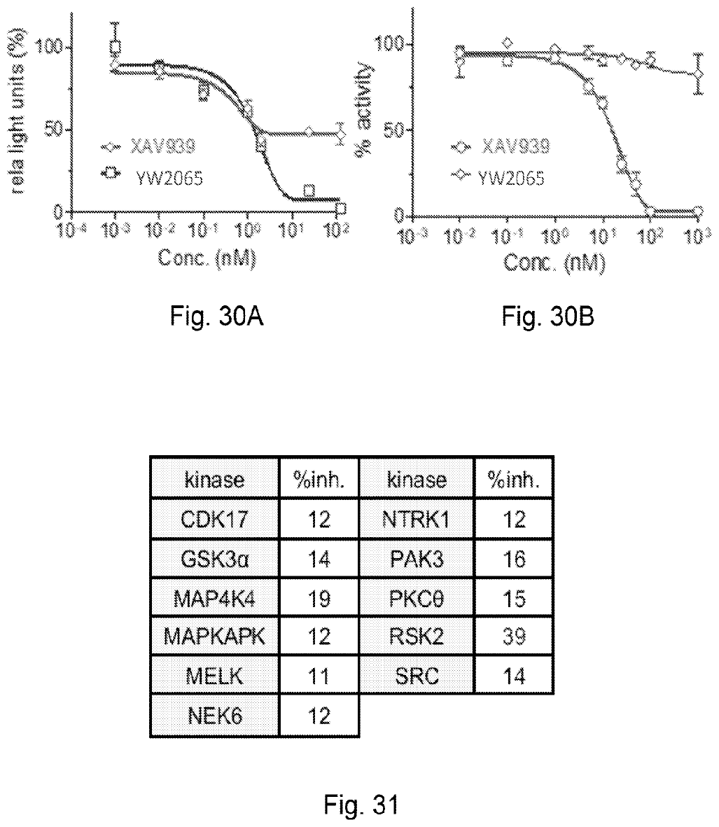

Effects of YW2065 on mitochondrial function. AMPK can be activated by falling energy status that is a function of mitochondria. Mitochondrial can be suppressed by Axin over-expression. TNKS inhibitors XAV939 and Pyr have been reported to inhibit mitochondria. Unbiased analyses were performed for the proteomic changes by YW1128 (YW2065 analog) and XAV939 in HEK293 cells (FIG. 28A). There were significant overlapping changes in pathways, particularly in the Wnt signaling pathway, by these two compounds, consistent with their shared mechanism of Axin stabilization. Mitochondrial dysfunction was identified as the most significantly affected pathway by both compounds (FIG. 28B). The effects of YW2065 on mitochondrial function in SW480 cells was further assessed. YW2065 reduced O₂ consumption rate (FIG. 29A) and ATP production (FIG. 29B), and also increased mitochondrial fragmentation (FIG. 29C) and ROS generation (FIG. 29D). However, in reporter assays, XAV939 could not fully inhibit Wnt signaling activity in SW480 cells (FIG. 30A), indicating its limited efficacy as previously reported, which is likely due to TNKS polymerization. In contrast, YW2065 could abolish Wnt activity at high concentrations. Importantly, while XAV939 is a TNKS inhibitor, a preliminary biochemical assay (BPS Bioscience) suggested YW2065 was not (FIG. 30B). To further confirm target specificity, YW2065 was tested against a panel of 273 kinases. The results indicated that YW2065 inhibited none of the tested kinases with over 40% at 1000 μM. It displayed a moderate inhibitory activity (39% inhibition) for RPS6KA3, and weak activities against 10 other kinases (FIG. 31).

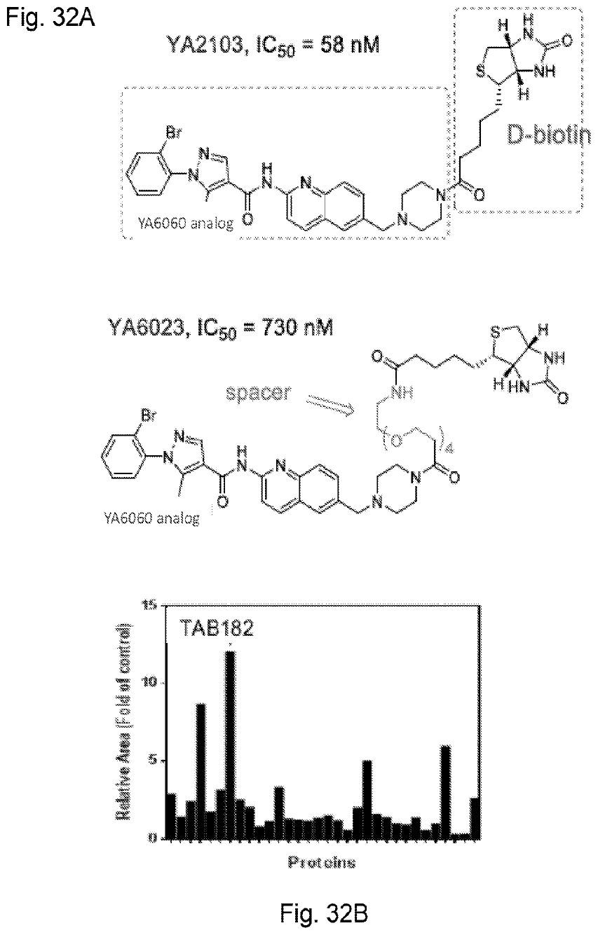

TAB182 as a protein target for YA6060 analogs. Previously, CK1α was reported as a direct target for Pyr; however, this conclusion was challenged. CK1α was not confirmed as the target for YA6060 analogs by either SPR binding assay, or functional validation via CK1α knockdown in the reporter assay, or pulldown experiments. Two biotinylated YA6060 analogs YA2103 and YA6023 containing different length of spacers were synthesized, while both analogs maintained inhibitory activities in the reporter assay (FIG. 32A). Incubation of these analogs and streptavidin beads with HEK293 lysates allowed for pulling down the proteins bound to these analogs. TAB182 was identified as one of the top proteins by unbiased protein profiling via LC-MS/MS (FIG. 32B) and confirmed by immunoblotting (FIG. 32C). In further validation, TAB182 knockdown (FIG. 32D) caused a significant decrease in Wnt signaling activity as assessed by TOPflash luciferase assay (FIG. 14E). Moreover, YW2065 did not inhibit Wnt signaling in the presence of TAB182 knockdown, in contrast to scramble siRNA treatment where it had significant inhibition (FIG. 32F). Under TAB182 knockdown, the increase of Axin protein by YW2065 was no longer observed (FIG. 32G). Consistently, TAB182 knockdown stabilized the base level of Axin protein. Importantly, TAB182 knockdown also led to AMPK activation (FIG. 32H). While not wishing to be bound by any particular theory, together these data strongly support TAB182 as a target for these compounds.

Assessing the anti-CRC efficacy of selected compounds. CRC is of a major interest for targeting Wnt pathway. In part, the goal of this study is to evaluate the efficacy and safety of selected compounds with dual activities of Wnt inhibition and AMPK activation against CRC.

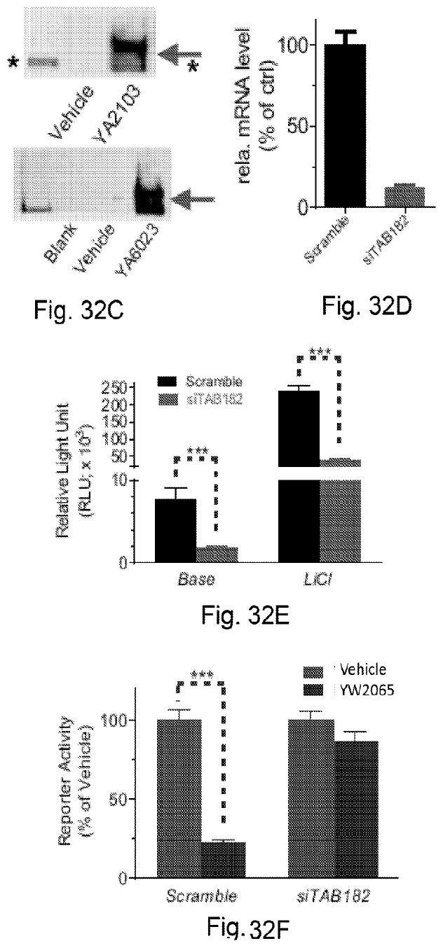

Effects of selected compounds on CRC cell growth and proliferation. YA6060 is further tested in CRC cell models. To represent the heterogeneity of oncogenic Wnt signaling in human CRC, six widely studied CRC cell lines with different genetic status of CTNNB1 (encoding β-catenin) and APC genes are chosen (FIG. 33). Previous Axin stabilizers (TNKS inhibitors) have been reported to suppress several APC-mutated CRC cell lines including SW480 and SW620, while several other APC-mutated lines including HT29 are resistant. The sensitivity to AXIN stabilization has been shown to be dependent on APC mutation form in CRC cells. The CRC cells harboring a constitutively active mutation in β-catenin, such as HCT116 and SW48, or having no Wnt/β-catenin signal-related mutations, such as RKO, are resistant to Axin-mediated Wnt inhibition. Besides the six CRC cell lines, wild-type, full-length APC are introduced to restore normal Wnt signaling in SW480 cells (SW480APC) which are used as an additional control paired with SW480 cells. The cells are treated by vehicle or YA6060, with or without Axin knockdown as another layer of control (by len-shAxin;

FIG. 23C).

The assays for outcome measurement have been established (FIG. 22A-FIG. 22E and FIG. 23A-FIG. 23B). Briefly, i) Wnt signaling inhibition is determined by the reporter assay, immunoblotting and RT-PCR; ii) Cell viability by CCK-8 kit (Sigma); iii) AMPK activation by immunoblotting; iv) Cell proliferation by colony formation assay; and v) Cell cycle and apoptosis by flow cytometry. Because of the dual activities of Wnt inhibition and AMPK activation by the active compounds, it is expected that these compounds will inhibit CRC cell proliferation and growth, as indicated by an increased cytotoxicity, reduced cell colony formation, and enhanced apoptosis with each active compound relative to inactive ones and vehicle. Shown in FIG. 23C, the comparison between len-shAxin and control treatment is expected to show the specificity of active compounds on Axin stabilization. SW480 and SW620 are expected to be more sensitive to active compounds as compared to HT29, HCT116, SW48, RKO, and SW480APC cells.

Effects of YW2065 analogs on CRC initiation and growth in vivo. The upregulation of Wnt signaling pathway is the primary transforming event in CRC. Thus, Axin stabilization provides a sound chemo-preventive strategy for patients with high risk of colon neoplasia. The chemo-preventative potential of YA6060 is tested. Apc^{min/+} mice are widely used model of gastrointestinal (GI) neoplasia, due to an upregulated Wnt signaling by truncated Apc proteins. Of note, Pyr and Axin stabilizers have been reported to inhibit intestinal polyp formation in Apc^{min/+} mice. As outlined in FIG. 33, 4 groups (n=15/group) of 6-week-old Apc^{min/+} (2 groups) and wild-type (2 groups) C57BL/6 male mice (Jackson Labs) are treated by compound or vehicle via oral gavage every 48 h for 10 weeks as described previously.

After 11 weeks, mice are euthanized and tumor numbers and volumes are determined by investigators blinded to treatment. Cell proliferation is determined by immunohistochemistry (IHC) with anti-Ki67 antibody; cell apoptosis in tumor tissues by IHC for cleaved caspase-3; Axin, and β-catenin, p-AMPK/AMPK, and p-ACC/ACC by immunoblotting and/or IHC; and the expression of Wnt target genes by RT-PCR. Blood biochemistry is analyzed to determine liver and kidney functions. General histology of major tissues is examined to evaluate toxic effects. The primary statistical endpoints are tumor numbers and volumes. Data is analyzed by ANOVA followed by a post-hoc test (see Statistical section). Compared to vehicle treatment, an active compound is expected to significantly attenuate both adenoma number and volume in Apc^{min/+} mice without notable toxicity and that cell proliferation will be reduced in adenoma, whereas apoptosis will be increased. Moreover, it is expected that the active compounds stabilize Axin protein and inhibit Wnt signaling as measured by protein and gene expression changes in the pathway in adenoma tissues.

Effects of YA6060 on CRC invasion and metastasis in vivo. The above Apc^{min/+} mouse model has limitations such as the location of polyp formation, which is limited in the small intestine, and no progression from these adenomas to metastasis. In this example, YA6060 is tested to see if the compound attenuates colon cancer progression, specifically tumor growth, invasion, and metastasis in the established murine model of CRC invasion and metastasis (FIG. 25A-FIG. 25D). To facilitate longitudinal follow-up of CRC progression in real time by BLI (FIG. 25C), stable luciferase-expressing HT-29-luc-D6 cells are used (Perkin Elmer, Inc.). Orthotopic cecal implantation of HT-29-luc-D6 tumor fragments has been shown to robustly produce metastatic lesions in liver (81%), lung (90%) and mesenteric lymph nodes (95%). HT-29-luc-D6 xenografts are first generated in nude mice. At 3-4 weeks, tumors are resected and equal amounts of tumor fragments (˜30 mg) are implanted onto the cecal serosal surfaces of nude mice as described (FIG. 25A-FIG. 25D). One week later, the mice who received tumor implantation undergo estimation of primary tumor size by BLI and those with a similar size are randomly assigned and treated daily by i.p. injection of an YA6060 (n=15) or vehicle (n=15) for 6-8 weeks (FIG. 33). The mice received sham surgery are treated as controls.

Local tumor growth and distant metastasis (i.e., liver, lungs and lymph nodes) are measured quantitatively each week in real-time by BLI (FIG. 25C). In instances where the metastases in the liver are poorly identified by BLI in late weeks due to the strong signal from proximity of the primary tumor, their measuring will rely on later post-mortem examination. After 6-8 weeks, all mice are euthanized and the cecum, liver and lungs excised and weighed. Cecal tumor volume, the number and size of distant metastases re measured by investigators blinded to treatment. Portions of each tumor are stored in liquid nitrogen for molecular experiments and fixed in formalin for H&E staining and IHC. Other analyses are conducted as described above for Apc^{min/+} mice. The primary statistical endpoint is cecal tumor volume, with the number and size of distant metastases as the secondary main endpoints. It is expected that new Axin stabilizers will significantly slow, if not abolish, tumor progression and dissemination in the advanced CRC model.

It is expected that YA6060 shows specific efficacy against CRC cells, demonstrating its efficacy in suppression of intestinal carcinogenesis, and for the first time, a small molecule that can be an effective therapy for advanced CRC. CRC are highly heterogeneous in genetics. In a non-limiting example, additional CRC cell lines are chosen to explore a broad clinical implication for compounds. In a non-limiting example, compounds of the disclosure are used together with current CRC therapeutics to achieve synergistic effects. Although YW2065 killed CRC cells at an IC₅₀ comparable to those of first-line CRC chemotherapy (5-FU and oxaliplatin), the in vivo anti-cancer efficacy in mice was modest (FIG. 24A-24D). In some embodiments, the combinatory effects of the compounds of the disclosure are examined in combination with current anti-CRC agents. In a non-limiting example, the somatic mutation in the Apc gene effects physiology in Apc^{min/+} mice. For example, metabolic disorders have been recently reported in this strain. If Apc^{min/+} mice are more susceptible to adverse effects from our compounds, azoxymethane (AOM)-treated mice can be used as an alternative. In some embodiments, for the “surgical cecal-transplantation” model, HT-29 cells are injected directly into the cecal wall, although cecal wall injection generates more colon-draining lymphatic lesions and slightly fewer hepatic metastases. In a non-limiting example, although HT-29 cells were sensitive to YA6060 treatment (FIG. 22E), other cell lines can be used for the advanced CRC model. In a non-limiting example, a syngeneic model is used (FIG. 25C-FIG. 25D), although murine MC38 cells are less clinically relevant. Other CRC animal models include genetically engineered mouse models (GEMMs), albeit most do not develop distant metastases. In a non-limiting example, the distant metastases are the secondary endpoints, and specialized metastatic models could be studied in the future.

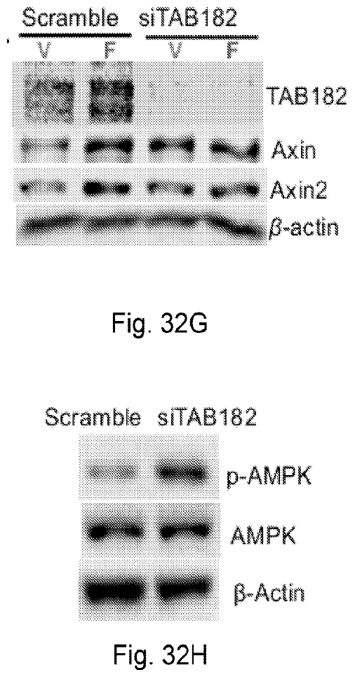

Investigating the mechanism of action of new compound. The goal of this aim is to gain molecular insights into the pharmacological action of compounds of the disclosure with dual activities of Wnt inhibition and AMPK activation. It is hypothesized that Wnt inhibition and AMPK activation via Axin stabilization is the effective mechanism for YA6060 to treat CRC (FIG. 34). While testing this hypothesis, the protein targets for the compounds are confirmed

Role of Wnt inhibition and AMPK activation in conferring the effects of YA6060 on CRC cell proliferation and growth. It is determined if Axin stabilizers depend on Wnt inhibition, AMPK activation, or both to inhibit CRC cell proliferation and growth. The Wnt signaling activity is determined by reporter assay and by analyzing the effector protein and target gene expression with immunoblotting and RT-PCR. The protein levels of p-AMPK, total AMPK, the downstream p-ACC, total ACC are determined by immunoblotting. Wnt activity is modulated via Axin knockdown with len-shAxin (FIG. 23C), lentivirus encoding the constitutively active β-catenin mutant S33Y (FIG. 23C), len-CA-β-cat from Sigma, and disruption of β-catenin/LEF/TCF complexes with iCRT3 (Calbiochem®). The adenovirus encoding a dominant-negative mutant AMPKα1 (ad-DNα1) and the inhibitor Compound C are used to suppress AMPK activity. The role of AMPK in metformin action has been reported. Metformin is of a great interest in the prevention and treatment of cancers including CRC. Here metformin and additional AMPK activators are used as positive controls for AMPK activation and their effects on CRC cell proliferation and growth will be determined. To further validate the role of AMPK, the downstream p53 and mTOR pathways are also examined. Apoptosis-related proteins, including phosphorylated p53, p21^{WAF1}, Bax, Bcl-2, pro-caspase 3, and active caspase 3 re tested using immunoblotting; the mTOR pathway activity and related autophagy by measuring levels of phosphop-mTOR, phosphop-70S6K, cyclin D, and conversion of LC3-I to LC3-II. Antibodies to proteins are commercially available (Cell Signaling; EMD Millipore; Epitomics). It is expected that the effects by YA6060 on cell viability, proliferation, and apoptosis will be reduced, if not fully abolished, in Axin-null/knockdown, len-CA-β-cat, ad-DNα1 and/or Compound C-treated cells compared to control cells, but increased in iCRT3 and/or metformin-treated cells. Consistent changes in Wnt target gene expression and AMPK downstream (p53 and mTOR) pathways are expected. The relative contribution from Wnt inhibition and AMPK activation to the efficacies by YA6060 is determined. The anti-CRC efficacies by YA6060 is expected to be superior to sole Wnt inhibitor iCRT3 and sole AMPK activators (metformin).

Mechanism by which YA6060 activates AMPK in CRC cells—Axin stabilization-mediated Wnt inhibition is well known. Three hypotheses are tested to address the mechanism underlying Axin stabilization-mediated AMPK activation by YA6060 (FIG. 34). (1) It is hypothesized that YA6060 inhibits mitochondrial respiration, leading to a falling energy status which activates AMPK. (2) It is hypothesized that these YA6060 enhances the formation of Axin-AMPK-LKB1 complex that is crucial to AMPK activation. (3) It is hypothesized that Axin stabilization by YA6060 may result from its indirect TNKS inhibition which causes less LKB1 degradation and subsequently increased AMPK activation. SW480 cells are mainly used. The cells are treated by vehicle control or YA6060, with and without a condition of enhanced or blocked nuclear β-catenin signaling, Axin knockdown as described above, or TNKS knockdown (by len-shTNKS1/2 shRNA). The outcome assays include: i) Mitochondrial function and morphology are analyzed as described herein (FIG. 29A-FIG. 29D); ii) The formation of Axin-AMPK-LKB1 complex by co-immunoprecipitation (co-IP) and microscopic co-localization; iii) Total AMPK and p-AMPK by immunoblotting; iv) Total LKB1 and p-LKB1 by immunoblotting. It is anticipated that YA6060 will inhibit mitochondrial function, leading to a reduced ATP production. len-shAxin, len-shTNKS1/2, and len-CA-β-cat are expected to alleviate, while iCRT3 to aggravate, the mitochondrial inhibition by the active compounds, confirming a mechanism via canonical β-catenin signaling. Meanwhile, it is anticipated that YA6060 will increase the formation of Axin-AMPK-LKB1 complex because of increased Axin expression, increased LKB1 expression, and AMPK redistribution. The complex formation is expected to be diminished by lenti-shAxin and len-shTNKS1/2. Because AMP promotes interaction between Axin and AMPK, it is hypothesized that lenti-CA-β-cat and iCRT3 will have an impact on the complex formation by affecting mitochondrial energy metabolism. A less pronounced alleviation by len-CA-β-cat than those by len-shAxin and len-shTNKS1/2 in YA6060-induced AMPK activation is expected, consistent with major effects from Axin and its upstream TNKS on the complex formation. Overall, the data supports the hypothesis that the Axin stabilizers of the disclosure activate AMPK synergistically via mitochondrial inhibition and enhanced interaction between Axin, AMPK and LKB1.

Confirmation of protein targets for YA6060-TAB182 and other positive hits that were identified in the preliminary target search are further examined (FIG. 32B). Studies will begin with TAB182 because this protein binds to TNKS which stabilizes Axin and have served as a target for Wnt inhibition. Experiments are proposed to determine: i) the binding affinity of YA6060 to TAB182 protein and its mutants by using SPR; ii) the effects of TAB182 silencing, overexpression, and mutation on the anti-CRC cell growth and proliferation by YA6060 as described above; iii) the effects of the same TAB182 manipulation on YA6060-induced AMPK activation, cellular responses and molecular events, as described above iv) the potential interaction between TAB182, TNKS, and Axin, and the impact by YA6060. Expressing plasmids for TAB182 and its mutants are used, and the protein expression, purification, and subsequent SPR analysis are examined. TAB182 silencing, overexpression, and mutants are established (FIG. 32A-FIG. 32H). It is expected that TAB182 is identified as an important direct target for Axin stabilizers. In a non-limiting example, overall similar approaches to examine other potential targets can also be used (FIG. 32B).

It is expected that this example demonstrates the efficacy of YA6060 against CRC is, at least partially, via AMPK activation that is due to mitochondrial inhibition involved with Wnt signaling and enhanced formation of Axin-AMPK-LKB1 complex. The identified direct target of YA6060 advances the understanding of signal transduction via Axin, providing novel strategy and targets to develop CRC therapy. In a non-limiting example, if the efficacy of YA6060 was independent of AMPK activation, the preliminary data and the direct target identified through these studies can be used to formulate a new and testable hypothesis, such as mitochondrial dysfunction-induced apoptosis, or TAB182-mediated cancer development and drug sensitivity. In a non-limiting example, a traditional label-based method is used instead of SPR, which widely used in characterizing interaction of small molecules with their protein targets. In a non-limiting example, additional conditions for biotinylated chemical affinity chromatography can be explored, followed by MS protein identification. In a non-limiting example, thermal proteome profiling (TPP) is used, which combines the measurement of protein thermal stability with quantitative proteomics and has recently been used to identify drug targets.

For animal studies, the sample size is expected to provide sufficient power for statistical analyses (at the P<0.05 level; α=0.05, β=0.1), which is estimated by Russ Lenth's Power and Sample Size Calculation, with the data of tumor number, growth and size measurement from previous publications. Statistical analyses are conducted with GraphPad software. T tests and analysis of variance (ANOVA) followed by a post-hoc test will be employed in this application. A P value less than 0.05 is considered as statistically different. All chromosomal or plasmid borne assays are checked by PCR following the assay. RT-PCR is performed in triplicate on a minimum of 3 biological replicates. Purified proteins are analyzed by SDS-PAGE and checked by MALDI-TOF mass spectrometry to ensure homogeneity or removal of affinity tags. All Western blots are run on 3 separate experiments with purified protein as a marker and normalized to total protein or to RNA polymerase alpha subunit as a loading control. For proteomic studies all experiments are performed on a minimum of 5 biological replicates and 3 technical replicates per sample. Verification of instrumentation sensitivity is determined by running internal standards as a control to assess day to day variation of instrumentation. Peptide assignments are set at 1% false detection rate (FDR) and relative abundance will be considered significant >1.5 fold and p<0.05. All SMPs and inhibitors are verified by NMR and high accuracy mass spectrometry prior to use.

### Example 5: Synthesizing and Evaluating New Compounds that Inhibit Wnt Signaling and Activate AMPK

The limitation of compound YA6060 includes its high hygroscopicity and potential metabolic instability. It is hypothesized that simultaneous inhibition of Wnt signaling by targeting the tankyrase-binding protein TAB182 and activation of the AMPK pathway, is a novel therapeutic strategy for the treatment of CRC. This Example describes the synthesis of non-hygroscopic and metabolically stable YA6060 analogs or CRC-targeting YA6060 hybrids and testing them using established assays, to validate and characterize potent compound with dual activity of Wnt inhibition and AMPK activation. In a non-limiting example, the compounds are Wnt inhibitors with significantly enhanced aqueous solubility (>10,000 fold) and bioavailability (>3 fold) while maintaining the inhibitory potency against the Wnt signaling pathway via Axin stabilization. First, new compounds that inhibit Wnt signaling and activate AMPK are synthesized and evaluated: two classes of compounds based on YA6060 are synthesized including i) acceptably non-hygroscopic and metabolically stable YA6060 analogs and ii) folate receptor (FR)-targeted folic acid (FA)-YA6060 hybrids. Wnt inhibition of the new compounds is assayed by the TOPflash reporter assays, confirming the mechanism of action by immunoblots and RT-PCR, and FR-mediated uptake is assessed using FR-positive cells. Solubility, cytotoxicity, permeability and metabolic stability of selected compounds is determined in vitro by testing the pharmacokinetic (PK) properties and in vivo toxicity (general tolerance and organ-specific toxicity) of selected compounds using C57/BL6 mice.

The anti-CRC efficacy of selected compounds is assessed by assessing the activity of selected compounds in killing CRC cells with different genetic mutations of Wnt signaling effectors, and their effects on colony formation, cell cycle, and apoptosis of CRC cells are examined. The inhibitory activity of selected compounds for CRC initiation/growth using Apc^{min/+} mice is studied. The effects of selected compounds in blocking CRC invasion and metastasis are evaluated using an advanced HT-29-luc-D6 tumor Cecal implantation mouse model for CRC invasion and metastasis.

The mechanism of action of new leads is investigated by examining the role of Wnt signaling and AMPK pathways in conferring the effects of selected compounds on CRC cell proliferation and growth using immunoblotting, immunohistochemistry (IHC) and RT-PCR. The mechanism by which selected compounds activate the AMPK pathway in CRC cells is studied, and the direct protein targets of the optimal compounds are identified and confirmed using a combination of pull-down experiments, genetic manipulation, and thermo protein profiling (TPP) strategies.

This Example provides evidence to access the therapeutic potential of simultaneously targeting Wnt signaling and AMPK pathways as novel anti-CRC therapeutic strategy. Although the YA6060 indicated an excellent activity and safety profile, it is highly hygroscopic and readily absorbs moisture from the environment, which complicated its handling process and shortened the compound's shelf life. In addition, the chemical structure of YA6060 contains multiple C—H bonds that are liable to P450-mediated oxidations in the liver. Therefore, novel YA6060 analogs with acceptable non-hygroscopicity along with excellent metabolic stability are highly desirable for its further development.

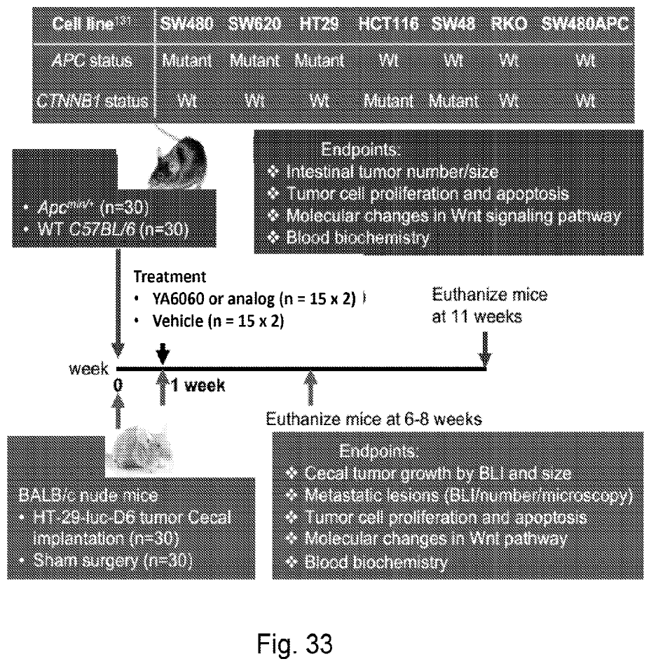

Using YA6060 as a template, new compounds are synthesized that can simultaneously inhibit Wnt signaling pathway and activate AMPK pathway, with CRC selectivity, desirable PK/Tox properties and physiochemical profiles (FIG. 35). 10 new YA6060 analogs are synthesized to address its limitation including potential metabolic instability and high hygroscopicity. Five CRC-targeting FA-YA6060 hybrids that can selectively target FRα/β overexpressed on CRC cells are synthesized. The inhibitory potency of the synthesized compounds is determined using TOPflash luciferase reporter assay, and confirmed by Western blots and RT-PCR experiments. The uptake of FA-YA6060 hybrids via the FRs is determined. For the six selected compounds, studies to access their solubility, ADME and bioavailability are performed. In addition, PK and toxicity tests of the compounds are performed to identify three candidates that are further evaluated. Differential killing activity of compounds for Wnt-dependent over Wnt-independent CRC cells is determined. The in vivo efficacy of the compounds in CRC initiation and growth, as well as invasion and metastasis, is investigated. The mechanism of action of top candidates towards Wnt inhibition and AMPK activation is investigated. The direct protein target of selected compounds are confirmed using pull-down experiments by biotinylated ligands, and proteomic thermo protein profiling (TPP) approaches.

New compounds that inhibit Wnt signaling and activate AMPK are synthesized and evaluated. In order to discover the metabolically stable, acceptably non-hygroscopic, and CRC-selective compounds with dual activity of Wnt signaling inhibition and AMPK activation, medicinal chemistry efforts are devoted to synthesizing new compounds of three classes: fluoro-containing P450-inert YA6060 analogs, non-hygroscopic salts of YA6060, and FR-targeting FA-YA6060 hybrids. The dual activity of synthesized analogs/hybrids for Wnt signaling and AMPK pathways is confirmed. The ADME, bioavailability, Tox and solubility of selected compounds are determined. These efforts yield three selected compounds for further evaluation.

New compound design and synthesis—Based on preliminary data of YA6060, five fluorine-containing YA6060 analogs are synthesized to block the potential P450-based oxidative metabolism. Different salts of the compound employing five different counter ions are generated. Five FA-YA6060 hybrids to target the FR-overexpressing CRC cells are synthesized.

Non-hygroscopic salts of YA6060. YA6060 is an HCl salt with high hygroscopicity. YA6060 can readily absorb moisture from the environment that limits the stability and the further therapeutic application of the compound. Non-hygroscopic salts of YA6060 are generated by screening the anionic of the neutral amino parent YA6060 with a panel of acids that has been reported to form acceptably non-hygroscopic salts. Specific acids include valproic (1), maleic (2), tartaric (3), oxalic (4) and pamoic (5) acids, to get the corresponding salts.

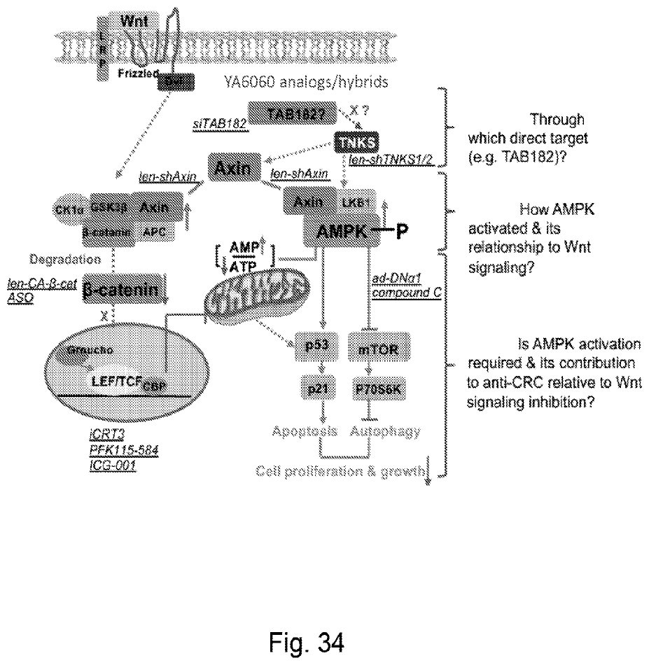

Fluorine-containing YA6060 analogs. Five YA6060 analogs (6-10) are synthesized by substituting the P450-labile hydrogen atoms with metabolically inert F-atoms (FIG. 36). The synthesis of proposed triazole compounds starts with aniline derivatives using previously reported methods. Diazotization of the amino group of substituted anilines using NaNO₂ and aqueous HCl, followed by the treatment of the intermediate with NaN₃ gave azides, which underwent cyclization with β-ketoester in EtONa/EtOH yielded trizaole-3-carboxylic acids. Next, PyCIU-mediated coupling of the resulting acids with 6-bromoquinolin-2-amine in dichloroethane provided target triazole amides. Next, Pd-catalyzed cross-coupling of the bromo-arene with potassium (4-Boc-piperazin-1-yl)methyltrifluoroborate yields Boc-protected products. Finally, the Boc-protecting group is removed using TFA to give the final compounds.

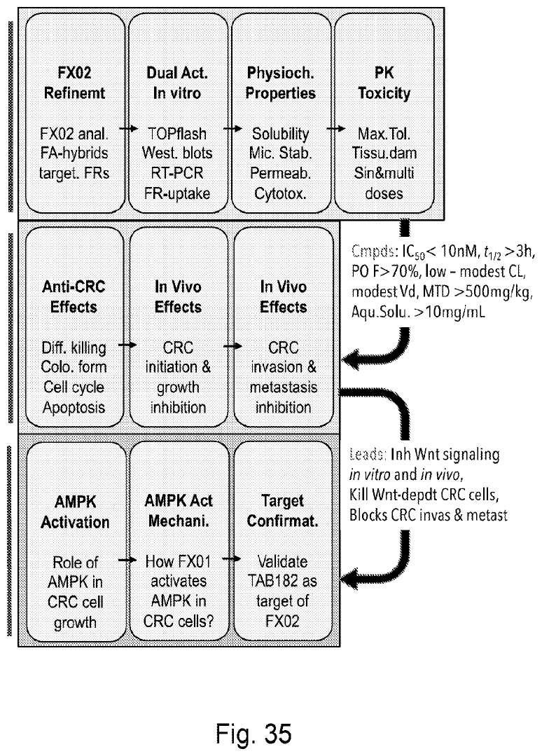

CRC cell-targeting FA-YA6060 hybrids. Preliminary SAR results in the have shown that extension of the piperazine group of YA6060 is well-tolerated in maintaining the dual activity of compounds (FIG. 32A). Thus, four FA-YA6060 hybrids are synthesized that employ various PEG-spacers (n=0, 1, 2, 3 and 4; FIG. 37). The synthesis of hybrids 11-15 begin with YA6060. Alkylation of YA6060 with PET-bromide 16 provides compound 18. Removal of the Boc-protecting group of compound 18 using TFA yields the primary amine 19, which then reacts with NHS-folate 20 to provide the hybrids 11-15.

In vitro evaluation of drug properties for new compounds. A panel of assays to assess YA6060 analogs/hybrids was established (FIG. 35). The new compounds (HPLC purity, >95%) are first subject to TOPflash reporter assay to screen their inhibitory potency against the Wnt signaling in HEK293 cells. The analogs with an IC₅₀<100 nM are then validated by immunoblotting and RT-PCR to determine their capability of Axin stabilization and AMPK activation. The FR-mediated uptake of synthesized hybrids is assessed by an LC-MS-based method employing the FR-positive M109 cells.

Determination of physiochemical properties. The profiles of six selected analogs is estimated in silico, and selected Axin stabilizers are evaluated in in vitro assays: the aqueous solubility is determined using a multiscreen solubility filter plate (Millipore, Billerica, MA) coupled with LC-MS/MS, as performed for the already synthesized YA6060 analogs; the cell viability assays on selected analogs is measured using HEK293 cells; the intestinal permeability by determining the transport from the apical to the basolateral (A to B) in Caco-2 monolayers; the metabolic stability by incubating the compounds with mouse microsomes and liver tissue S9 fraction and analyzing by LC-MS/MS. Compounds with acceptable values according to drug property criteria (FIG. 35) are expected to be generated.

PK/Tox. The four selected compounds from in vitro evaluation above are subjected to PK and toxicity assessment in C57/BL6 mice. As for lead YA6060 PK (FIG. 6A-FIG. 6D), a single dose of new compounds is administered by oral gavage or tail vein injection; blood samples are collected over 24 h. Compound concentrations is determined by LC/MS/MS. PK analysis is carried out on Phoenix Winnolin using non-compartmental analysis and compartment models. Compound accumulation in major tissues, including liver, kidney, heart, brain and intestine, are quantified. Toxicity studies are performed: i) mice are exposed by daily i.v. with increasing doses (range to be selected later based on PK results) or vehicle over 2 weeks; ii) mice are exposed to a uniform daily i.v. of the compound or vehicle over the course of 3 weeks to determine cumulative effects. For study ii, a half number of mice per group are sacrificed immediately after the 3-week course and the remaining after a 2-week washout period to assess delayed toxicity. In toxicity studies, blood is collected upon euthanasia for liver and renal chemistries and complete blood counts, and major organs harvested and examined macro- and microscopically by veterinarian pathologists. It is expected that an understanding of PK properties, such as bioavailability, AUC, clearance, and toxic effects, such as general tolerance and specific toxicity in the liver, kidney, other major organs, and particularly Wnt-dependent tissues, such as intestine, stomach, and skin is established for these selected compounds.

The characterization of YA6060 analogs/hybrids with dual mechanism of Wnt inhibition and AMPK activation provide a platform for the development of more druglike and CRC-selective compounds. The robust suite of tests including the Western blots, RT-PCR, and FR uptake assays will further validate compounds with IC₅₀ values (<10 nM) (FIG. 15) is expected to address any issues regarding false positives. In a non-limiting example, chemistry efforts provide several compounds (e.g. six compounds) with varying degrees of potency, and candidates are selected (e.g. three candidates) based on those that further demonstrate favorable, aqueous solubility, oral bioavailability and ADMET profiles. Additional YA6060 salts with acceptable hygroscopicity that are investigated may also include phosphonic, benzoic, citric, salicylic, succinic methanesulfonic, malic, maleic, and p-toluenesulfonic acids. A desirable therapeutic window, particularly with an acceptable toxicity in those Wnt/β-catenin-dependent normal tissues, for the selected compounds is anticipated. It has been demonstrated that YA6060 analogs do not cause any clinical toxicity in mice (FIG. 8). It is also expected that the FA-hybrids demonstrate good CRC-specificity. Biguanide-YA6060 hybrids are expected to show a desirable safety profile in healthy mice and CRC models. Biguanide-containing compounds are excellent substrates for organic cation transports (OCTs) that have been shown to express in CRC cells and tissues. Moreover, biguanide drug metformin is the most well-known activator of AMPK, which can reinforce the mechanism of compounds of the disclosure in activating AMPK. 15 new YA6060 analogs/hybrids are expected to be prepared for analysis, and the top 20% of compounds are subjected to mechanistic studies and in vivo efficacy in mice CRC models as described above.

Assessing the anti-CRC efficacy of selected compounds. CRC is of a major interest for targeting Wnt pathway. In part, the goal of this study is to evaluate the efficacy and safety of selected compounds with dual activities of Wnt inhibition and AMPK activation against CRC.

Effects of selected compounds on CRC cell growth and proliferation. YA6060 and selected analogues are further tested in CRC cell models. To represent the heterogeneity of oncogenic Wnt signaling in human CRC, six widely studied CRC cell lines with different genetic status of CTNNB1 (encoding β-catenin) and APC genes are chosen (FIG. 33). Previous Axin stabilizers (TNKS inhibitors) have been reported to suppress several APC-mutated CRC cell lines including SW480 and SW620, while several other APC-mutated lines including HT29 are resistant. The sensitivity to AXIN stabilization has been shown to be dependent on APC mutation form in CRC cells. The CRC cells harboring a constitutively active mutation in β-catenin, such as HCT116 and SW48, or having no Wnt/β-catenin signal-related mutations, such as RKO, are resistant to Axin-mediated Wnt inhibition. Besides the six CRC cell lines, wild-type, full-length APC are introduced to restore normal Wnt signaling in SW480 cells (SW480APC) which are used as an additional control paired with SW480 cells. The cells are treated by vehicle or YA6060, with or without Axin knockdown as another layer of control (by len-shAxin; FIG. 23C).

The assays for outcome measurement have been established (FIG. 22A-FIG. 22E and FIG. 23A-FIG. 23B). Briefly, i) Wnt signaling inhibition is determined by the reporter assay, immunoblotting and RT-PCR; ii) Cell viability by CCK-8 kit (Sigma); iii) AMPK activation by immunoblotting; iv) Cell proliferation by colony formation assay; and v) Cell cycle and apoptosis by flow cytometry. Because of the dual activities of Wnt inhibition and AMPK activation by the active compounds, it is expected that these compounds will inhibit CRC cell proliferation and growth, as indicated by an increased cytotoxicity, reduced cell colony formation, and enhanced apoptosis with each active compound relative to inactive ones and vehicle. Shown in FIG. 23C, the comparison between len-shAxin and control treatment is expected to show the specificity of active compounds on Axin stabilization. SW480 and SW620 are expected to be more sensitive to active compounds as compared to HT29, HCT116, SW48, RKO, and SW480APC cells.

Effects of FX01 analogs on CRC initiation and growth in vivo. The upregulation of Wnt signaling pathway is the primary transforming event in CRC. Thus, Axin stabilization provides a sound chemo-preventive strategy for patients with high risk of colon neoplasia. The chemo-preventative potential of YA6060 and selected analogues is tested. Apc^{min/+} mice are widely used model of gastrointestinal (GI) neoplasia, due to an upregulated Wnt signaling by truncated Apc proteins. Of note, Pyr and Axin stabilizers have been reported to inhibit intestinal polyp formation in Apc^{min/+} mice. As outlined in FIG. 33, 4 groups (n=15/group) of 6-week-old Apc^{min/+} (2 groups) and wild-type (2 groups) C57BL/6 male mice (Jackson Labs) are treated by compound or vehicle via oral gavage every 48 h for 10 weeks as described previously.

After 11 weeks, mice are euthanized and tumor numbers and volumes are determined by investigators blinded to treatment. Cell proliferation is determined by immunohistochemistry (IHC) with anti-Ki67 antibody; cell apoptosis in tumor tissues by IHC for cleaved caspase-3; Axin, and β-catenin, p-AMPK/AMPK, and p-ACC/ACC by immunoblotting and/or IHC; and the expression of Wnt target genes by RT-PCR. Blood biochemistry is analyzed to determine liver and kidney functions. General histology of major tissues is examined to evaluate toxic effects. The primary statistical endpoints are tumor numbers and volumes. Data is analyzed by ANOVA followed by a post-hoc test (see Statistical section). Compared to vehicle treatment, an active compound is expected to significantly attenuate both adenoma number and volume in Apc^{min/+} mice without notable toxicity and that cell proliferation will be reduced in adenoma, whereas apoptosis will be increased. Moreover, it is expected that the active compounds stabilize Axin protein and inhibit Wnt signaling as measured by protein and gene expression changes in the pathway in adenoma tissues.

Effects of YA6060 analogues on CRC invasion and metastasis in vivo. The above Apc^{min/+} mouse model has limitations such as the location of polyp formation, which is limited in the small intestine, and no progression from these adenomas to metastasis. In this example, YA6060 and selected analogues are tested to see if the compound attenuates colon cancer progression, specifically tumor growth, invasion, and metastasis in the established murine model of CRC invasion and metastasis (FIG. 25A-FIG. 25D). To facilitate longitudinal follow-up of CRC progression in real time by BLI (FIG. 25C), stable luciferase-expressing HT-29-luc-D6 cells are used (Perkin Elmer, Inc.). Orthotopic cecal implantation of HT-29-luc-D6 tumor fragments has been shown to robustly produce metastatic lesions in liver (81%), lung (90%) and mesenteric lymph nodes (95%). HT-29-luc-D6 xenografts are first generated in nude mice. At 3-4 weeks, tumors are resected and equal amounts of tumor fragments (˜30 mg) are implanted onto the cecal serosal surfaces of nude mice as described (FIG. 25A-FIG. 25D). One week later, the mice who received tumor implantation undergo estimation of primary tumor size by BLI and those with a similar size are randomly assigned and treated daily by i.p. injection of an YA6060 (n=15) or vehicle (n=15) for 6-8 weeks (FIG. 33). The mice received sham surgery are treated as controls.

Local tumor growth and distant metastasis (i.e., liver, lungs and lymph nodes) are measured quantitatively each week in real-time by BLI (FIG. 25C). In instances where the metastases in the liver are poorly identified by BLI in late weeks due to the strong signal from proximity of the primary tumor, their measuring will rely on later post-mortem examination. After 6-8 weeks, all mice are euthanized and the cecum, liver and lungs excised and weighed. Cecal tumor volume, the number and size of distant metastases re measured by investigators blinded to treatment. Portions of each tumor are stored in liquid nitrogen for molecular experiments and fixed in formalin for H&E staining and IHC. Other analyses are conducted as described above for Apc^{min/+} mice. The primary statistical endpoint is cecal tumor volume, with the number and size of distant metastases as the secondary main endpoints. It is expected that new Axin stabilizers will significantly slow, if not abolish, tumor progression and dissemination in the advanced CRC model.

It is expected that YA6060 analogues/hybrids show specific efficacy against CRC cells, demonstrating its efficacy in suppression of intestinal carcinogenesis, and for the first time, a small molecule that can be an effective therapy for advanced CRC. CRC are highly heterogeneous in genetics. In a non-limiting example, additional CRC cell lines are chosen to explore a broad clinical implication for compounds. In a non-limiting example, compounds of the disclosure are used together with current CRC therapeutics to achieve synergistic effects. Although FX01 killed CRC cells at an IC₅₀ comparable to those of first-line CRC chemotherapy (5-FU and oxaliplatin), the in vivo anti-cancer efficacy in mice was modest (FIG. 24A-24D). In some embodiments, the combinatory effects of the compounds of the disclosure are examined in combination with current anti-CRC agents. In a non-limiting example, the somatic mutation in the Apc gene effects physiology in Apc^{min/+} mice. For example, metabolic disorders have been recently reported in this strain. If Apc^{min/+} mice are more susceptible to adverse effects from our compounds, azoxymethane (AOM)-treated mice can be used as an alternative. In some embodiments, for the “surgical cecal-transplantation” model, HT-29 cells are injected directly into the cecal wall, although cecal wall injection generates more colon-draining lymphatic lesions and slightly fewer hepatic metastases. In a non-limiting example, although HT-29 cells were sensitive to YA6060 treatment (FIG. 22E), other cell lines can be used for the advanced CRC model. In a non-limiting example, a syngeneic model is used (FIG. 25C-FIG. 25D), although murine MC38 cells are less clinically relevant. Other CRC animal models include genetically engineered mouse models (GEMMs), albeit most do not develop distant metastases. In a non-limiting example, the distant metastases are the secondary endpoints, and specialized metastatic models could be studied in the future.

Investigating the mechanism of action of new compound. The goal of this aim is to gain molecular insights into the pharmacological action of compounds of the disclosure with dual activities of Wnt inhibition and AMPK activation. It is hypothesized that Wnt inhibition and AMPK activation via Axin stabilization is the effective mechanism for YA6060 analogues to treat CRC (FIG. 34). While testing this hypothesis, the protein targets for the compounds are confirmed

Role of Wnt inhibition and AMPK activation in conferring the effects of YA6060 analogues/hybrids on CRC cell proliferation and growth. It is determined if Axin stabilizers depend on Wnt inhibition, AMPK activation, or both to inhibit CRC cell proliferation and growth. The Wnt signaling activity is determined by reporter assay and by analyzing the effector protein and target gene expression with immunoblotting and RT-PCR. The protein levels of p-AMPK, total AMPK, the downstream p-ACC, total ACC are determined by immunoblotting. Wnt activity is modulated via Axin knockdown with len-shAxin (FIG. 23C), lentivirus encoding the constitutively active β-catenin mutant S33Y (FIG. 23C), len-CA-β-cat from Sigma, and disruption of β-catenin/LEF/TCF complexes with iCRT3 (Calbiochem®). The adenovirus encoding a dominant-negative mutant AMPKα1 (ad-DNα1) and the inhibitor Compound C are used to suppress AMPK activity. The role of AMPK in metformin action has been reported. Metformin is of a great interest in the prevention and treatment of cancers including CRC. Here metformin and additional AMPK activators are used as positive controls for AMPK activation and their effects on CRC cell proliferation and growth will be determined. To further validate the role of AMPK, the downstream p53 and mTOR pathways are also examined. Apoptosis-related proteins, including phosphorylated p53, p21^{WAF1}, Bax, Bcl-2, pro-caspase 3, and active caspase 3 re tested using immunoblotting; the mTOR pathway activity and related autophagy by measuring levels of phosphop-mTOR, phosphop-70S6K, cyclin D, and conversion of LC3-I to LC3-II. Antibodies to proteins are commercially available (Cell Signaling; EMD Millipore; Epitomics). It is expected that the effects by YA6060 on cell viability, proliferation, and apoptosis will be reduced, if not fully abolished, in Axin-null/knockdown, len-CA-β-cat, ad-DNα1 and/or Compound C-treated cells compared to control cells, but increased in iCRT3 and/or metformin-treated cells. Consistent changes in Wnt target gene expression and AMPK downstream (p53 and mTOR) pathways are expected. The relative contribution from Wnt inhibition and AMPK activation to the efficacies by YA6060 is determined. The anti-CRC efficacies by YA6060 analogues is expected to be superior to sole Wnt inhibitor iCRT3 and sole AMPK activators (metformin).

Mechanism by which YA6060 analogues/hybrids activate AMPK in CRC cells—Axin stabilization-mediated Wnt inhibition is well known. Three hypotheses are tested to address the mechanism underlying Axin stabilization-mediated AMPK activation by YA6060 (FIG. 34). (1) It is hypothesized that YA6060 analogues/hybrids inhibit mitochondrial respiration, leading to a falling energy status which activates AMPK. (2) It is hypothesized that these YA6060 analogues/hybrids enhance the formation of Axin-AMPK-LKB1 complex that is crucial to AMPK activation. (3) It is hypothesized that Axin stabilization by YA6060 analogues/hybrids may result from its indirect TNKS inhibition which causes less LKB1 degradation and subsequently increased AMPK activation. SW480 cells are mainly used. The cells are treated by vehicle control or YA6060 analogues/hybrids, with and without a condition of enhanced or blocked nuclear β-catenin signaling, Axin knockdown as described above, or TNKS knockdown (by len-shTNKS1/2 shRNA). The outcome assays include: i) Mitochondrial function and morphology are analyzed as described herein (FIG. 29A-FIG. 29D); ii) The formation of Axin-AMPK-LKB1 complex by co-immunoprecipitation (co-IP) and microscopic co-localization; iii) Total AMPK and p-AMPK by immunoblotting; iv) Total LKB1 and p-LKB1 by immunoblotting. It is anticipated that YA6060 analogues/hybrids will inhibit mitochondrial function, leading to a reduced ATP production. len-shAxin, len-shTNKS1/2, and len-CA-β-cat are expected to alleviate, while iCRT3 to aggravate, the mitochondrial inhibition by the active compounds, confirming a mechanism via canonical β-catenin signaling. Meanwhile, it is anticipated that YA6060 analogues/hybrids will increase the formation of Axin-AMPK-LKB1 complex because of increased Axin expression, increased LKB1 expression, and AMPK redistribution. The complex formation is expected to be diminished by lenti-shAxin and len-shTNKS1/2. Because AMP promotes interaction between Axin and AMPK, it is hypothesized that lenti-CA-β-cat and iCRT3 will have an impact on the complex formation by affecting mitochondrial energy metabolism. A less pronounced alleviation by len-CA-β-cat than those by len-shAxin and len-shTNKS1/2 in YA6060 analog-induced AMPK activation is expected, consistent with major effects from Axin and its upstream TNKS on the complex formation. Overall, the data supports the hypothesis that the Axin stabilizers of the disclosure activate AMPK synergistically via mitochondrial inhibition and enhanced interaction between Axin, AMPK and LKB1.

Confirmation of protein targets for YA6060 analogues-TAB182 and other positive hits that were identified in the preliminary target search are further examined (FIG. 32B). Studies begin with TAB182 because this protein binds to TNKS which stabilizes Axin and have served as a target for Wnt inhibition. Experiments are proposed to determine: i) the binding affinity of YA6060 analogues to TAB182 protein and its mutants by using SPR; ii) the effects of TAB182 silencing, overexpression, and mutation on the anti-CRC cell growth and proliferation by YA6060 analogues as described above; iii) the effects of the same TAB182 manipulation on YA6060 analogue-induced AMPK activation, cellular responses and molecular events, as described above iv) the potential interaction between TAB182, TNKS, and Axin, and the impact by YA6060 analogues. Expressing plasmids for TAB182 and its mutants are used, and the protein expression, purification, and subsequent SPR analysis are examined. TAB182 silencing, overexpression, and mutants are established (FIG. 32A-FIG. 32H). It is expected that TAB182 is identified as an important direct target for Axin stabilizers. In a non-limiting example, overall similar approaches to examine other potential targets can also be used (FIG. 32B).

It is expected that this example demonstrates the efficacy of YA6060 analogues against CRC is, at least partially, via AMPK activation that is due to mitochondrial inhibition involved with Wnt signaling and enhanced formation of Axin-AMPK-LKB1 complex. The identified direct target of YA6060 analogues advances the understanding of signal transduction via Axin, providing novel strategy and targets to develop CRC therapy. In a non-limiting example, if the efficacy of YA6060 analogs was independent of AMPK activation, the preliminary data and the direct target identified through these studies can be used to formulate a new and testable hypothesis, such as mitochondrial dysfunction-induced apoptosis, or TAB182-mediated cancer development and drug sensitivity. In a non-limiting example, a traditional label-based method is used instead of SPR, which widely used in characterizing interaction of small molecules with their protein targets. In a non-limiting example, additional conditions for biotinylated chemical affinity chromatography can be explored, followed by MS protein identification. In a non-limiting example, thermal proteome profiling (TPP) is used, which combines the measurement of protein thermal stability with quantitative proteomics and has recently been used to identify drug targets.

For animal studies related to the YA6060 analogues/hybrids, the animal numbers are proposed based on preliminary studies and prior experience in conducting mouse PK and toxicity studies. For animal studies, the sample size is expected to provide sufficient power for statistical analyses (at the P<0.05 level; α=0.05, β=0.1), which is estimated by Russ Lenth's Power and Sample Size Calculation, with the data of tumor number, growth and size measurement from previous publications. Statistical analyses are conducted with GraphPad software. T tests and analysis of variance (ANOVA) followed by a post-hoc test will be employed in this application. A P value less than 0.05 is considered as statistically different. All chromosomal or plasmid borne assays are checked by PCR following the assay. RT-PCR is performed in triplicate on a minimum of 3 biological replicates. Purified proteins are analyzed by SDS-PAGE and checked by MALDI-TOF mass spectrometry to ensure homogeneity or removal of affinity tags. All Western blots are run on 3 separate experiments with purified protein as a marker and normalized to total protein or to RNA polymerase alpha subunit as a loading control. For proteomic studies all experiments are performed on a minimum of 5 biological replicates and 3 technical replicates per sample. Verification of instrumentation sensitivity is determined by running internal standards as a control to assess day to day variation of instrumentation. Peptide assignments are set at 1% false detection rate (FDR) and relative abundance will be considered significant >1.5 fold and p<0.05. All SMPs and inhibitors are verified by NMR and high accuracy mass spectrometry prior to use.

This Example demonstrates that small molecules with dual activity of Wnt inhibition and AMPK activation via the Axin stabilization are a valid strategy for the development of novel anti-cancer agents. The knowledge and novel small molecules greatly benefit research in understanding the pathophysiology of not only Wnt/β-catenin signaling but also AMPK pathways.

A number of patent and non-patent publications are cited herein in order to describe the state of the art to which this disclosure pertains. The entire disclosure of each of these publications is incorporated by reference herein.

While certain embodiments of the present disclosure have been described and/or exemplified above, various other embodiments will be apparent to those skilled in the art from the foregoing disclosure. The present disclosure is, therefore, not limited to the particular embodiments described and/or exemplified, but is capable of considerable variation and modification without departure from the scope and spirit of the appended claims.

Moreover, as used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed disclosure. All compounds, compositions, formulations, and methods described herein that embody the present disclosure can, in alternate embodiments, be more specifically defined by any of the transitional terms “comprising,” “consisting essentially of,” and “consisting of.”

### REFERENCES

1. Obianom O N, Ai Y., J. Med. Chem. 62:724-741.
2. Yang W, Li Y. et al. J. Med. Chem. 62: 11151-11164.
3. Xue and Shu, US 2019/0071424 A1
4. sNeuschwander-Tetri, B. A. & Caldwell, S. H. Nonalcoholic steatohepatitis: summary of an AASLD Single Topic Conference. Hepatology 37, 1202-1219 (2003).
5. Lazo, M., et al. Prevalence of nonalcoholic fatty liver disease in the United States: the Third National Health and Nutrition Examination Survey, 1988-1994. Am J Epidemiol 178, 38-45 (2013).
6. Marra, F., Gastaldelli, A., Svegliati Baroni, G., Tell, G. & Tiribelli, C. Molecular basis and mechanisms of progression of non-alcoholic steatohepatitis. Trends Mol Med 14, 72-81 (2008).
7. Angulo, P. Nonalcoholic fatty liver disease. N Engl J Med 346, 1221-1231 (2002).
8. Brunt, E. M. & Tiniakos, D. G. Pathological features of NASH. Front Biosci 10, 1475-1484 (2005).
9. Kleiner, D. E., et al. Design and validation of a histological scoring system for nonalcoholic fatty liver disease. Hepatology 41, 1313-1321 (2005).
10. Kim, D., Kim, W. R., Kim, H. J. & Therneau, T. M. Association between noninvasive fibrosis markers and mortality among adults with nonalcoholic fatty liver disease in the United States. Hepatology 57, 1357-1365 (2013).
11. Williams, C. D., et al. Prevalence of nonalcoholic fatty liver disease and nonalcoholic steatohepatitis among a largely middle-aged population utilizing ultrasound and liver biopsy: a prospective study. Gastroenterology 140, 124-131 (2011).
12. Kim, C. H. & Younossi, Z. M. Nonalcoholic fatty liver disease: a manifestation of the metabolic syndrome. Cleve Clin J Med 75, 721-728 (2008).
13. Bellentani, S., Dalle Grave, R., Suppini, A. & Marchesini, G. Behavior therapy for nonalcoholic fatty liver disease: The need for a multidisciplinary approach. Hepatology 47, 746-754 (2008).
14. Musso, G., Gambino, R., Cassader, M. & Pagano, G. A meta-analysis of randomized trials for the treatment of nonalcoholic fatty liver disease. Hepatology 52, 79-104 (2010).
15. Obianom, O. N., et al. Triazole-Based Inhibitors of the Wnt/beta-Catenin Signaling Pathway Improve Glucose and Lipid Metabolisms in Diet-Induced Obese Mice. J Med Chem 62, 727-741 (2019).
16. Yang, W., et al. Pyrazole-4-Carboxamide (YW2065): A Therapeutic Candidate for Colorectal Cancer via Dual Activities of Wnt/beta-Catenin Signaling Inhibition and AMP-Activated Protein Kinase (AMPK) Activation. J Med Chem 62, 11151-11164 (2019).
17. Zhou, S., et al. Pyrvinium Treatment Confers Hepatic Metabolic Benefits via beta-Catenin Downregulation and AMPK Activation. Pharmaceutics 13(2021).
18. Bellentani, S. The epidemiology of non-alcoholic fatty liver disease. Liver Int 37 Suppl 1, 81-84 (2017).
19. Agopian, V. G., et al. Liver transplantation for nonalcoholic steatohepatitis: the new epidemic. Ann Surg 256, 624-633 (2012).
20. Younossi, Z. M., et al. The economic and clinical burden of nonalcoholic fatty liver disease in the United States and Europe. Hepatology 64, 1577-1586 (2016).
21. Thiagarajan, P. & Aithal, G. P. Drug Development for Nonalcoholic Fatty Liver Disease: Landscape and Challenges. J Clin Exp Hepatol 9, 515-521 (2019).
22. Sumida, Y., Okanoue, T. & Nakajima, A. Phase 3 drug pipelines in the treatment of non-alcoholic steatohepatitis. Hepatol Res 49, 1256-1262 (2019).
23. Huang, S. M., et al. Tankyrase inhibition stabilizes axin and antagonizes Wnt signalling. Nature 461, 614-620 (2009).
24. Mariotti, L., et al. Tankyrase Requires SAM Domain-Dependent Polymerization to Support Wnt-beta-Catenin Signaling. Mol Cell 63, 498-513 (2016).
25. Obianom, O. N., et al. Incorporation of a Biguanide Scaffold Enhances Drug Uptake by Organic Cation Transporters 1 and 2. Mol Pharm 14, 2726-2739 (2017).
26. Hansen, H. H., et al. Mouse models of nonalcoholic steatohepatitis in preclinical drug development. Drug Discov Today 22, 1707-1718 (2017).
27. Tsuchida, T., et al. A simple diet- and chemical-induced murine NASH model with rapid progression of steatohepatitis, fibrosis and liver cancer. J Hepatol 69, 385-395 (2018).
28. Kinzler, K. W. & Vogelstein, B. Lessons from hereditary colorectal cancer. Cell 87, 159-70 (1996).
29. Niemann, S. et al. Homozygous WNT3 mutation causes tetra-amelia in a large consanguineous family. Am J Hum Genet 74, 558-63 (2004).
30. Rodova, M., Islam, M. R., Maser, R. L. & Calvet, J. P. The polycystic kidney disease-1 promoter is a target of the beta-catenin/T-cell factor pathway. J Biol Chem 277, 29577-83 (2002).
31. Miyaoka, T., Seno, H. & Ishino, H. Increased expression of Wnt-1 in schizophrenic brains. Schizophr Res 38, 1-6 (1999).
32. Katsu, T. et al. The human frizzled-3 (FZD3) gene on chromosome 8p21, a receptor gene for Wnt ligands, is associated with the susceptibility to schizophrenia. Neurosci Lett 353, 53-6 (2003).
33. Kozlovsky, N., Belmaker, R. H. & Again, G. GSK-3 and the neurodevelopmental hypothesis of schizophrenia. Eur Neuropsychopharmacol 12, 13-25 (2002).
34. Mudher, A. & Lovestone, S. Alzheimer's disease-do tauists and baptists finally shake hands?Trends Neurosci 25, 22-6 (2002).
35. Caricasole, A. et al. The Wnt pathway, cell-cycle activation and beta-amyloid: novel therapeutic strategies in Alzheimer's disease? Trends Pharmacol Sci 24, 233-8 (2003).
36. Oh, D. Y. & Olefsky, J. M. Medicine. Wnt fans the flames in obesity. Science 329, 397-8 (2010).
37. Bordonaro, M. Role of Wnt signaling in the development of type 2 diabetes. Vitam Horm 80, 563-81 (2009).
38. Schinner, S. Wnt-signalling and the metabolic syndrome. Horm Metab Res 41, 159-63 (2009).
39. Neuschwander-Tetri, B. A. & Caldwell, S. H. Nonalcoholic steatohepatitis: summary of an AASLD Single Topic Conference. Hepatology 37, 1202-1219 (2003).
40. Lazo, M., et al. Prevalence of nonalcoholic fatty liver disease in the United States: the Third National Health and Nutrition Examination Survey, 1988-1994. Am J Epidemiol 178, 38-45 (2013).
41. Marra, F., Gastaldelli, A., Svegliati Baroni, G., Tell, G. & Tiribelli, C. Molecular basis and mechanisms of progression of non-alcoholic steatohepatitis. Trends Mol Med 14, 72-81 (2008).
42. Angulo, P. Nonalcoholic fatty liver disease. N Engl J Med 346, 1221-1231 (2002).
43. Brunt, E. M. & Tiniakos, D. G. Pathological features of NASH. Front Biosci 10, 1475-1484 (2005).
44. Kleiner, D. E., et al. Design and validation of a histological scoring system for nonalcoholic fatty liver disease. Hepatology 41, 1313-1321 (2005).
45. Kim, D., Kim, W. R., Kim, H. J. & Therneau, T. M. Association between noninvasive fibrosis markers and mortality among adults with nonalcoholic fatty liver disease in the United States. Hepatology 57, 1357-1365 (2013).
46. Williams, C. D., et al. Prevalence of nonalcoholic fatty liver disease and nonalcoholic steatohepatitis among a largely middle-aged population utilizing ultrasound and liver biopsy: a prospective study. Gastroenterology 140, 124-131 (2011).
47. Kim, C. H. & Younossi, Z. M. Nonalcoholic fatty liver disease: a manifestation of the metabolic syndrome. Cleve Clin J Med 75, 721-728 (2008).
48. Bellentani, S., Dalle Grave, R., Suppini, A. & Marchesini, G. Behavior therapy for nonalcoholic fatty liver disease: The need for a multidisciplinary approach. Hepatology 47, 746-754 (2008).
49. Musso, G., Gambino, R., Cassader, M. & Pagano, G. A meta-analysis of randomized trials for the treatment of nonalcoholic fatty liver disease. Hepatology 52, 79-104 (2010).
50. Obianom, O. N., et al. Triazole-Based Inhibitors of the Wnt/beta-Catenin Signaling Pathway Improve Glucose and Lipid Metabolisms in Diet-Induced Obese Mice. J Med Chem 62, 727-741 (2019).
51. Yang, W., et al. Pyrazole-4-Carboxamide (YW2065): A Therapeutic Candidate for Colorectal Cancer via Dual Activities of Wnt/beta-Catenin Signaling Inhibition and AMP-Activated Protein Kinase (AMPK) Activation. J Med Chem 62, 11151-11164 (2019).
52. Zhou, S., et al. Pyrvinium Treatment Confers Hepatic Metabolic Benefits via beta-Catenin Downregulation and AMPK Activation. Pharmaceutics 13(2021).
53. Bellentani, S. The epidemiology of non-alcoholic fatty liver disease. Liver Int 37 Suppl 1, 81-84 (2017).
54. Agopian, V. G., et al. Liver transplantation for nonalcoholic steatohepatitis: the new epidemic. Ann Surg 256, 624-633 (2012).
55. Younossi, Z. M., et al. The economic and clinical burden of nonalcoholic fatty liver disease in the United States and Europe. Hepatology 64, 1577-1586 (2016).
56. Thiagarajan, P. & Aithal, G. P. Drug Development for Nonalcoholic Fatty Liver Disease: Landscape and Challenges. J Clin Exp Hepatol 9, 515-521 (2019).
57. Sumida, Y., Okanoue, T. & Nakajima, A. Phase 3 drug pipelines in the treatment of non-alcoholic steatohepatitis. Hepatol Res 49, 1256-1262 (2019).
58. Huang, S. M., et al. Tankyrase inhibition stabilizes axin and antagonizes Wnt signalling. Nature 461, 614-620 (2009).
59. Mariotti, L., et al. Tankyrase Requires SAM Domain-Dependent Polymerization to Support Wnt-beta-Catenin Signaling. Mol Cell 63, 498-513 (2016).
60. Obianom, O. N., et al. Incorporation of a Biguanide Scaffold Enhances Drug Uptake by Organic Cation Transporters 1 and 2. Mol Pharm 14, 2726-2739 (2017).
61. Hansen, H. H., et al. Mouse models of nonalcoholic steatohepatitis in preclinical drug development. Drug Discov Today 22, 1707-1718 (2017).
62. Tsuchida, T., et al. A simple diet- and chemical-induced murine NASH model with rapid progression of steatohepatitis, fibrosis and liver cancer. J Hepatol 69, 385-395 (2018).
63. Pouletty et al., WO 2020127843.
64. Chen et al., WO 2020028392.
65. Siegel, R. L., Miller, K. D. & Jemal, A. Cancer statistics, 2015. CA Cancer J Clin 65, 5-29 (2015).
66. Siegel, R., Desantis, C. & Jemal, A. Colorectal cancer statistics, 2014. CA Cancer J Clin 64, 104-17 (2014).
67. Modjtahedi, H. & Essapen, S. Epidermal growth factor receptor inhibitors in cancer treatment: advances, challenges and opportunities. Anticancer Drugs 20, 851-5 (2009).
68. Overman, M. J. & Hoff, P. M. EGFR-targeted therapies in colorectal cancer. Dis Colon Rectum 50, 1259-70 (2007).
69. Venook, A. P. Epidermal growth factor receptor-targeted treatment for advanced colorectal carcinoma. Cancer 103, 2435-46 (2005).
70. Caldwell, G. M. et al. The Wnt antagonist sFRP1 in colorectal tumorigenesis. Cancer Res 64, 883-8 (2004).
71. Groden, J. et al. Identification and characterization of the familial adenomatous polyposis coli gene. Cell 66, 589-600 (1991).
72. Kinzler, K. W. et al. Identification of FAP locus genes from chromosome 5q21. Science 253, 661-5 (1991).
73. Nishisho, I. et al. Mutations of chromosome 5q21 genes in FAP and colorectal cancer patients. Science 253, 665-9 (1991).
74. Su, L. K., Vogelstein, B. & Kinzler, K. W. Association of the APC tumor suppressor protein with catenins. Science 262, 1734-7 (1993).
75. Suzuki, H. et al. Epigenetic inactivation of SFRP genes allows constitutive WNT signaling in colorectal cancer. Nat Genet 36, 417-22 (2004).
76. Comprehensive molecular characterization of human colon and rectal cancer. Nature 487, 330-7 (2012).
77. Pineda, C. T. et al. Degradation of AMPK by a cancer-specific ubiquitin ligase. Cell 160, 715-28 (2015).
78. Ma, L. et al. Control of nutrient stress-induced metabolic reprogramming by PKCzeta in tumorigenesis. Cell 152, 599-611 (2013).
79. Kerr, E. M., Gaude, E., Turrell, F. K., Frezza, C. & Martins, C. P. Mutant Kras copy number defines metabolic reprogramming and therapeutic susceptibilities. Nature 531, 110-3 (2016).
80. Jain, M. et al. Metabolite profiling identifies a key role for glycine in rapid cancer cell proliferation. Science 336, 1040-4 (2012).
81. Wodarz, A. & Nusse, R. Mechanisms of Wnt signaling in development. Annu Rev Cell Dev Biol 14, 59-88 (1998).
82. Ohishi, K. et al. 9-Hydroxycanthin-6-one, a beta-Carboline Alkaloid from Eurycoma longifolia, Is the First Wnt Signal Inhibitor through Activation of Glycogen Synthase Kinase 3beta without Depending on Casein Kinase 1alpha. J Nat Prod (2015).
83. Lu, D. et al. Salinomycin inhibits Wnt signaling and selectively induces apoptosis in chronic lymphocytic leukemia cells. Proc Natl Acad Sci USA 108, 13253-7 (2011).
84. Hao, J. et al. Selective small molecule targeting beta-catenin function discovered by in vivo chemical genetic screen. Cell Rep 4, 898-904 (2013).
85. Gwak, J. et al. Small molecule-based disruption of the Axin/beta-catenin protein complex regulates mesenchymal stem cell differentiation. Cell Res 22, 237-47 (2012).
86. Huang, S. M. et al. Tankyrase inhibition stabilizes axin and antagonizes Wnt signalling. Nature 461, 614-20 (2009).
87. Henderson, W. R., Jr. et al. Inhibition of Wnt/beta-catenin/CREB binding protein (CBP) signaling reverses pulmonary fibrosis. Proc Natl Acad Sci USA 107, 14309-14 (2010).
88. Toume, K. et al. Xylogranin B: a potent Wnt signal inhibitory limonoid from Xylocarpus granatum. Org Lett 15, 6106-9 (2013).
89. Lepourcelet, M. et al. Small-molecule antagonists of the oncogenic Tcf/beta-catenin protein complex. Cancer Cell 5, 91-102 (2004).
90. Park, H. Y. et al. Calotropin: A Cardenolide from Calotropis gigantea that Inhibits Wnt Signaling by Increasing Casein Kinase 1 alpha in Colon Cancer Cells. Chembiochem 15, 872-878 (2014).
91. Thorne, C. A. et al. Small-molecule inhibition of Wnt signaling through activation of casein kinase 1 alpha. Nature Chemical Biology 6, 829-836 (2010).
92. Cha, P. H. et al. Small-molecule binding of the axin RGS domain promotes beta-catenin and Ras degradation. Nat Chem Biol 12, 593-600 (2016).
93. Liu, J. et al. Targeting Wnt-driven cancer through the inhibition of Porcupine by LGK974. Proc Natl Acad Sci USA 110, 20224-9 (2013).
94. Madan, B. et al. Wnt addiction of genetically defined cancers reversed by PORCN inhibition. Oncogene 35, 2197-207 (2016).
95. Chen, B. et al. Small molecule-mediated disruption of Wnt-dependent signaling in tissue regeneration and cancer. Nat Chem Biol 5, 100-7 (2009).
96. Kahn, M. Can we safely target the WNT pathway? Nat Rev Drug Discov 13, 513-32 (2014).
97. Zimmerman, Z. F., Moon, R. T. & Chien, A. J. Targeting Wnt pathways in disease. Cold Spring Harb Perspect Biol 4(2012).
98. Steinberg, G. R. & Carling, D. AMP-activated protein kinase: the current landscape for drug development. Nat Rev Drug Discov 18, 527-551 (2019).
99. Li, W., Saud, S. M., Young, M. R., Chen, G. & Hua, B. Targeting AMPK for cancer prevention and treatment. Oncotarget 6, 7365-78 (2015).
100. Thent, Z. C. et al. Is metformin a therapeutic paradigm for colorectal cancer: Insight into the molecular pathway? Curr Drug Targets (2016).
101. Bahrami, A. et al. Therapeutic Potential of Targeting Wnt/beta-Catenin Pathway in Treatment of Colorectal Cancer: Rational and Progress. J Cell Biochem 118, 1979-1983 (2017).
102. Lu, B., Green, B. A., Farr, J. M., Lopes, F. C. & Van Raay, T. J. Wnt Drug Discovery: Weaving Through the Screens, Patents and Clinical Trials. Cancers (Basel) 8(2016).
103. Zhang, X. & Hao, J. Development of anticancer agents targeting the Wnt/beta-catenin signaling. Am J Cancer Res 5, 2344-60 (2015).
104. Lenz, H. J. & Kahn, M. Safely targeting cancer stem cells via selective catenin coactivator antagonism. Cancer Sci 105, 1087-92 (2014).
105. Blagodatski, A., Poteryaev, D. & Katanaev, V. L. Targeting the Wnt pathways for therapies. Mol Cell Ther 2, 28 (2014).
106. Cheng, D. et al. Discovery of Pyridinyl Acetamide Derivatives as Potent, Selective, and Orally Bioavailable Porcupine Inhibitors. ACS Medicinal Chemistry Letters 7, 676-680 (2016).
107. Chen, B. et al. Small molecule-mediated disruption of Wnt-dependent signaling in tissue regeneration and cancer. Nature Chemical Biology 5, 100 (2009).
108. Lepourcelet, M. et al. Small-molecule antagonists of the oncogenic Tcf/beta-catenin protein complex. Cancer Cell 5, 91-102 (2004).
109. Gonsalves, F. C. et al. An RNAi-based chemical genetic screen identifies three small-molecule inhibitors of the Wnt/wingless signaling pathway. Proc Natl Acad Sci USA 108, 5954-63 (2011).
110. Emami, K. H. et al. A small molecule inhibitor of beta-catenin/CREB-binding protein transcription [corrected]. Proc Natl Acad Sci USA 101, 12682-7 (2004).
111. Voronkov, A. et al. Structural basis and SAR for G007-LK, a lead stage 1,2,4-triazole based specific tankyrase 1/2 inhibitor. J Med Chem 56, 3012-23 (2013).
112. Shultz, M. D. et al. Identification of NVP-TNKS656: the use of structure-efficiency relationships to generate a highly potent, selective, and orally active tankyrase inhibitor. J Med Chem 56, 6495-511 (2013).
113. Mariotti, L. et al. Tankyrase Requires SAM Domain-Dependent Polymerization to Support Wnt-beta-Catenin Signaling. Mol Cell 63, 498-513 (2016).
114. Sbodio, J. I. & Chi, N. W. Identification of a tankyrase-binding motif shared by IRAP, TAB182, and human TRF1 but not mouse TRF1. NuMA contains this RXXPDG motif and is a novel tankyrase partner. J Biol Chem 277, 31887-92 (2002).
115. Xu, D. et al. USP25 regulates Wnt signaling by controlling the stability of tankyrases. Genes Dev 31, 1024-1035 (2017).
116. Eisemann, T. et al. Tankyrase-1 Ankyrin Repeats Form an Adaptable Binding Platform for Targets of ADP-Ribose Modification. Structure 24, 1679-1692 (2016).
117. Zhong, Y. et al. Tankyrase Inhibition Causes Reversible Intestinal Toxicity in Mice with a Therapeutic Index <1. Toxicol Pathol 44, 267-78 (2016).
118. Mariotti, L., Pollock, K. & Guettler, S. Regulation of Wnt/beta-catenin signalling by tankyrase-dependent poly(ADP-ribosyl)ation and scaffolding. Br J Pharmacol 174, 4611-4636 (2017).
119. Zhu, W., Groh, M., Haupenthal, J. & Hartmann, R. W. A detective story in drug discovery: elucidation of a screening artifact reveals polymeric carboxylic acids as potent inhibitors of RNA polymerase. Chemistry 19, 8397-400 (2013).
120. Smith, T. C., Kinkel, A. W., Gryczko, C. M. & Goulet, J. R. Absorption of pyrvinium pamoate. Clin Pharmacol Ther 19, 802-6 (1976).
121. Xu, W. et al. The antihelmintic drug pyrvinium pamoate targets aggressive breast cancer. PLoS One 8, e71508 (2013).
122. Zhou, G. et al. Role of AMP-activated protein kinase in mechanism of metformin action. J Clin Invest 108, 1167-74 (2001).
123. Jenkins, Y. et al. AMPK activation through mitochondrial regulation results in increased substrate oxidation and improved metabolic parameters in models of diabetes. PLoS One 8, e81870 (2013).
124. Hawley, S. A. et al. The Na+/Glucose Cotransporter Inhibitor Canagliflozin Activates AMPK by Inhibiting Mitochondrial Function and Increasing Cellular AMP Levels. Diabetes 65, 2784-94 (2016).
125. Hardie, D. G. AMPK: a target for drugs and natural products with effects on both diabetes and cancer. Diabetes 62, 2164-72 (2013).
126. Yang, W. et al. Pyrazole-4-Carboxamide (YW2065): A Therapeutic Candidate for Colorectal Cancer via Dual Activities of Wnt/beta-Catenin Signaling Inhibition and AMP-Activated Protein Kinase (AMPK) Activation. J Med Chem 62, 11151-11164 (2019).
127. Polakis, P. Drugging Wnt signalling in cancer. EMBO J 31, 2737-46 (2012).
128. Salic, A., Lee, E., Mayer, L. & Kirschner, M. W. Control of beta-catenin stability: reconstitution of the cytoplasmic steps of the wnt pathway in Xenopus egg extracts. Mol Cell 5, 523-32 (2000).
129. Lee, E., Salic, A., Kruger, R., Heinrich, R. & Kirschner, M. W. The roles of APC and Axin derived from experimental and theoretical analysis of the Wnt pathway. PLoS Biol 1, E10 (2003).
130. Ahmed, D. et al. Epigenetic and genetic features of 24 colon cancer cell lines. Oncogenesis 2, e71 (2013).
131. Grossmann, T. N. et al. Inhibition of oncogenic Wnt signaling through direct targeting of beta-catenin. Proc Natl Acad Sci USA 109, 17942-7 (2012).
132. Findlay, V. J. et al. SNAI2 modulates colorectal cancer 5-fluorouracil sensitivity through miR145 repression. Mol Cancer Ther 13, 2713-26 (2014).
133. Varghese, V. et al. FOXM1 modulates 5-FU resistance in colorectal cancer through regulating TYMS expression. Sci Rep 9, 1505 (2019).
134. Hata, T. et al. Role of p21waf1/cip1 in effects of oxaliplatin in colorectal cancer cells. Mol Cancer Ther 4, 1585-94 (2005).
135. Temmink, O. H. et al. Mechanism of trifluorothymidine potentiation of oxaliplatin-induced cytotoxicity to colorectal cancer cells. Br J Cancer 96, 231-40 (2007).
136. Fidler, I. J. Orthotopic implantation of human colon carcinomas into nude mice provides a valuable model for the biology and therapy of metastasis. Cancer Metastasis Rev 10, 229-43 (1991).
137. Funahashi, Y., Koyanagi, N., Sonoda, J., Kitoh, K. & Yoshimatsu, K. Rapid development of hepatic metastasis with high incidence following orthotopic transplantation of murine colon 38 carcinoma as intact tissue in syngeneic C57BL/6 mice. J Surg Oncol 71, 83-90 (1999).
138. Zabala, M. et al. Evaluation of bioluminescent imaging for noninvasive monitoring of colorectal cancer progression in the liver and its response to immunogene therapy. Mol Cancer 8, 2 (2009).
139. Shin, J. H., Kim, H. W., Rhyu, I. J. & Kee, S. H. Axin is expressed in mitochondria and suppresses mitochondrial ATP synthesis in HeLa cells. Exp Cell Res 340, 12-21 (2016).
140. Pate, K. T. et al. Wnt signaling directs a metabolic program of glycolysis and angiogenesis in colon cancer. EMBO J 33, 1454-73 (2014).
141. Tomitsuka, E., Kita, K. & Esumi, H. An anticancer agent, pyrvinium pamoate inhibits the NADH-fumarate reductase system—a unique mitochondrial energy metabolism in tumour microenvironments. J Biochem 152, 171-83 (2012).
142. Venerando, A., Girardi, C., Ruzzene, M. & Pinna, L. A. Pyrvinium pamoate does not activate protein kinase CK1, but promotes Akt/PKB down-regulation and GSK3 activation. Biochem J 452, 131-7 (2013).
143. Obianom, O. N. et al. Triazole-Based Inhibitors of the Wnt/beta-Catenin Signaling Pathway Improve Glucose and Lipid Metabolisms in Diet-Induced Obese Mice. J Med Chem 62, 727-741 (2019).
144. Ohishi, T. et al. Tankyrase-Binding Protein TNKS1BP1 Regulates Actin Cytoskeleton Rearrangement and Cancer Cell Invasion. Cancer Res 77, 2328-2338 (2017).
145. Tan, W. et al. Overexpression of TNKS1BP1 in lung cancers and its involvement in homologous recombination pathway of DNA double-strand breaks. Cancer Med 6, 483-493 (2017).
146. Zou, L. H. et al. TNKS1BP1 functions in DNA double-strand break repair though facilitating DNA-PKcs autophosphorylation dependent on PARP-1. Oncotarget 6, 7011-22 (2015).
147. Gupta, D., Bhatia, D., Dave, V., Sutariya, V. & Varghese Gupta, S. Salts of Therapeutic Agents: Chemical, Physicochemical, and Biological Considerations. Molecules 23(2018).
148. Ilyas, M., Tomlinson, I. P., Rowan, A., Pignatelli, M. & Bodmer, W. F. Beta-catenin mutations in cell lines established from human colorectal cancers. Proc Natl Acad Sci USA 94, 10330-4 (1997).
149. Berg, K. C. G. et al. Multi-omics of 34 colorectal cancer cell lines—a resource for biomedical studies. Mol Cancer 16, 116 (2017).
150. Waaler, J. et al. A novel tankyrase inhibitor decreases canonical Wnt signaling in colon carcinoma cells and reduces tumor growth in conditional APC mutant mice. Cancer Res 72, 2822-32 (2012).
151. Lau, T. et al. A novel tankyrase small-molecule inhibitor suppresses APC mutation-driven colorectal tumor growth. Cancer Res 73, 3132-44 (2013).
152. Okada-Iwasaki, R. et al. The Discovery and Characterization of K-756, a Novel Wnt/beta-Catenin Pathway Inhibitor Targeting Tankyrase. Mol Cancer Ther 15, 1525-34 (2016).
153. Tanaka, N. et al. APC Mutations as a Potential Biomarker for Sensitivity to Tankyrase Inhibitors in Colorectal Cancer. Mol Cancer Ther 16, 752-762 (2017).
154. Faux, M. C. et al. Restoration of full-length adenomatous polyposis coli (APC) protein in a colon cancer cell line enhances cell adhesion. J Cell Sci 117, 427-39 (2004).
155. Li, L. et al. SLC13A5 is a novel transcriptional target of the pregnane X receptor and sensitizes drug-induced steatosis in human liver. Mol Pharmacol 87, 674-82 (2015).
156. Peng, Z., Raufman, J. P. & Xie, G. Src-mediated cross-talk between farnesoid X and epidermal growth factor receptors inhibits human intestinal cell proliferation and tumorigenesis. PLoS One 7, e48461 (2012).
157. Xie, G., Peng, Z. & Raufman, J. P. Src-mediated aryl hydrocarbon and epidermal growth factor receptor cross talk stimulates colon cancer cell proliferation. Am J Physiol Gastrointest Liver Physiol 302, G1006-15 (2012).
158. Li, B. et al. Repurposing the FDA-approved pinworm drug pyrvinium as a novel chemotherapeutic agent for intestinal polyposis. PLoS One 9, e101969 (2014).
159. Raufman, J. P. et al. Genetic ablation of M3 muscarinic receptors attenuates murine colon epithelial cell proliferation and neoplasia. Cancer Res 68, 3573-8 (2008).
160. Peng, Z., Heath, J., Drachenberg, C., Raufman, J. P. & Xie, G. Cholinergic muscarinic receptor activation augments murine intestinal epithelial cell proliferation and tumorigenesis. BMC Cancer 13, 204 (2013).
161. Fernandez, Y. et al. Bioluminescent imaging of animal models for human colorectal cancer tumor growth and metastatic dissemination to clinically significant sites. Journal of Molecular Biology and Molecular Imaging 2, 1019 (2015).
162. Mouradov, D. et al. Colorectal cancer cell lines are representative models of the main molecular subtypes of primary cancer. Cancer Res 74, 3238-47 (2014).
163. Liu, Z. et al. Extensive metabolic disorders are present in APC(min) tumorigenesis mice. Mol Cell Endocrinol 427, 57-64 (2016).
164. Cespedes, M. V. et al. Orthotopic microinjection of human colon cancer cells in nude mice induces tumor foci in all clinically relevant metastatic sites. Am J Pathol 170, 1077-85 (2007).
165. Hung, K. E. et al. Development of a mouse model for sporadic and metastatic colon tumors and its use in assessing drug treatment. Proc Natl Acad Sci USA 107, 1565-70 (2010).
166. Politi, K. & Pao, W. How genetically engineered mouse tumor models provide insights into human cancers. J Clin Oncol 29, 2273-81 (2011).
167. Roper, J. & Hung, K. E. Priceless GEMMs: genetically engineered mouse models for colorectal cancer drug development. Trends Pharmacol Sci 33, 449-55 (2012).
168. Wege, H. et al. Forced activation of beta-catenin signaling supports the transformation of hTERT-immortalized human fetal hepatocytes. Mol Cancer Res 9, 1222-31 (2011).
169. Xie, Z., Zhang, J., Wu, J., Viollet, B. & Zou, M. H. Upregulation of mitochondrial uncoupling protein-2 by the AMP-activated protein kinase in endothelial cells attenuates oxidative stress in diabetes. Diabetes 57, 3222-30 (2008).
170. Chen, L. et al. OCT1 is a high-capacity thiamine transporter that regulates hepatic steatosis and is a target of metformin. Proc Natl Acad Sci USA 111, 9983-8 (2014).
171. Shu, Y. et al. Effect of genetic variation in the organic cation transporter 1 (OCT1) on metformin action. J Clin Invest 117, 1422-31 (2007).
172. Chang, H. W. et al. Knockdown of beta-catenin controls both apoptotic and autophagic cell death through LKB1/AMPK signaling in head and neck squamous cell carcinoma cell lines. Cell Signal 25, 839-47 (2013).
173. Li, N. et al. Tankyrase disrupts metabolic homeostasis and promotes tumorigenesis by inhibiting LKB1-AMPK signalling. Nat Commun 10, 4363 (2019).
174. Yu, T. et al. Decreasing mitochondrial fission prevents cholestatic liver injury. J Biol Chem 289, 34074-88 (2014).
175. Galloway, C. A. et al. Transgenic control of mitochondrial fission induces mitochondrial uncoupling and relieves diabetic oxidative stress. Diabetes 61, 2093-104 (2012).
176. Yu, T., Robotham, J. L. & Yoon, Y. Increased production of reactive oxygen species in hyperglycemic conditions requires dynamic change of mitochondrial morphology. Proc Natl Acad Sci USA 103, 2653-8 (2006).
177. Seimiya, H. & Smith, S. The telomeric poly(ADP-ribose) polymerase, tankyrase 1, contains multiple binding sites for telomeric repeat binding factor 1 (TRF1) and a novel acceptor,
182-kDa tankyrase-binding protein (TAB182). J Biol Chem 277, 14116-26 (2002).
178. Burkhard, K. A., Chen, F. & Shapiro, P. Quantitative analysis of ERK2 interactions with substrate proteins: roles for kinase docking domains and activity in determining binding affinity. J Biol Chem 286, 2477-85 (2011).
179. Helmerhorst, E., Chandler, D. J., Nussio, M. & Mamotte, C. D. Real-time and Label-free Bio-sensing of Molecular Interactions by Surface Plasmon Resonance: A Laboratory Medicine Perspective. Clin Biochem Rev 33, 161-73 (2012).
180. Huber, K. V. et al. Proteome-wide drug and metabolite interaction mapping by thermal-stability profiling. Nat Methods 12, 1055-7 (2015).
181. Lenth, R. V. Java Applets for Power and Sample Size [Computer software]. www.stat.uiowa.edu/˜rlenth/Power.
182. Kamen, B. A., Wang, M. T., Streckfuss, A. J., Peryea, X. & Anderson, R. G. Delivery of folates to the cytoplasm of MA104 cells is mediated by a surface membrane receptor that recycles. J Biol Chem 263, 13602-9 (1988).
183. Matherly, L. H. & Goldman, D. I. Membrane transport of folates. Vitam Horm 66, 403-56 (2003).
184. Zhao, R., Matherly, L. H. & Goldman, I.D. Membrane transporters and folate homeostasis: intestinal absorption and transport into systemic compartments and tissues. Expert Rev Mol Med 11, e4 (2009).
185. Rijnboutt, S. et al. Endocytosis of GPI-linked membrane folate receptor-alpha. J Cell Biol 132, 35-47 (1996).
186. Shia, J. et al. Immunohistochemical expression of folate receptor alpha in colorectal carcinoma: patterns and biological significance. Hum Pathol 39, 498-505 (2008).
187. Teng, L., Xie, J., Teng, L. & Lee, R. J. Clinical translation of folate receptor-targeted therapeutics. Expert Opin Drug Deliv 9, 901-8 (2012).
188. Kelemen, L. E. The role of folate receptor alpha in cancer development, progression and treatment: cause, consequence or innocent bystander? Int J Cancer 119, 243-50 (2006).
189. Matsue, H. et al. Folate receptor allows cells to grow in low concentrations of 5-methyltetrahydrofolate. Proc Natl Acad Sci USA 89, 6006-9 (1992).
190. Zhao, R., Diop-Bove, N., Visentin, M. & Goldman, I.D. Mechanisms of membrane transport of folates into cells and across epithelia. Annu Rev Nutr 31, 177-201 (2011).
191. Sharma, M. et al. Folic acid conjugated guar gum nanoparticles for targeting methotrexate to colon cancer. J Biomed Nanotechnol 9, 96-106 (2013).
192. Cheung, A. et al. Targeting folate receptor alpha for cancer treatment. Oncotarget 7, 52553-52574 (2016).

What is claimed is:

1. A compound of the formula (I): 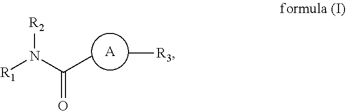
wherein ring A is a 5-membered substituted or unsubstituted heterocycle or heteroaryl;
wherein R₁ is selected from substituted or unsubstituted cycloalkyl, substituted or unsubstituted aryl, substituted or unsubstituted heterocycle, and substituted or unsubstituted heteroaryl;
R₂ is selected from H and substituted or unsubstituted alkyl; and
R₃ is selected from substituted or unsubstituted aryl and substituted or unsubstituted heteroaryl;
or a pharmaceutically acceptable salt thereof.

2. The compound of claim 1, wherein ring A is selected from substituted or unsubstituted triazole, substituted or unsubstituted tetrazole, substituted or unsubstituted pyrazole, substituted or unsubstituted pyrrole, and substituted or unsubstituted thiazole.

3. The compound of claim 1, wherein the compound of formula (I) is a compound of any one of formula (10)-(15): 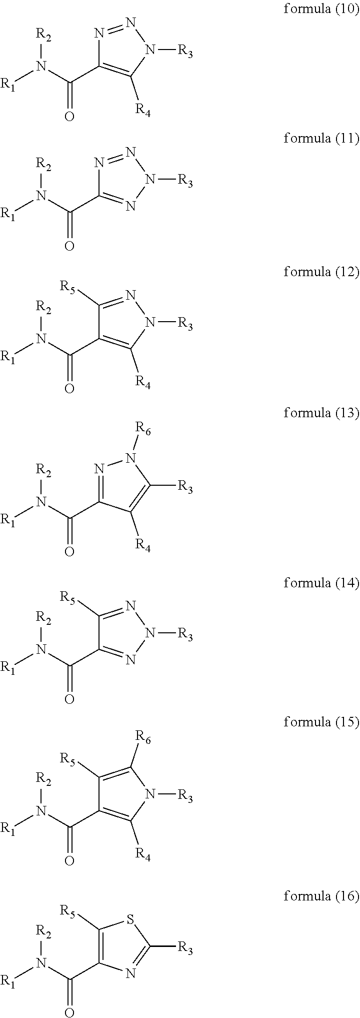
wherein each R₄, R₅, and R₆ is independently selected from H and substituted or unsubstituted alkyl; or a pharmaceutically acceptable salt thereof.

4. The compound of any one of claims 1-3, wherein R₁ is selected from substituted or unsubstituted quinoline, substituted or unsubstituted quinoxaline, substituted or unsubstituted benzothiazole, substituted or unsubstituted isoquinoline, substituted or unsubstituted pyridine, substituted or unsubstituted quinazoline, substituted or unsubstituted 1,5-naphthyridine, substituted or unsubstituted 1,8-naphthyridine, substituted or unsubstituted thiazole, and substituted or unsubstituted benzoxazole.

5. The compound of any one of claims 1-3, wherein R₁ is selected from

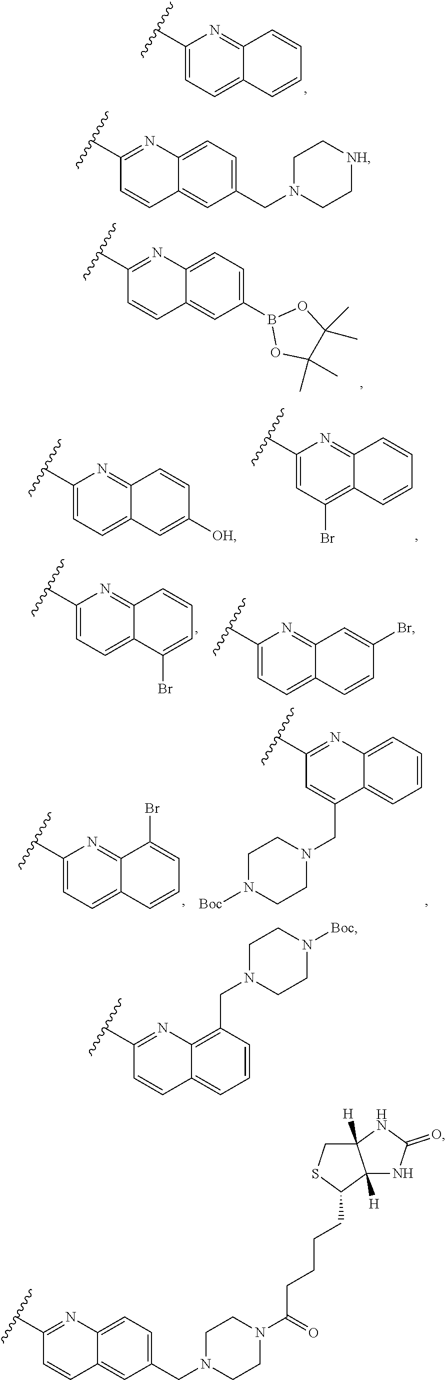

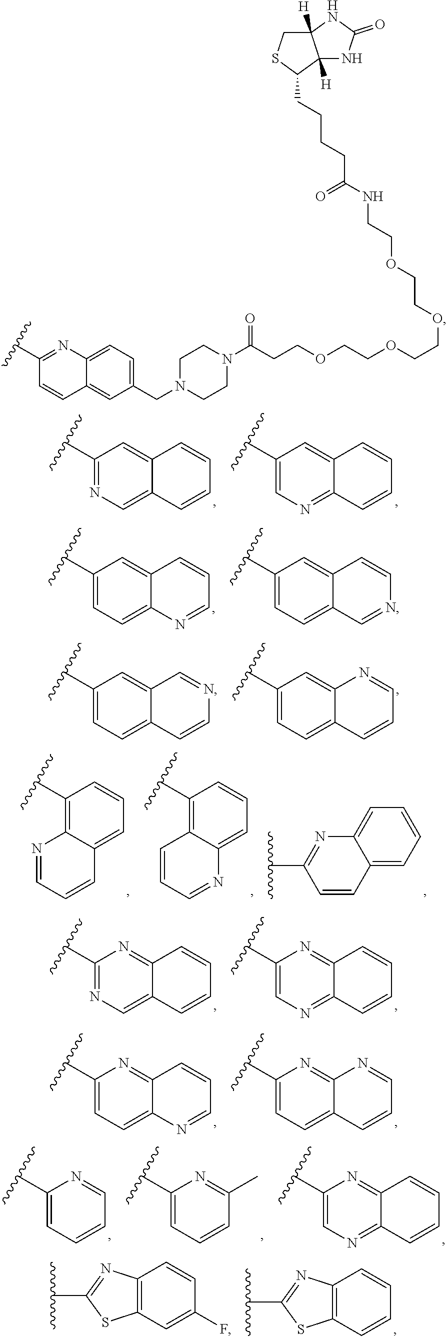

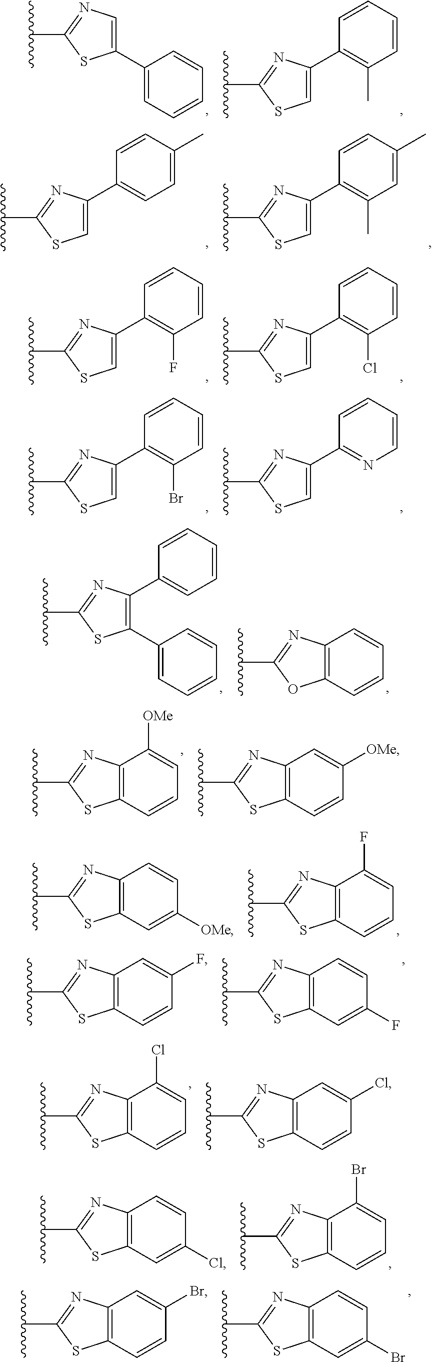

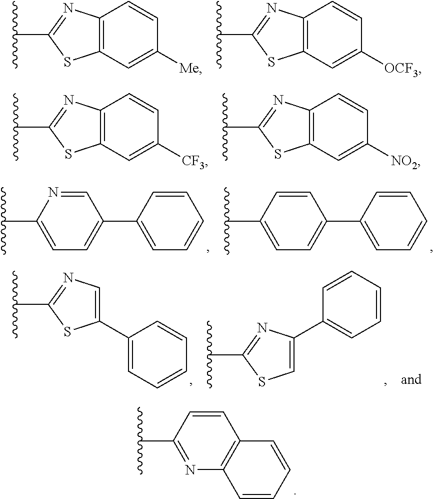

6. The compound of any one of claims 1-5, wherein R₃ is selected from substituted or unsubstituted pyridyl, unsubstituted aryl, and aryl substituted with one or more groups selected from halo, —CN, alkyl, fluroroalkyl, alkoxy, and fluoroalkoxy.

7. The compound of any one of claims 1-5, wherein R₃ is selected from

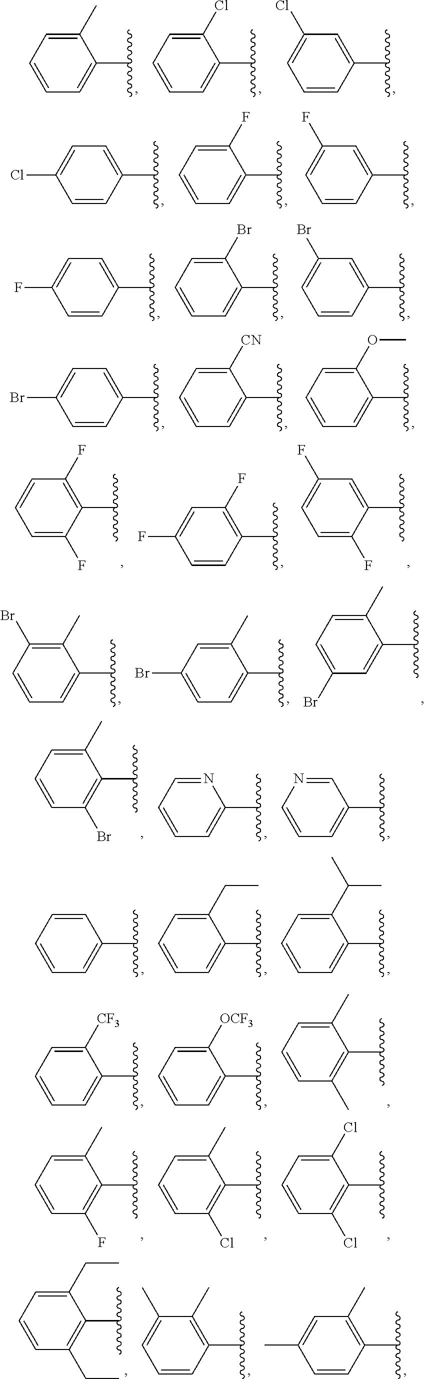

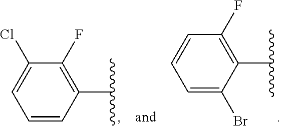 [SMILES: *c1cccc(Cl)c1F ; *c1c(F)cccc1Br]

8. The compound of any one of claims 3-7, wherein each R₄, R₅, and R₆ is independently selected from H, methyl, ethyl, i-propyl, t-butyl, and —CF₃.

9. The compound of claim 1, wherein the compound of formula (I) is a compound of any one of formula (20)-(29): 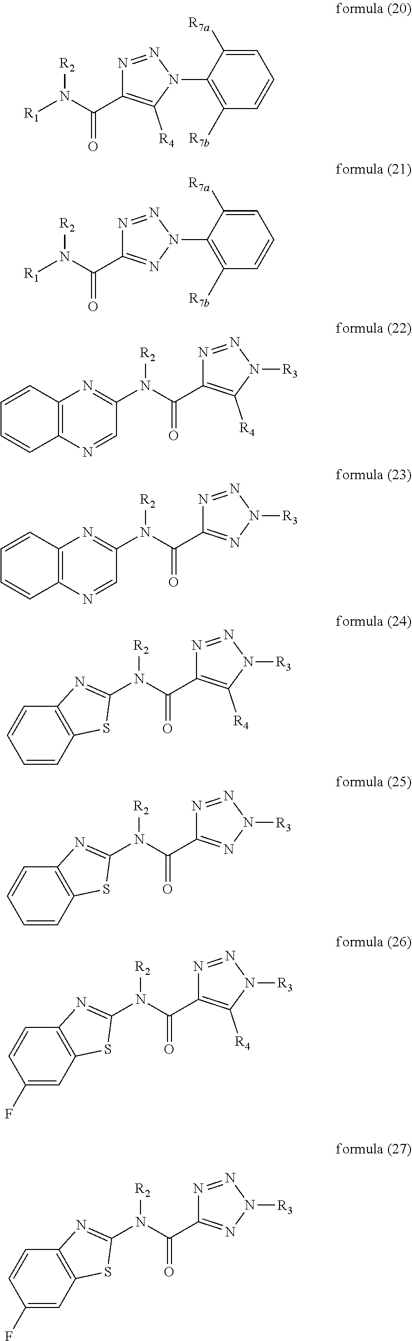 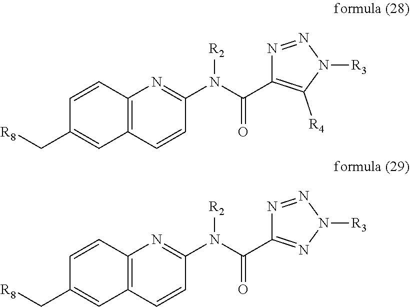
wherein R₄ is selected from H and substituted or unsubstituted alkyl;
each R₇ₐ and R_{7b} is independently selected from H, substituted or unsubstituted alkyl, halo, —CN, fluroroalkyl, alkoxy; and
R₈ is a substituted or unsubstituted heterocycle, or a pharmaceutically acceptable salt thereof.

10. The compound of any one of claims 1-9, wherein R₁ is selected from

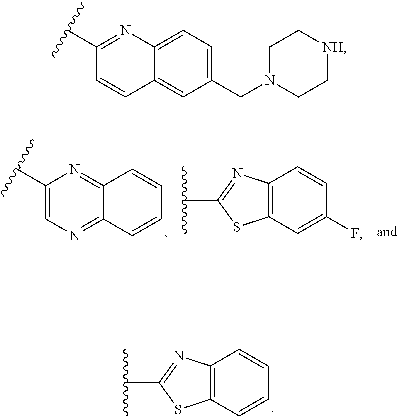

11. The compound of any one of claims 1-10, wherein R₂ is H.

12. The compound of any one of claims 1-11, wherein R₃ is selected from

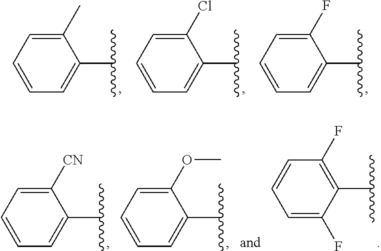

13. The compound of any one of claims 3-12, wherein R₄ is selected from methyl and ethyl.

14. The compound of any one of claims 9-13, wherein R₈ is

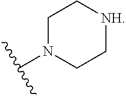 [SMILES: *N1CCNCC1]

15. The compound of claim 1, wherein the compound is a compound of any one of formula 1001-1126, or a pharmaceutically acceptable salt thereof:

| Formula No. | Structure |
|---|---|
| 1001 (YA6060) | 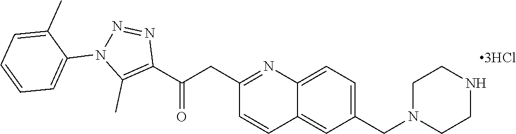 [SMILES: Cc1ccccc1-n1nnc(C(=O)Cc2ccc3cc(CN4CCNCC4)ccc3n2)c1C] |
| 1002 (YA10951) | 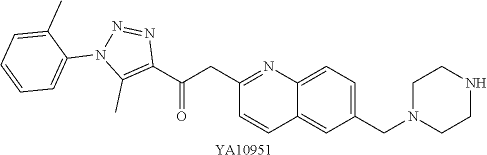 |
| 1003 (YA6150) | 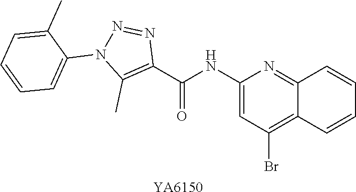 |
| 1004 (YA6167) | 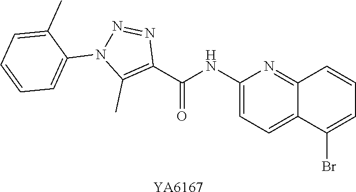 |
| 1005 (YA1126) | 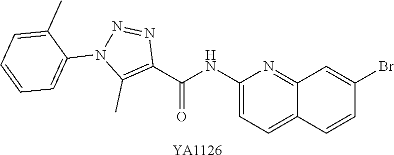 |
| 1006 (YA6146) | 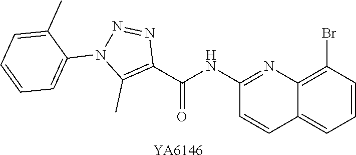 |
| 1007 (YA6159) | 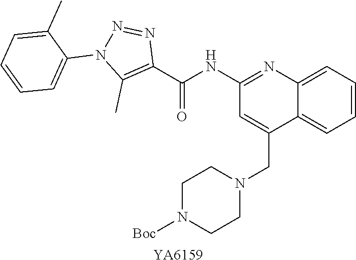 |
| 1008 (YA6160) | 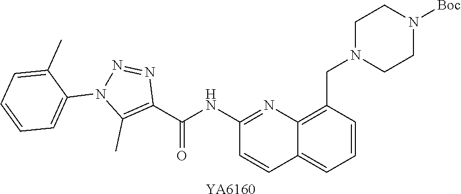 |
| 1009 (YA4182) | 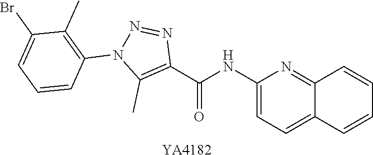 |
| 1010 (YA4179) | 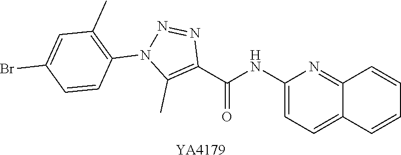 |
| 1011 (YA1130) | 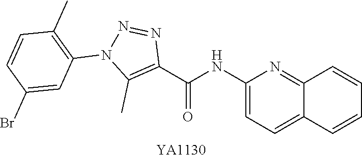 |
| 1012 (YA1128) | 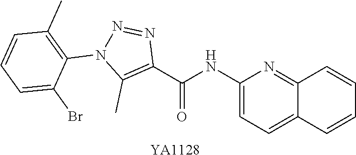 |
| 1013 (YA6023) | 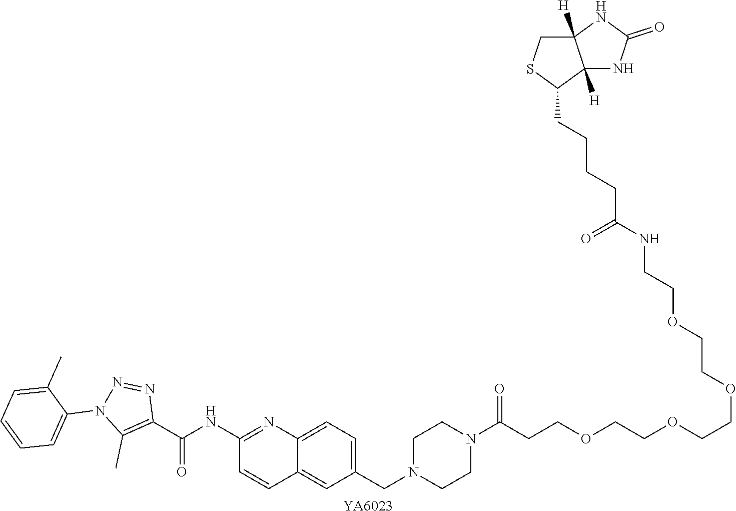 |
| 1014 (YA1103) | 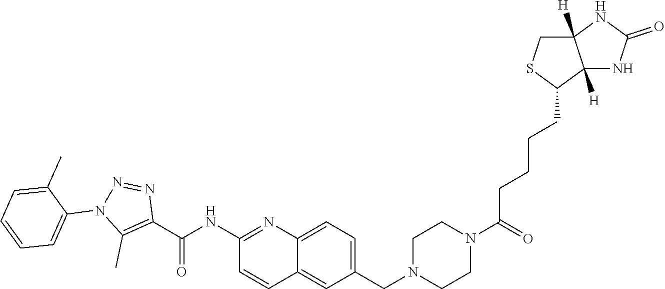 [SMILES: Cc1ccccc1-n1nnc(C(=O)Nc2ccc3cc(CN4CCN(C(=O)CCCC[C@@H]5SC[C@@H]6NC(=O)N[C@@H]65)CC4)ccc3n2)c1C] |
| 1015 (YA6164) | 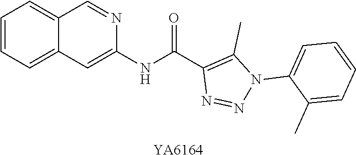 |
| 1016 (YA6161) | 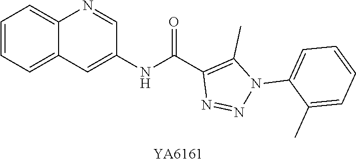 |
| 1017 (YA6056) | 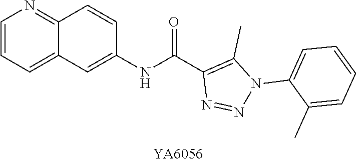 |
| 1018 (YA6169-2) | 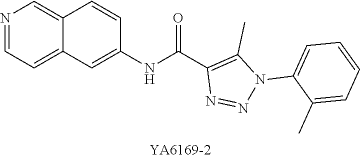 [SMILES: Cc1ccccc1-n1nnc(C(=O)Nc2ccc3cnccc3c2)c1C ; "YA6169-2"] |
| 1019 (YA6169-1) | 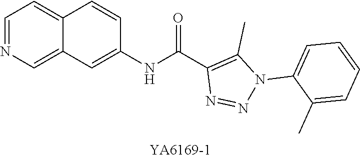 [SMILES: Cc1ccccc1-n1nnc(C(=O)Nc2ccc3ccncc3c2)c1C ; "YA6169-1"] |
| 1020 (YA6162) | 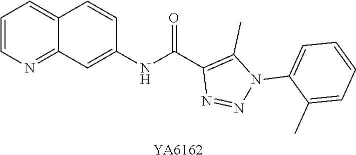 |
| 1021 (YA6055) | 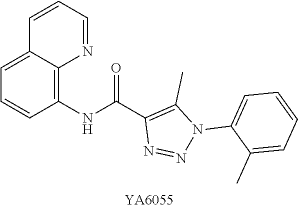 |
| 1022 (YA6169-3) | 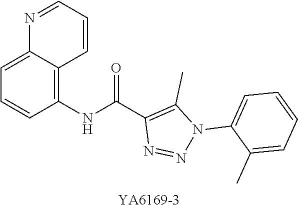 [SMILES: Cc1ccccc1-n1nnc(C(=O)Nc2cccc3ncccc23)c1C ; "YA6169-3"] |
| 1023 (YA6061) | 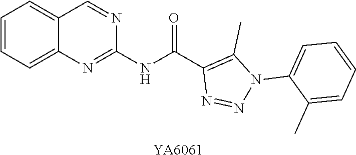 |
| 1024 (YA6045) | 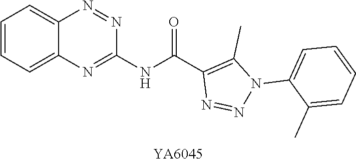 |
| 1025 (YA6147) | 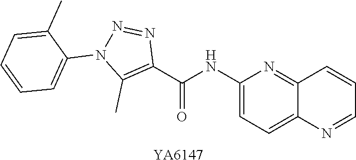 |
| 1026 (YA6047) | 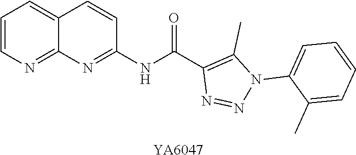 |
| 1027 (YA7013-1) | 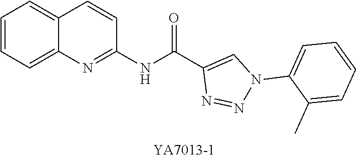 [SMILES: Cc1ccccc1-n1cc(C(=O)Nc2ccc3ccccc3n2)nn1 ; "YA7013-1"] |
| 1028 (YA4114) | 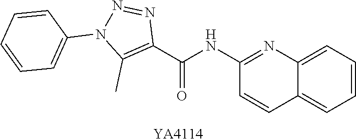 |
| 1029 (YA1144) | 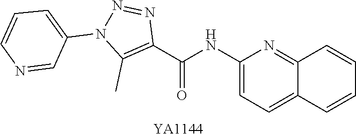 |
| 1030 (YA4112) | 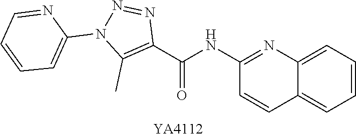 |
| 1031 (YA1144-1) | 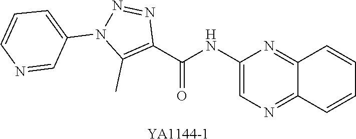 [SMILES: Cc1c(C(=O)Nc2cnc3ccccc3n2)nnn1-c1cccnc1 ; "YA1144-1"] |
| 1032 (YA6136) | 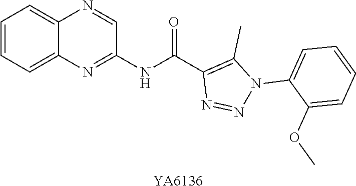 |
| 1033 (YA6137) | 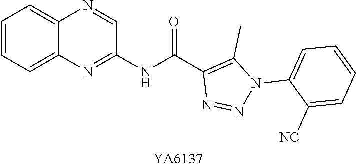 |
| 1034 (YA6138) | 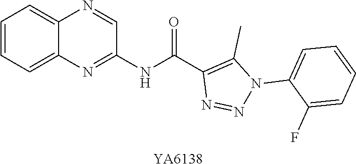 |
| 1035 (YA6139) | 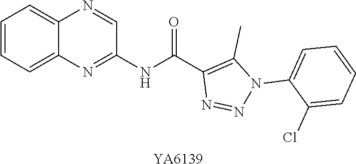 |
| 1036 (YA6141) | 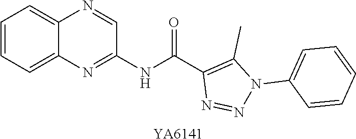 |
| 1037 (YA70031) | 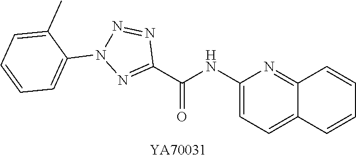 |
| 1038 (YA6171) | 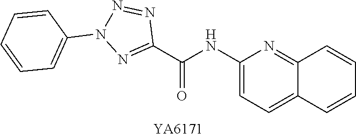 |
| 1039 (YA70032) | 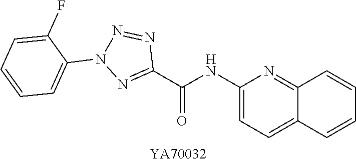 |
| 1040 (YA70033) | 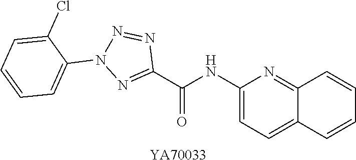 |
| 1041 (YA4184) | 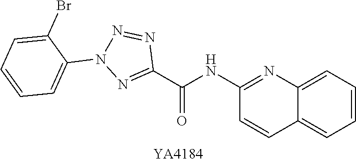 |
| 1042 (YA70043) | 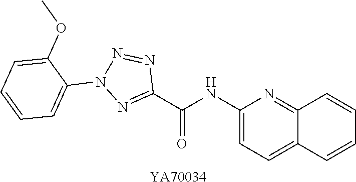 |
| 1043 (YA70035) | 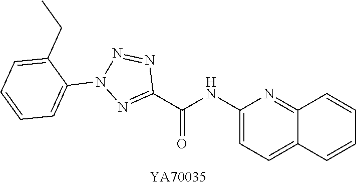 |
| 1044 (YA70036) | 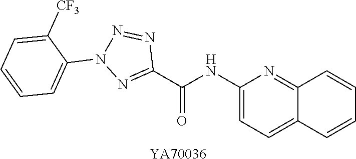 |
| 1045 (YA70037) | 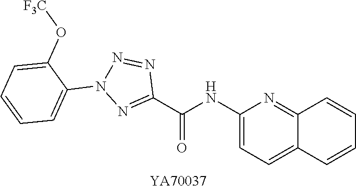 |
| 1046 (YA70038) | 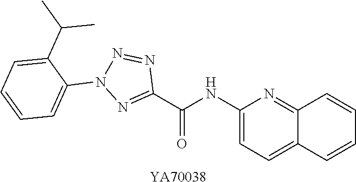 |
| 1047 (YA70041) | 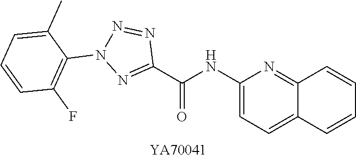 |
| 1048 (YA70042) | 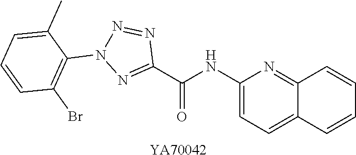 |
| 1049 (YA70043) | 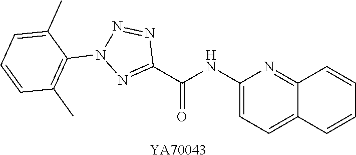 |
| 1050 (YA70044) | 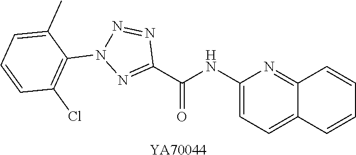 |
| 1051 (YA70045) | 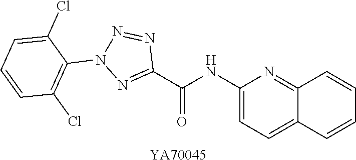 |
| 1052 (YA70046) | 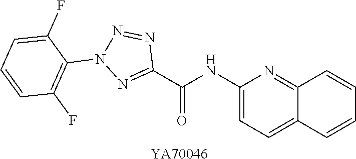 |
| 1053 (YA70047) | 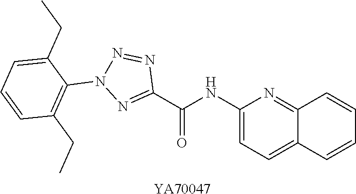 |
| 1054 (YA6177) | 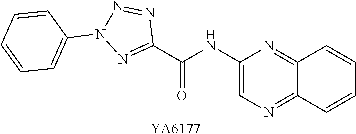 |
| 1055 (YA7083) | 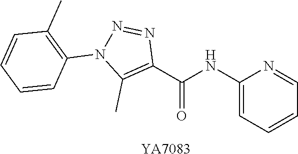 |
| 1056 (YA7084) | 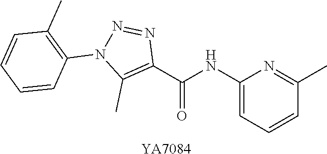 |
| 1057 (YA4117) | 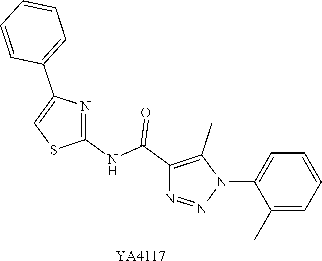 |
| 1058 (YA4085) | 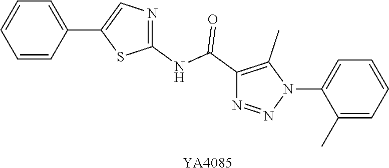 |
| 1059 (YA4133) | 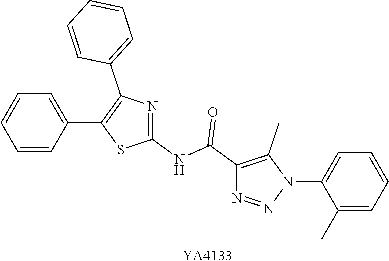 |
| 1060 (YA4105) | 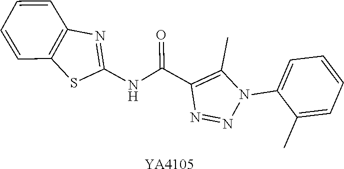 |
| 1061 (YA4061) | 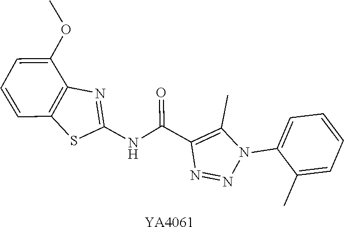 |
| 1062 (YA4045) | 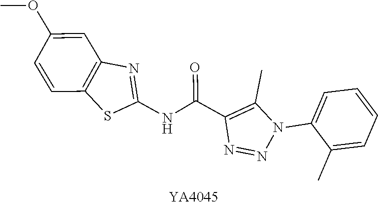 |
| 1063 (YA4091) | 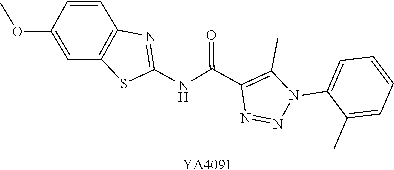 |
| 1064 (YA4093) | 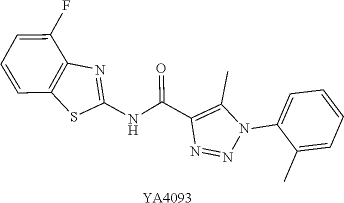 |
| 1065 (YA4101) | 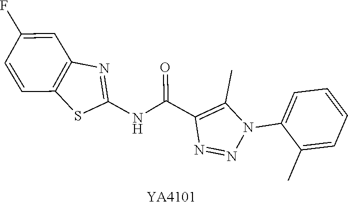 |
| 1066 (YA4103) | 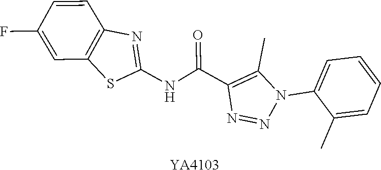 |
| 1067 (YA4095) | 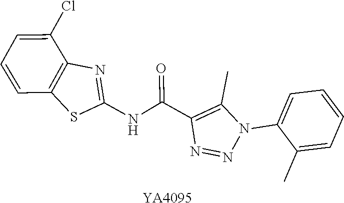 |
| 1068 (YA4013) | 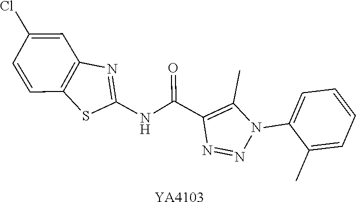 |
| 1069 (YA4071) | 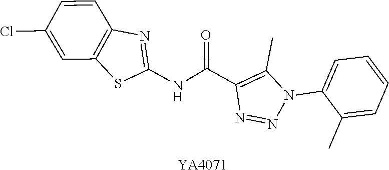 |
| 1070 (YA4097) | 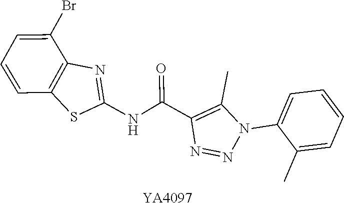 |
| 1071 (YA4047) | 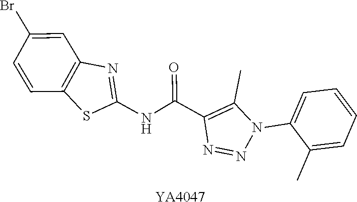 |
| 1072 (YA4059) | 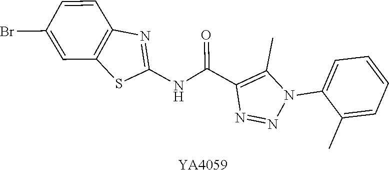 |
| 1073 (YA4069) | 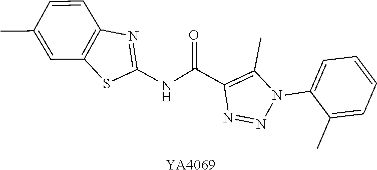 |
| 1074 (YA4065) | 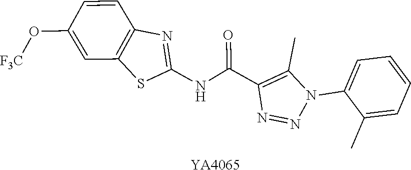 |
| 1075 (YA4063) | 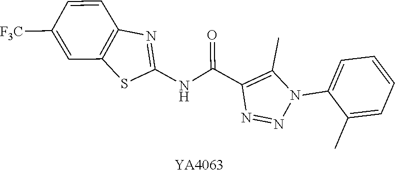 |
| 1076 (YA4057) | 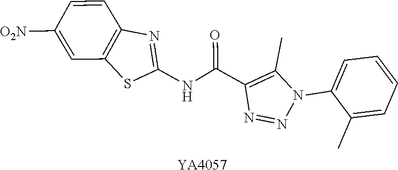 |
| 1077 (YA6029) | 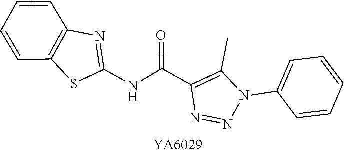 |
| 1078 (YA4029) | 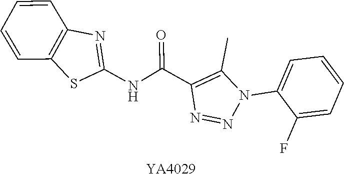 |
| 1079 (YA1072) | 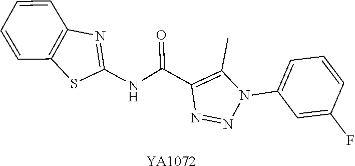 |
| 1080 (YA1076) | 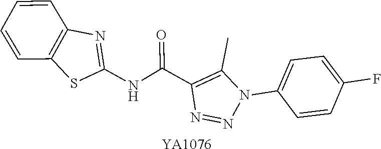 |
| 1081 (YA6027) | 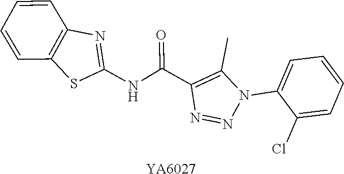 |
| 1082 (YA4055) | 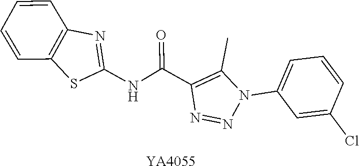 |
| 1083 (YA1074) | 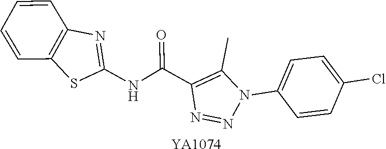 |
| 1084 (YA4003) | 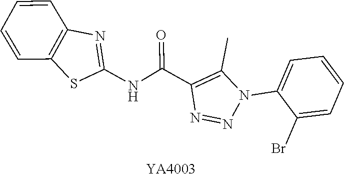 |
| 1085 (YA1050) | 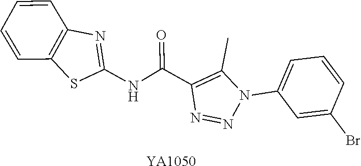 |
| 1086 (YA1086) | 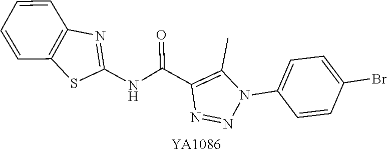 |
| 1087 (YA6089) | 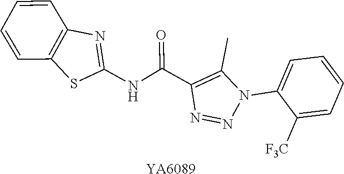 |
| 1088 (YA1038) | 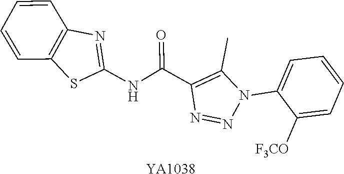 |
| 1089 (YA1046) | 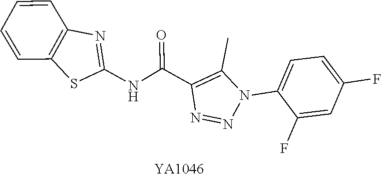 |
| 1090 (YA1084) | 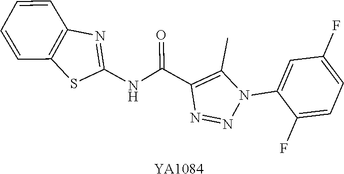 |
| 1091 (YA6090) | 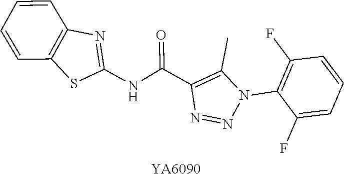 |
| 1092 (YA1066) | 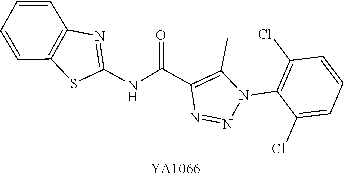 |
| 1093 (YA1042) | 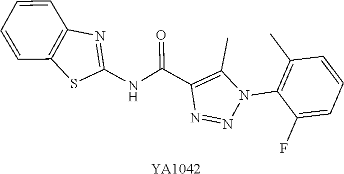 |
| 1094 (YA1068) | 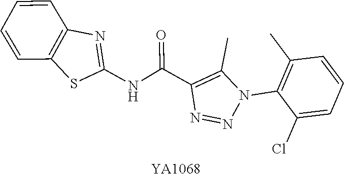 |
| 1095 (YA1064) | 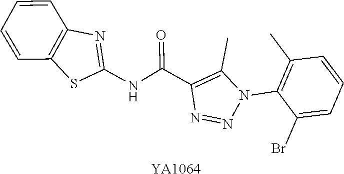 |
| 1096 (YA4001) | 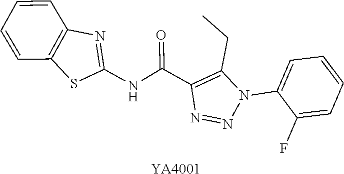 |
| 1097 (YA4005) | 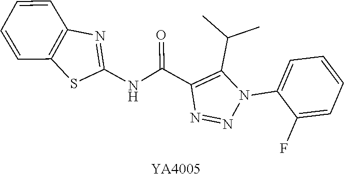 |
| 1098 (YA6176) | 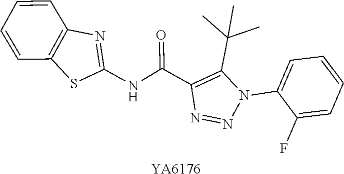 |
| 1099 (YA6168) | 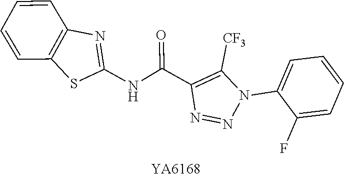 |
| 1100 (YA4155) | 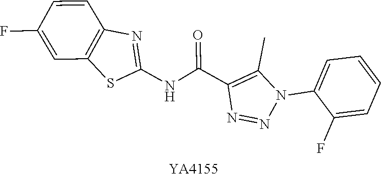 |
| 1101 (YA4165) | 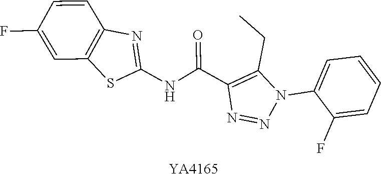 |
| 1102 (YA6172) | 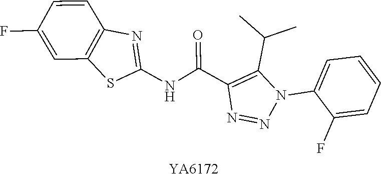 |
| 1103 (YA6174) | 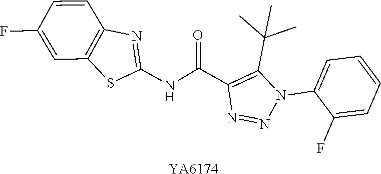 |
| 1104 (YA6175) | 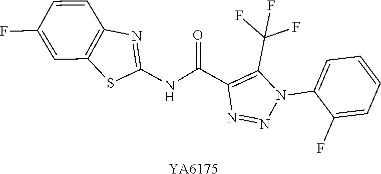 |
| 1105 (YA1124) | 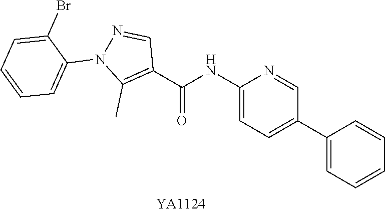 |
| 1106 (YA1122) | 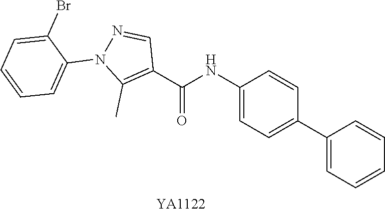 |
| 1107 (YA6152) | 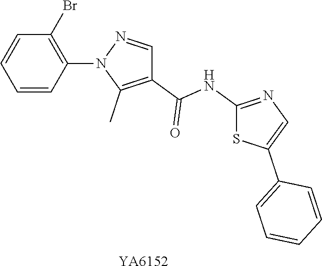 |
| 1108 (YA4087) | 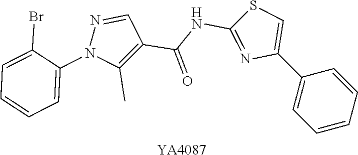 |
| 1109 (YA4033) | 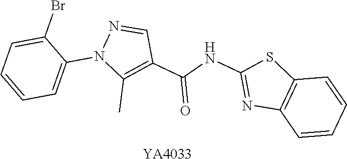 |
| 1110 (YA7018) | 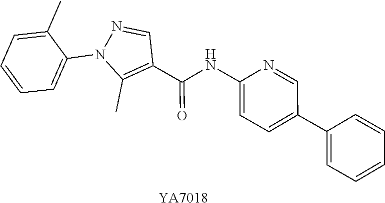 |
| 1111 (YA7016) | 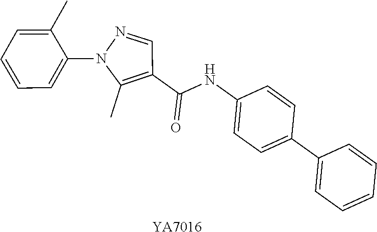 |
| 1112 (YA7019) | 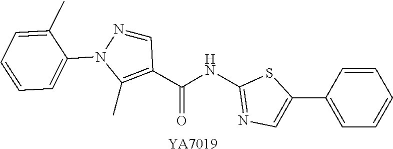 |
| 1113 (YA7015) | 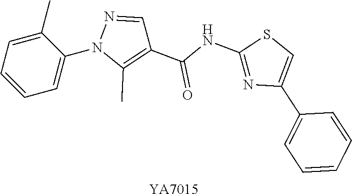 |
| 1114 (YA6179) | 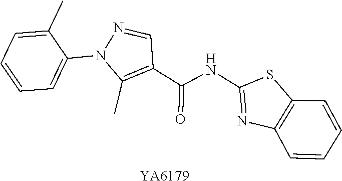 |
| 1115 (YA4011) | 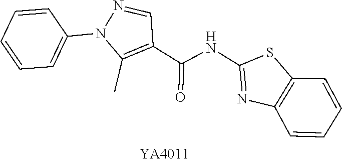 |
| 1116 (YA6149) | 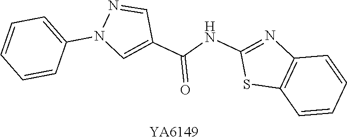 |
| 1117 (YA6143) | 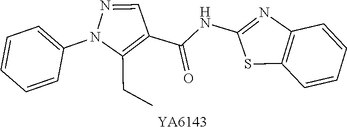 |
| 1118 (YA6144) | 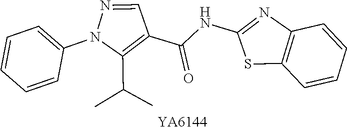 |
| 1119 (YA4009) | 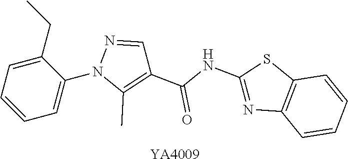 |
| 1120 (YA7020) | 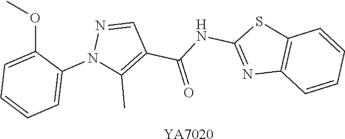 |
| 1121 (YA4007) | 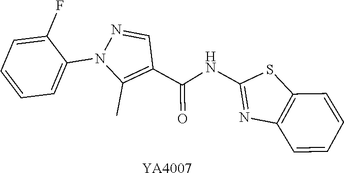 |
| 1122 (YA4025) | 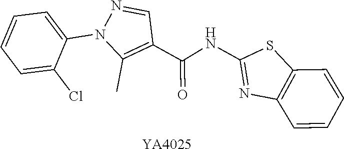 |
| 1123 (YA4021) | 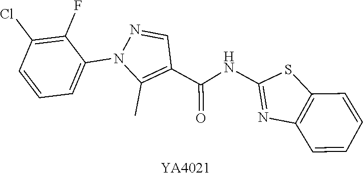 |
| 1124 (YA4019) | 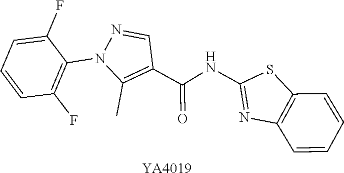 |
| 1125 (YA4017) | 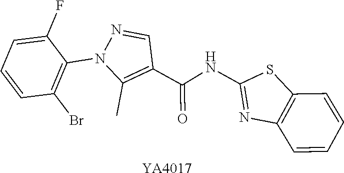 |
| 1126 (YA4023) | 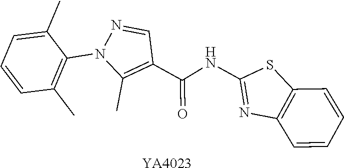 |

16. The compound of claim 1, wherein the compound is a compound of any one of formula 1001, 1002, 1024, 1032-1036, 1078, 1096, 1100, 1101, 1114, 1121, 1122, or 1124, or a pharmaceutically acceptable salt thereof:

| Formula No. | Structure |
|---|---|
| 1001 (YA6060) | 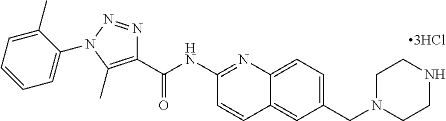 [SMILES: Cc1ccccc1-n1nnc(C(=O)Nc2ccc3cc(CN4CCNCC4)ccc3n2)c1C] |
| 1002 (YA10951) | 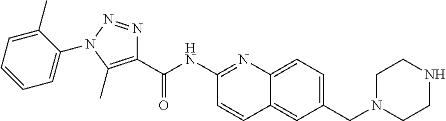 [SMILES: Cc1ccccc1-n1nnc(C(=O)Nc2ccc3cc(CN4CCNCC4)ccc3n2)c1C] |
| 1024 (YA6045) | 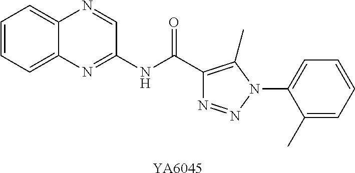 |
| 1032 (YA6136) | 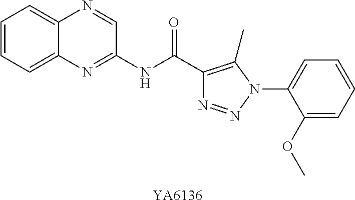 |
| 1033 (YA6137) | 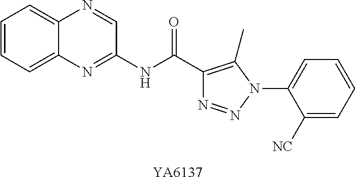 |
| 1034 (YA6138) | 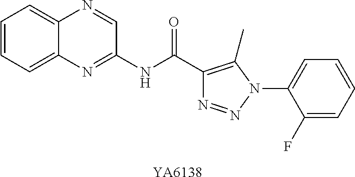 |
| 1035 (YA6139) | 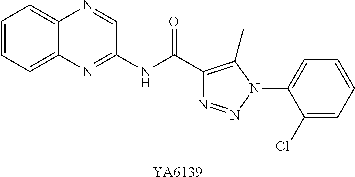 |
| 1036 (YA6141) | 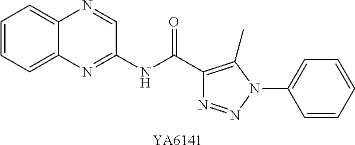 |
| 1078 (YA4029) | 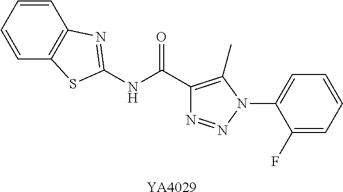 |
| 1096 (YA4001) | 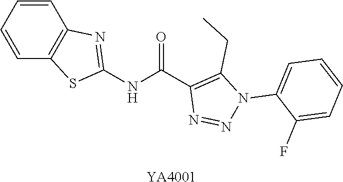 |
| 1100 (YA4155) | 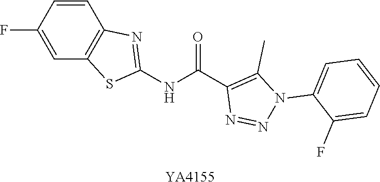 |
| 1101 (YA4165) | 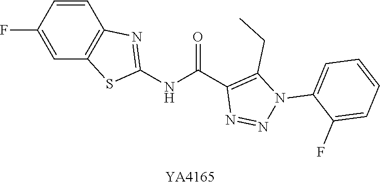 |
| 1114 (YA6179) | 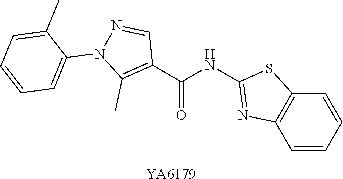 |
| 1121 (YA4007) | 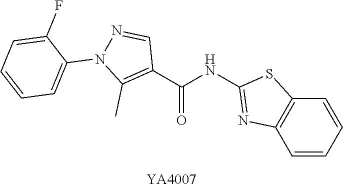 |
| 1122 (YA4025) | 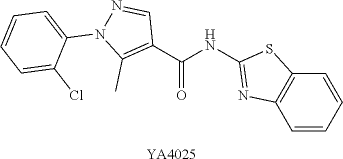 |
| 1124 (YA4019) | 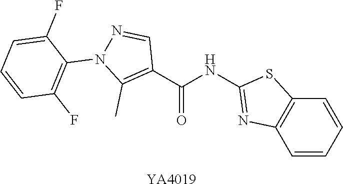 |

17. The compound of claim 1, wherein the compound is a compound of formula 1001.

18. The compound of any one of claims 1-17, wherein the pharmaceutically acceptable salt is selected from valproic acid, maleic acid, tartaric acid, oxalic acid, pamoic acid, phosphonic acid, benzoic acid, citric acid, salicylic acid, succinic acid, methanesulfonic acid, malic acid, and p-toluenesulfonic acid.

19. A compound of the formula (II): 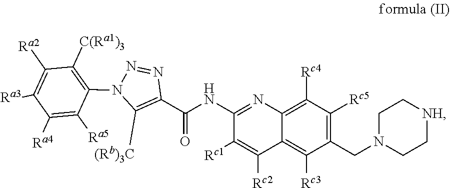
wherein in formula (II):
R^{a1}, R^{a2}, R^{a3}, R^{a4}, R^{a5}, R^{b}, R^{c1}, R^{c2}, R^{c3}, R^{c4}, and R^{c5} at each occurrence is independently selected from H, halo, alkyl, alkoxy, and aryl,
with the proviso that one or more R^{a1}, R^{a2}, R^{a3}, R^{a4}, R^{a5}, R^{b}, R^{c1}, R^{c2}, R^{c3}, R^{c4}, and R^{c5} is fluoro;
or a pharmaceutically acceptable salt thereof.

20. The compound of claim 19, wherein one or more R^{a1} is fluoro.

21. The compound of claim 20, wherein each occurrence of R^{a1} is fluoro.

22. The compound of any one of claims 19-22, wherein R^{a3} is fluoro.

23. The compound of any one of claims 19-22, wherein one or more R^{b} is fluoro.

24. The compound of claim 23, wherein each occurrence of R^{b} is fluoro.

25. The compound of any one of claims 19-24, wherein R^{c2} is fluoro.

26. The compound of any one of claims 19-25, wherein R^{c5} is fluoro.

27. The compound of claim 19, wherein the compound comprises only one fluoro group.

28. The compound of claim 27, wherein the only one fluoro group is at R^{a2}, R^{a3}, R^{a4}, R^{a5}, R^{c1}, R^{c2}, R^{c3}, R^{c4}, or R^{c5}.

29. The compound of claim 28, wherein the remaining R^{a2}, R^{a3}, R^{a4}, R^{a5}, R^{c1}, R^{c2}, R^{c3}, R^{c4}, and R^{c5} are hydrogen, and each occurrence of Rai is hydrogen, and each occurrence of R^{b} is hydrogen.

30. The compound of claim 19, wherein the compound comprises exactly three fluoro groups, wherein the three fluoro groups are at each occurrence of Rai or at each occurrence of R^{b}.

31. The compound of claim 30, wherein the remaining R^{a1} or R^{b} are at each occurrence hydrogen, and R^{a2}, R^{a3}, R^{a4}, R^{a5}, R^{c1}, R^{c2}, R^{c3}, R^{c4}, or R^{c5} are each hydrogen.

32. The compound of claim 19, wherein the compound of formula (II) is a compound of any one of formula 2001-2031, or a pharmaceutically acceptable salt thereof:

| Formula No. | Structure |
|---|---|
| 2001 | 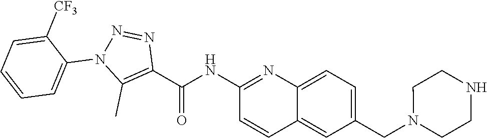 [SMILES: Cc1c(C(=O)Nc2ccc3cc(CN4CCNCC4)ccc3n2)nnn1-c1ccccc1C(F)(F)F] |
| 2002 | 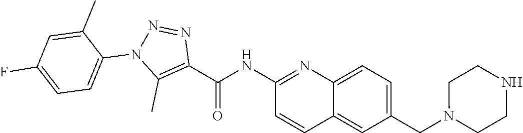 [SMILES: Cc1cc(F)ccc1-n1nnc(C(=O)Nc2ccc3cc(CN4CCNCC4)ccc3n2)c1C] |
| 2003 | 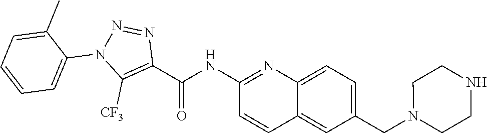 [SMILES: Cc1ccccc1-n1nnc(C(=O)Nc2ccc3cc(CN4CCNCC4)ccc3n2)c1C(F)(F)F] |
| 2004 | 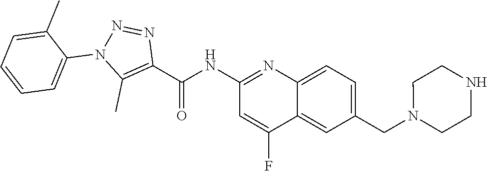 [SMILES: Cc1ccccc1-n1nnc(C(=O)Nc2cc(F)c3cc(CN4CCNCC4)ccc3n2)c1C] |
| 2005 | 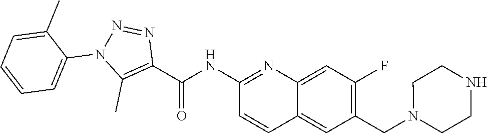 [SMILES: Cc1ccccc1-n1nnc(C(=O)Nc2ccc3cc(CN4CCNCC4)c(F)cc3n2)c1C] |
| 2006 | 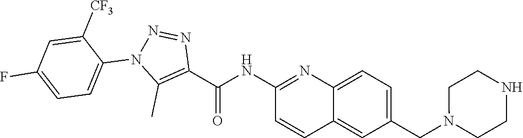 [SMILES: Cc1c(C(=O)Nc2ccc3cc(CN4CCNCC4)ccc3n2)nnn1-c1ccc(F)cc1C(F)(F)F] |
| 2007 | 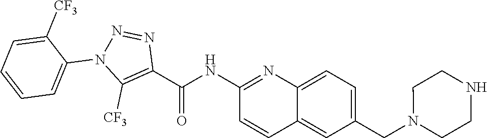 [SMILES: O=C(Nc1ccc2cc(CN3CCNCC3)ccc2n1)c1nnn(-c2ccccc2C(F)(F)F)c1C(F)(F)F] |
| 2008 | 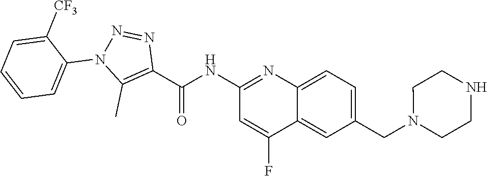 [SMILES: Cc1c(C(=O)Nc2cc(F)c3cc(CN4CCNCC4)ccc3n2)nnn1-c1ccccc1C(F)(F)F] |
| 2009 | 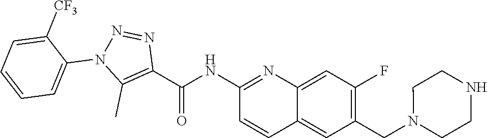 [SMILES: Cc1c(C(=O)Nc2ccc3cc(CN4CCNCC4)c(F)cc3n2)nnn1-c1ccccc1C(F)(F)F] |
| 2010 | 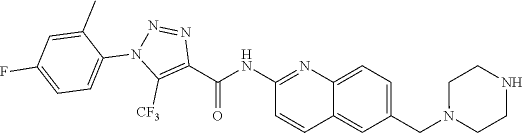 [SMILES: Cc1cc(F)ccc1-n1nnc(C(=O)Nc2ccc3cc(CN4CCNCC4)ccc3n2)c1C(F)(F)F] |
| 2011 | 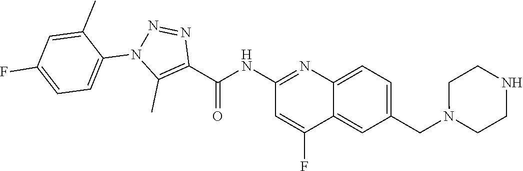 [SMILES: Cc1cc(F)ccc1-n1nnc(C(=O)Nc2cc(F)c3cc(CN4CCNCC4)ccc3n2)c1C] |
| 2012 | 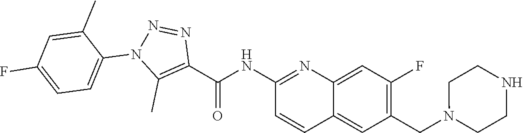 [SMILES: Cc1cc(F)ccc1-n1nnc(C(=O)Nc2ccc3cc(CN4CCNCC4)c(F)cc3n2)c1C] |
| 2013 | 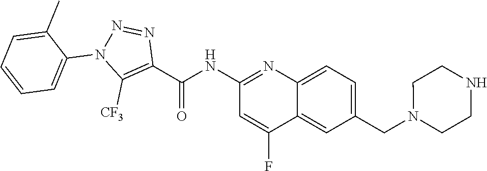 [SMILES: Cc1ccccc1-n1nnc(C(=O)Nc2cc(F)c3cc(CN4CCNCC4)ccc3n2)c1C(F)(F)F] |
| 2014 | 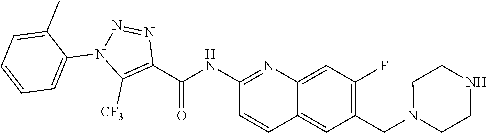 [SMILES: Cc1ccccc1-n1nnc(C(=O)Nc2ccc3cc(CN4CCNCC4)c(F)cc3n2)c1C(F)(F)F] |
| 2015 | 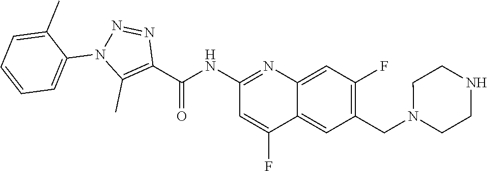 [SMILES: Cc1ccccc1-n1nnc(C(=O)Nc2cc(F)c3cc(CN4CCNCC4)c(F)cc3n2)c1C] |
| 2016 | 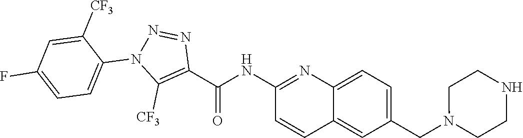 [SMILES: O=C(Nc1ccc2cc(CN3CCNCC3)ccc2n1)c1nnn(-c2ccc(F)cc2C(F)(F)F)c1C(F)(F)F] |
| 2017 | 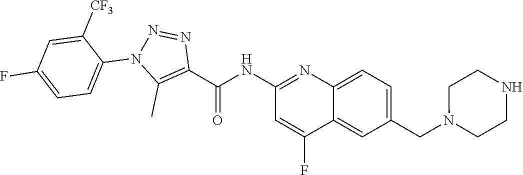 [SMILES: Cc1c(C(=O)Nc2cc(F)c3cc(CN4CCNCC4)ccc3n2)nnn1-c1ccc(F)cc1C(F)(F)F] |
| 2018 | 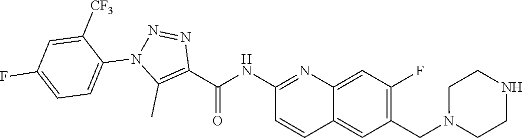 [SMILES: Cc1c(C(=O)Nc2ccc3cc(CN4CCNCC4)c(F)cc3n2)nnn1-c1ccc(F)cc1C(F)(F)F] |
| 2019 | 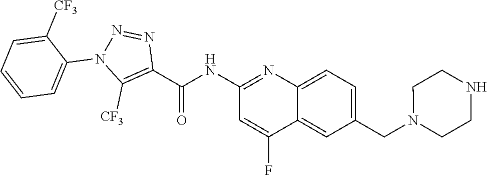 [SMILES: O=C(Nc1cc(F)c2cc(CN3CCNCC3)ccc2n1)c1nnn(-c2ccccc2C(F)(F)F)c1C(F)(F)F] |
| 2020 | 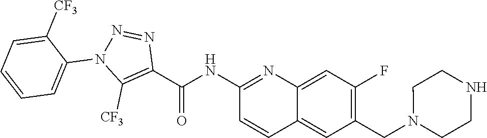 [SMILES: O=C(Nc1ccc2cc(CN3CCNCC3)c(F)cc2n1)c1nnn(-c2ccccc2C(F)(F)F)c1C(F)(F)F] |
| 2021 | 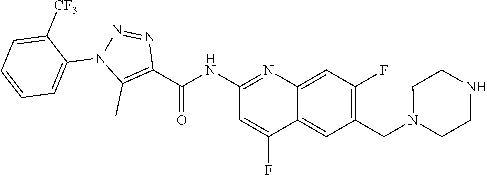 [SMILES: Cc1c(C(=O)Nc2cc(F)c3cc(CN4CCNCC4)c(F)cc3n2)nnn1-c1ccccc1C(F)(F)F] |
| 2022 | 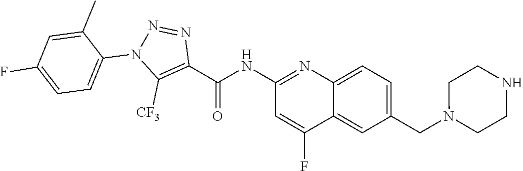 [SMILES: Cc1cc(F)ccc1-n1nnc(C(=O)Nc2cc(F)c3cc(CN4CCNCC4)ccc3n2)c1C(F)(F)F] |
| 2023 | 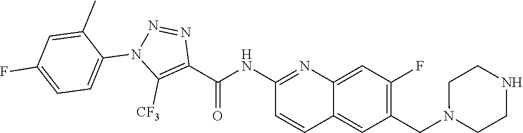 [SMILES: Cc1cc(F)ccc1-n1nnc(C(=O)Nc2ccc3cc(CN4CCNCC4)c(F)cc3n2)c1C(F)(F)F] |
| 2024 | 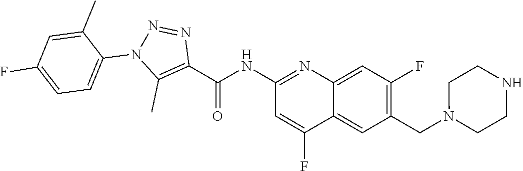 [SMILES: Cc1cc(F)ccc1-n1nnc(C(=O)Nc2cc(F)c3cc(CN4CCNCC4)c(F)cc3n2)c1C] |
| 2025 | 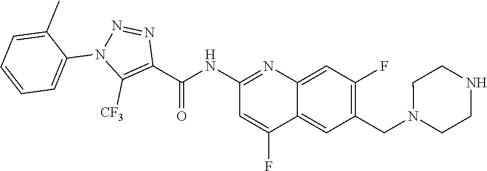 [SMILES: Cc1ccccc1-n1nnc(C(=O)Nc2cc(F)c3cc(CN4CCNCC4)c(F)cc3n2)c1C(F)(F)F] |
| 2026 | 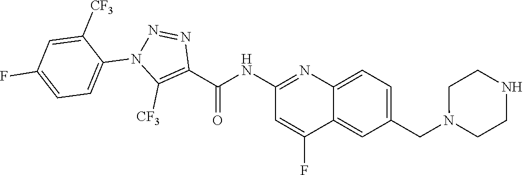 [SMILES: O=C(Nc1cc(F)c2cc(CN3CCNCC3)ccc2n1)c1nnn(-c2ccc(F)cc2C(F)(F)F)c1C(F)(F)F] |
| 2027 | 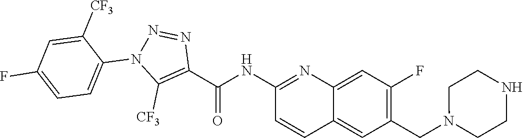 [SMILES: O=C(Nc1ccc2cc(CN3CCNCC3)c(F)cc2n1)c1nnn(-c2ccc(F)cc2C(F)(F)F)c1C(F)(F)F] |
| 2028 | 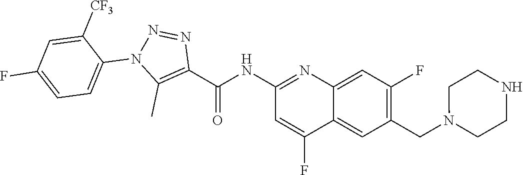 [SMILES: Cc1c(C(=O)Nc2cc(F)c3cc(CN4CCNCC4)c(F)cc3n2)nnn1-c1ccc(F)cc1C(F)(F)F] |
| 2029 | 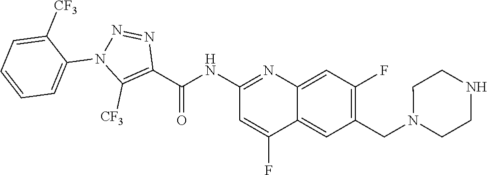 [SMILES: O=C(Nc1cc(F)c2cc(CN3CCNCC3)c(F)cc2n1)c1nnn(-c2ccccc2C(F)(F)F)c1C(F)(F)F] |
| 2030 | 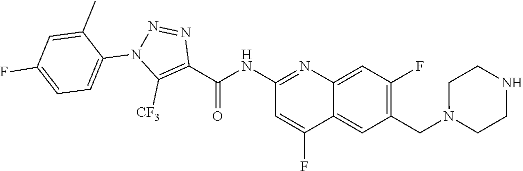 [SMILES: Cc1cc(F)ccc1-n1nnc(C(=O)Nc2cc(F)c3cc(CN4CCNCC4)c(F)cc3n2)c1C(F)(F)F] |
| 2031 | 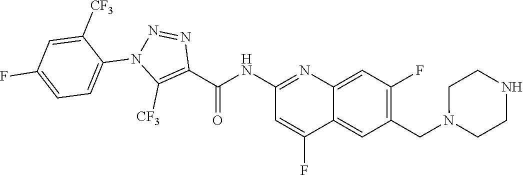 [SMILES: O=C(Nc1cc(F)c2cc(CN3CCNCC3)c(F)cc2n1)c1nnn(-c2ccc(F)cc2C(F)(F)F)c1C(F)(F)F] |

33. The compound of claim 32, wherein the compound of formula (II) is a compound of any one of formula 2001-2005, or a pharmaceutically acceptable salt thereof.

34. A compound of the formula (III): 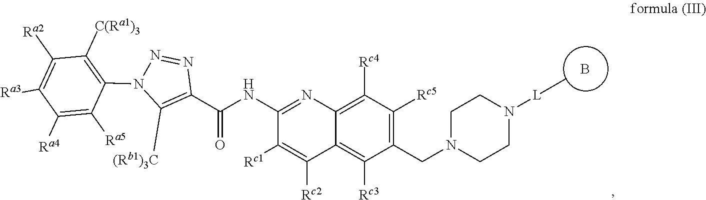
wherein in formula (III):
R^{a1}, R^{a2}, R^{a3}, R^{a4}, R^{a5}, R^{b1}, R^{c1}, R^{c2}, R^{c3}, R^{c4}, and R^{c5} at each occurrence is independently selected from hydrogen, halo, alkyl, alkoxy, and aryl;
L is a linking group; and
B is a targeting moiety,
or a pharmaceutically acceptable salt thereof.

35. The compound of claim 34, wherein R^{a1}, R^{a2}, R^{a3}, R^{a4}, R^{a5}, R^{b}, R^{c1}, R^{c2}, R^{c3}, R^{c4}, and R^{c5} are at each occurrence hydrogen.

36. The compound of claim 34 or 35, wherein L comprises one or more linking groups selected from optionally substituted —C₁₋₁₀ alkyl-, —O—C₁₋₁₀ alkyl-, —C₁₋₁₀ alkenyl-, —O—C₁₋₁₀ alkenyl-, —C₁₋₁₀ cycloalkenyl-, —O—C₁₋₁₀ cycloalkenyl-, —C₁₋₁₀ alkynyl-, —O—C₁₋₁₀ alkynyl-, —C₁₋₁₀ aryl-, —O—C₁₋₁₀—, -aryl-, —O—, —S—, —S—S—, —S(O)_{w}—, —C(O)—, —C(O)O—, —OC(O)—, —C(O)S—, —SC(O)—, —OC(O)O—, —N(R^{b})—, —C(O)N(R^{b})—, —N(R^{b})C(O)—, —OC(O)N(R^{b})—, —N(R^{b})C(O)O—, —SC(O)N(R^{b})—, —N(R^{b})C(O)S—, —N(R^{b})C(O)N(R^{b})—, —N(R^{b})C(NR^{b})N(R^{b})—, —N(R^{b})S(O)_{w}—, —S(O)_{w}N(R^{b})—, —S(O)_{w}O—, —OS(O)_{w}—, —OS(O)_{w}O—, —O(O)P(OR^{b})O—, (O)P(O—)₃, —O(S)P(OR^{b})O—, and (S)P(O—)₃, wherein w is 1 or 2, and R^{b} is independently hydrogen, optionally substituted alkyl, or optionally substituted aryl.

37. The compound of claim 36, wherein L comprises one or more linking groups selected from —C₁₋₁₀ alkyl-N(R^{b})—, —O—C₁₋₁₀ alkyl-, —O—, —N(R^{b})—, and —S—S—.

38. The compound of claim 36 or 37, wherein L comprises one or more linking group selected from

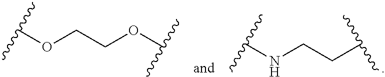 [SMILES: *OCCO* ; *CCN*]

39. The compound of any one of claims 36-38, wherein L comprises 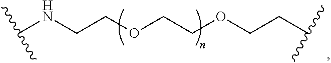
wherein n is an integer selected from 0-5.

40. The compound of any one of claims 36-38, wherein L comprises 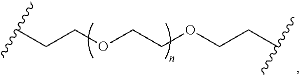
wherein n is an integer selected from 0-5.

41. The compound of any one of claims 34-40, wherein the linker is a cleavable linker.

42. The compound of any one of claims 34-40, wherein the linker is a non-cleavable linker.

43. The compound of any one of claims 34-42, wherein the targeting moiety comprises one or more moieties selected from biotin, folic acid, and biguanide.

44. The compound of claim 43, wherein the targeting moiety is selected from:

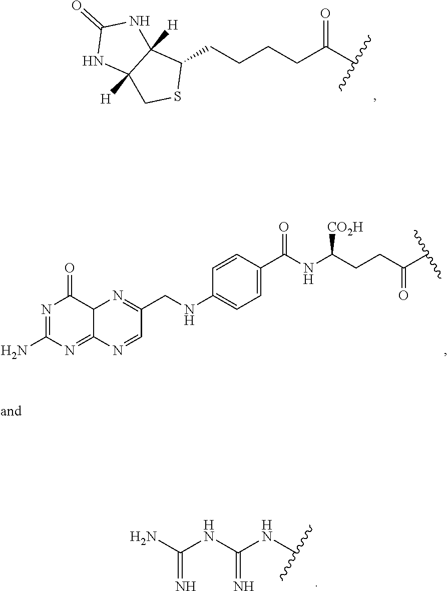 [SMILES: *C(=O)CCCC[C@@H]1SC[C@@H]2NC(=O)N[C@@H]21 ; *C(=O)CC[C@@H](NC(=O)c1ccc(NCC2=NC3C(=O)N=C(N)N=C3N=C2)cc1)C(=O)O ; *NC(=N)NC(=N)N]

45. The compound of claim 34, wherein the compound of formula (III) is a compound of any one of formula 3001-3018, or a pharmaceutically acceptable salt thereof:

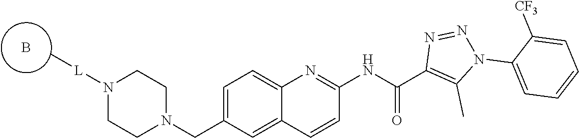

| Formula | | |
|---|---|---|
| No. | B | |
| 3001 | 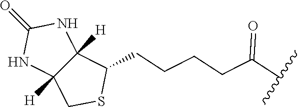 [SMILES: *C(=O)CCCC[C@@H]1SC[C@@H]2NC(=O)N[C@@H]21] | |
| 3002 | 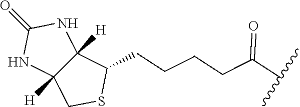 [SMILES: *C(=O)CCCC[C@@H]1SC[C@@H]2NC(=O)N[C@@H]21] | |
| 3003 | 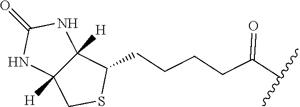 [SMILES: *C(=O)CCCC[C@@H]1SC[C@@H]2NC(=O)N[C@@H]21] | |
| 3004 | 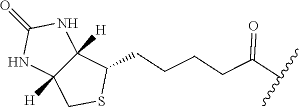 [SMILES: *C(=O)CCCC[C@@H]1SC[C@@H]2NC(=O)N[C@@H]21] | |
| 3005 | 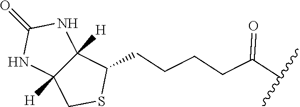 [SMILES: *C(=O)CCCC[C@@H]1SC[C@@H]2NC(=O)N[C@@H]21] | |
| 3006 | 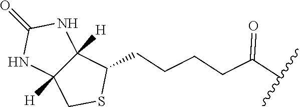 [SMILES: *C(=O)CCCC[C@@H]1SC[C@@H]2NC(=O)N[C@@H]21] | |
| 3007 | 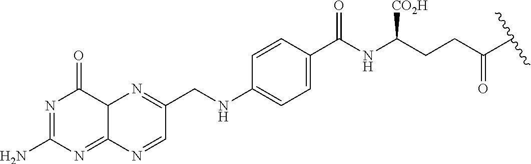 [SMILES: *C(=O)CC[C@@H](NC(=O)c1ccc(NCC2=NC3C(=O)N=C(N)N=C3N=C2)cc1)C(=O)O] | |
| 3008 | 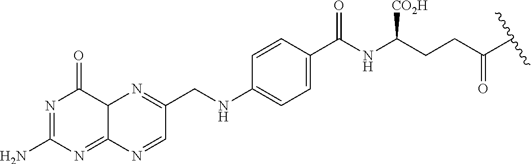 [SMILES: *C(=O)CC[C@@H](NC(=O)c1ccc(NCC2=NC3C(=O)N=C(N)N=C3N=C2)cc1)C(=O)O] | |
| 3009 | 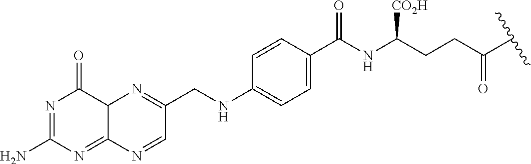 [SMILES: *C(=O)CC[C@@H](NC(=O)c1ccc(NCC2=NC3C(=O)N=C(N)N=C3N=C2)cc1)C(=O)O] | |
| 3010 | 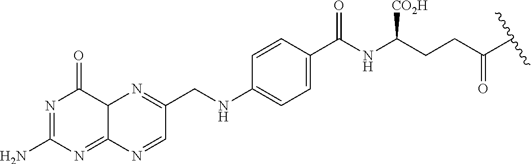 [SMILES: *C(=O)CC[C@@H](NC(=O)c1ccc(NCC2=NC3C(=O)N=C(N)N=C3N=C2)cc1)C(=O)O] | |
| 3011 | 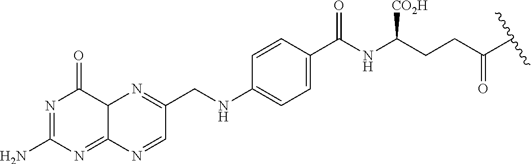 [SMILES: *C(=O)CC[C@@H](NC(=O)c1ccc(NCC2=NC3C(=O)N=C(N)N=C3N=C2)cc1)C(=O)O] | |
| 3012 | 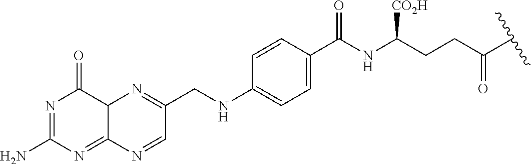 [SMILES: *C(=O)CC[C@@H](NC(=O)c1ccc(NCC2=NC3C(=O)N=C(N)N=C3N=C2)cc1)C(=O)O] | |
| 3013 | 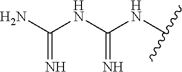 [SMILES: *NC(=N)NC(=N)N] | |
| 3014 | 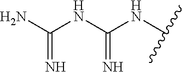 [SMILES: *NC(=N)NC(=N)N] | |
| 3015 | 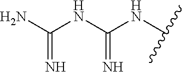 [SMILES: *NC(=N)NC(=N)N] | |
| 3016 | 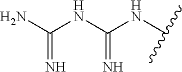 [SMILES: *NC(=N)NC(=N)N] | |
| 3017 | 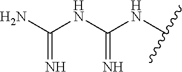 [SMILES: *NC(=N)NC(=N)N] | |
| 3018 | 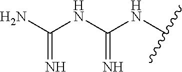 [SMILES: *NC(=N)NC(=N)N] | |
| Formula | | |
| No. | L | n |
| 3001 | 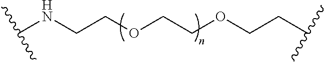 | 0 |
| 3002 | 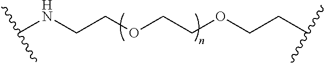 | 1 |
| 3003 | 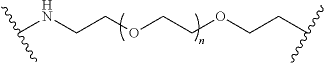 | 2 |
| 3004 | 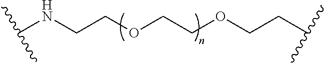 | 3 |
| 3005 | 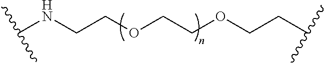 | 4 |
| 3006 | 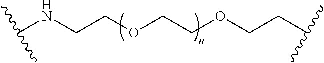 | 5 |
| 3007 | 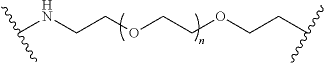 | 0 |
| 3008 | 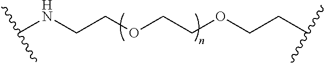 | 1 |
| 3009 | 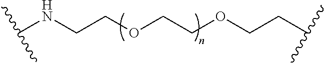 | 2 |
| 3010 | 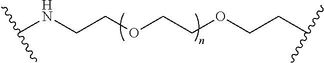 | 3 |
| 3011 | 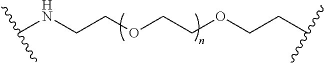 | 4 |
| 3012 | 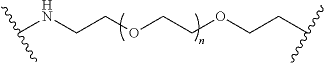 | 5 |
| 3013 | 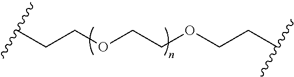 | 0 |
| 3014 | 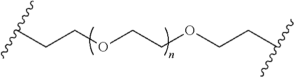 | 1 |
| 3015 | 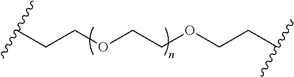 | 2 |
| 3016 | 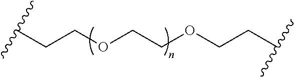 | 3 |
| 3017 | 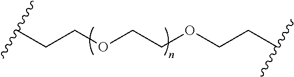 | 4 |
| 3018 | 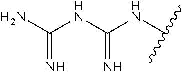 [SMILES: *NC(=N)NC(=N)N] | 5 |

46. A pharmaceutical composition comprising one or more of compounds ofany one of claims 1-45 or a pharmaceutically acceptable salt thereof, and a physiologically compatible carrier medium.

47. A pharmaceutical composition for treating or preventing a disease or disorder alleviated by inhibiting Wnt/β-catenin signaling, the pharmaceutical composition comprising one or more compounds according to any one of claims 1-45, or a pharmaceutically acceptable salt thereof, and a physiologically compatible carrier medium.

48. A pharmaceutical composition for treating or preventing a disease or disorder alleviated by activating adenosine monophosphate-activated kinase (AMPK) signaling, the pharmaceutical composition comprising one or more compounds according to any one of claims 1-45, or a pharmaceutically acceptable salt thereof, and a physiologically compatible carrier medium.

49. A pharmaceutical composition for treating or preventing a disease or disorder alleviated by both inhibiting Wnt/β-catenin signaling and activating adenosine monophosphate-activated kinase (AMPK) signaling, the pharmaceutical composition comprising one or more compounds according to any one of claims 1-45, or a pharmaceutically acceptable salt thereof, and a physiologically compatible carrier medium.

50. The pharmaceutical composition of any one of claims 47-49, wherein the disease or disorder is cancer or a metabolic disease.

51. The pharmaceutical composition of any one of claims 47-49, wherein the disease or disorder is selected from the group consisting of type 2 diabetes, obesity, hyperlipidemia, fatty liver disease, adrenocortical cancer, hepatocellular cancer, hepatoblastoma, malignant melanoma, ovarian cancer, Wilm's tumor, Barrett's esophageal cancer, prostate cancer, colon cancer, colorectal cancer, rectal cancer, pancreatic cancer, bladder cancer, breast cancer, gastric cancer, head & neck cancer, lung cancer, mesothelioma, cervical cancer, uterine cancer, myeloid leukemia cancer, lymphoid leukemia cancer, pilometricoma cancer, medulloblastoma cancer, glioblastoma, and familial adenomatous polyposis.

52. The pharmaceutical composition of claim 51, wherein the breast cancer is triple negative breast cancer.

53. A pharmaceutical composition for treating liver fibrosis, the pharmaceutical composition comprising one or more compounds of any one of claims 1-52, or a pharmaceutically acceptable salt thereof, and a physiologically compatible carrier medium.

54. A pharmaceutical composition for treating colorectal cancer (CRC), the pharmaceutical composition comprising one or more compounds of any one of claims 1-45, or a pharmaceutically acceptable salt thereof, and a physiologically compatible carrier medium.

55. A pharmaceutical composition for treating alcoholic fatty liver disease (ALD) or non-alcoholic fatty liver disease (NAFLD), the pharmaceutical composition comprising one or more compounds of any one of claims 1-45, or a pharmaceutically acceptable salt thereof, and a physiologically compatible carrier medium.

56. The pharmaceutical composition of claim 55, wherein the non-alcoholic fatty liver disease is selected from the group consisting of simple fatty liver (steatosis), non-alcoholic steatohepatitis (NASH), and liver cirrhosis.

57. The pharmaceutical composition of any one of claims 46-56, comprising one or more additional therapeutic agents.

58. The pharmaceutical composition of claim 57, wherein the additional therapeutic agent is selected from the group consisting of a RAF inhibitor, an MEK inhibitor, an ERK inhibitor, a VEGFR inhibitor, EGFR inhibitor, and a combination thereof.

59. The pharmaceutical composition of claim 58, wherein the VEGFR inhibitor is selected from the group consisting of Bevacizumab (AVASTIN), Aflibercept (ZALTRAP), Regorafenib (STIVARGA), and a combination thereof.

60. The pharmaceutical composition of claim 58, wherein the EGFR inhibitor is selected from the group consisting of Cetuximab (ERBITUX), Panitumumab (VECTIBIX), Gefitinib, and a combination thereof.

61. The pharmaceutical composition of claim 57, wherein the additional therapeutic agent is selected from angiotensin II receptor antagonists, angiotensin converting enzyme (ACE) inhibitors, caspase inhibitors, cathepsin B inhibitors, CCR2 chemokine antagonists, CCR5 chemokine antagonists, chloride channel stimulators, cholesterol solubilizers, diacylglycerol O-acyltransferase 1 (DGAT1) inhibitors, dipeptidyl peptidase IV (DPPIV) inhibitors, farnesoid X receptor (FXR) agonists, FXR/TGR5 dual agonists, galectin-3 inhibitors, glucagon-like peptide 1 (GLPl) agonists, glutathione precursors, hepatitis C virus NS3 protease inhibitors, HMG CoA reductase inhibitors, 1 Iβ-hydroxy steroid dehydrogenase (I Iβ-HSDl) inhibitors, IL-Iβ antagonists, IL-6 antagonists, IL-10 agonists, IL-17 antagonists, ileal sodium bile acid cotransporter inhibitors, leptin analogs, 5-lipoxygenase inhibitors, LPL gene stimulators, lysyl oxidase homolog 2 (LOXL2) inhibitors, PDE3 inhibitors, PDE4 inhibitors, phospholipase C (PLC) inhibitors, PPARa agonists, PPARy agonists, PPAR5 agonists, Rho associated protein kinase 2 (ROCK2) inhibitors, sodium glucose transporter-2 (SGLT2) inhibitors, stearoyl CoA desaturase-1 inhibitors, thyroid hormone receptor β agonists, tumor necrosis factor a (TNFa) ligand inhibitors, transglutaminase inhibitors, transglutaminase inhibitor precursors, PTP1b inhibitors, and ASK1 inhibitors.

62. The pharmaceutical composition of claim 57, wherein the additional therapeutic agent is selected from capecitabine; cetuximab; bevacizumab; a MEK inhibitor such as N-[(R)-2,3-dihydroxy-propoxy]-3,4-difluoro-2-(2-fluoro-4-iodo-phenylamino)-benzamide, or a pharmaceutically acceptable salt thereof; a FOLFOX4 combination including oxaliplatin, 5-fluorouracil and leucovorin; and a FOLFIRI combination include irinotecan, 5-fluorouracil and leucovorin and the like.

63. A method of treating or preventing a disease or disorder alleviated by inhibiting Wnt/β-catenin signaling in a patient in need of said treatment or prevention, the method comprising administering a therapeutically effective amount of one or more compounds of any one of claims 1-45, or a pharmaceutically acceptable salt thereof.

64. A method of treating or preventing a disease or disorder alleviated by activating adenosine monophosphate-activated kinase (AMPK) signaling in a patient in need of said treatment or prevention, the method comprising administering a therapeutically effective amount of one or more compounds of any one of claims 1-45, or a pharmaceutically acceptable salt thereof.

65. A method of treating or preventing a disease or disorder alleviated by both inhibiting Wnt/β-catenin signaling and activating adenosine monophosphate-activated kinase (AMPK) signaling in a patient in need of said treatment or prevention, the method comprising administering a therapeutically effective amount of one or more compounds of any one of claims 1-45, or a pharmaceutically acceptable salt thereof.

66. The method of any one of claims 63-65, wherein the disease or disorder is cancer or a metabolic disease.

67. The method of any one of claims 63-65, wherein the disease or disorder is selected from the group consisting of type 2 diabetes, obesity, hyperlipidemia, fatty liver disease, adrenocortical cancer, hepatocellular cancer, hepatoblastoma, malignant melanoma, ovarian cancer, Wilm's tumor, Barrett's esophageal cancer, prostate cancer, colon cancer, colorectal cancer, rectal cancer, pancreatic cancer, bladder cancer, breast cancer, gastric cancer, head & neck cancer, lung cancer, mesothelioma, cervical cancer, uterine cancer, myeloid leukemia cancer, lymphoid leukemia cancer, pilometricoma cancer, medulloblastoma cancer, glioblastoma, and familial adenomatous polyposis.

68. The method of any one of claims 63-67, wherein the disease or disorder is type 2 diabetes.

69. The method of any one of claims 63-67, wherein the disease or disorder is colon cancer and/or colorectal cancer.

70. The method of any one of claims 63-67, wherein the disease or disorder is fatty liver disease.

71. The method of claim 70, wherein the fatty liver disease comprises a nonalcoholic fatty liver disease (NAFLD) or nonalcoholic steatohepatitis (NASH).

72. The method of claim 67, wherein the breast cancer is triple negative breast cancer.

73. A method of treating or preventing liver fibrosis in a patient in need of said treatment or prevention, the method comprising administering a therapeutically effective amount of one or more compounds of any one of claims 1-45, or a pharmaceutically acceptable salt of said one or more compounds.

74. A method of treating or preventing fatty liver disease in a patient in need of said treatment or prevention, the method comprising administering a therapeutically effective amount of one or more compounds of any one of claims 1-45, or a pharmaceutically acceptable salt of said one or more compounds.

75. The method of claim 74, wherein the disease comprises alcoholic fatty liver disease (ALD) or a non-alcoholic fatty liver disease (NAFLD).

76. The method of claim 74 or 75, wherein the disease is selected from the group consisting of simple fatty liver (steatosis), non-alcoholic steatohepatitis (NASH), and cirrhosis.

77. The method of any one of claims 74-76, wherein the disease is non-alcoholic steatohepatitis (NASH).

78. The method of any one of claims 63-77, wherein the one or more compounds are administered orally.

79. The method of any one of claims 63-78, wherein the one or more compounds are administered in combination with one or more additional therapeutic agents.

80. The method of claim 79, wherein the additional therapeutic agent is selected from the group consisting of a RAF inhibitor, an MEK inhibitor, an ERK inhibitor, a VEGFR inhibitor, EGFR inhibitor, and a combination thereof.

81. The method of claim 80, wherein the VEGFR inhibitor is selected from the group consisting of Bevacizumab (AVASTIN), Aflibercept (ZALTRAP), Regorafenib (STIVARGA), and a combination thereof.

82. The method of claim 80, wherein the EGFR inhibitor is selected from the group consisting of Cetuximab (ERBITUX), Panitumumab (VECTIBIX), Gefitinib, and a combination thereof.

83. The method of claim 79, wherein the additional therapeutic agent is selected from angiotensin II receptor antagonists, angiotensin converting enzyme (ACE) inhibitors, caspase inhibitors, cathepsin B inhibitors, CCR2 chemokine antagonists, CCR5 chemokine antagonists, chloride channel stimulators, cholesterol solubilizers, diacylglycerol O-acyltransferase 1 (DGAT1) inhibitors, dipeptidyl peptidase IV (DPPIV) inhibitors, farnesoid X receptor (FXR) agonists, FXR/TGR5 dual agonists, galectin-3 inhibitors, glucagon-like peptide 1 (GLPl) agonists, glutathione precursors, hepatitis C virus NS3 protease inhibitors, HMG CoA reductase inhibitors, 1 Iβ-hydroxy steroid dehydrogenase (I Iβ-HSD1) inhibitors, IL-Iβ antagonists, IL-6 antagonists, IL-10 agonists, IL-17 antagonists, ileal sodium bile acid cotransporter inhibitors, leptin analogs, 5-lipoxygenase inhibitors, LPL gene stimulators, lysyl oxidase homolog 2 (LOXL2) inhibitors, PDE3 inhibitors, PDE4 inhibitors, phospholipase C (PLC) inhibitors, PPARa agonists, PPARy agonists, PPAR5 agonists, Rho associated protein kinase 2 (ROCK2) inhibitors, sodium glucose transporter-2 (SGLT2) inhibitors, stearoyl CoA desaturase-1 inhibitors, thyroid hormone receptor β agonists, tumor necrosis factor a (TNFa) ligand inhibitors, transglutaminase inhibitors, transglutaminase inhibitor precursors, PTP1b inhibitors, and ASK1 inhibitors.

84. The method of claim 79, wherein the additional therapeutic agent is selected from capecitabine; cetuximab; bevacizumab; a MEK inhibitor such as N-[(R)-2,3-dihydroxy-propoxy]-3,4-difluoro-2-(2-fluoro-4-iodo-phenylamino)-benzamide, or a pharmaceutically acceptable salt thereof; a FOLFOX4 combination including oxaliplatin, 5-fluorouracil and leucovorin; and a FOLFIRI combination include irinotecan, 5-fluorouracil and leucovorin and the like.
